(19) United States
(12) Patent Application Publication
(10) Pub. No.: US 2023/0042913 A1
(43) Pub. Date: Feb. 9, 2023

(54) A COMBINATION OF, OR A BISPECIFIC BINDING MOLECULE TO, AN IMMUNE CHECKPOINT MOLECULE ANTAGONIST AND A RANK-L (NF-KB LIGAND) ANTAGONIST FOR CANCER THERAPY OR PROPHYLAXIS AND USES THEREOF

(71) Applicant: The Council of the Queensland Institute of Medical Research, Herston, Queensland (AU)

(72) Inventors: Bill DOUGALL, Herston, Queensland (AU); Michele TENG, Herston, Queensland (AU); Elizabeth AHERN, Herston, Queensland (AU); Mark SMYTH, Herston, Queensland (AU)

(21) Appl. No.: 16/619,891

(22) PCT Filed: Jun. 5, 2018
(86) PCT No.: PCT/AU2018/050557

§ 371 Date: Dec. 5, 2019

(30) Foreign Application Priority Data
Jun. 5, 2017 (AU) 2017902125

(51) Int. Cl. C07K 16/28; A61P 35/04

(52) U.S. Cl. CPC C07K 16/2875; C07K 16/2878; A61P 35/04; C07K 16/2818; C07K 2317/76; C07K 2317/31; A61K 2039/507

(57) ABSTRACT

Disclosed are agents for treating or preventing cancers. More particularly, the present invention discloses therapeutic combinations comprising antagonists of receptor of NF-κB (RANK) ligand and immune checkpoint molecules in methods and compositions for treating or inhibiting the development, progression or recurrence of cancers, including metastatic cancers.

### PRIORITY AND CROSS REFEENCE TO RELATED APPLICATIONS

This application is the U.S. National Phase Application under 35 U.S.C. § 371 of International Application No. PCT/AU2018/050557, filed Jun. 5, 2018, designating the U.S. and published in English as WO 2018/223182 Al on Dec. 13, 2018, which claims priority to Australian Provisional Application No. 2017902125 entitled “Agents for cancer therapy or prophylaxis and uses therefor” filed 5 Jun. 2017, the contents of which are incorporated herein by reference in their entirety.

### SEQUENCE LISTING IN ELECTRONIC FORMAT

The present application includes an Electronic Sequence Listing as an ASCII text filed via EFS-Web. The Electronic Sequence Listing is provided as a file entitled JAM184003APCSEQLIST.txt, created and last saved on Sep. 7, 2022, which is 442,799 bytes in size. The information in the Electronic Sequence Listing is incorporated herein by reference in its entirety.

### FIELD OF THE INVENTION

This invention relates generally to agents for treating or preventing cancers. More particularly, the present invention relates to therapeutic combinations comprising antagonists of receptor of NF-κB (RANK) ligand and immune checkpoint molecules in methods and compositions for treating or inhibiting the development, progression or recurrence of cancers, including metastatic cancers.

### BACKGROUND OF THE INVENTION

The National Cancer Institute has estimated that in the United States alone, one in three people will be diagnosed with cancer during their lifetime. Moreover, approximately 50% to 60% of people diagnosed with cancer will eventually succumb to the disease. The widespread occurrence of this disease underscores the need for improved anti-cancer therapies, particularly for the treatment of malignant cancer.

Immunotherapy has recently begun to show great promise in the treatment of cancer and considerable progress in the treatment of metastatic melanoma has been made, with the approval of immune checkpoint molecule-blocking antibodies. Ipilimumab, an anti-cytotoxic T-lymphocyte-associated antigen 4 (CTLA-4) antibody acts to up-regulate anti-tumor immunity and was the first agent to be associated with an improvement in overall survival in a phase 3 study involving patients with metastatic melanoma (see, Wolchok et al., 2013, New Engl J Med, 369:122-133; Smyth et al., 2016, J. Clin. Oncol. 34(12):e104-106). For reasons which have not yet been fully elucidated, ipilimumab was associated with responses in only 10% and 15% of patients (Wolchock et al., 2013, supra; Smyth et al.2016, supra), and approximately 30% of treated patients had long-term survival (Bostwick et al., 2015, J Immunoth Cancer, 3:19). Accordingly, despite the rapid progress in developing monotherapies and combination treatments, the disease burden attributable to cancer has not significantly ablated.

Combining a monoclonal antibody (MAb) directed to the immune checkpoint molecule programmed death 1 (PD-1) with anti-CTLA4 produced superior tumor responses and survival benefit in advanced melanoma, as compared to the use of PD-1 alone. This demonstrates the importance of combination immunotherapy targeting non-redundant mechanisms of immune evasion by tumors (see, Larkin et al., 2015, N Eng J Med, 373:23-24; Postow et al., 2015, N Eng J Med, 372:2006-2017; and Wolchok et al., 2013, supra). However, one challenge in the immunotherapy of solid and hematological malignancies is the discovery of new targets for patients who display primary resistance to current immunotherapy combinations.

Receptor of NF-κB (RANK) and its ligand (RANKL) are members of the tumor necrosis factor receptor and ligand superfamilies, respectively, with closest homology to CD40 and CD4OL. RANK (also known as TNFRSF11a) and RANKL (TNFSF11) are currently best known in clinical practice for their role in bone homeostasis, as the differentiation of osteoclasts from the monocyte-macrophage lineage requires RANKL interaction with RANK expressed on the myeloid osteoclast precursors (see, Dougall et al., 1999, Genes Dev., 13:2412-24; and Kong et al., 1999, Nature, 397:315-23)). The fully human IgG2 anti-RANKL antibody (denosumab) is widely used in clinical practice as a potent and well-tolerated anti-resorptive agent for the prevention of skeletal-related events arising from bone metastases, and the management of giant cell tumor of bone and osteoporosis (see, Branstetter et al., 2012, Clin Cancer Res, 18:4415-4424); and Fizazi et al., 2011, Lancet, 377:813-22). Intriguingly, denosumab increased overall survival in a post-hoc exploratory analysis of a phase 3 trial in patients with non-small cell lung cancer and bone metastases, compared with zoledronic acid (see, Scagliotti et al., 2012, J. Thorac. Oncol., 7:1823-9). RANKL was initially identified as a dendritic cell-specific survival factor which was upregulated by activated T-cells and interacted with RANK on the surface of mature dendritic cells (DCs) to prevent apoptosis (see, Wong et al., 1997, J Exp Med, 186:2075-2080 and Hochweller et al., 2005, Eur. J. Immunol., 35:1086-96).

### SUMMARY OF THE INVENTION

The present invention is predicated in part on the surprising finding that antagonizing a receptor activator of NF-κB (RANK) ligand (RANKL) and an immune checkpoint molecule (ICM), including an ICM that a regulatory T (Treg) cell lacks expression of or expresses at a low level, results in a synergistic enhancement in the immune response to a cancer. This finding has been reduced to practice in methods and compositions for stimulating or augmenting immunity, for inhibiting the development or progression of immunosuppression or tolerance to a tumor, or for inhibiting the development, progression or recurrence of cancer as described hereafter.

Accordingly, in one aspect, the present invention provides a therapeutic combination comprising, consisting, or consisting essentially of a RANKL antagonist and at least one ICM antagonist. The therapeutic combination may be in the form of a single composition (e.g., a mixture) comprising each of the RANKL antagonist and the at least one ICM antagonist. Alternatively, the RANKL antagonist and the at least one ICM antagonist may be provided as discrete components in separate compositions.

The at least one ICM antagonist suitably antagonizes an ICM selected from the group consisting of: programmed death 1 receptor (PD-1), programmed death ligand 1 (PD-L1), programmed death ligand 2 (PD-L2), cytotoxic T-lymphocyte-associated antigen 4 (CTLA-4), A2A adenosine receptor (AZAR), A2B adenosine receptor (A2BR), B7-H3 (CD276), V-set domain-containing T-cell activation inhibitor 1 (VTCN1), B- and T-lymphocyte attenuator (BTLA), indoleamine 2,3-dioxygenase (IDO), killer-cell immunoglobulin-like receptor (KIR), lymphocyte activation gene-3 (LAG3), T cell immunoglobulin domain and mucin domain 3 (TIM-3), V-domain Ig suppressor of T cell activation (VISTA), 5′-nucleotidase (CD73), tactile (CD96), poliovirus receptor (CD155), DNAX Accessory Molecule-1 (DNAM-1), poliovirus receptor-related 2 (CD112), cytotoxic and regulatory T-cell molecule (CRTAM), tumor necrosis factor receptor superfamily member 4 (TNFRS4; OX40; CD134), tumor necrosis factor (ligand) superfamily, member 4 (TNFSF4; OX40 ligand (OX4OL), natural killer cell receptor 2B4 (CD244), CD160, glucocorticoid-induced TNFR-related protein (GITR), glucocorticoid-induced TNFR-related protein ligand (GITRL), inducible costimulator (ICOS), galectin 9 (GAL-9), 4-1BB ligand (4-1BBL; CD137L), 4-1BB (4-1BB; CD137), CD70 (CD27 ligand (CD27L)), CD28, B7-1 (CD80), B7-2 (CD86), signal-regulatory protein (SIRP-1), integrin associated protein (IAP; CD47); B-lymphocyte activation marker (BLAST-1; CD48), natural killer cell receptor 2B4 (CD244); CD40, CD40 ligand (CD4OL), herpesvirus entry mediator (HVEM), transmembrane and immunoglobulin domain containing 2 (TMIGD2), HERV-H LTR-associating 2 (HHLA2), vascular endothelial growth inhibitor (VEGI), tumor necrosis factor receptor superfamily member 25 (TNFRS25), inducible T-cell co-stimulator ligand (ICOLG; B7RP1) and T cell immunoreceptor with Ig and ITIM (immunoreceptor tyrosine-based inhibition motif) domains (TIGIT). In some embodiments, the at least one ICM antagonist is selected from a PD-1 antagonist, a PD-L1 antagonist and a CTLA4 antagonist. In some embodiments, the at least one ICM antagonist is other than or excludes a CTLA-4 antagonist. In some embodiments, the at least one ICM antagonist comprises a PD-1 antagonist. In some embodiments, the at least one ICM antagonist comprises a PD-L1 antagonist. In certain embodiments, the at least one ICM antagonist comprises a PD-1 antagonist and a PD-L1 antagonist. In other embodiments, the at least one ICM antagonist comprises a PD-1 antagonist and a CTLA4 antagonist. In other embodiments, the at least one ICM antagonist comprises a PD-L1 antagonist and a CTLA4 antagonist. In specific embodiments, the ICM antagonist antagonizes an ICM that a Treg cell lacks expression of or expresses at a low level. In some of the same and other embodiments, the ICM antagonist antagonizes an ICM (e.g., PD-1 or PD-L1) that is expressed at a lower level on a Treg than CTLA4. In some of the same and other embodiments, the ICM antagonist antagonizes an ICM (e.g., PD-1 or PD-L1) that is expressed at a higher level on an immune effector cell (e.g., an effector T cell, macrophage, dendritic cell, B cell, etc.) than on a Treg. In representative examples of these embodiments, the at least one ICM antagonist antagonizes an ICM selected from one or both of PD-1 and PD-L1.

The RANKL antagonist may be a direct RANKL antagonist that binds specifically to RANKL, or an indirect RANKL antagonist that binds specifically to RANKL's cognate receptor, RANK.

Numerous RANKL and ICM antagonists are known in the art, any of which may be used in the practice of the present invention. In various embodiments, the antagonists are antagonist antigen-binding molecules.

In some of these embodiments, the RANKL antagonist is an anti-RANKL antigen-binding molecule that binds specifically to RANKL. In illustrative examples of this type, the anti-RANKL antigen-binding molecule binds specifically to one or more amino acids of the amino acid sequence TEYLQLMVY [SEQ ID NO:1] (i.e., residues 233-241 of the native RANKL sequence set forth in SEQ ID NO:2).

In some embodiments, the anti-RANKL antigen-binding molecule is a monoclonal antibody (MAb). A non-limiting example of an anti-RANKL antigen-binding molecule is the MAb denosumab or an antigen-binding fragment thereof. Suitably, the anti-RANKL antigen-binding molecule comprises a heavy chain amino acid sequence as set forth in SEQ ID NO:3 or an antigen-binding fragment thereof and/or a light chain amino acid sequence as set forth in SEQ ID NO:4 or an antigen-binding fragment thereof.

In other embodiments, the anti-RANKL antigen-binding molecule competes with denosumab for binding to RANKL.

In some embodiments, the RANKL antagonist antagonizes RANK. For example, the RANK antagonist (e.g., an anti-RANK antigen-binding molecule or antagonist peptide) may bind specifically to, or comprise, consist or consist essentially of, an amino acid sequence corresponding to at least a portion of a cysteine-rich domain (CRD) selected from CDR2 (i.e., residues 44-85) and CRD3 (i.e., residues 86-123). In non-limiting examples of this type, the RANK antagonist (e.g., an anti-RANK antigen-binding molecule or antagonist peptide) binds specifically to, or comprises, consists or consists essentially of, an amino acid sequence corresponding to at least a portion of RANK CRD3, representative examples of which include YCWNSDCECCY [SEQ ID NO:5], YCWSQYLCY [SEQ ID NO: 6].

In other embodiments, the RANK antagonist is an anti-RANK antigen-binding molecule that binds specifically to one or more amino acids of the amino acid sequence: VSKTEIEEDSFRQMPTEDEYMDRPSQPTDQLLFLTEPGSKSTPPFSEPLEVGENDSLSQCFTGTQSTVGSESCNC TEPLCRTDWTPMS [SEQ ID NO:7] (i.e., residues 330-417 of the native RANK sequence set forth in SEQ ID NO:8). Suitably, the anti-RANK antigen-binding molecule is a monoclonal antibody (MAb). By way of example, the anti-RANK antigen-binding molecule may be selected from the MAbs 64C1385, as well as N-1H8 and N-2B10 (Taylor et al. Appl Immunohistochem Mol Morphol. 2017;25(5):299-307; Branstetter et al. J Bone Oncol. 2015;4(3):59-68), or an antigen-binding fragment thereof. In other embodiments, the anti-RANK antigen-binding molecule may compete with MAbs 64C1385, N-1H8 or N-2B10 for binding to RANK. In some embodiments, the anti-RANK antigen-binding molecule is a short chain Fv (scFv) antigen-binding molecule as disclosed for example by Newa et al. (Mol Pharm. 11(1):81-9 (2014)), or an antigen-binding fragment thereof.

Suitably, a respective ICM antagonist is an anti-ICM antigen-binding molecule. In specific embodiments, the anti-ICM antigen-bind molecule is selected from an anti-PD-1 antigen-binding molecule, an anti-PD-L1 antigen-binding molecule and an anti-CTLA4 antigen-binding molecule.

The anti-PD-1 antigen-binding molecule may be a MAb, non-limiting examples of which include nivolumab, pembrolizumab, pidilizumab, and MEDI-0680 (AMP-514), AMP-224, JS001-PD-1, SHR-1210, Gendor PD-1, PDR001, CT-011, REGN2810, BGB-317 or an antigen-binding fragment thereof. Alternatively, the anti-PD-1 antigen-binding molecule may be one that competes with nivolumab, pembrolizumab, pidilizumab, or MEDI-0680 for binding to PD-1.

In some embodiments, the anti-PD-1 antigen-binding molecule binds specifically to one or more amino acids of the amino acid sequence SFVLNWYRMSPSNQTDKLAAFPEDR [SEQ ID NO:9] (i.e., residues 62 to 86 of the native PD-1 sequence set forth in SEQ ID NO:10) and/or in the amino acid sequence SGTYLCGAISLAPKAQIKE [SEQ ID NO:11] (i.e., residues 118 to 136 of the native PD-1 sequence set forth in SEQ ID NO:10). In some of the same embodiments and other embodiments, the anti-PD-1 antigen-binding molecule binds specifically to one or more amino acids of the amino acid sequence NWYRMSPSNQTDKLAAFPEDRSQPGQDCRFRV [SEQ ID NO:12] (i.e., corresponding to residue 66 to 97 of the native PD-1 sequence set forth in SEQ ID NO:10).

In some embodiments, the anti-PD-L1 antigen-binding molecule is a MAb, non-limiting examples of which include durvalumab (MEDI4736), atezolizumab (Tecentriq), avelumab, BMS-936559/MDX-1105, MSB0010718C, LY3300054, CA-170, GNS-1480 and MPDL3280A, or an antigen-binding fragment thereof. In illustrative examples of this type, the anti-PD-L1 antigen-binding molecule binds specifically to one or more amino acids in amino acid sequence SKKQSDTHLEET [SEQ ID NO:13] (i.e., residues 279 to 290 of the full length native PD-L1 amino acid sequence set forth in SEQ ID NO:14). Alternatively, the anti-PD-L1 antigen-binding molecule may be one that competes with any one of durvalumab (MEDI4736), atezolizumab (Tecentriq), avelumab, BMS-936559/MDX-1105, MSB0010718C, LY3300054, CA-170, GNS-1480 and MPDL3280A for binding to PD-L1.

In some embodiments, the anti-CTLA4 antigen-binding molecule is a MAb, representative examples of which include ipilimumab and tremelimumab, or an antigen-binding fragment thereof. Alternatively, the anti-CTLA4 antigen-binding molecule may be one that competes with ipilimumab or tremelimumab for binding to CTLA4. In illustrative examples of this type, the anti-CTLA4 antigen-binding molecule binds specifically to one or more amino acids in an amino acid sequence selected from YASPGKATEVRVTVLRQA [SEQ ID NO:15] (i.e., residues 25 to 42 of the full-length native CTLA4 amino acid sequence set forth in SEQ ID NO:16), DSQVTEVCAATYMMGNELTFLDD [SEQ ID NO:17] (i.e., residues 43 to 65 of the native CTLA4 sequence set forth in SEQ ID NO:16), and VELMYPPPYYLGIG [SEQ ID NO:18] (i.e., residues 96 to 109 of the native CTLA4 sequence set forth in SEQ ID NO:16).

In some embodiments, the therapeutic combination comprises, consists or consists essentially of an anti-RANKL antigen-binding molecule and an anti-PD-1 antigen-binding molecule. In other embodiments, the therapeutic combination comprises, consists or consists essentially of an anti-RANKL antigen-binding molecule and an anti-PD-L1 antigen-binding molecule. In still other embodiments, the therapeutic combination comprises, consists or consists essentially of an anti-RANKL antigen-binding molecule, an anti-PD-1 antigen-binding molecule and an anti-PD-L1 antigen-binding molecule. In still other embodiments, the therapeutic combination comprises, consists or consists essentially of an anti-RANKL antigen-binding molecule, an anti-PD-1 antigen-binding molecule and an anti-CTLA4 antigen-binding molecule. In other embodiments, the therapeutic combination comprises, consists or consists essentially of an anti-RANK antigen-binding molecule and an anti-PD-L1 antigen-binding molecule.

In some embodiments in which the RANKL or ICM antagonist is an antigen-binding molecule, the antigen-binding molecule is linked to an immunoglobulin constant chain (e.g., an IgG1, IgG2a, IgG2b, IgG3, or IgG4 constant chain). The immunoglobulin constant chain may comprise a light chain selected from a κ light chain or λ light chain; and a heavy chain selected from a γ1 heavy chain, γ2 heavy chain, γ3 heavy chain, and γ4 heavy chain.

In certain embodiments, the therapeutic combination comprises, consists or consists essentially of a RANKL antagonist and two or more different ICM antagonists. In representative examples of this type, the therapeutic combination comprises, consists or consists essentially of a RANKL antagonist and at least two of a CTLA4 antagonist, a PD-1 antagonist and a PD-L1 antagonist.

Antagonist components of the therapeutic combination may be in the form of discrete components. Alternatively, they may be fused or otherwise conjugated (either directly or indirectly) to one another.

In specific embodiments, the therapeutic combination is in the form of a multispecific antagonist agent, comprising the RANKL antagonist and the at least one ICM antagonist. The multispecific agent may be a complex of two or more polypeptides. Alternatively, the multispecific agent may be a single chain polypeptide. The RANKL antagonist may be conjugated to the N-terminus or to the C-terminus of an individual ICM antagonist. The RANKL antagonist and the ICM antagonist may be connected directly or by an intervening linker (e.g., a polypeptide linker). In advantageous embodiments, the multispecific antagonist agent comprises at least two antigen-binding molecules. Suitably, such multispecific antigen-binding molecules are in the form of recombinant molecules, including chimeric, humanized and human antigen-binding molecules.

In a related aspect, the present invention provides multispecific antigen-binding molecules for antagonizing RANKL and at least one ICM. These multispecific antigen-binding molecules generally comprise, consist or consist essentially of an antibody or antigen-binding fragment thereof that binds specifically to RANKL or to RANK and for a respective ICM, an antibody or antigen-binding fragment thereof that binds specifically to that ICM. The antibody and/or antigen-binding fragments may be connected directly or by an intervening linker (e.g., a chemical linker or a polypeptide linker). An individual multispecific antigen-binding molecule may be in the form of a single chain polypeptide in which the antibodies or antigen-binding fragments are operably connected. Alternatively, it may comprise a plurality of discrete polypeptide chains that are linked to or otherwise associated with one another to form a complex. In some of the same and other embodiments, the multispecific antigen-binding molecules are bivalent, trivalent, or tetravalent.

The at least one ICM is suitably selected from PD-1, PD-L1, PD-L2, CTLA-4, A2AR, A2BR, CD276, VTCN1, BTLA, IDO, KIR, LAG3, TIM-3, VISTA, CD73, CD96, CD155, DNAM-1, CD112, CRTAM, OX40, OX4OL, CD244, CD160, GITR, GITRL, ICOS, GAL-9, 4-1BBL, 4-1BB, CD27L, CD28, CD80, CD86, SIRP-1, CD47, CD48, CD244, CD40, CD4OL, HVEM, TMIGD2, HHLA2, VEGI, TNFRS25, ICOLG and TIGIT. In specific embodiments, the ICM antagonist antagonizes an ICM that a Treg cell lacks expression of or expresses at a low level. In some of the same and other embodiments, the ICM antagonist antagonizes an ICM (e.g., PD-1 or PD-L1) that is expressed at a lower level on a Treg than CTLA4. In some of the same and other embodiments, the ICM antagonist antagonizes an ICM (e.g., PD-1 or PD-L1) that is expressed at a higher level on an immune effector cell (e.g., an effector T cell, macrophage, dendritic cell, B cell, etc.) than on a Treg. In representative examples of these embodiments, the at least one ICM antagonist antagonizes an ICM selected from one or both of PD-1 and PD-L1. In specific embodiments in which the multispecific antigen-binding molecule is bispecific, the anti-ICM antibody or antigen-binding fragment thereof is other than an anti-CTLA-4 antibody or antigen-binding fragment thereof.

Antigen-binding fragments that are contemplated for use in multispecific antigen-binding molecules may be selected from Fab, Fab′, F(ab′)₂, and Fv molecules and complementarity determining regions (CDRs). In some embodiments, individual antibodies or antigen-binding fragments thereof comprise a constant domain that is independently selected from the group consisting of IgG, IgM, IgD, IgA, and IgE. Non-limiting examples of multispecific antigen-binding molecules suitably comprise a tandem scFv (taFv or scFv₂), diabody, dAb₂/VHH2, knobs-in-holes derivative, Seedcod-IgG, heteroFc-scFv, Fab-scFv, scFv-Jun/Fos, Fab′-Jun/Fos, tribody, DNL-F(ab)₃, scFv₃-C_{H}1/C_{L}, Fab-scFv₂, IgG-scFab, IgG-scFv, scFv-IgG, scFv₂-Fc, F(ab′)₂-scFv₂, scDB-Fc, scDb-C_{H}3, Db-Fc, scFv₂-H/L, DVD-Ig, tandAb, scFv-dhlx-scFv, dAb₂-IgG, dAb-IgG, dAb-Fc-dAb, tandab, DART, BiKE, TriKE, mFc-VH, crosslinked MAbs, Cross MAbs, MAb2, FIT-Ig, electrostatically matched antibodies, symmetric IgG-like antibodies, LUZ-Y, Fab-exchanged antibodies, or a combination thereof.

Suitable antigen-binding fragments may be linked to an immunoglobulin constant chain (e.g., IgG1, IgG2a, IgG2b, IgG3, and IgG4). In representative examples of this type, the immunoglobulin constant chain may comprise a light chain selected from a κ light chain and λlight chain, and/or a heavy chain selected from a γ1 heavy chain, γ2 heavy chain, γ3 heavy chain, and γ4 heavy chain.

In some embodiments in which the RANKL antagonist is a direct RANKL antagonist, the multispecific antigen-binding molecule comprises an anti-RANKL antibody or antigen-binding fragment thereof that binds specifically to one or more amino acids of the amino acid sequence TEYLQLMVY [SEQ ID NO:1] (i.e., residues 233-241 of the native RANKL sequence set forth in SEQ ID NO:2). In other embodiments in which the RANKL antagonist is an indirect RANKL antagonist, the multispecific antigen-binding molecule may comprise an anti-RANK antibody or antigen-binding fragment thereof that binds specifically to an extracellular region of RANK (i.e., corresponding to residues 30 to 212 of the human RANK sequence set forth in SEQ ID NO:8).

In some embodiments in which the multispecific antigen-binding molecule antagonizes PD-1, the anti-PD-1 antibody or antigen-binding fragment thereof binds specifically to one or more amino acids of an amino acid sequence selected from SFVLNWYRMSPSNQTDKLAAFPEDR [SEQ ID NO:9] (i.e., residues 62 to 86 of the native human PD-1 sequence set forth in SEQ ID NO:10), SGTYLCGAISLAPKAQIKE [SEQ ID NO:11] (i.e., residues 118 to 136 of the native human PD-1 sequence set forth in SEQ ID NO:10) and NWYRMSPSNQTDKLAAFPEDRSQPGQDCRFRV [SEQ ID NO:12] (i.e., corresponding to residue 66 to 97 of the native human PD-1 sequence set forth in SEQ ID NO:10).

In some of the same embodiments and other embodiments the anti-PD-1 antibody or antigen-binding fragment thereof comprises a heavy chain and a light chain of a MAb selected from nivolumab, pembrolizumab, pidilizumab, and MEDI-0680 (AMP-514), AMP-224, JS001-PD-1, SHR-1210, Gendor PD-1, PDR001, CT-011, REGN2810, BGB-317 or antigen-binding fragments thereof.

In some embodiments in which the multispecific antigen-binding molecule antagonizes PD-L1, the anti-PD-L1 antibody or antigen-binding fragment thereof binds specifically to one or more amino acids of the amino acid sequence SKKQSDTHLEET [SEQ ID NO:13] (i.e., residues 279 to 290 of the native human PD-L1 amino acid sequence as set forth in SEQ ID NO:14). Illustrative antibodies and antigen-binding fragments of this type include those that comprise a heavy chain and a light chain of a MAb selected from durvalumab (MEDI4736), atezolizumab (Tecentriq), avelumab, BMS-936559/MDX-1105, MSB0010718C, LY3300054, CA-170, GNS-1480 and MPDL3280A, or antigen-binding fragments thereof.

In some embodiments in which the multispecific antigen-binding molecule antagonizes CTLA4, the anti-CTLA4 antibody or antigen-binding fragment thereof binds specifically to one or more amino acids of an amino acid sequence selected from YASPGKATEVRVTVLRQA [SEQ ID NO:15] (i.e., residues 25 to 42 of the full-length native PD-CTLA4 amino acid sequence set forth in SEQ ID NO:16), DSQVTEVCAATYMMGNELTFLDD [SEQ ID NO:17] (i.e., residues 43 to 65 of the native CTLA4 sequence set forth in SEQ ID NO:16), and VELMYPPPYYLGIG [SEQ ID NO:18] (i.e., residues 96 to 109 of the native CTLA4 sequence set forth in SEQ ID NO:16). Illustrative antibodies and antigen-binding fragments of this type include those that comprise a heavy chain and a light chain of a MAb selected from ipilimumab and tremelimumab, or antigen-binding fragments thereof.

In some embodiments, the multispecific antigen-binding molecule comprises, consists or consists essentially of an anti-RANKL antigen-binding molecule and an anti-PD-1 antigen-binding molecule. In other embodiments, the multispecific antigen-binding molecule comprises, consists or consists essentially of an anti-RANKL antigen-binding molecule and an anti-PD-L1 antigen-binding molecule. In still other embodiments, the multispecific antigen-binding molecule comprises, consists or consists essentially of an anti-RANKL antigen-binding molecule, an anti-PD-1 antigen-binding molecule and an anti-PD-L1 antigen-binding molecule. In still other embodiments, the multispecific antigen-binding molecule comprises, consists or consists essentially of an anti-RANKL antigen-binding molecule, an anti-PD-1 antigen-binding molecule and an anti-CTLA4 antigen-binding molecule. In other embodiments, the multispecific antigen-binding molecule comprises, consists or consists essentially of an anti-RANK antigen-binding molecule and an anti-PD-L1 antigen-binding molecule.

In another aspect, the present invention provides methods of producing a therapeutic combination as broadly described above and elsewhere herein. These methods generally comprise combining an anti-RANKL or anti-RANK antigen-binding molecule and at least one anti-ICM antigen-binding molecule to thereby produce the therapeutic combination. In some embodiments, the methods comprise generating an antigen-binding molecule that binds specifically to a target polypeptide (e.g., RANKL, RANK or an ICM) of the therapeutic combination (e.g., by immunizing an animal with an immunizing polypeptide comprising an amino acid sequence corresponding to an the target polypeptide; and identifying and/or isolating a B cell from the animal, which binds specifically to the target polypeptide or at least one region thereof; and producing the antigen-binding molecule expressed by that B cell). In non-limiting examples, the methods further comprise derivatizing the antigen-binding molecule so generated to produce a derivative antigen-binding molecule with the same epitope-binding specificity as the antigen-binding molecule. The derivative antigen-binding molecule may be selected from antibody fragments, illustrative examples of which include Fab, Fab′, F(ab′)₂, Fv), single chain (scFv) and domain antibodies (including, for example, shark and camelid antibodies), and fusion proteins comprising an antibody, and any other modified configuration of an immunoglobulin molecule that comprises an antigen binding/recognition site.

In some embodiments, the therapeutic combination or multispecific antigen-binding molecule is contained in a delivery vehicle (e.g., a liposome, a nanoparticle, a microparticle, a dendrimer or a cyclodextrin).

In still another aspect, the present invention provides constructs that comprise nucleic acid sequence encoding a multispecific antigen-binding molecule as broadly described above and elsewhere herein in operable connection with one or more control sequences. Suitable constructs are preferably in the form of an expression construct, representative examples of which include plasmids, cosmids, phages, and viruses.

Still another aspect of the invention provides host cells that contain constructs as broadly described above and elsewhere herein.

In another aspect, the present invention provides pharmaceutical compositions comprising the therapeutic combination or multispecific antigen-binding molecule as broadly described above, and a pharmaceutically acceptable carrier or diluent. In some embodiments, the compositions further comprise at least one ancillary agent selected from a chemotherapeutic agent (e.g., selected from antiproliferative/antineoplastic drugs, cytostatic agents, agents that inhibit cancer cell invasion, inhibitors of growth factor function, anti-angiogenic agents, vascular damaging agents, etc.), or an immunotherapeutic agent (e.g., cytokines, cytokine-expressing cells, antibodies, etc.).

Still another aspect of the present invention provides methods for stimulating or augmenting immunity in a subject. These methods generally comprise, consist or consist essentially of administering to the subject an effective amount of the therapeutic combination or multispecific antigen-binding molecule as broadly described above, to thereby stimulate or augment immunity in the subject. In embodiments in which the RANKL antagonist and the at least one ICM antagonist of the therapeutic combination are provided as discrete components, the components are suitably administered concurrently to the subject. In illustrative examples of this type, the RANKL antagonist is administered simultaneously with the at least one ICM antagonist. In other illustrative examples, the RANKL antagonist and the at least one ICM antagonist are administered sequentially. For instance, the RANKL antagonist may be administered prior to administration of the at least one ICM antagonist. Suitably, the RANKL antagonist is administered after administration of the at least one ICM antagonist.

Typically, the stimulated or augmented immunity comprises a beneficial host immune response, illustrative examples of which include any one or more of the following: reduction in tumor size; reduction in tumor burden; stabilization of disease; production of antibodies against an endogenous or exogenous antigen; induction of the immune system; induction of one or more components of the immune system; cell-mediated immunity and the molecules involved in its production; humoral immunity and the molecules involved in its production; antibody-dependent cellular cytotoxicity (ADCC) immunity and the molecules involved in its production; complement-mediated cytotoxicity (CDC) immunity and the molecules involved in its production; natural killer cells; cytokines and chemokines and the molecules and cells involved in their production; antibody-dependent cytotoxicity; complement-dependent cytotoxicity; natural killer cell activity; and antigen-enhanced cytotoxicity. In representative examples of this type, the stimulated or augmented immunity includes a pro-inflammatory immune response.

Yet another aspect of the present invention provides methods for inhibiting the development or progression of immunosuppression or tolerance to a tumor in a subject. These methods generally comprise, consist or consist essentially of contacting the tumor with the therapeutic combination or multispecific antigen-binding molecule as broadly described above, to thereby inhibit the development or progression of immunosuppression or tolerance to the tumor in the subject. Suitably, the therapeutic combination or multispecific antigen-binding molecule also contacts an antigen-presenting cell (e.g., a dendritic cell) that presents a tumor antigen to the immune system.

A further aspect of the present invention provides methods for inhibiting the development, progression or recurrence of a cancer in a subject. These methods generally comprise, consist or consist essentially of administering to the subject an effective amount of a therapeutic combination or multispecific antigen-binding molecule as broadly described above and elsewhere herein, to thereby inhibit the development, progression or recurrence the cancer in the subject.

In a related aspect, the present invention provides methods for treating a cancer in a subject. These methods generally comprise, consist or consist essentially of administering to the subject an effective amount of a therapeutic combination or multispecific antigen-binding molecule as broadly described above and elsewhere herein, to thereby treat the cancer.

Non-limiting examples of cancers that may be treated in accordance with the present invention include melanoma, breast cancer, colon cancer, ovarian cancer, endometrial and uterine carcinoma, gastric or stomach cancer, pancreatic cancer, prostate cancer, salivary gland cancer, lung cancer, hepatocellular cancer, glioblastoma, cervical cancer, liver cancer, bladder cancer, hepatoma, rectal cancer, colorectal cancer, kidney cancer, vulval cancer, thyroid cancer, hepatic carcinoma, anal carcinoma, penile carcinoma, testicular cancer, oesophageal cancer, tumors of the biliary tract, head and neck cancer, and squamous cell carcinoma. In some particular embodiments, the cancer is a metastatic cancer.

In any of the above aspects involving administration of the therapeutic combination or multispecific antigen-binding molecule to a subject, the subject has suitably reduced or impaired responsiveness to immunomodulatory agents, for example a subject that has reduced or impaired responsiveness to ICM molecule antagonists (e.g., an anti-PD-1 or anti-PD-L1 immunotherapy).

In some of the methods of the invention, an effective amount of an ancillary anti-cancer agent is concurrently administered to the subject. Some suitable ancillary anti-cancer agents include a chemotherapeutic agent, external beam radiation, a targeted radioisotope, and a signal transduction inhibitor. However, any other known anti-cancer agent is equally as applicable for use with the methods of the present invention.

In a further aspect, the present invention provides kits for stimulating or augmenting immunity, for inhibiting the development or progression of immunosuppression or tolerance to a tumor, or for treating a cancer in a subject. These kits comprise any one or more of the therapeutic combinations, pharmaceutical compositions, and multispecific antigen-binding molecules as broadly described above and elsewhere herein.

### BRIEF DESCRIPTION OF THE DRAWINGS

FIG. 1 a graphical representation depicting suppression of experimental lung metastases by combination anti-CTLA4 and anti-RANKL is NK cell- and IFN-γ-dependent. Groups of 5-10 C57BL/6 wild type (WT) or gene-targeted mice (as indicated) were injected i.v. with B16F10 melanoma cells (2×10⁵) (A-C). Groups of 5-10 C57BL/6 wild type (WT) were injected i.v. with RM1 prostate cancer cells (1×10⁴) (D). Mice were treated on day −1, 0 and 2 (relative to tumor inoculation) with cIg, anti-CTLA4 (UC10-4F10, hamster IgG) and/or anti-RANKL (IK22/5) (all 200 μg/mouse i.p.) as indicated. (B) Some groups of mice were additionally treated on days −1, 0 and 7 with anti-CD8β or anti-asGM1 (all 100 μg/mouse i.p. each). Metastatic burden was quantified in the lungs after 14 days by counting colonies on the lung surface. Means±SEM of each group are shown. Improved metastatic control of the combination was statistically significant as indicated (one way ANOVA, Tukey's multiple comparisons; *P<0.05,**P<0.01,****P<0.0001).

FIG. 2 is a graphical representation showing that isotype of anti-CTLA4 affects its efficacy to combine with anti-RANKL to suppress experimental lung metastases. Groups of 5-8 C57BL/6 wild type (WT) mice were injected i.v. with B16F10 melanoma cells (2×10⁵) as indicated (A, B). Mice were treated on day −1, 0 and 2 (relative to tumor inoculation) with cIg (1D12, mouse IgG2a), various isotypes of anti-CTLA4 (UC10-4F10 (hamster IgG), 9D9 (mouse IgG2a, IgG2b, IgG1 or IgG1 D265A) and/or cIg (2A3, rat IgG2a) or anti-RANKL (IK22/5) (all 200 μg/mouse i.p.) as indicated. Metastatic burden was quantified in the lungs after 14 days by counting colonies on the lung surface. Means±SEM of each group are shown. (A) is a pooled result from two independent experiments. Improved metastatic control of the combination versus anti-CTLA4 alone (IgG2a and hamster isotypes) was statistically significant as indicated (one way ANOVA, Tukey's multiple comparisons; *P<0.05,**P<0.01,***P<0.001,****P<0.0001). (B) Improved metastatic control with anti-CTLA4-IgG2a isotype alone, and with various combinations, is significant where indicated (one way ANOVA, Sidak's multiple comparisons where monotherapy anti-CTLA4 is compared with cIg, and with combination with anti-RANKL, or combination with cIg; *P<0.05,***P<0.001,****P<0.0001). Experiments were performed once.

FIG. 3 is a graphical representation showing that anti-CTLA4 combined with anti-RANKL suppresses B16F10 subcutaneous tumor. Groups of 5 C57BL/6 wild type (WT) mice were injected s.c. with B16F10 melanoma cells (1×10⁵). Mice were treated on days 3, 7, 9 and 11 (relative to tumor inoculation) with cIg, anti-CTLA4 (UC10-4F10, hamster Ig, 200 jig i.p. Means ±SEM of each group are shown. Graph is a representative growth curve from seven independent experiments.

FIG. 4 is a graphical representation showing that the IgG2a isotype of anti-CTLA4 combines most effectively with anti-RANKL to suppress B16F10 subcutaneous tumor. Groups of 5 C57BL/6 wild type (WT) mice were injected s.c. with B16F10 melanoma cells (1×10⁵). Mice were treated on (A) days 6, 8, 10 and 12, or (B) days 3, 7, 9 and 11 (relative to tumor inoculation) with cIg, anti-CTLA4 (9D9, mouse IgG2a or IgG1-D265A, 50 μg i.p. as indicated) and/or anti-RANKL (IK22/5, 200 μg i.p.) as indicated. Means±SEM of each group are shown. Superior control of subcutaneous tumor growth with combination anti-CTLA4 and anti-RANKL was statistically significant where indicated (one way ANOVA, Tukey's multiple comparisons; *P<0.05,**P<0.01,****P<0.0001). Differences between combination anti-CTLA4-IgG2a and anti-RANKL, either as monotherapy, or cIg were significant as indicated (Kruskal-Wallis test, Dunn's multiple comparisons, where monotherapy arms or cIg were compared with combination anti-CTLA4-IgG2a and anti-RANKL; *P<0.05,**P<0.01,****P<0.0001). When anti-CTLA4-IgGl-D265A combined with anti-RANKL was compared with either as monotherapy, no significant difference was found. (C) B16F10 subcutaneous tumor growth is maximally suppressed by the combination of anti-CTLA4-IgG2a and anti-RANKL therapy. Predicted mixed effects repeated measures tumor data (log scale) comparing treatment groups for seven independent pooled experiments (with 5-6 mice per group) is shown in the graph. Superior growth suppression with combination anti-RANKL and anti-CTLA4 (mIgG2a) compared to monotherapy or control, and with anti-RANKL and anti-CTLA4 (mIgGl-D265A) with control but not monotherapy, was significant as indicated in the table shown (pairwise comparisons; **P<0.01,****P<0.0001).

FIG. 5 is a graphical representation depicting the expression of RANKL and RANK in the B16F10 tumor microenvironment (TME). Groups of C57BL/6 wild type (WT) were injected s.c. with B16F10 melanoma cells (1×10⁵) (A-B). Mice were treated on day 3, 7, and if experiment ongoing also day 11 and 15 relative to tumor inoculation with cIg (1-1, rat IgG2a, 200 μg i.p. or anti-RANKL (IK22/5, 200 μg i.p.) as indicated. Two independent experiments, each with 3-5 mice per group, are combined in each of (A-B). Significant differences in RANKL expression between T-cell subsets and organ site, at time points indicated, are indicated in (A) (one way ANOVA, Tukey's multiple comparisons; *P<0.05,**P<0.01,****P<0.0001). (B) tumors were analysed at day 16; no significant differences were seen between cIg and a-RANKL treated groups as indicated.

FIG. 6 is a graphical representation depicting the efficacy of combination anti-CTLA4-IgG2a and anti-RANKL therapy is FcR-, IFNγ-, Batfβ- and CD8⁺ T cell-dependent. Groups of C57BL/6 wild type (WT) or gene-targeted mice as indicated were injected s.c. with B16F10 melanoma cells (1×10⁵) (A-D). Mice were treated on day 3, 7, and if experiment ongoing also day 11 and 15 relative to tumor inoculation with cIg (1-1, rat IgG2a, 200 μg i.p.+1D12, mouse IgG2a or IgG1 to match anti-CTLA4 isotype, 50 μg i.p.), anti-CTLA4 (9D9 mouse IgG2a or IgG1-D265A as indicated, 50 μg i.p.) and/or anti-RANKL (IK22/5, 200 μg i.p.) as indicated. Some mice were treated i.p. on days −1, 0 and 7 with anti-CD8β) or anti-asGM1 (all 100 μg/mouse i.p. each as indicated in (B). Means±SEM of 4-9 mice per group are shown. Differences between groups in tumor growth curves is significant where indicated (one way ANOVA, Tukey's multiple comparisons; **P<0.01,***P<0.001,****P<0.0001). The WT groups treated with cIg and combination a-CTLA4 with a-RANKL are the same in (A) and (C) but are displayed in different graphs for ease of interpretation. Combination efficacy of anti-RANKL with anti-CTLA4 is FcR- and Batf3-dependent.

FIG. 7 is a graphical representation showing that combined anti-RANKL and anti-CTLA4 therapy results in increased recruitment of CD8⁺ T cells into tumors. Groups of 4-8 C57BL/6 wild type (WT) mice were injected s.c. with B16F10 melanoma cells (1×10⁵). Data is pooled from 2-5 independent experiments (A-H). Mice were treated on (A-E, G-H) days 3, 7, and 11 and 15 or (F) days 3 and 7 relative to tumor inoculation with cIg (1-1, rat IgG2a, 200 μg i.p. +1D12, mouse IgG2a, 50 μg i.p.), anti-CTLA4 (9D9, IgG2a, 50 μg i.p.) and/or anti-RANKL (IK22/5, 200 μg i.p.) as indicated. Mice were sacrificed at (A-E, G-H) end stage relative to ethical end-point for size (day 16) or (F) day 9, and tumors processed for FACS analysis. Increased (A) CD45⁺ TILs proportion of total live cells, (E) proportion of CD8⁺Ki-67⁺ T cells , ratio of CD8⁺ T cells to Tregs (defined as TCRβ⁺CD4⁺, FoxP3⁺) at (F) day 9 and (G) day 15-16, and (H) ratio of CD8⁺ T cells to CD11b⁺GR1^{hi} cells is significant where indicated (one-way ANOVA, Dunnett's multiple comparisons where each group is compared with anti-CTLA4-IgG2a+anti-RANKL combination therapy; *P<0.05,**P<0.01,***P<0.001,****P<0.0001). (B) Increased CD8⁺ T cells as a proportion of total CD45⁺ TILs is significant where indicated (Kruskal-Wallis test, Dunn's multiple comparisons where each group is compared with combination 9D9 IgG2a+IK22/5 treatment; *P<0.05,****P<0.0001). (C) Increased number of intratumor CD8⁺ T cells is increased with combination therapy (one-way ANOVA, Dunnett's multiple comparisons where each group is compared with anti-CTLA4-IgG2a+anti-RANKL combination therapy; **P<0.01,***P<0.001). (D) Decreased Tregs with anti-CTLA4-IgG2a compared with cIg or anti-RANKL treatment is significant as shown (one-way ANOVA, Tukey's multiple comparisons; **P<0.01).

FIG. 8 is a graphical representation showing that anti-RANKL improves the efficacy of anti-CTLA4 by increasing T cell cytokine polyfunctionality. Groups of 4-8 C57BL/6 wild-type (WT) mice were inoculated s.c. with B16F10 melanoma cells (1×10⁵). (A-E) Mice were treated on days 3, 7, 11 and 15 relative to tumor inoculation with cIg (1-1, rat IgG2a, 200 μg i.p. +1D12, mouse IgG2a, 50 μg i.p.), anti-CTLA4 (9D9, IgG2a, 50 μg i.p.) and/or anti-RANKL (IK22/5, 200 μg i.p.) as indicated. Mice were sacrificed on day 16 relative to tumor inoculation and tumors processed and stimulated ex-vivo before ICS was performed. Increased proportion of CD8⁺ T cells with positive staining for (A) IFNy, (B) IFNy and IL-2 or (C) IFNy, IL-2 and TNFa co-expression (“triple positive”); and (D) increased proportion of CD4⁺ T cells expressing IFNy with combination therapy is significant as indicated (Kruskal-Wallis test, Dunn's multiple comparisons where each group is compared with combination 9D9 IgG2a+IK22/5 treatment; *P<0.05,**P <0.01,***P<0.001,****P<0.0001). 2-3 pooled independent experiments are displayed in (A-D). Mean proportion of CD8⁺ T cell expressing zero, one, two or three cytokines (of IFNy, IL-2 and TNFa) from two pooled experiments is shown for each of four treatment groups as indicated (E).

FIG. 9 is a graphical representation showing that co-blockade of PD-1/PD-L1 and RANKL results in synergistic suppression of metastasis. Groups of C57BL/6 WT mice were injected intravenously with (A, C) B16F10 melanoma or (B, D) RM1 prostate carcinoma (2×105 cells). Mice were treated on day -1, 0 and 2 (relative to tumor inoculation) with cIg (2A3, 200 μg i.p.), (A-B) anti-PD-1 (RMP1-14, 200 μg i.p.) or (C-D) anti-PD-L1 (10F.9G2, 200 μg i.p.), and/or anti-RANKL (IK22/5, 200 μg i.p.) as indicated. Metastatic burden was quantified in the lungs after 14 days by counting colonies on the lung surface. Means±SEM of 5 mice per group are shown. Two experiments are pooled in (A). Improved metastatic control of the combination was statistically significant as indicated (one way ANOVA, Tukey's multiple comparisons; *P<0.05,**P<0.01,***P<0.001,****P<0.0001).

FIG. 10 is a graphical representation showing that anti-PD-1 and anti-RANKL MAbs restrain subcutaneous growth of tumors. Groups of (A) C57BL/6 or (B) BALB/c WT male mice were injected subcutaneously with (A) MC38 or (B) CT26 colon carcinoma (1×105 cells) on day 0. Mice were then treated i.p. on days 6, 9, 12 and 15 with either cIg (2A3 or 1-1, 250 mg i.p.); anti-PD-1 alone (RMP1-14, 250 mg i.p.); anti-RANKL alone (IK22/5, 200 mg i.p.) or their combinations as indicated. Tumor growth was measured using a digital caliper, and tumor sizes are presented as mean±SEM for 5-6 mice per group. Reduced s.c. tumor growth is significant where indicated (one way ANOVA, Tukey's multiple comparisons; *P<0.05,**P<0.01,***P<0.001,****P <0.0001).

FIG. 11 is a graphical representation showing the ability of anti-RANKL to suppress subcutaneous tumor growth is dependent on BatF3, but is not dependent on Fc receptor expression. Groups of C57BI/6 or gene-targeted mice as indicated were injected subcutaneously with MCA1956 fibrosarcoma cells (1×10⁶). Mice were treated on days 3, 7, 11 and 15 relative to tumor inoculation with anti-RANKL (IK22/5, 200 ug ip) or cIg (1-1, 200 ug ip). Means +/− SEM of the 5-7 mice per group are shown. Tumor size was significantly different where indicated using one way ANOVA, Sidak's multiple comparisons, comparing treatment groups in like genotypes (* p <0.05).

FIG. 12 is a graphical representation showing co-expression of RANK and PD-L1 in infiltrating myeloid cells from tumors. C57BI/6 were injected subcutaneously with MCA1956 fibrosarcoma cells (1×10⁶ cells). Tumors were allowed to grow for 22 days without any treatment until they reached approximately 50mm³. Tumors were collected, single-cell suspensions were generated and flow cytometry was performed. In panel A, PDL-1 and CD103 expression were analysed in RANK-positive gated CD11c+/MHCII+DC, indicating nearly 100% of RANK-positive DC express both PD-L1 and CD103. In panel B, CD206 and RANK expression was analysed on CD11b+, F480+macrophages, indicating that 52% of tumor infiltrating macrophages co-expressed RANK and CD206.

FIG. 13 is a schematic representation of DNA vectors encoding exemplary RAN KL-PD-1 bispecific antibodies. (A) Representation of an expression vector encoding a RANKL-PD-1 diabody. (B) Representation of DNA constructs encoding RANKL-PD-1 tribodies. The first construct encodes a PD-1 Fab L domain and a first RANKL scFv domain, and the second construct encodes a PD-1 Fab Fd domain and a second RANKL scFv domain. Thus, the resulting tribody will have two RANKL-binding fragments, and a single PD-1 binding fragment. (C) Representation of DNA constructs encoding PD-1-RANKL tribodies. The first construct encodes a RANKL Fab L domain and a first PD-1 scFv domain, and the second construct encodes a RANKL Fab Fd domain and a second scFv domain.

FIG. 14 is a cartoon representation of an exemplary bispecific anti-RANKL, anti-PD-1 tribody.

FIG. 15 is a graphical representation showing that efficacy of anti-RANKL combination therapy is not completely dependent on Treg depletion. Groups of C57BL/6 FoxP3-DTR mice were injected s.c. with (A-C) B16F10 melanoma cells (1×10⁵) or (D, E) RM-1 prostate carcinoma cells (5×10⁴). Mice were treated on (A,B) day 3, 7, 11 and 15, or (C-E) days 3, 7 and 11 relative to tumor inoculation with cIg (1-1; rat IgG2a, 200 μg i.p.+1D12; mouse IgG2a, 50 μg i.p.), DT (250 ng i.p. on (A) day 3 only or (C-E) days 3 and 7 only) and/or anti-RANKL (IK22-5; rat IgG2a, 200 μg i.p.) as indicated. (A, B) B16F10 s.c. tumor growth (displayed as mean±SEM), (C) B16F10 tumor rejections (defined as complete regression of established subcutaneous tumor after treatment, assessed at day 15 after tumor inoculation), (D) RM-1 tumor growth (displayed as mean±SEM), and (E) Tregs as a proportion of total TILs for the experiment shown in (D), are shown for 4-6 mice per group. Statistical significance where indicated was determined by one way ANOVA, Tukey's multiple comparisons (** p<0.01,***p<0.001,****p<0.0001).

FIG. 16 is a graphical representation showing that RANKL identifies PD-1^{hi} expressing T cells in TME. (A-C) Groups of BALB/c wild type (WT) mice (n=10/group) were injected s.c. with 2×10⁵ CT26 colon carcinoma cells. On day 10 after tumor inoculation, mice were randomized into groups bearing equivalent median tumor size and were treated i.p with a single dose of antibody as indicated: cIg (200 μg), anti-CTLA4 (9D9, mIgG2a isotype, 50 μg), anti-PD-1 (200 μg) or the indicated combinations. Three days after treatment, tumors were harvested and processed for flow cytometry gating on live CD45.2 cells of leukocyte morphology. (A) Proportion of RANKL^{λ} (black bars) or RANKL⁻ (grey bars) CD8⁺ T cell TILs expressing PD-1, (B) expression level of PD-1 (expressed as geometric MFI, gMFI) by RANKL⁺ (black bars) or RANKL⁻ (grey bars) CD8⁺ T cell TILs, and (C) expression level of CTLA4 (expressed as geometric MFI, gMFI) by RANKL⁻ (black bars) or RANKL⁻ (grey bars) CD8⁺ T cell TILs. Statistical differences were determined by one way ANOVA with Tukey's post-test analysis, except in (C), where Mann-Whitney test was used to compare within-treatment groups (*p<0.05,**p<0.01,***p<0.001,****p<0.0001).

FIG. 17 is a graphical representation depicting co-targeting of RANKL with PD-1/PD-L1 alone or in combination with CTLA-4 suppresses subcutaneous tumor growth. Groups of BALB/c (A, B) wild type (WT) or TRAMP transgenic I mice (n=5-17/group) were injected s.c. either with 1×105 CT26 (A, B), or with 1×106 Tramp-C1 prostate carcinoma I on day 0, and tumor growth was monitored. Mice were treated i.p. on (A-C) days 10, 14, 18 and 22 or I 20, 24, 28 and 32 (relative to tumor inoculation) with the following antibodies: cIg (to a total of 250-350 μg), anti-CTLA4 (9D9 mIgG2a, 50 μg), anti-PD-1 (clone RMP1-14; A, D: 250 μg; C: 100 μg), anti-PD-L1 (clone 10F.9G2; 100 μg), anti-RANKL (clone IK22.5; 200 μg) or their combinations as indicated.

Tumor sizes presented as mean±SEM. (A) is representative of 2-3 independent experiments, all other experiments were performed once. Statistical differences between indicated groups were determined by one way ANOVA with Tukey's post-test analysis on the final day of measurement unless indicated otherwise (*p<0.05,**p<0.01,***p<0.001,****p<0.0001). In (C), significant differences in tumor sizes between dual-antibody and triple-antibody combinations at day 30 were assessed; not shown on graph is the following comparison at day 22: anti-PD-1 vs anti-PD-1+anti-RANKL (****); #: at day 35, significant difference between the two remaining groups were determined by an unpaired t-test (*p<0.05). In (B), # indicates significant difference for the indicated comparison determined by an unpaired t-test (*p<0.05). In (A, C) parentheses: tumor rejection rates (no parentheses indicate no rejections). In (A), rejection rates for two identical experiments were pooled and presented in italicized parentheses; significant differences between rejection rates indicated groups were determined by Chi-squared (χ2) analysis (Fisher's exact test; **p<0.01).

FIG. 18 is a graphical representation showing that favorable early alterations in RANKL expression within the TME are seen after first treatment with ICB. (A-C) Groups of BALB/c wild type (WT) mice (n=5-10/group) were injected s.c. with 2×10⁵ CT26 colon carcinoma cells. On day 10 after tumor inoculation, mice were randomized into groups bearing equivalent median tumor size and were treated i.p with a single dose of antibody as indicated: cIg (200 μg), anti-CTLA4 (9D9, mIgG2a isotype, 50 μg), anti-PD-1 (clone RMP1-14; 200 μg) or the indicated combinations. Three days after treatment, tumors were harvested and processed for flow cytometry gating on (A-D) live CD45.2 cells of leukocyte morphology, or (E) on live single CD45.2+ cells excluded from the lymphocyte gate. (A) Proportion of CD8⁺ T cell TILs expressing RANKL, (B) proportion of gp70-specific CD8⁺ T cell TILs expressing RANKL, and (C) proportion of CD4⁺ T cell TILs expressing RANKL displayed for indicated treatment groups. Means±SEM are shown. Statistical differences were determined by one way ANOVA with Tukey's post-test analysis, except in (A), where Kruskal-Wallis test with Dunn's post-test analysis was used, (*p<0.05,**p<0.01, ***p<0.001,****p<0.0001).

FIG. 19 is a graphical representation depicting unique alterations in TME after anti-PD-1 vs. anti-CTLA4 treatment. (A,B) Groups of BALB/c wild type (WT) mice (n=5-10/group) were injected s.c. with 2×10⁵ CT26 colon carcinoma cells. On day 10 after tumor inoculation, mice were randomized into groups bearing equivalent median tumor size and were treated i.p with a single dose of antibody as indicated: cIg (200 μg), anti-CTLA4 (9D9, mIgG2a isotype, 50 μg), anti-PD-1 (clone RMP1-14; 200 μg) or the indicated combinations. Three days after treatment, tumors were harvested and processed for flow cytometry gating on (A-D) live CD45.2 cells of leukocyte morphology, or (E) on live single CD45.2+cells excluded from the lymphocyte gate. (A) Geometric mean fluorescent intensity (gMFI) PD-1 expression by gp70-specific CD8⁺ T cell TILs shown for indicated treatment groups. (B) Proportion of cells expressing PD-L1 shown for indicated treatment groups. Means±SEM are shown. Statistical differences were determined by one way ANOVA with Tukey's post-test analysis, except in (B), where statistical differences were determined by Mann-Whitney test for indicated comparisons (*p<0.05,**p<0.01,***p<0.001,****p<0.0001). Additionally, in (A), statistical difference in gMFI for indicated comparison of anti-PD-1 alone or combined with anti-RANKL was determined by Mann-Whitney test (#p<0.05).

FIG. 20 is a graphical representation showing that co-targeting of RANKL in combination with CTLA-4 suppresses subcutaneous tumor growth. Groups of BALB/c mice (n=5-17/group) were injected s.c. either with 1×10⁵ CT26 on day 0, and tumor growth was monitored. Mice were treated i.p. on days 10, 14, 18 and 22 relative to tumor inoculation with the following antibodies: cIg (to a total of 250-350 μg), anti-CTLA4 (9D9 mIgG2a, 50 μg), anti-PD-1 (clone RMP1-14; A, D: 250 μg; C: 100 μg), anti-PD-L1 (clone 10F.9G2; 100 μg), anti-RANKL (clone IKK22.5; 200 μg) or their combinations as indicated. Tumor sizes presented as mean±SEM. Statistical differences between indicated groups were determined by one way ANOVA with Tukey's post-test analysis on the final day of measurement unless indicated otherwise (*p<0.05,**p<0.01,***p<0.001,****p<0.0001). # indicates significant difference for the indicated comparison determined by an unpaired t-test (*p<0.05).

FIG. 21 is a graphical representation showing that optimal anti-tumor efficacy of anti-PD-1 and anti-RANKL is affected by sequencing of antibody administration. Groups of C57BI/6 wild type (WT) mice (n=5/group) were injected s.c. with 5×10⁵ 3LL lung carcinoma cells. For concurrent treatment groups (black symbols), mice were treated i.p. on days 8, 12, 16 and 20 relative to tumor inoculation (as indicated by arrows) with cIg (1-1, 100 μg), anti-PD-1 (RMP1-14, 100 μg) and/or anti-RANKL (IK22/5, 100 μg) as indicated. For sequential treatment groups (colored symbols), where treatment order is indicated in figure legend, mice were treated i.p. on days 8 and 12 (first antibody) and days 16 and 20 (second antibody) respectively (relative to tumor inoculation) with cIg (1-1, 200 μg), anti-PD-1 (RMP1-14, 200 μg) and/or anti-RANKL (IK22/5, 200 μg) as indicated. Mean±SEM tumor size is shown for each treatment group. Statistical differences between groups at day 22 were determined by one way ANOVA with Tukey's post-test analysis, and key comparisons are shown (*p<0.05,**p<0.01,****p<0.0001). Two independent experiments have been pooled.

FIG. 22 is a graphical representation showing that RANKL identifies PD1^{hi} expressing T cells in TME. Of BALB/c wild type (WT) mice (n=10/group) were injected s.c. with 2×10⁵ CT26 colon carcinoma cells. On day 13 after tumor inoculation, tumors were harvested and processed for flow cytometry gating on live CD45.2 cells of leukocyte morphology. PD-1 expression was analysed on either RANKL+cells or RANKL-cells CD8⁺ T cell TILs.

FIG. 23 is a schematic representation showing an illustrative generation and characterization of an anti-RANKL/PD-1 FIT-Ig. (A) Schematic diagram of anti-RANKL/PD-1 FIT-Ig with antigen binding domains labelled. “A” sequences indicate denosumab antibody sequence and “B” sequence indicate nivolumab antibody sequences (B) Design of the three DNA constructs encoding RAN KL/PD-1 FIT-Ig. “A” sequences indicate denosumab antibody sequence and “B” sequence indicate nivolumab.

FIG. 24 is a schematic representation showing an illustrative generation and characterization of an anti-RANKL/CTLA4 FIT-Ig. (A) Schematic diagram of anti-RANKL/CTLA4 FIT-Ig with antigen binding domains labelled. “A” sequences indicate denosumab antibody sequence and “B” sequence indicate ipilimumab antibody sequences (B) Design of the three DNA constructs encoding anti-RANKL/CTLA4 FIT-Ig. “A” sequences indicate denosumab antibody sequence and “B” sequence indicate ipilimumab antibody sequences.

FIG. 25 is a schematic representation showing an illustrative generation and characterization of an anti-RANKL/PD-L1 FIT-Ig. (A) Schematic diagram of anti-RANKL/PD-L1 FIT-Ig with antigen binding domains labelled. “A” sequences indicate denosumab antibody sequence and “B” sequence indicate atezolizumab antibody sequences (B) Design of the three DNA constructs encoding anti-RANKL/PD-L1 FIT-Ig. “A” sequences indicate denosumab antibody sequence and “B” sequence indicate atezolizumab antibody sequences.

FIG. 26 is a schematic representation of the bispecific anti-RANKL/PD-1 CrossMab antibody generated with four chains as indicated. Heavy chain antibody sequences are indicated in the clear/white boxes and light chain antibody sequences are indicated by grey boxes. The RMP1-14 C_{H1} and C_{L} sequences were interchanged and fused onto the human IgG1 Fc (termed RMP1-14 CH-CL-huIgGlFc) and the IK22-5 sequences were unchanged and fused onto human IgG1 Fc (IK22-5-huIgGlFc WT). Heterodimerization was further enhanced with the indicated “knob-in-hole” and additional S354C and Y349C mutations in the Fc domains. Each human Fc domain also had a D265A mutation.

FIG. 27 is a photographic representation showing analytical SDS-PAGE/Western blot analysis of the RMP1-14 CH-C_{L} X IK225 WT bispecific antibody CrossMAb expressed in transient ExpiCHO-S cell culture and purified by protein A affinity chromatography. Lane Ml: Protein Marker, TaKaRa, Cat. No.3452; Lane M2: Protein Marker, GenScript, Cat. No. M00521; Lane 1: Reducing condition; Lane 2: Non-reducing condition; Lane P: Human IgGl, Kappa (Sigma, Cat.No.I5154) as a positive control; Primary antibody: Goat Anti-Human IgG-HRP (GenScript, Cat. No.A00166); Primary antibody: Goat Anti-Human Kappa-HRP (SouthernBiotech, Cat. No. 2060-05).

FIG. 28 is a graphical representation showing detection by flow cytometry of mouse RANKL transiently expressed by HEK-293 cells. Single-cell suspensions of HEK-293 parental cells were untransfected or transiently transfected with the mouse RANKL construct and then surface stained in a two-step incubation procedure 48 hrs post transfection. Primary antibodies were either 2.5 μg of biotinylated murine RANK-Fc, 2.5 μg of biotinylated anti-RANKL/PD-1 bispecific antibody or biotinylated isotype control Ab (huIgG1 mAb control) and were incubated with HEK-293 cells for 30 minutes on ice. Secondly, incubation with a Streptavidin secondary antibody (APC from Biolegend) was used for detection of primary antibody binding for an additional 30 minutes on ice. Samples and data were analyzed on a Fortessa 4 (BD Biosciences) flow cytometer and analyzed with FlowJo v10 software (Tree Star, Inc.).

FIG. 29 is a graphical representation showing antibody competition of RANKL-RANK binding. HEK-293 cells transiently transfected with mouse RANKL were incubated with various concentrations of either anti-RANKL/PD-1 bispecific, anti-RANKL mAb IK22-5, rat IgG2a isotype control or human IgG1 isotype control for 30 minutes on ice. Secondly, cells were incubated with 2.5 μg of biotinylated recombinant murine RANK-Fc for an additional 30 minutes on ice. After two washes with FACS buffer (PBS+10% FCS), a final incubation with Streptavidin-APC for extra 30 minutes on ice. Samples and data were analyzed on a Fortessa 4 (BD Biosciences) flow cytometer and analyzed with FlowJo v10 software (Tree Star, Inc.). Representative FACS plots and summary data of inhibition of RANK-Fc binding of two independent experiments are shown.

FIG. 30 is a graphical representation showing antibody detection by flow cytometry of mouse PD-1 transiently expressed by HEK-293 cells. Single-cell suspensions of HEK-293 parental cells were untransfected or transiently transfected with the mouse PD-1 plasmid and then surface stained in a two-step incubation procedure 48 hrs post transfection. Primary antibodies were 2.5 μg of anti-RANKL/PD-1 bispecific antibody or isotype control Ab (huIgG1 mAb control) and were incubated with HEK-293 cells for 30 minutes on ice. Secondly, incubation with a goat anti-human secondary antibody (Alexa Fluor 647 from Thermo Fisher Scientific) was used for detection of antibody binding for an additional 30 minutes on ice. Samples and data were analyzed on a Fortessa 4 (BD Biosciences) flow cytometer and analyzed with FlowJo v10 software (Tree Star, Inc.). Staining with isotype control is indicated in the dark-grey shaded areas while staining with the anti-RANKL/PD-1 bispecific antibody is indicated in the light-grey shaded areas.

FIG. 31 is a graphical representation showing antibody competition of PD-1/PD-L1 binding. HEK-293 cells transiently transfected with mouse PD-1 were incubated with various concentrations of either anti-RANKL/PD-1 bispecific, anti-PD-1 mAb RMP1-14, rat IgG2a isotype control or human IgG1 isotype control for 30 minutes on ice. Secondly, cells were incubated with 2.5 μg of biotinylated recombinant murine PD-L1-Fc for an additional 30 minutes on ice. After two washes with FACS buffer (PBS+10% FCS), a final incubation with Streptavidin-APC for extra 30 minutes on ice. Samples and data were analyzed on a Fortessa 4 (BD Biosciences) flow cytometer and analyzed with FlowJo v10 software (Tree Star, Inc.). Representative FACS plots and summary data of inhibition of PD-L1-Fc binding of two independent experiments are shown.

FIG. 32 is a graphical representation showing inhibitory effects of anti-RANKL/PD-1 bispecific antibody on in vitro osteoclastogenesis. Murine bone marrow (BM) cells cultured in the presence or absence of anti-IK22-5 mAb as a positive control, huIgG1 isotype control or anti-RANKL/PD-1 bispecific antibody at concentrations from 1000ng/mL to 50 ng/mL. Culture of BM cells was performed in DMEM supplemented with CSF-1 and mouse RANKL. Seven days later, TRAP+multinucleated (more than three nuclei) cells were counted. Data are expressed as means±SEM of triplicate cultures.

FIG. 33 is a graphical representation showing that co-targeting of RANKL and PD-1 with bispecific anti-RANKL/PD-1 suppresses experimental melanoma metastasis to lung. Groups of C57BL/6 wild type (WT) mice (n=6-10/group) were injected i.v. with 2×10⁵ B16F10 melanoma cells. Mice were treated on days -1, 0 and 2 (relative to tumor inoculation) with cIg (200 μg i.p., recombinant Mac4-human IgG1 D265A), anti-RANKL (100 μg i.p., recombinant IK22.5-human IgG1 D265A), anti-PD-1 (100 μg i.p., recombinant RMP1-14-human IgG1 D265A), anti-RANKL+anti-PD-1 (100 μg i.p. each), anti-RANKL-PD-1 bispecific (50 to 200 μg i.p., human IgG1 D265A) as indicated. Metastatic burden was quantified in the lungs after 14 days by counting colonies on the lung surface. Means±SEM are shown. Statistical differences between the indicated groups were determined by one-way ANOVA with Dunnett's multiple comparison test (*p<0.05).

FIG. 34 is a graphical representation showing that co-targeting of RANKL and PD-1 with bispecific anti-RANKL/PD-1 suppresses experimental prostate cancer metastasis to lung. Groups of C57BL/6 wild type (WT) mice (n=6/group) were injected i.v. with 2×10⁵ RM-1 prostate carcinoma cells. Mice were treated on days -1, 0 and 2 (relative to tumor inoculation) with cIg (200 μg i.p., human IgG1 D265A), anti-RANKL (100 μg i.p., IK22.5 human IgG1 D265A), anti-PD-1 (100 μg i.p., human IgG1 D265A), anti-RANKL+anti-PD-1 (100 μg i.p. each), anti-RANKL-PD-1 bispecific (100 or 200 μg i.p., human IgG1 D265A) as indicated. Metastatic burden was quantified in the lungs after 14 days by counting colonies on the lung surface. Means±SEM are shown. Statistical differences between the indicated groups were determined by one-way ANOVA with Tukey's post-test analysis (**p<0.01,***p<0.001,****p<0.0001, ns =not significant).

FIG. 35 is a graphical representation showing that co-targeting of RANKL and PD-1 with bispecific anti-RANKL/PD-1 suppresses suppresses subcutaneous tumor growth of a lung cancer cell line 3LL. Groups of C57BI/6 wild type (WT) mice were injected s.c with 5×10⁵ 3LL lung carcinoma cells. Mice were treated i.p. on days 8, 12, 16 and 20 relative to tumor inoculation (as indicated by arrows) with cIg (400 μg i.p., rat IgG2a), anti-RANKL (100 μg i.p., IK22-5 rat IgG2a), anti-PD-1 (100 μg i.p., RMP1-14 rat IgG2a), anti-RANKL+anti-PD-1 (100 μg i.p. each IK22-5 and RMP1-14), and a dose titration of the anti-RANKL/PD-1 bispecific (100, 200 and 400 μg i.p., human IgG1 D265A) as indicated. Mean±SEM tumor size is shown for each treatment group.

FIG. 36 is a graphical representation showing that co-targeting of RANKL and PD-1 with bispecific anti-RANKL/PD-1 suppresses subcutaneous tumor growth of a colon carcinoma cell line CT26. Groups of BALB/c mice (n=5-17/group) were injected s.c. with 1×10⁵ CT26 on day 0, and tumor growth was monitored. Mice were treated i.p. on days 9, 17, 18 and 21 (relative to tumor inoculation) with the following antibodies: cIg (to a total of 300 μg), bispecific anti-RANKL/PD-1 (huIgG1D265A backbone; 100 μg or 200 μg, as indicated), anti-PD-1 (RMP1-14 100 μg); anti-RANKL (IK22-5, 100 μg) or their combinations as indicated. Tumor sizes presented as mean±SEM.

FIG. 37 is a graphical representation showing that co-targeting of RANKL and PD-1 with bispecific anti-RANKL/PD-1 enhances the anti-tumor efficacy of anti-CTLA4 treatment in the CT26 tumor model. Groups of BALB/c mice (n=5-17/group) were injected s.c. with 1×105 CT26 on day 0, and tumor growth was monitored. Mice were treated i.p. on days 9, 17, 18 and 21 (relative to tumor inoculation) with the following antibodies: cIg (to a total of 300 μg), bispecific anti-RANKL/PD-1 (huIgG1D265A backbone; 200 μg), anti-CTLA4 (UC10-4F10, 100 μg), anti-PD-1 (RMP1-14, 100 μg), anti-RANKL (IK22-5, 100 μg) or their combinations as indicated. Tumor sizes presented as mean±SEM.

FIG. 38 is a graphical representation showing that co-targeting of RANKL and PD-1 with bispecific anti-RANKL/PD-1 suppresses subcutaneous tumor growth of a breast cancer cell line AT3-OVA. Groups of C57BI/6 wild type (WT) (n=6/group) were injected s.c. with 1×10⁶ AT3-OVA on day 0, and tumor growth was monitored. Mice were treated i.p. on days 19,22,25 and 28 (relative to tumor inoculation) with the following antibodies: cIg (recombinant MAC4-huIgG1D265A backbone; 200 μg), bispecific anti-RANKL/PD-1 (huIgG1D265A backbone; 100 μg or 200 μg, as indicated), anti-PD-1 (recombinant RMP1-14-huIgG1D265A backbone; 100 μg); anti-RANKL (recombinant IK22-5-huIgG1D265A backbone; 100 μg) or their combinations as indicated. Tumor sizes presented as mean±SEM.

TABLE A
Brief Description of the Sequences

| SEQUENCE ID | | LENGTH |
|---|---|---|
| NUMBER | SEQUENCE | (aa) |
| SEQ ID NO: 1 | RANKL epitope 233-241 | 9 |
| SEQ ID NO: 2 | Native human RANKL (UniProt Acc. No. O14788) | 317 |
| SEQ ID NO: 3 | Denosumab heavy chain | 452 |
| SEQ ID NO: 4 | Denosumab light chain | 215 |
| SEQ ID NO: 5 | RANK CDR3 mimetic antagonist | 11 |
| SEQ ID NO: 6 | RANK CDR3 mimetic antagonist | 9 |
| SEQ ID NO: 7 | RANK epitope 330-417 | 88 |
| SEQ ID NO: 8 | Native human RANK (UniProt accession no. Q9Y6Q6) | 616 |
| SEQ ID NO: 9 | PD-1 epitope 62-86 | 25 |
| SEQ ID NO: 10 | Native human PD-1 (UniProt Acc. No. Q15116) | 155 |
| SEQ ID NO: 11 | PD-1 epitope 118-136 | 19 |
| SEQ ID NO: 12 | PD-1 epitope 66-97 | 32 |
| SEQ ID NO: 13 | PD-L1 epitope 279-290 | 12 |
| SEQ ID NO: 14 | Native human PD-L1 (UniProt accession no. Q9NZQ7) | 290 |
| SEQ ID NO: 15 | CTLA4 epitope 25-42 | 18 |
| SEQ ID NO: 16 | Native human CTLA4 (UniProt accession no. P16410) | 188 |
| SEQ ID NO: 17 | CTLA4 epitope 43-65 | 23 |
| SEQ ID NO: 18 | CTLA4 epitope 96-109 | 14 |
| SEQ ID NO: 19 | Denosumab heavy chain CDR1 | 5 |
| SEQ ID NO: 20 | Denosumab heavy chain CDR2 | 17 |
| SEQ ID NO: 21 | Denosumab heavy chain CDR3 | 13 |
| SEQ ID NO: 22 | Denosumab light chain CDR1 | 12 |
| SEQ ID NO: 23 | Denosumab light chain CDR2 | 7 |
| SEQ ID NO: 24 | Denosumab light chain CDR3 | 9 |
| SEQ ID NO: 25 | Denosumab V_{H} | 122 |
| SEQ ID NO: 26 | Denosumab V_{L} | 108 |
| SEQ ID NO: 27 | Denosumab heavy chain full-length | 464 |
| SEQ ID NO: 28 | Denosumab light chain full-length | 234 |
| SEQ ID NO: 29 | EP 1257648 anti-RANKL antibody CDR1 (V_{H}) | 6 |
| SEQ ID NO: 30 | EP 1257648 anti-RANKL antibody CDR2 (V_{H}) | 17 |
| SEQ ID NO: 31 | EP 1257648 anti-RANKL antibody CDR3 (V_{H}) | 17 |
| SEQ ID NO: 32 | EP 1257648 anti-RANKL antibody CDR1 (V_{L}) | 11 |
| SEQ ID NO: 33 | EP 1257648 anti-RANKL antibody CDR2 (V_{L}) | 7 |
| SEQ ID NO: 34 | EP 1257648 anti-RANKL antibody CDR3 (V_{L}) | 5 |
| SEQ ID NO: 35 | EP 1257648 anti-RANKL antibody heavy chain | 230 |
| SEQ ID NO: 36 | Antigen-binding fragment of SEQ ID NO: 35 | 126 |
| SEQ ID NO: 37 | EP 1257648 anti-RANKL antibody light chain | 215 |
| SEQ ID NO: 38 | Antigen-binding fragment of SEQ ID NO: 37 | 103 |
| SEQ ID NO: 39 | EP 1257648 anti-RANKL antibody CDR1 (V_{H}) | 6 |
| SEQ ID NO: 40 | EP 1257648 anti-RANKL antibody CDR2 (V_{H}) | 17 |
| SEQ ID NO: 41 | EP 1257648 anti-RANKL antibody CDR3 (V_{H}) | 17 |
| SEQ ID NO: 42 | EP 1257648 anti-RANKL antibody CDR1 (V_{L}) | 11 |
| SEQ ID NO: 43 | EP 1257648 anti-RANKL antibody CDR2 (V_{L}) | 7 |
| SEQ ID NO: 44 | EP 1257648 anti-RANKL antibody CDR3 (V_{L}) | 5 |
| SEQ ID NO: 45 | EP 1257648 anti-RANKL antibody heavy chain | 230 |
| SEQ ID NO: 46 | Antigen-binding fragment of SEQ ID NO: 35 | 126 |
| SEQ ID NO: 47 | EP 1257648 anti-RANKL antibody light chain | 215 |
| SEQ ID NO: 48 | Antigen-binding fragment of SEQ ID NO: 37 | 103 |
| SEQ ID NO: 49 | Anti-RANKL antibody CDR1 (V_{L}) | 11 |
| SEQ ID NO: 50 | Anti-RANKL antibody CDR1 (V_{L}) | 11 |
| SEQ ID NO: 51 | Anti-RANKL antibody CDR1 (V_{L}) | 12 |
| SEQ ID NO: 52 | Anti-RANKL antibody CDR1 (V_{L}) | 9 |
| SEQ ID NO: 53 | Anti-RANKL antibody CDR2 (V_{L}) | 7 |
| SEQ ID NO: 54 | Anti-RANKL antibody CDR2 (V_{L}) | 7 |
| SEQ ID NO: 55 | Anti-RANKL antibody CDR2 (V_{L}) | 7 |
| SEQ ID NO: 56 | Anti-RANKL antibody CDR2 (V_{L}) | 7 |
| SEQ ID NO: 57 | Anti-RANKL antibody CDR3 (V_{L}) | 5 |
| SEQ ID NO: 58 | Anti-RANKL antibody CDR3 (V_{L}) | 5 |
| SEQ ID NO: 59 | Anti-RANKL antibody CDR3 (V_{L}) | 5 |
| SEQ ID NO: 60 | Anti-RANKL antibody CDR3 (V_{L}) | 11 |
| SEQ ID NO: 61 | Anti-RANKL antibody CDR1 (V_{H}) | 5 |
| SEQ ID NO: 62 | Anti-RANKL antibody CDR1 (V_{H}) | 5 |
| SEQ ID NO: 63 | Anti-RANKL antibody CDR1 (V_{H}) | 5 |
| SEQ ID NO: 64 | Anti-RANKL antibody CDR2 (V_{H}) | 17 |
| SEQ ID NO: 65 | Anti-RANKL antibody CDR2 (V_{H}) | 17 |
| SEQ ID NO: 66 | Anti-RANKL antibody CDR2 (V_{H}) | 17 |
| SEQ ID NO: 67 | Anti-RANKL antibody CDR3 (V_{H}) | 17 |
| SEQ ID NO: 68 | Anti-RANKL antibody CDR3 (V_{H}) | 7 |
| SEQ ID NO: 69 | Anti-RANKL antibody CDR3 (V_{H}) | 14 |
| SEQ ID NO: 70 | Newa et al. anti-RANKL antibody CDR1 (V_{H}) | 7 |
| SEQ ID NO: 71 | Newa et al. anti-RANKL antibody CDR2 (V_{H}) | 5 |
| SEQ ID NO: 72 | Newa et al. anti-RANKL antibody CDR3 (V_{H}) | 7 |
| SEQ ID NO: 73 | Newa et al. anti-RANKL antibody CDR1 (V_{L}) | 11 |
| SEQ ID NO: 74 | Newa et al. anti-RANKL antibody CDR2 (V_{L}) | 7 |
| SEQ ID NO: 75 | Newa et al. anti-RANKL antibody CDR3 (V_{L}) | 9 |
| SEQ ID NO: 76 | Newa et al. anti-RANKL antibody V_{H} | 115 |
| SEQ ID NO: 77 | Newa et al. anti-RANKL antibody V_{H} | 116 |
| SEQ ID NO: 78 | Antigen-binding fragment of SEQ ID NO: 76 | 113 |
| SEQ ID NO: 79 | Antigen-binding fragment of SEQ ID NO: 77 | 114 |
| SEQ ID NO: 80 | Newa et al. anti-RANKL antibody V_{L} | 111 |
| SEQ ID NO: 81 | Antigen-binding fragment of SEQ ID NO: 80 | 107 |
| SEQ ID NO: 82 | Nivolumab CDR1 (V_{H}) | 5 |
| SEQ ID NO: 83 | Nivolumab CDR2 (V_{H}) | 17 |
| SEQ ID NO: 84 | Nivolumab CDR3 (V_{H}) | 5 |
| SEQ ID NO: 85 | Nivolumab CDR1 (V_{L}) | 11 |
| SEQ ID NO: 86 | Nivolumab CDR2 (V_{L}) | 7 |
| SEQ ID NO: 87 | Nivolumab CDR3 (V_{L}) | 9 |
| SEQ ID NO: 88 | Nivolumab heavy chain | 440 |
| SEQ ID NO: 89 | Nivolumab V_{H} | 113 |
| SEQ ID NO: 90 | Nivolumab light chain | 214 |
| SEQ ID NO: 91 | Nivolumab V_{L} | 107 |
| SEQ ID NO: 92 | Pembrolizumab CDR1 (V_{H}) | 5 |
| SEQ ID NO: 93 | Pembrolizumab CDR2 (V_{H}) | 17 |
| SEQ ID NO: 94 | Pembrolizumab CDR3 (V_{H}) | 11 |
| SEQ ID NO: 95 | Pembrolizumab CDR1 (V_{L}) | 15 |
| SEQ ID NO: 96 | Pembrolizumab CDR2 (V_{L}) | 7 |
| SEQ ID NO: 97 | Pembrolizumab CDR3 (V_{L}) | 9 |
| SEQ ID NO: 98 | Pembrolizumab heavy chain | 447 |
| SEQ ID NO: 99 | Pembrolizumab V_{H} | 120 |
| SEQ ID NO: 100 | Pembrolizumab light chain | 218 |
| SEQ ID NO: 101 | Pembrolizumab V_{L} | 111 |
| SEQ ID NO: 102 | Pidilizumab CDR1 (V_{H}) | 5 |
| SEQ ID NO: 103 | Pidilizumab CDR2 (V_{H}) | 17 |
| SEQ ID NO: 104 | Pidilizumab CDR3 (V_{H}) | 8 |
| SEQ ID NO: 105 | Pidilizumab CDR1 (V_{L}) | 10 |
| SEQ ID NO: 106 | Pidilizumab CDR2 (V_{L}) | 7 |
| SEQ ID NO: 107 | Pidilizumab CDR3 (V_{L}) | 9 |
| SEQ ID NO: 108 | Pidilizumab heavy chain | 447 |
| SEQ ID NO: 109 | Pidilizumab V_{H} | 117 |
| SEQ ID NO: 110 | Pidilizumab light chain | 213 |
| SEQ ID NO: 111 | Pidilizumab V_{L} | 106 |
| SEQ ID NO: 112 | WO2015/026634 anti-PD-1 CDR1 (V_{L}) | 15 |
| SEQ ID NO: 113 | WO2015/026634 anti-PD-1 CDR2 (V_{L}) | 7 |
| SEQ ID NO: 114 | WO2015/026634 anti-PD-1 CDR3 (V_{L}) | 9 |
| SEQ ID NO: 115 | WO2015/026634 anti-PD-1 CDR1 (V_{H}) | 5 |
| SEQ ID NO: 116 | WO2015/026634 anti-PD-1 CDR2 (V_{H}) | 17 |
| SEQ ID NO: 117 | WO2015/026634 anti-PD-1 CDR3 (V_{H}) | 11 |
| SEQ ID NO: 118 | WO2015/026634 anti-PD-1 CDR1 (V_{L}) | 15 |
| SEQ ID NO: 119 | WO2015/026634 anti-PD-1 CDR2 (V_{L}) | 7 |
| SEQ ID NO: 120 | WO2015/026634 anti-PD-1 CDR3 (V_{L}) | 9 |
| SEQ ID NO: 121 | WO2015/026634 anti-PD-1 CDR1 (V_{H}) | 5 |
| SEQ ID NO: 122 | WO2015/026634 anti-PD-1 CDR2 (V_{H}) | 17 |
| SEQ ID NO: 123 | WO2015/026634 anti-PD-1 CDR3 (V_{H}) | 11 |
| SEQ ID NO: 124 | WO2015/026634 anti-PD-1 V_{H} | 120 |
| SEQ ID NO: 125 | WO2015/026634 anti-PD-1 V_{L} | 111 |
| SEQ ID NO: 126 | WO2015/026634 anti-PD-1 V_{L} | 109 |
| SEQ ID NO: 127 | WO2015/026634 anti-PD-1 V_{L} | 111 |
| SEQ ID NO: 128 | WO2015/026634 anti-PD-1 heavy chain | 447 |
| SEQ ID NO: 129 | WO2015/026634 anti-PD-1 light chain | 218 |
| SEQ ID NO: 130 | WO2015/026634 anti-PD-1 light chain | 217 |
| SEQ ID NO: 131 | WO2015/026634 anti-PD-1 light chain | 218 |
| SEQ ID NO: 132 | Durvalumab CDR1 (V_{H}) | 5 |
| SEQ ID NO: 133 | Durvalumab CDR2 (V_{H}) | 16 |
| SEQ ID NO: 134 | Durvalumab CDR3 (V_{H}) | 12 |
| SEQ ID NO: 135 | Durvalumab CDR1 (V_{L}) | 12 |
| SEQ ID NO: 136 | Durvalumab CDR2 (V_{L}) | 11 |
| SEQ ID NO: 137 | Durvalumab CDR3 (V_{L}) | 9 |
| SEQ ID NO: 138 | Durvalumab heavy chain | 449 |
| SEQ ID NO: 139 | Durvalumab V_{H} | 120 |
| SEQ ID NO: 140 | Durvalumab light chain | 215 |
| SEQ ID NO: 141 | Durvalumab V_{L} | 108 |
| SEQ ID NO: 142 | Atezolizumab CDR1 (V_{H}) | 10 |
| SEQ ID NO: 143 | Atezolizumab CDR2 (V_{H}) | 18 |
| SEQ ID NO: 144 | Atezolizumab CDR3 (V_{H}) | 9 |
| SEQ ID NO: 145 | Atezolizumab CDR1 (V_{L}) | 11 |
| SEQ ID NO: 146 | Atezolizumab CDR2 (V_{L}) | 7 |
| SEQ ID NO: 147 | Atezolizumab CDR3 (V_{L}) | 9 |
| SEQ ID NO: 148 | Atezolizumab heavy chain | 448 |
| SEQ ID NO: 149 | Atezolizumab V_{H} | 118 |
| SEQ ID NO: 150 | Atezolizumab light chain | 214 |
| SEQ ID NO: 151 | Atezolizumab V_{L} | 107 |
| SEQ ID NO: 152 | Avelumab CDR1 (V_{H}) | 5 |
| SEO ID NO: 153 | Avelumab CDR2 (V_{H}) | 17 |
| SEQ ID NO: 154 | Avelumab CDR3 (V_{H}) | 11 |
| SEQ ID NO: 155 | Avelumab CDR1 (V_{L}) | 14 |
| SEQ ID NO: 156 | Avelumab CDR2 (V_{L}) | 7 |
| SEQ ID NO: 157 | Avelumab CDR3 (V_{L}) | 9 |
| SEQ ID NO: 158 | Avelumab heavy chain | 450 |
| SEQ ID NO: 159 | Avelumab V_{H} | 120 |
| SEQ ID NO: 160 | Avelumab light chain | 216 |
| SEQ ID NO: 161 | Avelumab V_{L} | 110 |
| SEQ ID NO: 162 | Ipilimumab CDR1 (V_{H}) | 5 |
| SEQ ID NO: 163 | Ipilimumab CDR2 (V_{H}) | 17 |
| SEQ ID NO: 164 | Ipilimumab CDR3 (V_{H}) | 9 |
| SEQ ID NO: 165 | Ipilimumab CDR1 (V_{L}) | 12 |
| SEQ ID NO: 166 | Ipilimumab CDR2 (V_{L}) | 7 |
| SEQ ID NO: 167 | Ipilimumab CDR3 (V_{L}) | 9 |
| SEQ ID NO: 168 | Ipilimumab heavy chain | 448 |
| SEQ ID NO: 169 | Ipilimumab V_{H} | 118 |
| SEQ ID NO: 170 | Ipilimumab light chain | 215 |
| SEQ ID NO: 171 | Ipilimumab V_{L} | 108 |
| SEQ ID NO: 172 | Tremelimumab CDR1 (V_{H}) | 10 |
| SEQ ID NO: 173 | Tremelimumab CDR2 (V_{H}) | 15 |
| SEQ ID NO: 174 | Tremelimumab CDR3 (V_{H}) | 16 |
| SEQ ID NO: 175 | Tremelimumab CDR1 (V_{L}) | 11 |
| SEQ ID NO: 176 | Tremelimumab CDR2 (V_{L}) | 7 |
| SEQ ID NO: 177 | Tremelimumab CDR3 (V_{L}) | 9 |
| SEQ ID NO: 178 | Tremelimumab heavy chain | 451 |
| SEQ ID NO: 179 | Tremelimumab V_{H} | 118 |
| SEQ ID NO: 180 | Tremelimumab light chain | 214 |
| SEQ ID NO: 181 | Tremelimumab V_{L} | 107 |
| SEQ ID NO: 182 | Native human B7-H3 (UniProt accession no. Q5ZPR3) | 534 |
| SEQ ID NO: 183 | Enoblituzumab CDR1 (V_{H}) | 4 |
| SEQ ID NO: 184 | Enoblituzumab CDR2 (V_{H}) | 16 |
| SEQ ID NO: 185 | Enoblituzumab CDR3 (V_{H}) | 13 |
| SEQ ID NO: 186 | Enoblituzumab CDR1 (V_{L}) | 11 |
| SEQ ID NO: 187 | Enoblituzumab CDR2 (V_{L}) | 7 |
| SEQ ID NO: 188 | Enoblituzumab CDR3 (V_{L}) | 9 |
| SEQ ID NO: 189 | Enoblituzumab heavy chain | 451 |
| SEQ ID NO: 190 | Enoblituzumab V_{H} | 121 |
| SEQ ID NO: 191 | Enoblituzumab light chain | 214 |
| SEQ ID NO: 192 | Enoblituzumab V_{L} | 107 |
| SEQ ID NO: 193 | Native human IDO (UniProt accession no. P14902) | 403 |
| SEQ ID NO: 194 | Human mature KIR2-DL1 (UniProt accession no. P43626) | 327 |
| SEQ ID NO: 195 | Lirilumab CDR1 (V_{H}) | 5 |
| SEQ ID NO: 196 | Lirilumab CDR2 (V_{H}) | 16 |
| SEQ ID NO: 197 | Lirilumab CDR3 (V_{H}) | 14 |
| SEQ ID NO: 198 | Lirilumab CDR1 (V_{L}) | 11 |
| SEQ ID NO: 199 | Lirilumab CDR2 (V_{L}) | 7 |
| SEQ ID NO: 200 | Lirilumab CDR3 (V_{L}) | 9 |
| SEQ ID NO: 201 | Lirilumab heavy chain | 450 |
| SEQ ID NO: 202 | Lirilumab V_{H} | 123 |
| SEQ ID NO: 203 | Lirilumab light chain | 214 |
| SEQ ID NO: 204 | Lirilumab V_{L} | 109 |
| SEQ ID NO: 205 | Human mature LAG-3 (UniProt accession no. P18627) | 503 |
| SEQ ID NO: 206 | BMS-986016 CDR1 (V_{H}) | 5 |
| SEQ ID NO: 207 | BMS-986016 CDR2 (V_{H}) | 16 |
| SEQ ID NO: 208 | BMS-986016 CDR3 (V_{H}) | 12 |
| SEQ ID NO: 209 | BMS-986016 CDR1 (V_{L}) | 11 |
| SEQ ID NO: 210 | BMS-986016 CDR2 (V_{L}) | 7 |
| SEQ ID NO: 211 | BMS-986016 CDR3 (V_{L}) | 9 |
| SEQ ID NO: 212 | BMS-986016 heavy chain | 447 |
| SEQ ID NO: 213 | BMS-986016 V_{H} | 120 |
| SEQ ID NO: 214 | BMS-986016 light chain | 214 |
| SEQ ID NO: 215 | BMS-986016 V_{L} | 107 |
| SEQ ID NO: 216 | Anti-RANKL-anti-PD-1 diabody | 477 |
| SEQ ID NO: 217 | Anti-RANKL-anti-PD-1 diabody | 477 |
| SEQ ID NO: 218 | Anti-RANKL-anti-PD-1 diabody | 477 |
| SEQ ID NO: 219 | Anti-RANKL-anti-PD-1 diabody | 489 |
| SEQ ID NO: 220 | Anti-RANKL-anti-PD-1 diabody | 489 |
| SEQ ID NO: 221 | Anti-RANKL-anti-PD-1 diabody | 488 |
| SEQ ID NO: 222 | Anti-RANKL-anti-PD-1 diabody | 488 |
| SEQ ID NO: 223 | Anti-RANKL-anti-PD-1 diabody | 488 |
| SEQ ID NO: 224 | Anti-RANKL-anti-PD-L1 diabody | 486 |
| SEQ ID NO: 225 | Anti-RANKL-anti-PD-L1 diabody | 486 |
| SEQ ID NO: 226 | Anti-RANKL-anti-PD-L1 diabody | 485 |
| SEQ ID NO: 227 | Anti-RANKL-anti-PD-L1 diabody | 485 |
| SEQ ID NO: 228 | Anti-RANKL-anti-PD-L1 diabody | 483 |
| SEQ ID NO: 229 | Anti-RANKL-anti-PD-L1 diabody | 483 |
| SEQ ID NO: 230 | Anti-RANKL-anti-PD-L1 diabody | 482 |
| SEQ ID NO: 231 | Anti-RANKL-anti-PD-L1 diabody | 482aa |
| SEQ ID NO: 232 | Anti-RANKL-anti-CTLA4 diabody | 483 |
| SEQ ID NO: 233 | Anti-RANKL-anti-CTLA4 diabody | 483 |
| SEQ ID NO: 234 | Anti-RANKL-anti-CTLA4 diabody | 482 |
| SEQ ID NO: 235 | Anti-RANKL-anti-CTLA4 diabody | 482 |
| SEQ ID NO: 236 | Anti-RANKL-anti-CTLA4 diabody | 482 |
| SEQ ID NO: 237 | Anti-RANKL-anti-CTLA4 diabody | 482 |
| SEQ ID NO: 238 | Anti-RANKL-anti-CTLA4 diabody | 481 |
| SEQ ID NO: 239 | Anti-RANKL-anti-CTLA4 diabody | 481 |
| SEQ ID NO: 240 | Denosumab CrossMAb C_{H1}-C_{L} huIgG2 KNOB mutation, heavy chain | 473 |
| SEQ ID NO: 241 | Denosumab CrossMAb C_{H1}-C_{L} light chain | 225 |
| SEQ ID NO: 242 | Nivolumab IgG2 Hole mutation, heavy chain | 439 |
| SEQ ID NO: 243 | Nivolumab light chain | 214 |
| SEQ ID NO: 244 | Denosumab CrossMAb V_{H}-V_{L} huIgG2 KNOB mutation, heavy chain | 452 |
| SEQ ID NO: 245 | Denosumab CrossMAb V_{H}-V_{L} light chain | 246 |
| SEQ ID NO: 246 | Nivolumab IgG2 Hole mutation, heavy chain | 439 |
| SEQ ID NO: 247 | Nivolumab light chain | 214 |
| SEQ ID NO: 248 | Denosumab CrossMAb Fab huIgG2 KNOB mutation, heavy chain | 461 |
| SEQ ID NO: 249 | Denosumab CrossMAb Fab light chain | 237 |
| SEQ ID NO: 250 | Nivolumab IgG2 Hole mutation, heavy chain | 439 |
| SEQ ID NO: 251 | Nivolumab light chain | 214 |
| SEQ ID NO: 252 | Denosumab CrossMAb C_{H1}-C_{L} huIgG4 KNOB mutation, heavy chain | 474 |
| SEQ ID NO: 253 | Denosumab CrossMAb C_{H1}-C_{L} light chain | 225 |
| SEQ ID NO: 254 | Nivolumab IgG₄ Hole mutation, heavy chain | 440 |
| SEQ ID NO: 255 | Nivolumab light chain | 214 |
| SEQ ID NO: 256 | Denosumab CrossMAb V_{H}-V_{L} huIgG4 KNOB mutation, heavy chain | 453 |
| SEQ ID NO: 257 | Denosumab CrossMAb V_{H}-V_{L} light chain | 246 |
| SEQ ID NO: 258 | Nivolumab IgG₄ Hole mutation, heavy chain | 440 |
| SEQ ID NO: 259 | Nivolumab light chain | 214 |
| SEQ ID NO: 260 | Denosumab CrossMAb Fab huIgG4 KNOB mutation, heavy chain | 462 |
| SEQ ID NO: 261 | Denosumab CrossMAb Fab light chain | 237 |
| SEQ ID NO: 262 | Nivolumab IgG₄ Hole mutation, heavy chain | 440 |
| SEQ ID NO: 263 | Nivolumab light chain | 214 |
| SEQ ID NO: 264 | Denosumab CrossMAb C_{H1}-C_{L} huIgG1 KNOB mutation, heavy chain | 477 |
| SEQ ID NO: 265 | Denosumab CrossMAb C_{H1}-C_{L} light chain | 225 |
| SEQ ID NO: 266 | Nivolumab IgG₁ Hole mutation, heavy chain | 443 |
| SEQ ID NO: 267 | Nivolumab light chain | 214 |
| SEQ ID NO: 268 | Denosumab CrossMAb V_{H}-V_{L} huIgG1 KNOB mutation, heavy chain | 456 |
| SEQ ID NO: 269 | Denosumab CrossMAb V_{H}-V_{L} light chain | 246 |
| SEQ ID NO: 270 | Nivolumab IgG₁ Hole mutation, heavy chain | 443 |
| SEQ ID NO: 271 | Nivolumab light chain | 214 |
| SEQ ID NO: 272 | Denosumab CrossMAb Fab huIgG1 KNOB mutation, heavy chain | 465 |
| SEQ ID NO: 273 | Denosumab CrossMAb Fab light chain | 237 |
| SEQ ID NO: 274 | Nivolumab IgG₁ Hole mutation, heavy chain | 443 |
| SEQ ID NO: 275 | Nivolumab light chain | 214 |
| SEQ ID NO: 276 | RANKL/PD-1 FIT-Ig construct #1 | 655 |
| SEQ ID NO: 277 | RANKL/PD-1 FIT-Ig construct #2 | 218 |
| SEQ ID NO: 278 | RANKL/PD-1 FIT-Ig construct #3 | 214 |
| SEQ ID NO: 279 | RANKL/CTLA4 FIT-Ig construct #1 | 663 |
| SEQ ID NO: 280 | RANKL/CTLA4 FIT-Ig construct #3 | 215 |
| SEQ ID NO: 281 | RANKL/PD-L1 FIT-Ig construct #1 | 663 |
| SEQ ID NO: 282 | RANKL/PD-L1 FIT-Ig construct #3 | 214 |
| SEQ ID NO: 283 | Heavy chain IK22-5 | 135 |
| SEQ ID NO: 284 | Light chain IK22-5 | 126 |
| SEQ ID NO: 285 | Heavy chain RMP1-14 | 138 |
| SEQ ID NO: 286 | Light chain RMP1-14 | 131 |
| SEQ ID NO: 287 | IK22-5-huIgG1Fc WT Heavy chain | 465 |
| SEQ ID NO: 288 | IK22-5-huIgG1Fc WT Light chain | 232 |
| SEQ ID NO: 289 | RMP1-14 C_{H}-C_{L}- huIgG1Fc Heavy chain | 473 |
| SEQ ID NO: 290 | RMP1-14 C_{H}-C_{L}- huIgG1Fc Light chain | 233 |

### DETAILED DESCRIPTION OF THE INVENTION

### 1. Definitions

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

By “about” is meant a quantity, level, value, number, frequency, percentage, dimension, size, amount, weight or length that varies by as much 15, 14, 13, 12, 11, 10, 9, 8, 7, 6, 5, 4, 3, 2 or 1% to a reference quantity, level, value, number, frequency, percentage, dimension, size, amount, weight or length.

The terms “administration concurrently” or “administering concurrently” or “co-administering” and the like refer to the administration of a single composition containing two or more actives, or the administration of each active as separate compositions and/or delivered by separate routes either contemporaneously or simultaneously or sequentially within a short enough period of time that the effective result is equivalent to that obtained when all such actives are administered as a single composition. By “simultaneously” is meant that the active agents are administered at substantially the same time, and desirably together in the same formulation. By “contemporaneously” it is meant that the active agents are administered closely in time, e.g., one agent is administered within from about one minute to within about one day before or after another. Any contemporaneous time is useful. However, it will often be the case that when not administered simultaneously, the agents will be administered within about one minute to within about eight hours and suitably within less than about one to about four hours. When administered contemporaneously, the agents are suitably administered at the same site on the subject. The term “same site” includes the exact location, but can be within about 0.5 to about 15 centimeters, preferably from within about 0.5 to about 5 centimeters. The term “separately” as used herein means that the agents are administered at an interval, for example at an interval of about a day to several weeks or months. The active agents may be administered in either order. The term “sequentially” as used herein means that the agents are administered in sequence, for example at an interval or intervals of minutes, hours, days or weeks. If appropriate the active agents may be administered in a regular repeating cycle.

As used herein, “and/or” refers to and encompasses any and all possible combinations of one or more of the associated listed items, as well as the lack of combinations when interpreted in the alternative (or).

The term “antagonist” is used in the broadest sense, and includes any molecule that partially or fully blocks, inhibits, stops, diminishes, reduces, impedes, impairs or neutralizes one or more biological activities or functions of RANKL or an ICM such as but not limited to binding, signaling, formation of a complex, proliferation, migration, invasion, survival or viability, in any setting including, in vitro, in situ, or in vivo. Likewise, the terms “antagonize”, “antagonizing” and the like are used interchangeably herein to refer to blocking, inhibiting stopping, diminishing, reducing, impeding, impairing or neutralizing an activity or function as described for example above and elsewhere herein. By way of example, “antagonize” can refer to a decrease of about 10%, 20%, 30%, 40%, 50%, 60%, 70%, 80%, 90% or 100% in an activity, or function .

The term “antibody”, as used herein, means any antigen-binding molecule or molecular complex comprising at least one complementarity determining region (CDR) that binds specifically to or interacts with a particular antigen (e.g., RANKL or ICM). The term “antibody” includes immunoglobulin molecules comprising four polypeptide chains, two heavy (H) chains and two light (L) chains inter-connected by disulfide bonds, as well as multimers thereof (e.g., IgM). Each heavy chain comprises a heavy chain variable region (which may be abbreviated as HCVR or V_{H}) and a heavy chain constant region. The heavy chain constant region comprises three domains, C_{H1}, C_{H2} and C_{H3}. Each light chain comprises a light chain variable region (which may be abbreviated as LCVR or V_{L}) and a light chain constant region. The light chain constant region comprises one domain (C_{L1}). The V_{H} and V_{L} regions can be further subdivided into regions of hypervariability, termed complementarity determining regions (CDRs), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. In different embodiments of the invention, the FRs of an antibody of the invention (or antigen-binding portion thereof) may be identical to the human germline sequences, or may be naturally or artificially modified. An amino acid consensus sequence may be defined based on a side-by-side analysis of two or more CDRs.

An antibody includes an antibody of any class, such as IgG, IgA, or IgM (or sub-class thereof), and the antibody need not be of any particular class. Depending on the antibody amino acid sequence of the constant region of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant regions that correspond to the different classes of immunoglobulins are called α, δ, ε, γ, and μ, respectively. The subunit structures and three-dimensional configurations of different classes of immunoglobulins are well known.

The terms “antigen-binding fragment”, “antigen-binding portion”, “antigen-binding domain” and “antigen-binding site” are used interchangeably herein to refer to a part of an antigen-binding molecule that participates in antigen-binding. These terms include any naturally occurring, enzymatically obtainable, synthetic, or genetically engineered polypeptide or glycoprotein that specifically binds an antigen to form a complex. Antigen-binding fragments of an antibody may be derived, e.g., from full antibody molecules using any suitable standard techniques such as proteolytic digestion or recombinant genetic engineering techniques involving the manipulation and expression of DNA encoding antibody variable and optionally constant domains. Such DNA is known and/or is readily available from, e.g., commercial sources, DNA libraries (including, e.g., phage-antibody libraries), or can be synthesized. The DNA may be sequenced and manipulated chemically or by using molecular biology techniques, for example, to arrange one or more variable and/or constant domains into a suitable configuration, or to introduce codons, create cysteine residues, modify, add or delete amino acids, etc.

Non-limiting examples of antigen-binding fragments include: (i) Fab fragments; (ii) F(ab′)₂ fragments; (iii) Fd fragments; (iv) Fv fragments; (v) single-chain Fv (scFv) molecules; (vi) dAb fragments; and (vii) minimal recognition units consisting of the amino acid residues that mimic the hypervariable region of an antibody (e.g., an isolated complementarity determining region (CDR) such as a CDR3 peptide), or a constrained FR3-CDR3-FR4 peptide. Other engineered molecules, such as domain-specific antibodies, single domain antibodies, domain-deleted antibodies, chimeric antibodies, CDR-grafted antibodies, diabodies, triabodies, tetrabodies, minibodies, nanobodies (e.g. monovalent nanobodies, bivalent nanobodies, etc.), small modular immunopharmaceuticals (SMIPs), and shark variable IgNAR domains, are also encompassed within the expression “antigen-binding fragment,” as used herein.

An antigen-binding fragment of an antibody will typically comprise at least one variable domain. The variable domain may be of any size or amino acid composition and will generally comprise at least one CDR which is adjacent to or in frame with one or more framework sequences. In antigen-binding fragments having a V_{H} domain associated with a V_{L} domain, the VH and V_{L} domains may be situated relative to one another in any suitable arrangement. For example, the variable region may be dimeric and contain V_{H}-V_{H}, V_{H}-V_{L} or V_{L}-V_{L} dimers. Alternatively, the antigen-binding fragment of an antibody may contain a monomeric V_{H} or V_{L} domain.

In certain embodiments, an antigen-binding fragment of an antibody may contain at least one variable domain covalently linked to at least one constant domain. Non-limiting, exemplary configurations of variable and constant domains that may be found within an antigen-binding fragment of an antibody of the present invention include: (i) V_{H}-C_{H1}; (ii) V_{H}-C_{H2}; (iii) V_{H}-C_{H3}; (iv) V_{H}-C_{H1}-C_{H2}; (V) V_{H}-C_{H1}-C_{H2}-CH3, (VI) V_{H}-C_{H2}-C_{H3}; (yip) V_{H}-C_{L}; (Viii) V_{L}-C_{H1}; (iX) V_{L}-C_{H2}, (X) V_{L}-C_{H3}; (xi) V_{L}-C_{H1}-C_{H2}; (XII) V_{L}-C_{H1}-C_{H2}-C_{H3}; (Xiii) V_{L}-C_{H2}-C_{H3}; and (xiv) V_{L}-C_{L}. In any configuration of variable and constant domains, including any of the exemplary configurations listed above, the variable and constant domains may be either directly linked to one another or may be linked by a full or partial hinge or linker region. A hinge region may consist of at least 2 (e.g., 5, 10, 15, 20, 40, 60 or more) amino acids which result in a flexible or semi-flexible linkage between adjacent variable and/or constant domains in a single polypeptide molecule. Moreover, an antigen-binding fragment of an antibody of the present invention may comprise a homo-dimer or hetero-dimer (or other multimer) of any of the variable and constant domain configurations listed above in non-covalent association with one another and/or with one or more monomeric V_{H} or V_{L} domain (e.g., by disulfide bond(s)).

As with full antibody molecules, antigen-binding fragments may be monospecific or multispecific (e.g., bispecific). A multispecific antigen-binding fragment of an antibody will typically comprise at least two different variable domains, wherein each variable domain is capable of specifically binding to a separate antigen or to a different epitope on the same antigen. Any multispecific antigen-binding molecule format, including the exemplary bispecific antigen-binding molecule formats disclosed herein, may be adapted for use in the context of an antigen-binding fragment of an antibody of the present invention using routine techniques available in the art.

As used herein, the term “antigen” and its grammatically equivalents expressions (e.g., “antigenic”) refer to a compound, composition, or substance that may be specifically bound by the products of specific humoral or cellular immunity, such as an antibody molecule or T-cell receptor. Antigens can be any type of molecule including, for example, haptens, simple intermediary metabolites, sugars (e.g., oligosaccharides), lipids, and hormones as well as macromolecules such as complex carbohydrates (e.g., polysaccharides), phospholipids, and proteins. Common categories of antigens include, but are not limited to, viral antigens, bacterial antigens, fungal antigens, protozoa and other parasitic antigens, tumor antigens, antigens involved in autoimmune disease, allergy and graft rejection, toxins, and other miscellaneous antigens.

By “antigen-binding molecule” is meant a molecule that has binding affinity for a target antigen. It will be understood that this term extends to immunoglobulins, immunoglobulin fragments and non-immunoglobulin derived protein frameworks that exhibit antigen-binding activity. Representative antigen-binding molecules that are useful in the practice of the present invention include antibodies and their antigen-binding fragments. The term “antigen-binding molecule” includes antibodies and antigen-binding fragments of antibodies.

The term “bispecific antigen-binding molecule” refers to a multi-specific antigen-binding molecule having the capacity to bind to two distinct epitopes on the same antigen or on two different antigens. A bispecific antigen-binding molecule may be bivalent, trivalent, or tetravalent. As used herein, “valent”, “valence”, “valencies”, or other grammatical variations thereof, mean the number of antigen-binding sites in an antigen-binding molecule. These antigen recognition sites may recognize the same epitope or different epitopes. Bivalent and bispecific molecules are described in, e.g., Kostelny et al. J Immunol 148 (1992):1547, Pack and Pluckthun Biochemistry 31 (1992) 1579, Gruber et al. J Immunol (1994) 5368, Zhu et al. Protein Sci 6 (1997):781, Hu et al. Cancer Res. 56 (1996):3055, Adams et al. Cancer Res. 53 (1993):4026, and McCartney, et al. Protein Eng. 8 (1995):301 . Trivalent bispecific antigen-binding molecules and tetravalent bispecific antigen-binding molecules are also known in the art. See, e.g., Kontermann RE (ed.), Springer Heidelberg Dordrecht London New York, pp. 199-216 (2011). A bispecific antigen-binding molecule may also have valencies higher than 4 and are also within the scope of the present invention. Such antigen-binding molecules may be generated by, for example, dock and lock conjugation method. (Chang, C.-H. et al. In: Bispecific Antibodies. Kontermann RE (2011), supra).

The phrase “binds specifically” or “specific binding” refers to a binding reaction between two molecules that is at least two times the background and more typically more than 10 to 100 times background molecular associations under physiological conditions. When using one or more detectable binding agents that are proteins, specific binding is determinative of the presence of the protein, in a heterogeneous population of proteins and other biologics. Thus, under designated immunoassay conditions, the specified antigen-binding molecule bind to a particular antigenic determinant, thereby identifying its presence. Specific binding to an antigenic determinant under such conditions requires an antigen-binding molecule that is selected for its specificity to that determinant. This selection may be achieved by subtracting out antigen-binding molecules that cross-react with other molecules. A variety of immunoassay formats may be used to select antigen-binding molecules such as immunoglobulins such that they are specifically immunoreactive with a particular antigen. For example, solid-phase ELISA immunoassays are routinely used to select antibodies specifically immunoreactive with a protein (see, e.g., Harlow & Lane, Antibodies, A Laboratory Manual (1988) for a description of immunoassay formats and conditions that can be used to determine specific immunoreactivity). Methods of determining binding affinity and specificity are also well known in the art (see, for example, Harlow and Lane, supra); Friefelder, “Physical Biochemistry: Applications to biochemistry and molecular biology” (W. H. Freeman and Co. 1976)).

The term “chimeric”, when used in reference to a molecule, means that the molecule contains portions that are derived from, obtained or isolated from, or based upon two or more different origins or sources. Thus, a polypeptide is chimeric when it comprises two or more amino acid sequences of different origin and includes (1) polypeptide sequences that are not found together in nature (i.e., at least one of the amino acid sequences is heterologous with respect to at least one of its other amino acid sequences), or (2) amino acid sequences that are not naturally adjoined.

By “coding sequence” is meant any nucleic acid sequence that contributes to the code for the polypeptide product of a gene or for the final mRNA product of a gene (e.g. the mRNA product of a gene following splicing). By contrast, the term “non-coding sequence” refers to any nucleic acid sequence that does not contribute to the code for the polypeptide product of a gene or for the final mRNA product of a gene.

As used herein, the term “complementarity determining regions” (CDRs; i.e., CDR1, CDR2, and CDR3) refers to the amino acid residues of an antibody variable domain the presence of which are necessary for antigen binding. Each variable domain typically has three CDR regions identified as CDR1, CDR2 and CDR3. Each complementarity determining region may comprise amino acid residues from a “complementarity determining region” as defined for example by Kabat (i.e., about residues 24-34 (L1), 50-56 (L2) and 89-97 (L3) in the light chain variable domain and 31-35 (H1), 50-65 (H2) and 95-102 (H3) in the heavy chain variable domain; Kabat et al., Sequences of Proteins of Immunological Interest, 5th Ed. Public Health Service, National Institutes of Health, Bethesda, Md. (1991)) and/or those residues from a “hypervariable loop” (i.e., about residues 26-32 (L1), 50-52 (L2) and 91-96 (L3) in the light chain variable domain and 26-32 (H1), 53-55 (H2) and 96-101 (H3) in the heavy chain variable domain; Chothia and Lesk J. Mol. Biol. 196:901-917 (1987)). In some instances, a complementarity determining region can include amino acids from both a CDR region defined according to Kabat and a hypervariable loop.

As used herein, the term “complex” refers to an assemblage or aggregate of molecules (e.g., peptides, polypeptides, etc.) in direct and/or indirect contact with one another. In specific embodiments, “contact”, or more particularly, “direct contact” means two or more molecules are close enough so that attractive noncovalent interactions, such as Van der Waal forces, hydrogen bonding, ionic and hydrophobic interactions, and the like, dominate the interaction of the molecules. In such embodiments, a complex of molecules (e.g., a peptide and polypeptide) is formed under conditions such that the complex is thermodynamically favored (e.g., compared to a non-aggregated, or non-complexed, state of its component molecules). The term “polypeptide complex” or “protein complex,” as used herein, refers to a trimer, tetramer, pentamer, hexamer, heptamer, octamer, nonamer, decamer, undecamer, dodecamer, or higher order oligomer.

Throughout this specification, unless the context requires otherwise, the words “comprise,” “comprises” and “comprising” will be understood to imply the inclusion of a stated step or element or group of steps or elements but not the exclusion of any other step or element or group of steps or elements. Thus, use of the term “comprising” and the like indicates that the listed elements are required or mandatory, but that other elements are optional and may or may not be present. By “consisting of” is meant including, and limited to, whatever follows the phrase “consisting of”. Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements. In some embodiments, the phrase “consisting essentially of” in the context of a recited subunit sequence (e.g., amino acid sequence) indicates that the sequence may comprise at least one additional upstream subunit (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more upstream subunits; e.g., amino acids) and/or at least one additional downstream subunit (e.g., 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50 or more upstream subunits; e.g., amino acids), wherein the number of upstream subunits and the number of downstream subunits are independently selectable.

As used herein, the terms “conjugated”, “linked”, “fused” or “fusion” and their grammatical equivalents, in the context of joining together of two more elements or components or domains by whatever means including chemical conjugation or recombinant means (e.g., by genetic fusion) are used interchangeably. Methods of chemical conjugation (e.g., using heterobifunctional crosslinking agents) are known in the art.

The term “constant domains” or “constant region” as used within the current application denotes the sum of the domains of an antibody other than the variable region. The constant region is not directly involved in binding of an antigen, but exhibits various immune effector functions.

The term “construct” refers to a recombinant genetic molecule including one or more isolated nucleic acid sequences from different sources. Thus, constructs are chimeric molecules in which two or more nucleic acid sequences of different origin are assembled into a single nucleic acid molecule and include any construct that contains (1) nucleic acid sequences, including regulatory and coding sequences that are not found together in nature (i.e., at least one of the nucleotide sequences is heterologous with respect to at least one of its other nucleotide sequences), or (2) sequences encoding parts of functional RNA molecules or proteins not naturally adjoined, or (3) parts of promoters that are not naturally adjoined. Representative constructs include any recombinant nucleic acid molecule such as a plasmid, cosmid, virus, autonomously replicating polynucleotide molecule, phage, or linear or circular single stranded or double stranded DNA or RNA nucleic acid molecule, derived from any source, capable of genomic integration or autonomous replication, comprising a nucleic acid molecule where one or more nucleic acid molecules have been operably linked. Constructs of the present invention will generally include the necessary elements to direct expression of a nucleic acid sequence of interest that is also contained in the construct, such as, for example, a target nucleic acid sequence or a modulator nucleic acid sequence. Such elements may include control elements such as a promoter that is operably linked to (so as to direct transcription of) the nucleic acid sequence of interest, and often includes a polyadenylation sequence as well. Within certain embodiments of the invention, the construct may be contained within a vector. In addition to the components of the construct, the vector may include, for example, one or more selectable markers, one or more origins of replication, such as prokaryotic and eukaryotic origins, at least one multiple cloning site, and/or elements to facilitate stable integration of the construct into the genome of a host cell. Two or more constructs can be contained within a single nucleic acid molecule, such as a single vector, or can be containing within two or more separate nucleic acid molecules, such as two or more separate vectors. An “expression construct” generally includes at least a control sequence operably linked to a nucleotide sequence of interest. In this manner, for example, promoters in operable connection with the nucleotide sequences to be expressed are provided in expression constructs for expression in an organism or part thereof including a host cell. For the practice of the present invention, conventional compositions and methods for preparing and using constructs and host cells are well known to one skilled in the art, see for example, Molecular Cloning: A Laboratory Manual, 3rd edition Volumes 1, 2, and 3. J. F. Sambrook, D. W. Russell, and N. Irwin, Cold Spring Harbor Laboratory Press, 2000.

By “control element” or “control sequence” is meant nucleic acid sequences (e.g., DNA) necessary for expression of an operably linked coding sequence in a particular host cell. The control sequences that are suitable for prokaryotic cells for example, include a promoter, and optionally a cis-acting sequence such as an operator sequence and a ribosome binding site. Control sequences that are suitable for eukaryotic cells include transcriptional control sequences such as promoters, polyadenylation signals, transcriptional enhancers, translational control sequences such as translational enhancers and internal ribosome binding sites (IRES), nucleic acid sequences that modulate mRNA stability, as well as targeting sequences that target a product encoded by a transcribed polynucleotide to an intracellular compartment within a cell or to the extracellular environment.

By “corresponds to” or “corresponding to” is meant a nucleic acid sequence that displays substantial sequence identity to a reference nucleic acid sequence (e.g., at least about 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence identity to all or a portion of the reference nucleic acid sequence) or an amino acid sequence that displays substantial sequence similarity or identity to a reference amino acid sequence (e.g., at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 97, 88, 89, 90, 91, 92, 93, 94, 95, 96, 97, 98, 99% or even up to 100% sequence similarity or identity to all or a portion of the reference amino acid sequence).

“Cytotoxic T-lymphocyte-associated protein 4 (CTLA4)” (also known as ALPSS, CD, CD152, CELIAC3, CTLA-4, GRD4, GSE, IDDM12), refers to a protein receptor that, functioning as an immune checkpoint, downregulates immune responses. CTLA4 is constitutively expressed in T regulatory cells (Tregs) but only upregulated in conventional T cells after activation. It acts as an “off” switch when bound to CD80 or CD86 on the surface of antigen-presenting cells. The term “CTLA4” as used herein includes human CTLA4 (hCTLA4), variants, isoforms, and species homologs of hCTLA4, and analogs having at least one common epitope with hCTLA4. The complete hCTLA4 sequence can be found under UniProt Accession No. P16410.

The term “DART” (dual affinity retargeting reagent) refers to an immunoglobulin molecule that comprises at least two polypeptide chains that associate (especially through a covalent interaction) to form at least two epitope-binding sites, which may recognize the same or different epitopes. Each of the polypeptide chains of a DART comprise an immunoglobulin light chain variable region and an immunoglobulin heavy chain variable region, but these regions do not interact to form an epitope binding site. Rather, the immunoglobulin heavy chain variable region of one (e.g., the first) of the DART polypeptide chains interacts with the immunoglobulin light chain variable region of a different (e.g., the second) DART polypeptide chain to form an epitope binding site. Similarly, the immunoglobulin light chain variable region of one (e.g., the first) of the DART polypeptide chains interacts with the immunoglobulin heavy chain variable region of a different (e.g., the second) DART polypeptide chain to form an epitope binding site. DARTs may be monospecific, bispecific, trispecific, etc., thus being able to simultaneously bind one, two, three or more different epitopes (which may be of the same or of different antigens). DARTs may additionally be monovalent, bivalent, trivalent, tetravalent, pentavalent, hexavalent, etc., thus being able to simultaneously bind one, two, three, four, five, six or more molecules. These two attributes of DARTs (i.e., degree of specificity and valency may be combined, for example to produce bispecific antibodies (i.e., capable of binding two epitopes) that are tetravalent (i.e., capable of binding four sets of epitopes), etc. DART molecules are disclosed in more detail in International PCT Publication Nos. WO 2006/113665, WO 2008/157379, and WO 2010/080538.

By “effective amount,” in the context of treating or preventing a disease or condition (e.g., a cancer) is meant the administration of an amount of active agent to a subject, either in a single dose or as part of a series or slow release system, which is effective for the treatment or prevention of that disease or condition. The effective amount will vary depending upon the health and physical condition of the subject and the taxonomic group of individual to be treated, the formulation of the composition, the assessment of the medical situation, and other relevant factors.

As used herein, the terms “encode”, “encoding” and the like refer to the capacity of a nucleic acid to provide for another nucleic acid or a polypeptide. For example, a nucleic acid sequence is said to “encode” a polypeptide if it can be transcribed and/or translated to produce the polypeptide or if it can be processed into a form that can be transcribed and/or translated to produce the polypeptide. Such a nucleic acid sequence may include a coding sequence or both a coding sequence and a non-coding sequence. Thus, the terms “encode”, “encoding” and the like include a RNA product resulting from transcription of a DNA molecule, a protein resulting from translation of a RNA molecule, a protein resulting from transcription of a DNA molecule to form a RNA product and the subsequent translation of the RNA product, or a protein resulting from transcription of a DNA molecule to provide a RNA product, processing of the RNA product to provide a processed RNA product (e.g., mRNA) and the subsequent translation of the processed RNA product.

The terms “epitope” and “antigenic determinant” are used interchangeably herein to refer to a region of an antigen that is bound by an antigen-binding molecule or antigen-binding fragment thereof. Epitopes can be formed both from contiguous amino acids (linear epitope) or non-contiguous amino acids juxtaposed by tertiary folding of a protein (conformational epitopes). Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance (see, e.g., Morris G.E., Epitope Mapping Protocols, Meth Mol Biol, 66 (1996)). A preferred method for epitope mapping is surface plasmon resonance. Bispecific antibodies may be bivalent, trivalent, or tetravalent. When used herein in the context of bispecific antibodies, the terms “valent”, “valence”, “valencies”, or other grammatical variations thereof, mean the number of antigen binding sites in an antibody molecule. These antigen recognition sites may recognize the same epitope or different epitopes. Bivalent and bispecific molecules are described in, for example, Kostelny et al., (1992) J Immunol 148:1547; Pack and Pluckthun (1992) Biochemistry 31:1579; Hollinger et al., 1993, supra, Gruber et al., (1994) J Immunol 5368, Zhu et al., (1997) Protein Sci 6:781; Hu et al., (1996) Cancer Res 56:3055; Adams et al., (1993) Cancer Res 53:4026; and McCartney et al., (1995) Protein Eng 8:301. Trivalent bispecific antibodies and tetravalent bispecific antibodies are also known in the art (see, e.g., Kontermann R E (ed.), Springer Heidelberg Dordrecht London New York, 199-216 (2011)). A bispecific antibody may also have valencies higher than 4and are also within the scope of the present invention. Such antibodies may be generated by, for example, dock and lock conjugation method (see, Chang, C.-H. et al. In: Bispecific Antibodies. Kontermann R E (ed.), Springer Heidelberg Dordrecht London New York, pp. 199-216 (2011)).

As used herein, the terms “function,” “functional” and the like refer to a biological, enzymatic, or therapeutic function.

“Framework regions” (FR) are those variable domain residues other than the CDR residues. Each variable domain typically has four FRs identified as FR1, FR2, FR3 and FR4. If the CDRs are defined according to Kabat, the light chain FR residues are positioned at about residues 1-23 (LCFR1), 35-49 (LCFR2), 57-88 (LCFR3), and 98-107 (LCFR4) and the heavy chain FR residues are positioned about at residues 1-30 (HCFR1), 36-49 (HCFR2), 66-94 (HCFR3), and 103-113 (HCFR4) in the heavy chain residues. If the CDRs comprise amino acid residues from hypervariable loops, the light chain FR residues are positioned about at residues 1-25 (LCFR1), 33-49 (LCFR2), 53-90 (LCFR3), and 97-107 (LCFR4) in the light chain and the heavy chain FR residues are positioned about at residues 1-25 (HCFR1), 33-52 (HCFR2), 56-95 (HCFR3), and 102-113 (HCFR4) in the heavy chain residues. In some instances, when the CDR comprises amino acids from both a CDR as defined by Kabat and those of a hypervariable loop, the FR residues will be adjusted accordingly. For example, when CDRH1 includes amino acids H26-H35, the heavy chain FR1 residues are at positions 1-25 and the FR2 residues are at positions 36-49.

As used herein, the term “higher” in reference to a measurement of a cellular marker, or biomarker, refers to a statistically significant and measurable difference in the level of a biomarker measurement compared with a reference level where the biomarker measurement is greater than the reference level. The difference is suitably at least about 10%, or at least about 20%, or of at least about 30%, or of at least about 40%, or at least about 50%, or at least about 60%, or at least about 70%, or at least about 80%, or at least about 90%.

As used herein, the term “lower” in reference to a measurement of a cellular marker, or biomarker, refers to a statistically significant and measurable difference in the level of a biomarker measurement compared with a reference level where the biomarker measurement is less than the reference level. The difference is suitably at least about 10%, or at least about 20%, or of at least about 30%, or of at least about 40%, or at least about 50%, or at least about 60%, or at least about 70%, or at least about 80%, or at least about 90%.

The term “immune checkpoint molecule” includes both receptors and ligands that function as an immune checkpoint. Immune checkpoints are the immune escape mechanism to prevent the immune system from attacking its own body. Immune checkpoint receptors are present on T cells, and interact with immune checkpoint ligands expressed on antigen-presenting cells. T cells recognize an antigen presented on the MHC molecule and are activated to generate an immune reaction, whereas an interaction between immune checkpoint receptor and ligand that occurs in parallel with the above controls the activation of T cells. Exemplary immune checkpoint molecule include, without limitation, PD-1, PD-L1, PD-L2, CTLA-4, A2AR, A2BR, B7-H3 CD276, VTCN1, BTLA, IDO, KIR, LAG3, TIM-3, VISTA, CD73, CD96, CD155, DNAM-1, CD112, CRTAM, TNFRS4 (0X40, CD134), TNFSF4 (OX4OL), CD244, CD160, GITR, GITRL, ICOS, GAL-9, 4-1BBL (CD137L), 4-1BB (CD137), CD70, CD27L, CD28, B7-1 (CD80), B7-2 (CD86), SIRP-1, IAP (CD47), BLAST-1 (CD48), CD244; CD40, CD4OL, HVEM, TMIGD2, HHLA2, VEGI, TNFRS25, ICOLG (B7RP1) and TIGIT. In specific embodiments, the immune checkpoint molecule is PD-1, PD-L1 or CTLA-4.

The term “immune effector cells” in the context of the present invention relates to cells which exert effector functions during an immune reaction. For example, such cells secrete cytokines and/or chemokines, kill microbes, secrete antibodies, recognize infected or cancerous cells, and optionally eliminate such cells. For example, immune effector cells comprise T cells (cytotoxic T cells, helper T cells, tumor infiltrating T cells), B-cells, natural killer (NK) cells, lymphokine-activated killer (LAK) cells, neutrophils, macrophages, and dendritic cells.

The term “immune effector functions” in the context of the present invention includes any functions mediated by components of the immune system that result, for example, in the killing of virally infected cells or tumor cells, or in the inhibition of tumor growth and/or inhibition of tumor development, including inhibition of tumor dissemination and metastasis. Preferably, the immune effector functions in the context of the present invention are T-cell mediated effector functions. Such functions comprise in the case of a helper T-cell (CD4⁺ T-cell) the recognition of an antigen or an antigen peptide derived from an antigen in the context of MHC class II molecules by T-cell receptors, the release of cytokines and/or the activation of CD8⁺ lymphocytes (CTLs) and/or B-cells, and in the case of CTL the recognition of an antigen or an antigen peptide derived from an antigen in the context of MHC class I molecules by T-cell receptors, the elimination of cells presented in the context of MHC class I molecules, i.e., cells characterized by presentation of an antigen with class I MHC, for example, via apoptosis or perforin-mediated cell lysis, production of cytokines such as IFN-γ and TNF-a, and specific cytolytic killing of antigen expressing target cells.

The term “immune system” refers to cells, molecular components and mechanisms, including antigen-specific and non-specific categories of the adaptive and innate immune systems, respectively, that provide a defense against damage and insults and matter, the latter comprised of antigenic molecules, including but not limited to tumors, pathogens, and self-reactive cells. By “adaptive immune system” refers to antigen-specific cells, molecular components and mechanisms that emerge over several days, and react with and remove a specific antigen. The adaptive immune system develops throughout a host's lifetime. The adaptive immune system is based on leukocytes, and is divided into two major sections: the humoral immune system, which acts mainly via immunoglobulins produced by B cells, and the cell-mediated immune system, which functions mainly via T cells.

By “linker” is meant a molecule or group of molecules (such as a monomer or polymer) that connects two molecules and often serves to place the two molecules in a desirable configuration. In specific embodiments, a “peptide linker” refers to an amino acid sequence that connects two proteins, polypeptides, peptides, domains, regions, or motifs and may provide a spacer function compatible with the spacing of antigen-binding fragments so that they can bind specifically to their cognate epitopes). In certain embodiments, a linker is comprised of about two to about 35 amino acids, for instance, or about four to about 20 amino acids or about eight to about 15 amino acids or about 15 to about 25 amino acids.

As used herein, the term “microenvironment” refers to the connective, supportive framework of a biological cell, tissue, or organ. As used herein, the term “tumor microenvironment” or “TME” refers to any and all elements of the tumor milieu that creates a structural and or functional environment for the malignant process to survive and/or expand and/or spread. Generally, the term “tumor microenvironment” or “TME” refers to the cellular environment in which the tumor exists, including the area immediately surrounding fibroblasts, leukocytes and endothelial cells and the extracellular matrix (ECM). Accordingly, cells of a tumor microenvironment comprise malignant cells in association with non-malignant cells that support their growth and survival. The non-malignant cells, also called stromal cells, occupy or accumulate in the same cellular space as malignant cells, or the cellular space adjacent or proximal to malignant cells, which modulate tumor cell growth or survival. The term “stromal cells” include fibroblasts, leukocytes and vascular cells. Non-malignant cells of the tumor microenvironment include fibroblasts, epithelial cells, vascular cells (including blood and lymphatic vascular endothelial cells and pericytes), resident and/or recruited inflammatory and immune (e.g., macrophages, dendritic cells, granulocytes, lymphocytes, etc.). These cells and especially activated fibroblasts actively participate in metastasis development.

The term “monoclonal antibody” (Mab), as used herein, refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Kohler et al., Nature 256: 495 (1975), and as modified by the somatic hybridization method as set forth above; or may be made by other recombinant DNA methods (such as those described in U.S. Patent No. 4,816,567).

The term “multispecific antigen-binding molecule” is used in its broadest sense and specifically covers an antigen-binding molecule with specificity for at least two (e.g., 2, 3, 4, etc.) different epitopes (i.e., is capable of specifically binding to two, or more, different epitopes on one antigen or is capable of specifically binding to epitopes on two, or more, different antigens).

“Negative”, “positive” and “low” expression levels as they apply to markers are defined as follows. Cells with negative expression (i.e., “-”) or that “lack expression” are defined herein as those cells expressing less than, or equal to, the 95th percentile of expression observed with an isotype control antibody in the channel of fluorescence in the presence of the complete antibody staining cocktail labeling for other proteins of interest in additional channels of fluorescence emission. Those skilled in the art will appreciate that this procedure for defining negative events is referred to as “fluorescence minus one,” or “FMO,” staining. Cells with expression greater than the 95th percentile of expression observed with an isotype control antibody using the FMO staining procedure described above are herein defined as “positive” (i.e., “+”). There are various populations of cells broadly defined as “positive.” For example, cells with low expression (i.e., “low” or “low”) are generally defined as those cells with observed expression above the 95th percentile determined using FMO staining with an isotype control antibody and within one standard deviation of the 95th percentile of expression observed with an isotype control antibody using the FMO staining procedure described above. The term “low” or “lo” in relation to an ICM (e.g., PD-1, PD-L1, etc.) refers to a cell or population of cells (e.g., Treg cells, including T cells in the tumor microenvironment) that expresses the ICM at a lower level than one or more other distinct cells or populations of cells (e.g., immune effector cells such as T-cells, B-cells, natural killer (NK) cells, NK T (NKT) cells, monocytes, macrophages, and dendritic cells (DCs); as well as tumor cells). For example, it is known that in the tumor microenvironment CTLA4 is expressed at a significantly higher level on Treg than PD-1 and PD-1 is expressed at a significantly higher level on immune effector cells, including effector T cells, than on Treg (Jacobs et al., 2009. Neuro-Oncology 11(4): 394-402).

The term “operably connected” or “operably linked” as used herein refers to a juxtaposition wherein the components so described are in a relationship permitting them to function in their intended manner. For example, a regulatory sequence (e.g., a promoter) “operably linked” to a nucleotide sequence of interest (e.g., a coding and/or non-coding sequence) refers to positioning and/or orientation of the control sequence relative to the nucleotide sequence of interest to permit expression of that sequence under conditions compatible with the control sequence. The control sequences need not be contiguous with the nucleotide sequence of interest, so long as they function to direct its expression. Thus, for example, intervening non-coding sequences (e.g., untranslated, yet transcribed, sequences) can be present between a promoter and a coding sequence, and the promoter sequence can still be considered “operably linked” to the coding sequence. Likewise, “operably connecting” a first antigen-binding fragment to a second antigen-binding fragment encompasses positioning and/or orientation of the antigen-binding fragments in such a way as to permit binding of each antigen-binding fragment to its cognate epitope.

By “pharmaceutically acceptable carrier” is meant a pharmaceutical vehicle comprised of a material that is not biologically or otherwise undesirable, i.e., the material may be administered to a subject along with the selected active agent without causing any or a substantial adverse reaction. Carriers may include excipients and other additives such as diluents, detergents, coloring agents, wetting or emulsifying agents, pH buffering agents, preservatives, and the like.

“Programmed Death-1 (PD-1)” (also known as CD279, PD1, SLEB2, hPD-1, hPD-I, and hSLE1) refers to an immuno-inhibitory receptor belonging to the CD28 family. PD-1 is expressed predominantly on previously activated T cells in vivo, and binds to two ligands, PD-L1 and PD-L2. The term “PD-1” includes fragments of PD-1, as well as related polypeptides, which include, but are not limited to, allelic variants, splice variants, derivative variants, substitution variants, deletion variants, and/or insertion variants, fusion polypeptides, and interspecies homologs. In certain embodiments, a PD-1 polypeptide includes terminal residues, such as, but not limited to, leader sequence residues, targeting residues, amino terminal methionine residues, lysine residues, tag residues and/or fusion protein residues. In preferred embodiments, “PD-1” includes human PD-1 (hPD-1), variants, isoforms, and species homologs of hPD-1, and analogs having at least one common epitope with hPD-1. The complete hPD-1 sequence can be found under GenBank Accession No. U64863.

“Programmed Death Ligand-1 (PD-L1)” (also known as CD274, B7-H, B7H1, PDCD1L1, PDCD1LG1, PDL1 and CD274 molecule) is one of two cell surface glycoprotein ligands for PD-1 (the other being PD-L2) that downregulate T cell activation and cytokine secretion upon binding to PD-1. The term “PD-L1” includes fragments of PD-L1, as well as related polypeptides, which include, but are not limited to, allelic variants, splice variants, derivative variants, substitution variants, deletion variants, and/or insertion variants, fusion polypeptides, and interspecies homologs. In certain embodiments, a PD-1 polypeptide includes terminal residues, such as, but not limited to, leader sequence residues, targeting residues, amino terminal methionine residues, lysine residues, tag residues and/or fusion protein residues. In preferred embodiments, “PD-L1” as used herein includes human PD-L1 (hPD-L1), variants, isoforms, and species homologs of hPD-L1, and analogs having at least one common epitope with hPD-L1. The complete hPD-L1 sequence can be found under GenBank Accession No. Q9NZQ7.

The terms “polypeptide,” “proteinaceous molecule”, “peptide” and “protein” are used interchangeably herein to refer to a polymer of amino acid residues and to variants and synthetic analogues of the same. Thus, these terms apply to amino acid polymers in which one or more amino acid residues is a synthetic non-naturally-occurring amino acid, such as a chemical analogue of a corresponding naturally-occurring amino acid, as well as to naturally-occurring amino acid polymers. These terms do not exclude modifications, for example, glycosylations, acetylations, phosphorylations and the like. Soluble forms of the subject proteinaceous molecules are particularly useful. Included within the definition are, for example, polypeptides containing one or more analogs of an amino acid including, for example, unnatural amino acids or polypeptides with substituted linkages.

“Receptor activator of NF-κB ligand (RANKL)” (also known as tumor necrosis factor ligand superfamily member 11 (TNFSF11), TNF-related activation-induced cytokine (TRANCE), osteoprotegrin ligand (OPGL) and osteoclast differentiation factor (ODF)) refers to a polypeptide that inter alia promotes formation of osteoclasts through binding to receptor activator of NF-κB (RANK). The term “RANKL” includes fragments of RANKL, as well as related polypeptides, which include, but are not limited to, allelic variants, splice variants, derivative variants, substitution variants, deletion variants, and/or insertion variants, fusion polypeptides, and interspecies homologs. In certain embodiments, a RANKL polypeptide includes terminal residues, such as, but not limited to, leader sequence residues, targeting residues, amino terminal methionine residues, lysine residues, tag residues and/or fusion protein residues. The term RANKL includes human RANKL (hRANKL), variants, isoforms, and species homologs of hRANKL, and analogs having at least one common epitope with hRANKL. The complete hRANKL sequence can be found under UniProt Accession No. 014788.

“Receptor activator of NF-κB (RANK)” (also known as tumor necrosis factor receptor superfamily, member 11a, NF-κB activator, CD265, FEO, LOH18CR1, ODFR, OFE, OPTB7, OSTS, PDB2, and TRANCER) refers to a polypeptide that is a receptor for RANK-Ligand (RANKL) and part of the RANK/RANKL/osteoprotegrin (OPG) signaling pathway that regulates osteoclast differentiation and activation. It is associated with bone remodeling and repair, immune cell function, lymph node development, thermal regulation, and mammary gland development. The term “RANK” includes fragments of RANK, as well as related polypeptides, which include, but are not limited to, allelic variants, splice variants, derivative variants, substitution variants, deletion variants, and/or insertion variants, fusion polypeptides, and interspecies homologs. In certain embodiments, a RANK polypeptide includes terminal residues, such as, but not limited to, leader sequence residues, targeting residues, amino terminal methionine residues, lysine residues, tag residues and/or fusion protein residues. The term RANK includes human RANK (hRANK, variants, isoforms, and species homologs of hRANK, and analogs having at least one common epitope with hRANK. The complete hRANK sequence can be found under UniProt Accession No. Q9Y6Q6.

As used herein, “recombinant” antigen-binding molecule means any antigen-binding molecule whose production involves expression of a non-native DNA sequence encoding the desired antibody structure in an organism, non-limiting examples of which include tandem scFv (taFv or scFv₂), diabody, dAb₂/VHH2, knob-into-holes derivatives, SEED-IgG, heteroFc-scFv, Fab-scFv, scFv-Jun/Fos, Fab′-Jun/Fos, tribody, DNL-F(ab)₃, scFv₃-C_{H1}/CL, Fab-scFv₂, IgG-scFab, IgG-scFv, scFv-IgG, scFv₂-Fc, F(ab′)₂-scFv₂, scDB-Fc, scDB-CH3, Db-Fc, scFv₂-H/L, DVD-Ig, tandAb, scFv-dhlx-scFv, dAb₂-IgG, dAb-IgG, dAb-Fc-dAb, CrossMabs, MAb2, FIT-Ig, and combinations thereof.

As used herein, the term “regulatory T cell” or “Treg” refers to a T cell that negatively regulates the activation of other T cells, including effector T cells, as well as innate immune system cells. Treg cells are characterized by sustained suppression of effector T cell responses. In some aspects, the Treg is a CD4⁺ CD25⁺ Foxp3⁺ T cell.

The terms “subject”, “patient”, “host” or “individual” used interchangeably herein, refer to any subject, particularly a vertebrate subject, and even more particularly a mammalian subject, for whom therapy or prophylaxis is desired. Suitable vertebrate animals that fall within the scope of the invention include, but are not restricted to, any member of the subphylum Chordata including primates (e.g., humans, monkeys and apes, and includes species of monkeys such from the genus Macaca (e.g., cynomologus monkeys such as Macaca fascicularis, and/or rhesus monkeys (Macaca mulatta)) and baboon (Papio ursinus), as well as marmosets (species from the genus Callithrix), squirrel monkeys (species from the genus Saimiri) and tamarins (species from the genus Saguinus), as well as species of apes such as chimpanzees (Pan troglodytes)), rodents (e.g., mice rats, guinea pigs), lagomorphs (e.g., rabbits, hares), bovines (e.g., cattle), ovines (e.g., sheep), caprines (e.g., goats), porcines (e.g., pigs), equines (e.g., horses), canines (e.g., dogs), felines (e.g., cats), avians (e.g., chickens, turkeys, ducks, geese, companion birds such as canaries, budgerigars etc.), marine mammals (e.g., dolphins, whales), reptiles (snakes, frogs, lizards etc.), and fish. A preferred subject is a human in need of eliciting an immune response to a cancer. However, it will be understood that the aforementioned terms do not imply that symptoms are present.

By “treatment, ” “treat,” “treated” and the like is meant to include both prophylactic and therapeutic treatment, including but not limited to preventing, relieving, altering, reversing, affecting, inhibiting the development or progression of, ameliorating, or curing (1) a disease or condition associated with the presence or aberrant expression of a target antigen, or (2) a symptom of the disease or condition, or (3) a predisposition toward the disease or condition, including conferring protective immunity to a subject.

As used herein, the term “therapeutic combination” refers to a combination of one or more active drug substances, i.e., compounds having a therapeutic utility when administered concurrently (i.e., combination therapy). Thus, the compounds may be in the form of a single composition, suitably comprising a mixture of the compounds, or in the form of separate compositions. Typically, each such compound in the therapeutic combinations of the present invention will be present in a pharmaceutical composition comprising that compound and a pharmaceutically acceptable carrier. The compounds in a therapeutic combination of the present invention are provided in dosage forms such that the beneficial effect of each therapeutic compound is realized by the subject at the desired time.

As used herein, the term “trispecific antibody” refers to an antibody that comprises at least a first antigen-binding domain with specificity for a first epitope, a second antigen-binding domain with specificity for a second epitope, and a third antigen-binding domain with specificity for a third epitope e.g., RANKL and any two of CTLA4, PD-1, and PD-L1. The first, second, and third epitopes are not the same (i.e., are different targets (e.g., proteins)), but can all be present (e.g., co-expressed) on a single cell or on at least two cells.

The term “tumor,” as used herein, refers to any neoplastic cell growth and proliferation, whether malignant or benign, and all pre-cancerous and cancerous cells and tissues. The terms “cancer” and “cancerous” refer to or describe the physiological condition in mammals that is typically characterized in part by unregulated cell growth. As used herein, the term “cancer” refers to non-metastatic and metastatic cancers, including early stage and late stage cancers. The term “precancerous” refers to a condition or a growth that typically precedes or develops into a cancer. By “non-metastatic” is meant a cancer that is benign or that remains at the primary site and has not penetrated into the lymphatic or blood vessel system or to tissues other than the primary site. Generally, a non-metastatic cancer is any cancer that is a Stage 0, I, or II cancer, and occasionally a Stage III cancer. By “early stage cancer” is meant a cancer that is not invasive or metastatic or is classified as a Stage 0, I, or II cancer. The term “late stage cancer” generally refers to a Stage III or Stage IV cancer, but can also refer to a Stage II cancer or a substage of a Stage II cancer. One skilled in the art will appreciate that the classification of a Stage II cancer as either an early stage cancer or a late stage cancer depends on the particular type of cancer. Illustrative examples of cancer include, but are not limited to, breast cancer, prostate cancer, ovarian cancer, cervical cancer, pancreatic cancer, colorectal cancer, lung cancer, hepatocellular cancer, gastric cancer, liver cancer, bladder cancer, cancer of the urinary tract, thyroid cancer, renal cancer, carcinoma, melanoma, brain cancer, non-small cell lung cancer, squamous cell cancer of the head and neck, endometrial cancer, multiple myeloma, rectal cancer, and esophageal cancer. In an exemplary embodiment, the cancer is selected from prostate, lung, pancreatic, breast, ovarian and bone cancer.

By “vector” is meant a nucleic acid molecule, preferably a DNA molecule derived, for example, from a plasmid, bacteriophage, or plant virus, into which a nucleic acid sequence may be inserted or cloned. A vector preferably contains one or more unique restriction sites and may be capable of autonomous replication in a defined host cell including a target cell or tissue or a progenitor cell or tissue thereof, or be integrable with the genome of the defined host such that the cloned sequence is reproducible. Accordingly, the vector may be an autonomously replicating vector, i.e., a vector that exists as an extrachromosomal entity, the replication of which is independent of chromosomal replication, e.g., a linear or closed circular plasmid, an extrachromosomal element, a minichromosome, or an artificial chromosome. The vector may contain any means for assuring self-replication. Alternatively, the vector may be one which, when introduced into the host cell, is integrated into the genome and replicated together with the chromosome(s) into which it has been integrated. A vector system may comprise a single vector or plasmid, two or more vectors or plasmids, which together contain the total DNA to be introduced into the genome of the host cell, or a transposon. The choice of the vector will typically depend on the compatibility of the vector with the host cell into which the vector is to be introduced. The vector may also include a selection marker such as an antibiotic resistance gene that can be used for selection of suitable transformants. Examples of such resistance genes are well known to those of skill in the art.

Each embodiment described herein is to be applied mutatis mutandis to each and every embodiment unless specifically stated otherwise.

### 2. Abbreviations

The following abbreviations are used throughout the application:

| | |
|---|---|
| aa = | amino acid(s) |
| CDR = | complementarity determining regions |
| CTLA4 | cytotoxic T-lymphocyte-associated protein 4 |
| Fc = | constant region |
| FR = | framework |
| h = | hour |
| ICM = | immune checkpoint molecule |
| Ig = | immunoglobulin |
| MAb = | monoclonal antibody |
| PD-1 = | programmed death 1 |
| PD-L1 = | programmed death ligand 1 |
| RANKL = | receptor activator of NF-κB ligand |
| s = | seconds |
| V_{H} = | heavy chain variable domain |
| V_{L} = | light chain variable domain |

### 3. Therapeutic Combinations

The present invention provides therapeutic combinations that are useful inter alia for stimulating or augmenting an immune response to a cancer in a subject. These compositions generally employ (1) a receptor activator of NF-κB (RANK) ligand (RANKL) antagonist, and (2) at least one immune checkpoint molecule (ICM) antagonist. The compositions take advantage of the newly identified synergy between these two pathways, which results in an increased localization of CD8⁺ T-cells at the site of a tumour or cancer. Advantageously, the synergistic compositions suitably stimulate an enhancement of effector cell function, including for example, an enhanced effector T-cell function includes the production of Th1-type cytokines (e.g., IFN-γ and/or IL-2) and increased proportion of polyfunctional T-cells.

In some preferred embodiments, the antagonists (i.e., RANKL antagonist and ICM antagonist(s)) of the invention are antigen-binding molecules. Suitable antigen-binding molecules may be selected from antibodies and their antigen-binding fragments, including recombinant antibodies, monoclonal antibodies (MAbs), chimeric antibodies, humanized antibodies, human antibodies, and antigen-binding fragments of such antibodies.

For application in humans, it is often desirable to reduce immunogenicity of antibodies originally derived from other species, like mouse. This can be done by construction of chimeric antibodies, or by a process called “humanization”. In this context, a “chimeric antibody” is understood to be an antibody comprising a domain (e.g., a variable domain) derived from one species (e.g., mouse) fused to a domain (e.g., the constant domains) derived from a different species (e.g., human).

“Humanized antibodies” refer to forms of antibodies that contain sequences from non-human (e.g., murine) antibodies as well as human antibodies. Such antibodies are chimeric antibodies which contain minimal sequence derived from non-human immunoglobulin. In general, the humanized antibody will comprise substantially all of at least one, and typically two, variable domains, in which all or substantially all of the hypervariable loops correspond to those of a non-human immunoglobulin and all or substantially all of the framework (FR) regions are those of a human immunoglobulin sequence. The humanized antibody optionally also will comprise at least a portion of an immunoglobulin constant region (Fc), typically that of a human immunoglobulin (see, Jones et al., Nature 321:522-525 (1986); Riechmann et al., Nature 332:323-329 (1988); and Presta, Curr Op Struct Biol 2:593-596 (1992)). Humanization can be essentially performed following the method of Winter et al. (see, Jones et al., supra; Riechmann et al., supra); and Verhoeyen et al., Science 239:1534-1536 (1988)), by substituting rodent CDRs or CDR sequences for the corresponding sequences of a human antibody. Furthermore, technologies have been developed for creating antibodies based on sequences derived from the human genome, for example by phage display or using transgenic animals (see, International Patent Publication No. WO 90/05144; Marks et al., (1991) By-passing immunisation. Human antibodies from V-gene libraries displayed on phage, J Mol Biol, 222, 581-597; Knappik et al., J Mol Biol 296: 57-86, 2000; Carmen and Jermutus, Concepts in antibody phage display, Briefings in Functional Genomics and Proteomics 2002 1(2):189-203; Lonberg and Huszar, Human antibodies from transgenic mice. Int Rev Immunol 1995; 13(1):65-93; Bruggemann and Taussig, Production of human antibody repertoires in transgenic mice, Curr Opin Biotechnol 1997 8(4): 455-8). Such antibodies are “human antibodies” in the context of the present invention.

The present invention also contemplates synthetic or recombinant antigen-binding molecules, production of which involves expression of a non-native DNA sequence encoding the desired antibody structure in an organism. In some embodiments, the synthetic or recombinant antigen-binding molecules are multispecific antigen-binding molecules, representative examples of which include tandem scFv (taFv or scFv₂), diabody, dAbz/VHH2, knobs-into-holes derivatives, SEED-IgG, heteroFc-scFv, Fab-scFv, scFv-Jun/Fos, Fab′-Jun/Fos, tribody, DNL-F(ab)₃, scFv₃-C_{H}1/CL, Fab-scFv₂, IgG-scFab, IgG-scFv, scFv-IgG, scFv₂-Fc, F(ab′)₂-scFv₂, scDB-Fc, scDb-C_{H}3, Db-Fc, scFv₂-H/L, DVD-Ig, tandAb, scFv-dhlx-scFv, dAb₂-IgG, dAb-IgG, dAb-Fc-dAb, and combinations thereof. In specific embodiments, the synthetic or recombinant antigen-binding molecules are selected from IgG-like antibodies (e.g., triomab/quadroma, Trion Pharma/Fresenius Biotech; knobs-into-holes, Genentech; CrossMAbs, Roche; electrostatically matched antibodies, AMGEN; LUZ-Y, Genentech; strand exchange engineered domain (SEED) body, EMD Serono; biolonic, Merus; and Fab-exchanged antibodies, Genmab), symmetric IgG-like antibodies (e.g., dual targeting (DT)-Ig, GSK/Domantis; two-in-one antibody, Genentech; crosslinked MAbs, karmanos cancer center; MAb2, F-star; and Coy X-body, Coy X/Pfizer), IgG fusions (e.g., dual variable domain (DVD)-Ig, Abbott; IgG-like bispecific antibodies, Eli Lilly; Ts2Ab, Medimmune/AZ; BsAb, ZymoGenetics; HERCULES, Biogen Idec; TvAb, Roche) Fc fusions (e.g., ScFv/Fc fusions, Academic Institution; SCORPION, Emergent BioSolutions/Trubion, ZymoGenetics/BMS; dual affinity retargeting technology (Fc-DART), MacroGenics; dual (ScFv)₂-Fab, National Research Center for Antibody Medicine) Fab fusions (e.g., F(ab)₂, Medarex/AMGEN; dual-action or Bis-Fab, Genentech; Dock-and-Lock (DNL), ImmunoMedics; bivalent bispecific, Biotechnol; and Fab-Fv, UCB-Celltech), ScFv- and diabody-based antibodies (e.g., bispecific T cell engagers (BiTEs), Micromet; tandem diabodies (Tandab), Affimed; DARTs, MacroGenics; Single-chain diabody, Academic; TCR-like antibodies, AIT, Receptor Logics; human serum albumin ScFv fusion, Merrimack; and COMBODIES, Epigen Biotech), IgG/non-IgG fusions (e.g., immunocytokins, EMDSerono, Philogen, ImmunGene, ImmunoMedics; superantigen fusion protein, Active Biotech; and immune mobilizing mTCR Against Cancer, ImmTAC) and oligoclonal antibodies (e.g., Symphogen and Merus).

Other non-limiting examples of multi-specific antigen-binding molecules include a Fabs-in-tandem immunoglobulins (FIT-Ig) (Gong et al., 2017. MAbs. 9(7):1118-1128. doi: 10.1080/19420862.2017.1345401. Epub 2017 Jul 10. PubMedPMID: 28692328; PubMed Central PMCID: PMC5627593), and are capable of binding two or more antigens. In the design of a FIT-Ig molecule, the two Fab domains from parental mAbs are fused directly in tandem in a crisscross orientation. The three fragments, when co-expressed in mammalian cells, assemble to form a tetravalent multi-specific FIT-Ig molecule. For instance, a bispecific binding protein could be constructed as a FIT-Ig using two parental monoclonal antibodies, mAb A (which binds to antigen A), and mAb B (which binds to antigen B). In the design of a FIT-Ig molecule, the two Fab domains from parental mAbs are fused directly in tandem in a crisscross orientation. The three fragments, when co-expressed in mammalian cells, assemble to form a tetravalent multi-specific FIT-Ig molecule. In representative embodiments, an FIT-Ig provides multi-specific antigen-binding molecules for antagonizing RANKL and at least one ICM. These multi-specific antigen-binding molecules generally comprise, consist or consist essentially of an antibody or antigen-binding fragment constructed as a FIT-Ig molecule thereof that binds specifically to RANKL or to RANK and for a respective ICM, an antibody or antigen-binding fragment thereof that binds specifically to that ICM. In some embodiments in which the RANKL antagonist is a direct RANKL antagonist, the multi-specific antigen-binding molecule comprises an anti-RANKL antibody or antigen-binding fragment thereof, which would be incorporated into a FIT-Ig molecule. In other embodiments in which the RANKL antagonist is an indirect RANKL antagonist, the multi-specific antigen-binding molecule comprises an anti-RANK antibody or antigen-binding fragment thereof, which would be incorporated into a FIT-Ig molecule. The at least one ICM is suitably selected from PD-1, PD-L1, or CTLA-4 and incorporated into a FIT-Ig molecule. In some embodiments in which the multi-specific antigen-binding molecule antagonizes PD-1, the multi-specific antigen-binding molecule comprises an anti-PD-1 antibody or antigen-binding fragment thereof. In some embodiments in which the multi-specific antigen-binding molecule antagonizes PD-L1, the multi-specific antigen-binding molecule comprises an anti-PD-L1 antibody or antigen-binding fragment thereof. In some embodiments in which the multi-specific antigen-binding molecule antagonizes CTLA4, the multi-specific antigen-binding molecule comprises an anti-CTLA4 antibody or antigen-binding fragment thereof.

Variable regions of antibodies are typically isolated as single-chain Fv (scFv) or Fab fragments. In some embodiments, the antigen-binding molecules comprise two or more scFv fragment. ScFv fragments are composed of V_{H} and V_{L} domains linked by a short 10-25 amino acid linker. Once isolated, scFv fragments can be linked with any flexible peptide linker known in the art (such as, for example, one or more repeats of Ala-Ala-Ala, Gly-Gly-Gly-Gly-Ser, etc.). The resultant polypeptide, a tandem scFv (taFv or scFv₂) can be arranged in various ways, with V_{H}-VL or V_{L}-V_{H} ordering for each scFv of the taFv. (Kontermann, supra).

In the present invention, an antibody may be characterized by having specific binding activity (Kₐ) for an antigen of at least about 10⁵ mot', 10⁶ mol⁻¹ or greater, preferably 10⁷ mol⁻¹ or greater, more preferably 10⁸ mol⁻¹ or greater, and most preferably 10⁹ mol⁻¹ or greater. The binding affinity of an antibody can be readily determined by one of ordinary skill in the art, for example, by Scatchard analysis (see, Scatchard, Ann. NY Acad. Sci. 51: 660-72, 1949).

### 3.1 Receptor Activator of NF-κB (RANK) Ligand (RANKL) Antagonists

The RANKL antagonists that are suitable for use in the therapeutic agents of the present invention, include any molecule that is capable of antagonizing RANKL (e.g., human RANKL). By way of an example, the RANKL antagonist may be a polypeptide, polynucleotide, antigen-binding molecule, carbohydrate, or small molecule. In some preferred embodiments, the RANKL antagonist is an anti-RANKL antigen-binding molecule (e.g., a MAb or an antigen-binding fragment thereof). Such anti-RANKL antigen-binding molecules specifically bind to a region or epitope of native RANKL, for example, native human RANKL with the following amino acid sequence:

[SEQ ID NO: 2]
MRRASRDYTKYLRGSEEMGGGPGAPHEGPLHAPPPPAPHQPPAASRSM
FVALLGLGLGQVVCSVALFFYFRAQMDPNRISEDGTHCIYRILRLHEN
ADFQDTTLESQDTKLIPDSCRRIKQAFQGAVQKELQHIVGSQHIRAEK
AMVDGSWLDLAKRSKLEAQPFAHLTINATDIPSGSHKVSLSSWYHDRG
WAKISNMTFSNGKLIVNQDGFYYLYANICFRHHETSGDLATEYLQLMV
YVTKTSIKIPSSHTLMKGGSTKYWSGNSEFHFYSINVGGFFKLRSGEE
ISIEVSNPSLLDPDQDATYFGAFKVRDID.

Suitably, the anti-RANKL antigen-binding molecules of the invention generally bind to a region or epitope of the extracellular domain of RANKL (i.e., corresponding to residues 69 to 317 of the human RANKL sequence set forth in SEQ ID NO:2). In some more specific embodiments, the anti-RANKL antigen-binding molecules suitably bind to a region of the receptor-binding domain of RANKL (i.e., corresponding to residues 162 to 317 of the human RANKL sequence set forth in SEQ ID NO:2). By way of an example, the anti-RANKL antigen-binding molecule specifically binds to one or more amino acids of the amino acid sequence TEYLQLMVY [SEQ ID NO:1] (i.e., residues 233 to 241 of the native human RANKL sequence set forth in SEQ ID NO:2).

Examples of known MAbs that bind specifically to human RANKL are described in U.S. Patent Appl Pub Nos. 2016/0333101 and 2012/0087923, the contents of which are incorporated herein by reference in their entirety.

One such anti-RANKL MAb that is suitable for use with the present invention is denosumab. Accordingly, in some embodiments, the anti-RANKL antigen-binding molecule comprises the fully human IgG2 MAb denosumab, or an antigen-binding fragment thereof. In some of the same embodiments and other embodiments, the anti-RANKL antigen-binding molecule comprises the CDR sequences as set forth in Table 1.

TABLE 1

| Heavy chain | | Light chain | |
|---|---|---|---|
| CDR1 | SYAMS | CDR1 | RASQSVRGRYLA |
| | [SEQ ID NO: 19] | | [SEQ ID NO: 22] |
| CDR2 | GITGSGGSTYYADSVKG | CDR2 | GASSRAT |
| | [SEQ ID NO: 20] | | [SEQ ID NO: 23] |
| CDR3 | DPGTTVIMSWFDP | CDR3 | QQYGSSPRT |
| | [SEQ ID NO: 21] | | [SEQ ID NO: 24] |

In non-limiting examples of this type, the anti-RANKL antigen-binding molecule comprises the heavy chain amino acid sequence of denosumab as set out for example below:

[SEQ ID NO: 3]
EVQLLESGGGLVQPGGSLRLSCAASGFTFSSYAMSWVRQAPGKGLEWV
SGITGSGGSTYYADSVKGRFTISRDNSKNTLYLQMNSLRAEDTAVYYC
AKDPGTTVIMSWFDPWGQGTLVTVSSASTKGPSVFPLAPSSKSTSGGT
AALGCLVKDYFPEPVTVSWNSGALTSGVHTFPAVLQSSGLYSLSSVVT
VPSSSLGTQTYICNVNHKPSNTKVDKKVEPKSCDKTHTCPPCPAPELL
GGPSVFLFPPKPKDTLMISRTPEVTCVVVDVSHEDPEVKFNWYVDGVE
VHNAKTKPREEQYNSTYRVVSVLTVLHQDWLNGKEYKCKVSNKALPAP
IEKTISKAKGQPREPQVYTLPPSRDELTKNQVSLTCLVKGFYPSDIAV
EWESNGQPENNYKTTPPVLDSDGSFFLYSKLTVDKSRWQQGNVFSCSV
MHEALHNHYTQKSLSLSPGK;

or an antigen-binding fragment thereof, an illustrative example of which comprises, consists or consists essentially of the amino acid sequence:

[SEQ ID NO: 25]
EVQLLESGGGLVQPGGSLRLSCAASGFTFSSYAMSWVRQAPGKGLEWV
SGITGSGGSTYYADSVKGRFTISRDNSKNTLYLQMNSLRAEDTAVYYC
AKDPGTTVIMSWFDPWGQGTLVTVSS.

In some of these examples and other examples, the anti-RANKL antigen-binding molecule comprises the light chain amino acid sequence of denosumab as set out below:

[SEQ ID NO: 4]
EIVLTQSPGTLSLSPGERATLSCRASQSVRGRYLAWYQQKPGQAPRLL
IYGASSRATGIPDRFSGSGSGTDFTLTISRLEPEDFAVFYCQQYGSSP
RTFGQGTKVEIKRTVAAPSVFIFPPSDEQLKSGTASVVCLLNNFYPRE
AKVQWKVDNALQSGNSQESVTEQDSKDSTYSLSSTLTLSKADYEKHKV
YACEVTHQGLSSPVTKSFNRGEC;

or an antigen-binding fragment thereof, an illustrative example of which comprises, consists or consists essentially of the amino acid sequence:

[SEQ ID NO: 26]
EIVLTQSPGTLSLSPGERATLSCRASQSVRGRYLAWYQQKPGQAPRLL
IYGASSRATGIPDRFSGSGSGTDFTLTISRLEPEDFAVFYCQQYGSSP
RTFGQGTKVEIK.

Full-length sequences for the heavy and light chains of denosumab are set out in SEQ ID NO:7 and 8, respectively:

[SEQ ID NO: 27]
MEFGLSWLFLVAILKGVOCEVQLLESGGGLVQPGGSLRLSCAASGFTF
SSYAMSWVRQAPGKGLEWVSGITGSGGSTYYADSVKGRFTISRDNSKN
TLYLQMNSLRAEOTAVYYCAKDPGTTVIMSWFOPWGQGTLVTVSSAST
KGPSVFPLAPCSRSTSESTAALGCLVKDYFPEPVTVSWNSGALTSGVH
TFPAVLQSSGLYSLSSVVTVPSSNFGTQTYTCNVDHKPSNTKVDKTVE
RKCCVECPPCPAPPVAGPSVFLFPPKPKDTLMISRTPEVTCVVVDVSH
EDPEVQFNWYVDGVEVHNAKTKPREEQFNSTFRVVSVLTVVHQDWLNG
KEYKCKVSNKGLPAPIEKTISKTKGQPREPQVYTLPPSREEMTKNQVS
LTCLVKGFYPSDIAVEWESNGQPENNYKTTPPMLDSDGSFFLYSKLTV
DKSRWQQGNVFSCSVMHEALHNHYTQKSLSLSPGK,
wherein the IgG₂ signal peptide is underlined;
and
[SEQ ID NO: 28]
METPAOLLFLLLLWLPDTTGEIVLTQSPGTLSLSPGERATLSCRASQS
VRGRYLAWYQQKPGQAPRLLIYGASSRATGIPDRFSGSGSGTDFTLTI
SRLEPEDFAVFYCQQYGSSPRTFGQGTKVEIKRTVAAPSVFIFPPSDE
QLKSGTASVVCLLNNFYPREAKVQWKVDNALQSGNSQESVTEQDSKDS
TYSLSSTLTLSKADYEKHKVYACEVTHQGLSSPVTKSFNRGEC,

wherein the kappa signal peptide is underlined.

Other illustrative anti-RANKL antigen-binding molecules that may be used in the practise of the present invention includes anti-RANKL-antigen-binding molecules disclosed in EP 1257648, the content of which is incorporated by reference herein in its entirety. In representative embodiments, the anti-RANKL antigen-binding molecule comprises the CDR sequences as set forth in Table 2.

TABLE 2

| Heavy chain | | Light chain | |
|---|---|---|---|
| CDR1 | NYAIYH | CDR1 | RASQSISRYLN |
| | [SEQ ID NO: 29] | | [SEQ ID NO: 32] |
| CDR2 | WINAGNGNTKFSQKFQG | CDR2 | GASSLQS |
| | [SEQ ID NO: 30] | | [SEQ ID NO: 33] |
| CDR3 | DSSNMVRGIIIAYYFDY | CDR3 | QHTRA |
| | [SEQ ID NO: 31] | | [SEQ ID NO: 34] |

In some of these embodiments, the anti-RANKL antigen-binding molecule comprises a heavy chain amino acid sequence as set out for example below:

[SEQ ID NO: 35]
AQVQLVQSGAEVRKPGASVKVSCKASGYDFSNYAIHWVRQAPGQRLEW
MGWINAGNGNTKFSQKFQGRITVTRDTAASTAYMELRSLRSEDTAVYY
CARDSSNMVRGIIIAYYFDYWGQGTLVTVSSASTKGPSVFPLAPSSKS
TSGGTAALGCLVKDYFPEPVTVSWNSGALTSGVHTFPAVLQSSGLYSL
SSVVTVPSSSLGTQTYICNVNHKPSNTKVDKKVEPKSC;

or an antigen-binding fragment thereof, an illustrative example of which comprises, consists or consists essentially of the amino acid sequence:

[SEQ ID NO: 36]
QVQLVQSGAEVRKPGASVKVSCKASGYDFSNYAIHWVRQAPGQRLEWM
GWINAGNGNTKFSQKFQGRITVTRDTAASTAYMELRSLRSEDTAVYYC
ARDSSNMVRGIIIAYYFDYWGQGTLVTVSS.

In some of these embodiments and other embodiments the anti-RANKL antigen-binding molecule comprises a light chain amino acid sequence as set out for example below:

[SEQ ID NO: 37]
SHSALEIVMTQSPSSLSASVGDRVTITCRASQSISRYLNWYQLKPGKA
PRLLIYGASSLQSGVPSRFSGSGSGAEFTLTISSLQPEDIATYYCQHT
RAFGQGTKVEIKRTVAAPSVFIFPPSDEQLKSGTASVVCLLNNFYPRE
AKVQWKVDNALQSGNSQESATEQDSKDSTYSLSSTLTLSKADYEKHKV
YACEVTHQGLSSPVTKSFNRGEC;

or an antigen-binding fragment thereof, a representative example of which comprises, consists or consists essentially of the amino acid sequence:

[SEQ ID NO: 38]
EIVMTQSPSSLSASVGDRVTITCRASQSISRYLNWYQLKPGKAPRLLI
YGASSLQSGVPSRFSGSGSGAEFTLTISSLQPEDIATYYCQHTRAFGQ
GTKVEIK.

In other representative embodiments, the anti-RANKL antigen-binding molecule comprises the CDR sequences as set forth in Table 3.

TABLE 3

| Heavy chain | | Light chain | |
|---|---|---|---|
| CDR1 | NYAIYH | CDR1 | RASQSVGSYLA |
| | [SEQ ID NO: 39] | | [SEQ ID NO: 42] |
| CDR2 | WINAGNGNTKFSQKFQG | CDR2 | DATNRAT |
| | [SEQ ID NO: 40] | | [SEQ ID NO: 43] |
| CDR3 | DSSNMVRGIIIAYYFDY | CDR3 | QHRRT |
| | [SEQ ID NO: 41] | | [SEQ ID NO: 44] |

In some of these embodiments, the anti-RANKL antigen-binding molecule comprises a heavy chain amino acid sequence as set out for example below:

[SEQ ID NO: 45]
AEVQLVQSGAEVRKPGASVKVSCKASGYDFSNYAIHWVRQAPGQRLEW
MGWINAGNGNTKFSQKFQGRITVTRDTAASTAYMELRSLRSEDTAVYY
CARDSSNMVRGIIIAYYFDYWGQGTLVTVSSASTKGPSVFPLAPSSKS
TSGGTAALGCLVKDYFPEPVTVSWNSGALTSGVHTFPAVLQSSGLYSL
SSVVTVPSSSLGTQTYICNVNHKPSNTKVDKKVEPKSC;

or an antigen-binding fragment thereof, an illustrative example of which comprises, consists or consists essentially of the amino acid sequence:

[SEQ ID NO: 46]
EVQLVQSGAEVRKPGASVKVSCKASGYDFSNYAIHWVRQAPGQRLEWM
GWINAGNGNTKFSQKFQGRITVTRDTAASTAYMELRSLRSEDTAVYYC
ARDSSNMVRGIIIAYYFDYWGQGTLVTVSS.

In some of these embodiments and other embodiments the anti-RANKL antigen-binding molecule comprises a light chain amino acid sequence as set out for example below:

[SEQ ID NO: 47]
SHSALEIVLTQSPATLSFSPGERATLSCRASQSVGSYLAWYQQRPGQA
PRPLIYDATNRATGIPTRFSGSGSGTDFTLTISSLEPEDFATYYCQHR
RTFGRGTKLEIKRTVAAPSVFIFPPSDEQLKSGTASVVCLLNNFYPRE
AKVQWKVDNALQSGNSQESVTEQDSKDSTYSLSSTLTLSKADYEKHKV
YACEVTHQGLSSPVTKSFNRGEC;

or an antigen-binding fragment thereof, a representative example of which comprises, consists or consists essentially of the amino acid sequence:

[SEQ ID NO: 48]
EIVLTQSPATLSFSPGERATLSCRASQSVGSYLAWYQQRPGQAPRPLI
YDATNRATGIPTRFSGSGSGTDFTLTISSLEPEDFATYYCQHRRTFGR
GTKLEIK.

In various embodiments, the anti-RANKL antigen-binding molecule comprises a variable light chain (V_{L}) amino acid sequence and a variable heavy chain (V_{H}) amino acid sequence wherein individual V_{L} chains comprise CDR amino acid sequences designated CDR1 (V_{L}), CDR2(V_{L}) and CDR3(VL) separated by framework amino acid sequences,

CDR1 (V_{L}) being selected from the group consisting of: RASQSISRYLN [SEQ ID NO:49]; RASQSVGSYLA [SEQ ID NO:50]; RASQSVSSSSLA [SEQ ID NO:51]; and SGDALPKQY [SEQ ID NO:52];

CDR2 (V_{L}) being selected from the group consisting of: GASSLQS [SEQ ID NO:53]; DATNRAT [SEQ ID NO:54]; GASSRAT [SEQ ID NO:55]; and EDSERPS [SEQ ID NO:56]; and

CDR3 (V_{L}) being selected from the group consisting of: QHTRA [SEQ ID NO:57]; QHRRT [SEQ ID NO:58]; QQYGA [SEQ ID NO:59]; and QSTDSSGTYVV [SEQ ID NO:60], wherein CDR1 (V_{L}), CDR2 (V_{L}) and CDR3 (V_{L}) are selected independently of each other; and

wherein each V_{H} chain comprises CDR amino acid sequences designated CDR1 (V_{H}), CDR2(V_{H}) and CDR3 (V_{H}) separated by framework amino acid sequences,

CDR1 (V_{H}) being selected from the group consisting of: NYAIH [SEQ ID NO:61]; NYPMH [SEQ ID NO:62]; and DXAMH [SEQ ID NO:63],

CDR2 (VH) being selected from the group consisting of: WINAGNGNTKFSQKFQG [SEQ ID NO:64]; VISYDGNNKYYADSVKG [SEQ ID NO:65]; and GISMNSGRIGYADSVKO [SEQ ID NO:66],

CDR3 (V_{H}) being selected from the group consisting of: DSSNMVRGIIIAYYFDY [SEQ ID NO:67]; GGGGFDY [SEQ ID NO:68]; and GGSTSARYSSGWYY [SEQ ID NO:69], wherein CDR1 (V_{H}), CDR2 (V_{H}) and CDR3 (VH) are selected independently of each other.

In specific embodiments, the anti-RANKL antigen-binding molecule comprises a V_{L} and a V_{H} chain, wherein:

the V_{L} chain comprises CDR1 having the sequence RASQSISRYLN [SEQ ID NO:49], CDR2 having the sequence GASSLQS [SEQ ID NO:53], and CDR3 having the sequence QHTRA [SEQ ID NO:57]; and

the V_{H} chain comprises CDR1 having the sequence NYAIH [SEQ ID NO:61], CDR2 having the sequence WINAGNGNTKFSQKFQG [SEQ ID NO:64], and CDR3 having the sequence DSSNMVRGIIIAYYFDY [SEQ ID NO:67], wherein CDR1, CDR2 and CDR3 on each V_{L} and V_{H} chain are separated by framework amino acid sequences.

In other embodiments, the RANKL antagonist is an indirect RANKL antagonist, which specifically binds to a RANKL binding-partner. By way of an example, the RANKL antagonist inhibits or abrogates the functional activity of RANK. RANK (also known as TNFRSF11A, Receptor activator of NFκB, and CD265) is a member of the tumour necrosis factor receptor (TNFR) molecular sub-family. RANK is constitutively expressed in skeletal muscle, thymus, liver, colon, small intestine, adrenal gland, osteoclast, mammary gland epithelial cells, prostate, vascular cells, and pancreas.

In some embodiments, the RANK antagonist comprises, consists or consists essentially of an amino acid sequence corresponding to a region of RANK that interacts with RANKL, representative examples of which comprise at least one CRD selected from CDR2 (i.e., residues 44-85) and CRD3 (i.e., residues 86-123). In non-limiting examples of this type, the RANK antagonist comprises, consists or consists essentially of an amino acid sequence corresponding to RANK CRD3, representative examples of which include YCWNSDCECCY [SEQ ID NO:5], YCWSQYLCY [SEQ ID NO:6].

In other embodiments, the RANK antagonist is an anti-RANK antigen-binding molecule (e.g., a MAb or an antigen-binding fragment thereof), which binds specifically to a region or epitope of native RANK, for example, native human RANK (UniProt accession no. Q9Y6Q6) with a representative full-length amino acid sequence:

[SEQ ID NO: 8]
MAPRARRRRPLFALLLLCALLARLQVALQIAPPCTSEKHYEHLGRCCN
KCEPGKYMSSKCTTTSDSVCLPCGPDEYLDSWNEEDKCLLHKVCDTGK
ALVAVVAGNSTTPRRCACTAGYHWSQDCECCRRNTECAPGLGAQHPLQ
LNKDTVCKPCLAGYFSDAFSSTDKCRPWTNCTFLGKRVEHHGTEKSDA
VCSSSLPARKPPNEPHVYLPGLIILLLFASVALVAAIIFGVCYRKKGK
ALTANLWHWINEACGRLSGDKESSGDSCVSTHTANFGQQGACEGVLLL
TLEEKTFPEDMCYPDQGGVCQGTCVGGGPYAQGEDARMLSLVSKTEIE
EDSFRQMPTEDEYMDRPSQPTDQLLFLTEPGSKSTPPFSEPLEVGEND
SLSQCFTGTQSTVGSESCNCTEPLCRTDWTPMSSENYLQKEVDSGHCP
HWAASPSPNWADVCTGCRNPPGEDCEPLVGSPKRGPLPQCAYGMGLPP
EEEASRTEARDQPEDGADGRLPSSARAGAGSGSSPGGQSPASGNVTGN
SNSTFISSGQVMNFKGDIIVVYVSQTSQEGAAAAAEPMGRPVQEETLA
RRDSFAGNGPRFPDPCGGPEGLREPEKASRPVQEQGGAKA.

The anti-RANK antigen-binding molecules of the invention generally bind to a region of the extracellular domain of RANK (e.g., corresponding to residues 30 to 212 of the human RANK sequence set forth in SEQ ID NO:8), a non-limiting example of which includes: VSKTEIEEDSFRQMPTEDEYMDRPSQPTDQLLFLTEPGSKSTPPFSEPLEVGENDSLSQCFTGTQSTVGSESCNC TEPLCRTDWTPMS [SEQ ID NO:7] (i.e., residues 330-417 of the native RANK sequence set forth in SEQ ID NO:8). In some embodiments of this type, the anti-RANK antigen-binding molecule is selected form the MAbs 64C1385 (Abcam) , N-1H8 and N-2B10, or an antigen-binding molecule thereof, including chimeric and humanized antigen-binding molecules. In other embodiments, the anti-RANK antigen-binding molecule competes with MAbs 64C1385, N-1H8 or N-2B10 for binding to RANK.

In some embodiments, the anti-RANK antigen-binding molecule is a short chain Fv (scFv) antigen-binding molecule as disclosed for example by Newa et al. (2014, supra), or an antigen-binding fragment thereof. Representative antigen-binding molecules of this type may comprise the CDR sequences as set forth in Table 4.

TABLE 4

| Heavy chain | | Light chain | |
|---|---|---|---|
| CDR1 | GFTFSSY | CDR1 | RASQSISSYLN |
| | [SEQ ID NO: 70] | | [SEQ ID NO: 73] |
| CDR2 | SGDGY | CDR2 | YASSLQS |
| | [SEQ ID NO: 71] | | [SEQ ID NO: 74] |
| CDR3 | NAYSFDY | CDR3 | QQGSSSPNT |
| | [SEQ ID NO: 72] | | [SEQ ID NO: 75] |

In more specific embodiments, the anti-RANK antigen-binding molecule comprises a heavy chain amino acid sequence:

[SEQ ID NO: 76]
MAEVQLLESGGGLVQPGGSLRLSCAASGFTFSSYAMSWVRQAPGKGLE
WVSAISGDGYYTDYADSVGRFTISRDNSKNTLYLQNSLRAEDTAVYYC
AKNAYSFDYWGQGTLVTVS
or
[SEQ ID NO: 77]
MAEVQLLESGGGLVQPGGSLRLSCAASGFTFSSYAMSWVRQAPGKGLE
WVSAISGDGYYTDYADSVGRFTISRDNSKNTLYLQMNSLRAEDTAVYY
CAKNAYSFDYWGQGTLVTVS;

or an antigen-binding fragment thereof, which comprises, consists or consists essentially of the amino acid sequence:

[SEQ ID NO: 78]
EVQLLESGGGLVQPGGSLRLSCAASGFTFSSYAMSWVRQAPGKGLEWV
SAISGDGYYTDYADSVGRFTISRDNSKNTLYLQNSLRAEDTAVYYCAK
NAYSFDYWGQGTLVTVS
or
[SEQ ID NO: 79]
EVQLLESGGGLVQPGGSLRLSCAASGFTFSSYAMSWVRQAPGKGLEWV
SAISGDGYYTDYADSVGRFTISRDNSKNTLYLQMNSLRAEDTAVYYCA
KNAYSFDYWGQGTLVTVS.

In some of the same and other embodiments, the anti-RANK antigen-binding molecule may comprise the light chain amino acid sequence:

[SEQ ID NO: 80]
DIQMTQSPSSLSASVGDRVTITCRASQSISSYLNWYQQKPGKAPKLLI
YYASSLQSGVPSRFSGSGSGTDFTLTISSLQPEDFATYYCQQGSSSPN
TFGQGTKVEIKRAAA;

or an antigen-binding fragment thereof, which comprises, consists or consists essentially of the amino acid sequence:

[SEQ ID NO: 81]
DIQMTQSPSSLSASVGDRVTITCRASQSISSYLNWYQQKPGKAPKLLI
YYASSLQSGVPSRFSGSGSGTDFTLTISSLQPEDFATYYCQQGSSSPN
TFGQGTKVEIK.

### 3.2 Immune Checkpoint Molecule (ICM) Antagonists.

Any suitable ICM antagonist that can be used in therapy is contemplated for use in the practice of the present invention. For example, suitable ICM antagonists include polypeptides, polynucleotides, carbohydrates, and small molecules. In some preferred embodiments, the ICM antagonist is an antigen-binding molecule.

The ICM that is antagonized by the therapeutic combinations of the present invention include any one or more of the inhibitory ICM selected from:

PD-1, PD-L1, PD-L2, CTLA-4, A2AR, A2BR, CD276, VTCN1, BTLA, IDO, KIR, LAG3, TIM-3, VISTA, CD73, CD96, CD155, DNAM-1, CD112, CRTAM, OX40, OX4OL, CD244, CD160, GITR, GITRL, ICOS, GAL-9, 4-1BBL, 4-1BB, CD27L, CD28, CD80, CD86, SIRP-1, CD47, CD48, CD244, CD40, CD4OL, HVEM, TMIGD2, HHLA2, VEGI, TNFRS25 and ICOLG. Suitably, in embodiments in which therapeutic combination comprises a RANKL antagonist and a single ICM antagonist, the ICM is other than CTLA-4.

In some preferred embodiments, an ICM antagonist included in the therapeutic combination is a PD-1 antagonist. In this regard, a “PD-1 antagonist” includes any chemical compound or biological molecule that blocks binding of PD-L1 (for example, PD-L1 expressed the surface of a cancer cell) to PD-1 that is expressed on an immune cell (for example, a T-cell, B-cell, or NKT cell). Alternative names or synonyms for PD-1 include PDCD1, PD1, CD279 and SLEB2. A representative mature amino acid sequence of human PD-1 (UniProt accession no. Q15116) is set out below:

[SEQ ID NO:]
PGWFLDSPDRPWNPPTFSPALLVVTEGDNATFTCSFSNTSESFVLNWY
RMSPSNQTDKLAAFPEDRSQPGQDCRFRVTQLPNGRDFHMSVVRARRN
DSGTYLCGAISLAPKAQIKESLRAELRVTERRAEVPTAHPSPSPRPAG
QFQTLVVGVVGGLLGSLVLLVWVLAVICSRAARGTIGARRTGQPLKED
PSAVPVFSVDYGELDFQWREKTPEPPVPCVPEQTEYATIVFPSGMGTS
SPARRGSADGPRSAQPLRPEDGHCSWPL.

Examples of MAbs that bind to human PD-1, and therefore of use in the present invention, are described in US Patent Publication Nos. US2003/0039653, US2004/0213795, US2006/0110383, US2007/0065427, US2007/0122378, US2012/237522, and International PCT Publication Nos. WO2004/072286, WO2006/121168, WO2006/133396, WO2007/005874, WO2008/083174, WO2008/156712, WO2009/024531, WO2009/014708, WO2009/114335, WO2010/027828, WO2010/027423, WO2010/036959, WO2010/029435, WO2010/029434, WO2010/063011, WO2010/089411, WO2011/066342, WO2011/110604, WO2011/110621, and WO2012/145493 (the entire contents of which is incorporated herein by reference). Specific MAbs that are useful for the purposes of the present invention include the anti-PD-1 MAbs nivolumab, pembrolizumab, and pidilizumab, as well as the humanized anti-PD-1 antibodies h409A11, h409A16, and h409A17 described in International Patent Publication No. WO2008/156712.

The anti-PD-1 antigen-binding molecules of the invention preferably bind to a region of the extracellular domain of PD-1. By way of example, the anti-PD-1 antigen-binding molecules may specifically bind to a region of the extracellular domain of human PD-1, which comprises one or both of the amino acid sequences SFVLNWYRMSPSNQTDKLAAFPEDR [SEQ ID NO:9] (i.e., residues 62 to 86 of the native PD-1 sequence set forth in SEQ ID NO:10) and SGTYLCGAISLAPKAQIKE [SEQ ID NO:11] (i.e., residues 118 to 136 of the native PD-1 sequence set forth in SEQ ID NO:10). In another example, the anti-PD-1 antigen-binding molecule binds to a region of the extracellular domain of human PD-1 that comprises the amino acid sequence NWYRMSPSNQTDKLAAFPEDRSQPGQDCRFRV [SEQ ID NO:12] (i.e., corresponding to residue 66 to 97 of the native human PD-1 sequence set forth in SEQ ID NO:10).

In certain embodiments, the anti-PD-1 antigen-binding molecule comprises the fully humanized IgG4 MAb nivolumab (as described in detail in U.S. Pat. No. 8,008,449 (referred to as “5C4”), which is incorporated herein by reference in its entirety) or an antigen-binding fragment thereof. In representative examples of this type, the anti-PD-1 antigen-binding molecule comprises the CDR sequences as set forth in Table 5.

TABLE 5

| Heavy chain | | Light chain | |
|---|---|---|---|
| CDR1 | NSGMH | CDR1 | RASQSVSSYLA |
| | [SEQ ID NO: 82] | | [SEQ ID NO: 85] |
| CDR2 | VIWYDGSKRYYADSVKG | CDR2 | DASNRAT |
| | [SEQ ID NO: 83] | | [SEQ ID NO: 86] |
| CDR3 | NDDYW | CDR3 | QQSSNWPRT |
| | [SEQ ID NO: 84] | | [SEQ ID NO: 87] |

In more specific embodiments, the anti-PD-1 antigen-binding molecule comprises a heavy chain amino acid sequence of nivolumab as set out for example below:

[SEQ ID NO: 88]
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSSASTKGPSVFPLAPCSRSTSESTAALGCLVKDYFPEPV
TVSWNSGALTSGVHTFPAVLQSSGLYSLSSVVTVPSSSLGTKTYTCNVDH
KPSNTKVDKRVESKYGPPCPPCPAPEFLGGPSVFLFPPKPKDTLMISRTP
EVTCVVVDVSQEDPEVQFNWYVDGVEVHNAKTKPREEQFNSTYRVVSVLT
VLHQDWLNGKEYKCKVSNKGLPSSIEKTISKAKGQPREPQVYTLPPSQEE
MTKNQVSLTCLVKGFYPSDIAVEWESNGQPENNYKTTPPVLDSDGSFFLY
SRLTVDKSRWQEGNVFSCSVMHEALHNHYTQKSLSLSLGK;

or an antigen-binding fragment thereof, which comprises, consists or consists essentially of the amino acid sequence:

[SEQ ID NO: 89]
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSS.

In some of the same and other embodiments, the anti-PD-1 antigen-binding molecule may comprise the light chain amino acid sequence of nivolumab as set out for example below:

[SEQ ID NO: 90]
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC;

or an antigen-binding fragment thereof, which comprises, consists or consists essentially of the amino acid sequence:

[SEQ ID NO: 91]
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIK.

In alternate embodiments, the anti-PD-1 antigen-binding molecule comprises the humanized IgG4 MAb pembrolizumab or an antigen-binding fragment thereof. In non-limiting examples of this type, the anti-PD-1 antigen-binding molecule comprises the CDR sequences as set forth in Table 6.

TABLE 6

| Heavy chain | | Light chain | |
|---|---|---|---|
| CDR1 | NYYMY [SEQ ID NO: 92] | CDR1 | RASKGVSTSGYSYLH |
| | | | [SEQ ID NO: 95] |
| CDR2 | GINPSNGGTNFNEKFKN | CDR2 | LASYLES] |
| | [SEQ ID NO: 93] | | [SEQ ID NO: 96 |
| CDR3 | RDYRFDMGFDY | CDR3 | QHSRDLPLT |
| | [SEQ ID NO: 94] | | [SEQ ID NO: 97] |

In some embodiments, the anti-PD-1 antigen-binding molecule competes with the MAb pembrolizumab for binding to PD-1.

In additional embodiments, the anti-PD-1 antigen-binding molecule comprises the heavy chain amino acid sequence of pembrolizumab as set out for example below:

[SEQ ID NO: 98]
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK;

or an antigen-binding fragment thereof, which comprises, consists or consists essentially of the amino acid sequence:

[SEQ ID NO: 99]
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSS.

Similarly, the anti-PD-1 antigen-binding molecule may comprise a light chain amino acid sequence of pembrolizumab as set out for example below:

[SEQ ID NO: 100]
EIVLTQSPATLSLSPGERATLSCRASKGVSTSGYSYLHWYQQKPGQAPRL
LIYLASYLESGVPARFSGSGSGTDFTLTISSLEPEDFAVYYCQHSRDLPL
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC;

or an antigen-binding fragment thereof, which comprises, consists or consists essentially of the amino acid sequence:

[SEQ ID NO: 101]
EIVLTQSPATLSLSPGERATLSCRASKGVSTSGYSYLHWYQQKPGQAPRL
LIYLASYLESGVPARFSGSGSGTDFTLTISSLEPEDFAVYYCQHSRDLPL
TFGGGTKVEIK.

In yet other embodiments of this type, the anti-PD-1 antigen-binding molecule comprises the MAb pidilizumab or an antigen-binding fragment thereof. In some related embodiments, the anti-PD-1 antigen-binding molecule comprises CDR sequences as set forth in Table 7.

TABLE 7

| Heavy chain | | Light chain | |
|---|---|---|---|
| CDR1 | NYGMN | CDR1 | SARSSVSYMH |
| | [SEQ ID NO: 102] | | [SEQ ID NO: 105] |
| CDR2 | WINTDSGESTYAEEFKG | CDR2 | RTSNLAS |
| | [SEQ ID NO: 103] | | [SEQ ID NO: 106] |
| CDR3 | VGYDALDY | CDR3 | QQRSSFPLT |
| | [SEQ ID NO: 104] | | [SEQ ID NO: 107] |

In more specific embodiments, the anti-PD-1 antigen-binding molecule comprises a heavy chain amino acid sequence of pidilizumab as set forth below:

[SEQ ID NO: 108]
QVQLVQSGSELKKPGASVKISCKASGYTFTNYGMNWVRQAPGQGLQWMGW
INTDSGESTYAEEFKGRFVFSLDTSVNTAYLQITSLTAEDTGMYFCVRVG
YDALDYWGQGTLVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKRVEPKSCDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSREEMTKNQVSLTCLVKGFYPSDIAVEWESNGQPENNYKTTPPVLDS
DGSFFLYSKLTVDKSRWQQGNVFSCSVMHEALHNHYTQKSLSLSPGK;

or an antigen-binding fragment thereof, which comprises, consists or consists essentially of the amino acid sequence:

[SEQ ID NO: 109]
QVQLVQSGSELKKPGASVKISCKASGYTFTNYGMNWVRQAPGQGLQWMGW
INTDSGESTYAEEFKGRFVFSLDTSVNTAYLQITSLTAEDTGMYFCVRVG
YDALDYWGQGTLVTVSS.

In some of the same and other embodiments, the anti-PD-1 antigen-binding molecule comprises the light chain amino acid sequence of pidilizumab as shown below:

[SEQ ID NO: 110]
EIVLTQSPSSLSASVGDRVTITCSARSSVSYMHWFQQKPGKAPKLWIYRT
SNLASGVPSRFSGSGSGTSYCLTINSLQPEDFATYYCQQRSSFPLTFGGG
TKLEIKRTVAAPSVFIFPPSDEQLKSGTASVVCLLNNFYPREAKVQWKVD
NALQSGNSQESVTEQDSKDSTYSLSSTLTLSKADYEKHKVYACEVTHQGL
SSPVTKSFNRGEC,

or an antigen-binding fragment thereof, which comprises, consists or consists essentially of the amino acid sequence:

[SEQ ID NO: 111]
EIVLTQSPSSLSASVGDRVTITCSARSSVSYMHWFQQKPGKAPKLWIYRT
SNLASGVPSRFSGSGSGTSYCLTINSLQPEDFATYYCQQRSSFPLTFGGG
TKLEIK.

Other suitable MAbs are described in the International Patent Publication No. WO2015/026634, which is hereby incorporated by reference herein in its entirety. These include MAbs, or antigen-binding fragments thereof, which comprise: (a) light chain CDRs with amino acid sequences: RASKSVSTSGFSYLH [SEQ ID NO:112], LASNLES [SEQ ID NO:113], and QHSWELPLT [SEQ ID NO:114] (CDR1, CDR2, and CDR3, respectively) and heavy chain CDRs with amino acid sequences SYYLY [SEQ ID NO:115], GVNPSNGGTNFSEKFKS [SEQ ID NO:116] and RDSNYDGGFDY [SEQ ID NO:117] (CDR1, CDR2, and CDR3, respectively); or (b) light chain CDRs with amino acid sequence RASKGVSTSGYSYLH [SEQ ID NO:118], LASYLES [SEQ ID NO:119], and QHSRDLPLT [SEQ ID NO:120] (CDR1, CDR2, and CDR3, respectively), and heavy chain CDRs with amino acid sequence NYYMY [SEQ ID NO:121], GINPSNGGTNFNEKFKN [SEQ ID NO:122], and RDYRFDMGFDY [SEQ ID NO:123] (CDR1, CDR2, and CDR3, respectively).

By way of an illustration, such MAbs may comprise (a) a heavy chain variable region comprising:

[SEQ ID NO: 124]
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSS,

or

a variant or antigen-binding fragment thereof; and

a light chain variable region comprising an amino acid sequence selected from:

[SEQ ID NO: 125]
EIVLTQSPATLSLSPGERATLSCRASKGVSTSGYSYLHWYQQKPGQAPRL
LIYLASYLESGVPARFSGSGSGTDFTLTISSLEPEDFAVYYCQHSRDLPL
TFGGGTKVEIK,
[SEQ ID NO: 126]
IVLTQSPLSLPVTPGEPASISCRASKGVSTSGYSYLHWYLQKPGQSPQLL
IYLASYLESGVPDRFSGSGSGTDFTLKISRVEAEDVGVYYCQHSRDLPLT
FGQGTKLEIK,
or
[SEQ ID NO: 127]
DIVMTQTPLSLPVTPGEPASISCRASKGVSTSGYSYLHWYLQKPGQSPQL
LIYLASYLESGVPDRFSGSGSGTAFTLKISRVEAEDVGLYYCQHSRDLPL
TFGQGTKLEIK,

or a variant or

antigen-binding fragment thereof.

In yet further exemplary embodiments the anti-PD-1 MAb may comprise the IgG1 heavy chain comprising:

[SEQ ID NO: 128]
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK

or a variant or antigen-binding fragment thereof;

and a light chain comprising any one of:

[SEQ ID NO: 129]
EIVLTQSPATLSLSPGERATLSCRASKGVSTSGYSYLHWYQQKPGQAPRL
LIYLASYLESGVPARFSGSGSGTDFTLTISSLEPEDFAVYYCQHSRDLPL
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC,
[SEQ ID NO: 130]
EIVLTQSPLSLPVTPGEPASISCRASKGVSTSGYSYLHWYLQKPGQSPQL
LIYLASYLESGVPDRFSGSGSGTDFTLKISRVEAEDVGVYYCQHSRDLPL
TFGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC
[SEQ ID NO: 131]
DIVMTQTPLSLPVTPGEPASISCRASKGVSTSGYSYLHWYLQKPGQSPQL
LIYLASYLESGVPDRFSGSGSGTAFTLKISRVEAEDVGLYYCQHSRDLPL
TFGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC,

or a variant or an antigen-binding fragment thereof.

In other embodiments, the ICM antagonist is a PD-L1 antagonist. Alternative names or synonyms for PD-L1 include PDCD1L1, PDL1, B7H1, B7-4, CD274, and B7-H. Generally, the PD-L1 antagonists specifically bind to the native amino acid sequence of human PD-L1 (UniProt accession no. Q9NZQ7) as set out below:

[SEQ ID NO: 14]
MRIFAVFIFMTYWHLLNAFTVTVPKDLYVVEYGSNMTIECKFPVEKQLDL
AALIVYWEMEDKNIIQFVHGEEDLKVQHSSYRQRARLLKDQLSLGNAALQ
ITDVKLQDAGVYRCMISYGGADYKRITVKVNAPYNKINQRILVVDPVISE
HELTCQAEGYPKAEVIWTSSDHQVLSGKTTTTNSKREEKLFNVTSTLRIN
TTTNEIFYCTFRRLDPEENHTAELVIPELPLAHPPNERTHLVILGAILLC
LGVALTFIFRLRKGRMMDVKKCGIQDTNSKKQSDTHLEET.

Suitably, the PD-L1 antagonist is an anti-PD-L1 antigen-binding molecule. By way of example, anti-PD-L1 antigen-binding molecules that are suitable for use with the present invention include the anti-PD-L1 MAbs durvalumab (MEDI4736), atezolizumab (Tecentriq), BMS-936559/MDX-1105, MSB0010718C, LY3300054, CA-170, GNS-1480, MPDL3280A, and avelumab. These and other anti-PD-L1 antibodies are described in International Publication Nos. WO2007/005874 and WO2010/077634, and U.S. Patent Nos. 8,217,149, and 8,779,108, the entirety of each of which is incorporated herein by reference. Further anti-PD-L1 MAbs are described in International PCT Patent Publication No. WO2016/007,235, the entire contents of which is also incorporated herein by reference.

The anti-PD-L1 antigen-binding molecules suitably bind to a region of the extracellular domain of PD-L1. By way of illustration, the anti-PD-L1 antigen-binding molecules may specifically bind to a region of the extracellular domain of human PD-L1 that comprises the amino acid sequence SKKQSDTHLEET [SEQ ID NO:13] (i.e., residues 279 to 290 of the native PD-L1 sequence set forth in SEQ ID NO:14). In certain embodiments, the anti-PD-L1 antigen-binding molecule comprises the fully humanized IgG1 MAb durvalumab (as described with reference to “MEDI4736” in International PCT Publication No. WO2011/066389, and U.S. Patent Publication No 2013/034559, which are incorporated herein by reference in their entirety) or an antigen-binding fragment thereof. In representative embodiments of this type, the anti-PD-L1 antigen-binding molecule comprises the CDR sequences as set forth in Table 8.

TABLE 8

| Heavy chain | | Light chain | |
|---|---|---|---|
| CDR1 | RYWMS [SEQ ID NO: 132] | CDR1 | RASQRVSSSYLA |
| | | | [SEQ ID NO: 135] |
| CDR2 | NIKQDGSEKYYVDSVK | CDR2 | DASSRATGIPD |
| | [SEQ ID NO: 133] | | [SEQ ID NO: 136] |
| CDR3 | EGGWFGELAFDY | CDR3 | QQYGSLPWT |
| | [SEQ ID NO: 134] | | [SEQ ID NO: 137] |

In more specific embodiments, the anti-PD-L1 antigen-binding molecule comprises the heavy chain amino acid sequence of durvalumab as set out for example below:

[SEQ ID NO: 138]
VQLVESGGGLVQPGGSLRLSCAASGFTFSRYWMSWVRQAPGKGLEWVANI
KQDGSEKYYVDSVKGRFTISRDNAKNSLYLQMNSLRAEDTAVYYCAREGG
WFGELAFDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPEFEGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPASIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPG,

or an antigen-binding fragment thereof, which comprises, consists or consists essentially of the amino acid sequence:

[SEQ ID NO: 139]
VQLVESGGGLVQPGGSLRLSCAASGFTFSRYWMSWVRQAPGKGLEWVANI
KQDGSEKYYVDSVKGRFTISRDNAKNSLYLQMNSLRAEDTAVYYCAREGG
WFGELAFDYWGQGTLVTVSS.

In some of the same and other embodiments, the anti-PD-L1 antigen-binding molecule may comprise the light chain amino acid sequence:

[SEQ ID NO: 140]
EIVLTQSPGTLSLSPGERATLSCRASQRVSSSYLAWYQQKPGQAPRLLIY
DASSRATGIPDRFSGSGSGTDFTLTISRLEPEDFAVYYCQQYGSLPWTFG
QGTKVEIKRTVAAPSVFIFPPSDEQLKSGTASVVCLLNNFYPREAKVQWK
VDNALQSGNSQESVTEQDSKDSTYSLSSTLTLSKADYEKHKVYACEVTHQ
GLSSPVTKSFNRGEC,

or an antigen-binding fragment thereof, which comprises, consists or consists essentially of the amino acid sequence:

[SEQ ID NO: 141]
EIVLTQSPGTLSLSPGERATLSCRASQRVSSSYLAWYQQKPGQAPRLLIY
DASSRATGIPDRFSGSGSGTDFTLTISRLEPEDFAVYYCQQYGSLPWTFG
QGTKVEIK.

Alternatively, the anti-PD-L1 antigen-binding molecule competes for binding to PD-L1 with the MAb durvalumab.

In other embodiments, the anti-PD-L1 antigen-binding molecule comprises the fully humanized IgG1 MAb atezolizumab (as described in U.S. Pat. No. 8,217148, the entire content of which is incorporated herein by reference) or an antigen-binding fragment thereof. In representative embodiments of this type, the anti-PD-L1 antigen-binding molecule comprises the CDR sequences as set forth in Table 9.

TABLE 9

| Heavy chain | | Light chain | |
|---|---|---|---|
| CDR1 | GFTFSX₁SWIH | CDR1 | RASQX₄X₅X₆TX₇X₈A |
| | [SEQ ID NO: 142] | | [SEQ ID NO: 145] |
| CDR2 | AWIX₂PYGGSX₃YYADSVKG | CDR2 | SASX₉LX₁₀S |
| | [SEQ ID NO: 143] | | [SEQ ID NO: 146] |
| CDR3 | RHWPGGFDY | CDR3 | QQX₁₁X₁₂X₁₃X₁₄PX₁₅T |
| | [SEQ ID NO: 144] | | [SEQ ID NO: 147] |

wherein X₁ is D or G; X₂ is S or L; X₃ is T or S; X₄ is D or V; X₅ is V or I; X₆ is S or N; X₇ is A or F; X₈ is V or L; X₉ is F or T; X₁₀ is Y or A; X₁₁ is Y, G, or F; X₁₂ is L, Y, or F; X₁₃ is Y, N, T, G, F or I; X₁₄ is H, V, P, T, or I; and X₁₅ is A, W, R, P, or T.

In more specific embodiments, the anti-PD-L1 antigen-binding molecule comprises the heavy chain amino acid sequence of atezolizumab as set forth for example below:

[SEQ ID NO: 148]
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSSASTKGPSVFPLAPSSKSTSGGTAALGCLVKDY
FPEPVTVSWNSGALTSGVHTFPAVLQSSGLYSLSSVVTVPSSSLGTQTYI
CNVNHKPSNTKVDKKVEPKSCDKTHTCPPCPAPELLGGPSVFLFPPKPKD
TLMISRTPEVTCVVVDVSHEDPEVKFNWYVDGVEVHNAKTKPREEQYAST
YRVVSVLTVLHQDWLNGKEYKCKVSNKALPAPIEKTISKAKGQPREPQVY
TLPPSREEMTKNQVSLTCLVKGFYPSDIAVEWESNGQPENNYKTTPPVLD
SDGSFFLYSKLTVDKSRWQQGNVFSCSVMHEALHNHYTQKSLSLSPGK,

or an antigen-binding fragment thereof, which comprises, consists or consists essentially of the amino acid sequence:

[SEQ ID NO: 149]
EVQLVESGGGLVQPGGSLRLSCAASGFTFSDSWIHWVRQAPGKGLEWVAW
ISPYGGSTYYADSVKGRFTISADTSKNTAYLQMNSLRAEDTAVYYCARRH
WPGGFDYWGQGTLVTVSS.

In some of the same and other embodiments, the anti-PD-L1 antigen-binding molecule comprises the light chain amino acid sequence of atezolizumab as provided for example below:

[SEQ ID NO: 150]
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC,

or an antigen-binding fragment thereof, which comprises, consists or consists essentially of the amino acid sequence:

[SEQ ID NO: 151]
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIK.

Alternatively, the anti-PD-L1 antigen-binding molecule competes for binding to PD-L1 with the MAb atezolizumab.

In other embodiments, the anti-PD-L1 antigen-binding molecule comprises the fully humanized IgG1 MAb avelumab (as described in U.S. Patent No. 8,217148, the entire contents of which is incorporated herein by reference) or an antigen-binding fragment thereof. In representative embodiments of this type, the anti-PD-L1 antigen-binding molecule comprises the CDR sequences as set forth in Table 10.

TABLE 10

| Heavy chain | | Light chain | |
|---|---|---|---|
| CDR1 | X₁YX₂MX₃ | CDR1 | TGTX₇X₈DVGX₉YNYVS |
| | [SEQ ID NO: 152] | | [SEQ ID NO: 155] |
| CDR2 | SIYPSGGX₄TFYADX₅VKG | CDR2 | X₁₀VX₁₁X₁₂RPS |
| | [SEQ ID NO: 153] | | [SEQ ID NO: 156] |
| CDR3 | IKLGTVTTVX₆Y | CDR3 | SSX₁₃X₁₄X₁₅X₁₆X₁₇RV |
| | [SEQ ID NO: 154] | | [SEQ ID NO: 157] |

wherein X₁ is M, I, or S; X₂ is R, K, L, M, or I; X₃ is F or M; X₄ is F or I; X₅ is S or T; X₆ is E or D; X7 is N or S; X₈ is T, R, or S; X₉ is A or G; X₁₀ is E or D; X₁₁ is I, N, or S; X₁₂ is D, H, or N; X₁₃ is F or Y; X₁₄ is N or S; X₁₅ is R, T, or S, X₁₆ is G or S, and X₁₇ is I or T.

In specific embodiments, the anti-PD-L1 antigen-binding molecule comprises the heavy chain amino acid sequence of avelumab as provided for example below:

[SEQ ID NO: 158]
EVQLLESGGGLVQPGGSLRLSCAASGFTFSSYIMMWVRQAPGKGLEWVSS
IYPSGGITFYADTVKGRFTISRDNSKNTLYLQMNSLRAEDTAVYYCARIK
LGTVTTVDYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPG
K,

or an antigen-binding fragment thereof, which comprises, consists or consists essentially of the amino acid sequence:

[SEQ ID NO: 159]
EVQLLESGGGLVQPGGSLRLSCAASGFTFSSYIMMWVRQAPGKGLEWVSS
IYPSGGITFYADTVKGRFTISRDNSKNTLYLQMNSLRAEDTAVYYCARIK
LGTVTTVDYWGQGTLVTVSS.

In some of the same and other embodiments, the anti-PD-L1 antigen-binding molecule comprises the light chain amino acid sequence of avelumab as set out for example below:

[SEQ ID NO: 160]
QSALTQPASVSGSPGQSITISCTGTSSDVGGYNYV
SWYQQHPGKAPKLMIYDVSNRPSGVSNRFSGSKSG
NTASLTISGLQAEDEADYYCSSYTSSSTRVFGTGT
KVTVLGQPKANPTVTLFPPSSEELQANKATLVCLI
SDFYPGAVTVAWKADGSPVKAGVETTKPSKQSNNK
YAASSYLSLTPEQWKSHRSYSCQVTHEGSTVEKTV
APTECS,

or an antigen-binding fragment thereof, which comprises, consists or consists essentially of the amino acid sequence:

[SEQ ID NO: 161]
QSALTQPASVSGSPGQSITISCTGTSSDVGGYNYV
SWYQQHPGKAPKLMIYDVSNRPSGVSNRFSGSKSG
NTASLTISGLQAEDEADYYCSSYTSSSTRVFGTGT
KVTVL.

Alternatively, the anti-PD-L1 antigen-binding molecule competes for binding to PD-L1 with the MAb avelumab.

In some embodiments, the ICM antagonist is an antagonist of CTLA4. Alternative names or synonyms for CTLA4 include ALPSS, CD, CD152, CELIAC3, CTLA-4, GRD4, GSE, IDDM12.

Generally, the CTLA4 antagonists bind specifically to the mature amino acid sequence of human CTLA4 (UniProt accession no. P16410) as set out for example below:

[SEQ ID NO: 16]
KAMHVAQPAVVLASSRGIASFVCEYASPGKATEVR
VTVLRQADSQVTEVCAATYMMGNELTFLDDSICT
GTSSGNQVNLTIQGLRAMDTGLYICKVELMYPPPY
YLGIGNGTQIYVIDPEPCPDSDFLLWILAAVSSGL
FFYSFLLTAVSLSKMLKKRSPLTTGVYVKMPPTEP
ECEKQFQPYFIPIN.

Suitably, the CTLA4 antagonist is an anti-CTLA4 antigen-binding molecule. By way of example, anti-CTLA4 antigen-binding molecules that are suitable for use with the present invention include the anti-CTLA4 MAbs ipilimumab (BMS-734016, MDX-010, MDX-101) and tremelimumab (ticilimumab, CP-675,206).

The anti-CTLA4 antigen-binding molecules suitably bind to a region of the extracellular domain of CTLA4. By way of illustration, the anti-CTLA4 antigen-binding molecules may specifically bind to a region of the extracellular domain of human CTLA4 that comprises any one or more of the amino acid sequences YASPGKATEVRVTVLRQA [SEQ ID NO:15] (i.e., residues 26 to 42 of the native CTLA4 sequence set forth in SEQ ID NO:16), DSQVTEVCAATYMMGNELTFLDD [SEQ ID NO:17] (i.e., residues 43 to 65 of the native CTLA4 sequence set forth in SEQ ID NO:16), and VELMYPPPYYLGIG [SEQ ID NO:18] (i.e., residues 96 to 109 of the native CTLA4 sequence set forth in SEQ ID NO:16). Alternatively or in addition, the anti-CTLA4 antigen-binding molecules may specifically bind to a region of the extracellular domain of human CTLA4 that comprises any one or more and preferably all of the following residues of the mature form of CTLA4: K1, A2, M3, E33, R35, Q41, S44, Q45, V46, E48, L91, 193, K95, E97, M99, P102, P103, Y104, Y105, L106, 1108, N110.

In certain embodiments, the anti-CTLA4 antigen-binding molecule comprises the human IgG1 MAb ipilimumab (as described for example in International Publication WO2014/209804 and U.S. Patent Publication No 2015/0283234, the entire contents of which are incorporated herein by reference) or an antigen-binding fragment thereof. In representative embodiments of this type, the anti-CTA4 antigen-binding molecule comprises the CDR sequences as set forth in Table 11.

TABLE 11

| Heavy chain | | Light chain | |
|---|---|---|---|
| CDR1 | SYTMH | CDR1 | RASQSVGSSYLA |
| | [SEQ ID NO: 162] | | [SEQ ID NO: 165] |
| CDR2 | FISYDGNNKYYADSVKG | CDR2 | GAFSRAT |
| | [SEQ ID NO: 163] | | [SEQ ID NO: 166] |
| CDR3 | TGWLGPFDY | CDR3 | QQYGSSPWT |
| | [SEQ ID NO: 164] | | [SEQ ID NO: 167] |

In more specific embodiments, the anti-CTLA4 antigen-binding molecule comprises the heavy chain amino acid sequence of ipilimumab as set out for example below:

[SEQ ID NO: 168]
QVQLVESGGGVVQPGRSLRLSCAASGFTFSSYTMH
WVRQAPGKGLEWVTFISYDGNNKYYADSVKGRFTI
SRDNSKNTLYLQMNSLRAEDTAIYYCARTGWLGPF
DYWGQGTLVTVSSASTKGPSVFPLAPSSKSTSGGT
AALGCLVKDYFPEPVTVSWNSGALTSGVHTFPAVL
QSSGLYSLSSVVTVPSSSLGTQTYICNVNHKPSNT
KVDKRVEPKSCDKTHTCPPCPAPELLGGPSVFLFP
PKPKDTLMISRTPEVTCVVVDVSHEDPEVKFNWYV
DGVEVHNAKTKPREEQYNSTYRVVSVLTVLHQDWL
NGKEYKCKVSNKALPAPIEKTISKAKGQPREPQVY
TLPPSRDELTKNQVSLTCLVKGFYPSDIAVEWESN
GQPENNYKTTPPVLDSDGSFFLYSKLTVDKSRWQQ
GNVFSC SVMHEALHNHYTQKSLSLSPGK,

or an antigen-binding fragment thereof, a non-limiting example of which comprises, consists or consists essentially of the amino acid sequence:

[SEQ ID NO: 169]
QVQLVESGGGVVQPGRSLRLSCAASGFTFSSYTMH
WVRQAPGKGLEWVTFISYDGNNKYYADSVKGRFTI
SRDNSKNTLYLQMNSLRAEDTAIYYCARTGWLGPF
DYWGQGTLVTVSS.

In some of the same and other embodiments, the anti-CTLA4 antigen-binding molecule comprises the light chain amino acid sequence of ipilimumab as set out for example below:

[SEQ ID NO: 170]
EIVLTQSPGTLSLSPGERATLSCRASQSVGSSYLA
WYQQKPGQAPRLLIYGAFSRATGIPDRFSGSGSGT
DFTLTISRLEPEDFAVYYCQQYGSSPWTFGQGTKV
EIKRTVAAPSVFIFPPSDEQLKSGTASVVCLLNNF
YPREAKVQWKVDNALQSGNSQESVTEQDSKDSTYS
LSSTLTLSKADYEKHKVYACEVTHQGLSSPVTKSF
NRGEC,

or an antigen-binding fragment thereof, a representative example of which comprises, consists or consists essentially of the amino acid sequence:

[SEQ ID NO: 171]
EIVLTQSPGTLSLSPGERATLSCRASQSVGSSYLA
WYQQKPGQAPRLLIYGAFSRATGIPDRFSGSGSGT
DFTLTISRLEPEDFAVYYCQQYGSSPWTFGQGTKV
EIK.

the anti-CTAL4 antigen-binding molecule comprises the human IgG2 MAb tremelimumab (as described for example in U.S. Patent Publication No 2009/0074787, the entire content of which is incorporated herein by reference) or an antigen-binding fragment thereof. In representative embodiments of this type, the anti-CTLA4 antigen-binding molecule comprises the CDR sequences as set forth in Table 12.

TABLE 12

| Heavy chain | | Light chain | |
|---|---|---|---|
| CDR1 | GFTFSSYGMH | CDR1 | RASQSINSYLD |
| | [SEQ ID NO: 172] | | [SEQ ID NO: 175] |
| CDR2 | VIWYDGSNKYYADSV | CDR2 | AASSLQS |
| | [SEQ ID NO: 173] | | [SEQ ID NO: 176] |
| CDR3 | DPRGATLYYYYYGMDV | CDR3 | QQYYSTPFT |
| | [SEQ ID NO: 174] | | [SEQ ID NO: 177] |

In more specific embodiments, the anti-CTLA4 antigen-binding molecule comprises the heavy chain amino acid sequence of tremelimumab as set out for example below:

[SEQ ID NO: 178]
QVQLVESGGGVVQPGRSLRLSCAASGFTFSSYGMH
WVRQAPGKGLEWVAVIWYDGSNKYYADSVKGRFTI
SRDNSKNTLYIQMNSLRAEDTAVYYCARDPRGATL
YYYYYGMDVWGQGTTVTVSSASTKGPSVFPLAPCS
RSTSESTAALGCLVKDYFPEPVTVSWNSGALTSGV
HTFPAVLQSSGLYSLSSVVTVPSSNFGTQTYTCNV
DHKPSNTKVDKTVERKCCVECPPCPAPPVAGPSVF
LFPPKPKDTLMISRTPEVTCVVVDVSHEDPEVQFN
WYVDGVEVHNAKTKPREEQFNSTFRVVSVLTVVHQ
DWLNGKEYKCKVSNKGLPAPIEKTISKTKGQPREP
QVYTLPPSREEMTKNQVSLTCLVKGFYPSDIAVEW
ESNGQPENNYKTTPPMLDSDGSFFLYSKLTVDKSR
WQQGNVFSCSVMHEALHNHYTQKSLSLSPGK,

or an antigen-binding fragment thereof, a non-limiting example of which comprises, consists or consists essentially of the amino acid sequence:

[SEQ ID NO: 179]
QVQLVESGGGVVQPGRSLRLSCAASGFTFSSYTMH
WVRQAPGKGLEWVTFISYDGNNKYYADSVKGRFTI
SRDNSKNTLYLQMNSLRAEDTAIYYCARTGWLGPF
DYWGQGTLVTVSS.

In some of the same and other embodiments, the anti-CTLA4 antigen-binding molecule comprises the light chain amino acid sequence of tremelimumab as set out for example below:

[SEQ ID NO: 180]
DIQMTQSPSSLSASVGDRVTITCRASQSINSYLDW
YQQKPGKAPKLLIYAASSLQSGVPSRFSGSGSGTD
FTLTISSLQPEDFATYYCQQYYSTPFTFGPGTKVE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC,

or an antigen-binding fragment thereof, a representative example of which comprises, consists or consists essentially of the amino acid sequence:

[SEQ ID NO: 181]
DIQMTQSPSSLSASVGDRVTITCRASQSINSYLDW
YQQKPGKAPKLLIYAASSLQSGVPSRFSGSGSGTD
FTLTISSLQPEDFATYYCQQYYSTPFTFGPGTKVE
IK.

In other embodiments, the ICM antagonist is a B7-H3 antagonist. Generally, the B7-H3 antagonists of the invention bind specifically to the native amino acid sequence of human B7-H3 (UniProt accession no. Q5ZPR3) as set out for example below:

[SEQ ID NO: 182]
MLRRRGSPGMGVHVGAALGALWFCLTGALEVQVPE
DPVVALVGTDATLCCSFSPEPGFSLAQLNLIWQLT
DTKQLVHSFAEGQDQGSAYANRTALFPDLLAQGNA
SLRLQRVRVADEGSFTCFVSIRDFGSAAVSLQVAA
PYSKPSMTLEPNKDLRPGDTVTITCSSYQGYPEAE
VFWQDGQGVPLTGNVTTSQMANEQGLFDVHSILRV
VLGANGTYSCLVRNPVLQQDAHSSVTITPQRSPTG
AVEVQVPEDPVVALVGTDATLRCSFSPEPGFSLAQ
LNLIWQLTDTKQLVHSFTEGRDQGSAYANRTALFP
DLLAQGNASLRLQRVRVADEGSFTCFVSIRDFGSA
AVSLQVAAPYSKPSMTLEPNKDLRPGDTVTITCSS
YRGYPEAEVFWQDGQGVPLTGNVTTSQMANEQGLF
DVHSVLRVVLGANGTYSCLVRNPVLQQDAHGSVTI
TGQPMTFPPEALWVTVGLSVCLIALLVALAFVCWR
KIKQSCEEENAGAEDQDGEGEGSKTALQPLKHSDS
KEDDGQEIA.

Suitably, the B7-H3 antagonist is an anti-B7-H3 antigen-binding molecule. By way of an example, an anti-B7-H3 antigen-binding molecule suitable for use with the present invention is the MAb enoblituzumab or an antigen-binding fragment thereof. In some embodiments the anti-B7-H3 antigen-binding molecule comprises CDR sequences as set forth in Table 13.

TABLE 13

| Heavy chain | | Light chain | |
|---|---|---|---|
| CDR1 | FGMH | CDR1 | KASQNVDTNVA |
| | [SEQ ID NO: 183] | | [SEQ ID NO: 186] |
| CDR2 | YISSDSSAIYYADTVK | CDR2 | SASYRYS |
| | [SEQ ID NO: 184] | | [SEQ ID NO: 187] |
| CDR3 | GRENIYYGSRLDY | CDR3 | QQYNNYPFT |
| | [SEQ ID NO: 185] | | [SEQ ID NO: 188] |

In more specific embodiments, the anti-B7-H3 antigen-binding molecule comprises the heavy chain amino acid sequence of enoblituzumab as set out for example below:

[SEQ ID NO: 189]
VQLVESGGGLVQPGGSLRLSCAASGFTFSSFGMHW
VRQAPGKGLEWVAYISSDSSAIYYADTVKGRFTIS
RDNAKNSLYLQMNSLRDEDTAVYYCGRGRENIYYG
SRLDYWGQGTTVTVSSASTKGPSVFPLAPSSKSTS
GGTAALGCLVKDYFPEPVTVSWNSGALTSGVHTFP
AVLQSSGLYSLSSVVTVPSSSLGTQTYICNVNHKP
SNTKVDKRVEPKSCDKTHTCPPCPAPELVGGPSVF
LLPPKPKDTLMISRTPEVTCVVVDVSHEDPEVKFN
WYVDGVEVHNAKTKPPEEQYNSTLRVVSVLTVLHQ
DWLNGKEYKCKVSNKALPAPIEKTISKAKGQPREP
QVYTLPPSREEMTKNQVSLTCLVKGFYPSDIAVEW
ESNGQPENNYKTTPLVLDSDGSFFLYSKLTVDKSR
WQQGNVFSCSVMHEALHNHYTQKSLSLSPGK,

or an antigen-binding fragment thereof, a representative example of which comprises, consists or consists essentially of the amino acid sequence:

[SEQ ID NO: 190]
VQLVESGGGLVQPGGSLRLSCAASGFTFSSFGMHW
VRQAPGKGLEWVAYISSDSSAIYYADTVKGRFTIS
RDNAKNSLYLQMNSLRDEDTAVYYCGRGRENIYYG
SRLDYWGQGTTVTVSS.

In some of the same and other embodiments, the anti-B7-H3 antigen-binding molecules comprise the light chain amino acid sequence of enoblituzumab as provided for example below.

[SEQ ID NO: 191]
DIQLTQSPSFLSASVGDRVTITCKASQNVDTNVAW
YQQKPGKAPKALIYSASYRYSGVPSRFSGSGSGTD
FTLTISSLQPEDFATYYCQQYNNYPFTFGQGTKLE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC,

or an antigen-binding fragment thereof, a representative example of which comprises, consists or consists essentially of the amino acid sequence:

[SEQ ID NO: 192]
DIQLTQSPSFLSASVGDRVTITCKASQNVDTNVAW
YQQKPGKAPKALIYSASYRYSGVPSRFSGSGSGTD
FTLTISSLQPEDFATYYCQQYNNYPFTFGQGTKLE
IK.

In some alternative embodiments, the anti-B7-H3 antigen-binding molecule competes for binding to B7-H3 with the MAb enoblituzumab.

In other embodiments, the ICM antagonist is an IDO antagonist. The mature amino acid sequence of human IDO (UniProt accession no. P14902) as set out for example below:

[SEQ ID NO: 193]
MAHAMENSWTISKEYHIDEEVGFALPNPQENLPDF
YNDWMFIAKHLPDLIESGQLRERVEKLNMLSIDHL
TDHKSQRLARLVLGCITMAYVWGKGHGDVRKVLPR
NIAVPYCQLSKKLELPPILVYADCVLANWKKKDPN
KPLTYENMDVLFSFRDGDCSKGFFLVSLLVEIAAA
SAIKVIPTVFKAMQMQERDTLLKALLEIASCLEKA
LQVFHQIHDHVNPKAFFSVLRIYLSGWKGNPQLSD
GLVYEGFWEDPKEFAGGSAGQSSVFQCFDVLLGIQ
QTAGGGHAAQFLQDMRRYMPPAHRNFLCSLESNPS
VREFVLSKGDAGLREAYDACVKALVSLRSYHLQIV
TKYILIPASQQPKENKTSEDPSKLEAKGTGGTDLM
NFLKTVRSTTEKSLLKEG.

Any IDO antagonist is suitable for use in the therapeutic agents of the present invention. Currently, three small molecule IDO inhibitors are undergoing development for clinical use: GDC-0919 (1-cyclohexyl-2-(5H-imidazo[5,1-a]isoindol-5-yl)ethanol), indoximod (1-methyl-D-tryptophan), and epacadostat (1,2,5-Oxadiazole-3-carboximidamide, 4-((2-((Aminosulfonyl)amino)ethyl)amino)-N-(3-bromo-4-fluorophenyl)-N′-hydroxy-, (C(Z))-). The molecular structure of each of these molecules is provided, below.

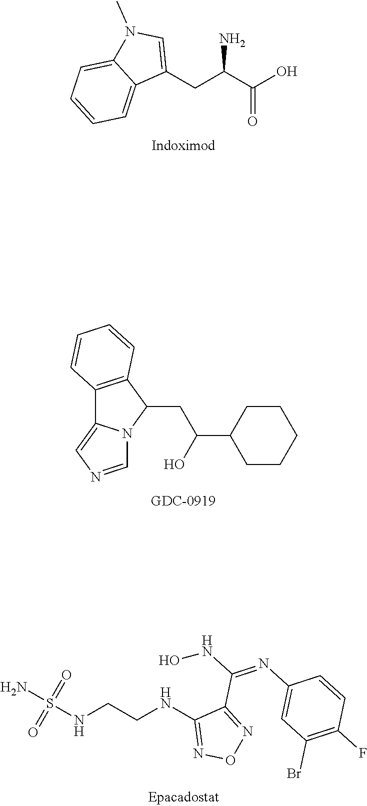 [SMILES: Cn1cc(C[C@@H](N)C(=O)O)c2ccccc21 ; OC(CC1c2ccccc2-c2cncn21)C1CCCCC1 ; NS(=O)(=O)NCCNc1nonc1/C(=N\c1ccc(F)c(Br)c1)NO]

In some embodiments, the ICM antagonist is a KIR antagonist. In preferred embodiments of this type, the KIR antagonist blocks the interaction between KIR2-DL-1, -2, and -3 and their ligands. The mature amino acid sequence of a human KIR, i.e., KIR2-DL1 (UniProt accession no. P436261 is provided for example below:

[SEQ ID NO: 194]
HEGVHRKPSLLAHPGPLVKSEETVILQCWSDVMFE
HFLLHREGMFNDTLRLIGEHHDGVSKANFSISRMT
QDLAGTYRCYGSVTHSPYQVSAPSDPLDIVIIGLY
EKPSLSAQPGPTVLAGENVTLSCSSRSSYDMYHLS
REGEAHERRLPAGPKVNGTFQADFPLGPATHGGTY
RCFGSFHDSPYEWSKSSDPLLVSVTGNPSNSWPSP
TEPSSKTGNPRHLHILIGTSVVIILFILLFFLLHR
WCSNKKNAAVMDQESAGNRTANSEDSDEQDPQEVT
YTQLNHCVFTQRKITRPSQRPKTPPTDIIVYTELP
NAESRSKVVSCP.

Anti-KIR antigen-binding molecules that are suitable for use in the invention can be generated using methods well known in the art. Alternatively, art-recognized KIR antigen-binding molecules can be used. For example, the anti-KIR antigen-binding molecule comprises the fully humanized MAb lirilumab or an antigen-binding fragment thereof as described for example in WO2014/066532, the entire content of which is hereby incorporated herein in its entirety. Suitably, the anti-KIR antigen-binding molecule comprises the CDR regions as set forth in Table 14.

TABLE 14

| Heavy chain | | Light chain | |
|---|---|---|---|
| CDR1 | FYAIS | CDR1 | RASQSVSSYLA |
| | [SEQ ID NO: 195] | | [SEQ ID NO: 198] |
| CDR2 | GFIPIFGAANYAQKFQ | CDR2 | DASNRAT |
| | [SEQ ID NO: 196] | | [SEQ ID NO: 199] |
| CDR3 | IPSGSYYYDYDMDV | CDR3 | QQRSNWMYT |
| | [SEQ ID NO: 197] | | [SEQ ID NO: 200] |

In representative embodiments of this type, the anti-KIR antigen-binding molecule may comprise the heavy chain variable domain amino acid sequence of lirilumab, as set out for example below:

[SEQ ID NO: 201]
QVQLVQSGAEVKKPGSSVKVSCKASGGTFSFYAIS
WVRQAPGQGLEWMGGFIPIFGAANYAQKFQGRVTI
TADESTSTAYMELSSLRSDDTAVYYCARIPSGSYY
YDYDMDVWGQGTTVTVSSASTKGPSVFPLAPCSRS
TSESTAALGCLVKDYFPEPVTVSWNSGALTSGVHT
FPAVLQSSGLYSLSSVVTVPSSSLGTKTYTCNVDH
KPSNTKVDKRVESKYGPPCPPCPAPEFLGGPSVFL
FPPKPKDTLMISRTPEVTCVVVDVSQEDPEVQFNW
YVDGVEVHNAKTKPREEQFNSTYRVVSVLTVLHQD
WLNGKEYKCKVSNKGLPSSIEKTISKAKGQPREPQ
VYTLPPSQEEMTKNQVSLTCLVKGFYPSDIAVEWE
SNGQPENNYKTTPPVLDSDGSFFLYSRLTVDKSRW
QEGNVFSCSVMHEALHNHYTQKSLSLSLGK,

or an antigen-binding fragment thereof, a representative example of which comprises, consists or consists essentially of the amino acid sequence:

[SEQ ID NO: 202]
QVQLVQSGAEVKKPGSSVKVSCKASGGTFSFYAIS
WVRQAPGQGLEWMGGFIPIFGAANYAQKFQGRVTI
TADESTSTAYMELSSLRSDDTAVYYCARIPSGSYY
YDYDMDVWGQGTTVTVSS.

In some of the same and other embodiments, the anti-KIR antigen-binding molecule may comprise the light chain variable domain amino acid sequence of lirilumab, as set out for example below:

[SEQ ID NO: 203]
EIVLTQSPVTLSLSPGERATLSCRASQSVSSYLAW
YQQKPGQAPRLLIYDASNRATGIPARFSGSGSGTD
FTLTISSLEPEDFAVYYCQQRSNWMYTFGQGTKLE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC,

or an antigen-binding fragment thereof, a representative example of which comprises, consists or consists essentially of the amino acid sequence:

[SEQ ID NO: 204]
EIVLTQSPVTLSLSPGERATLSCRASQSVSSYLAW
YQQKPGQAPRLLIYDASNRATGIPARFSGSGSGTD
FTLTISSLEPEDFAVYYCQQRSNWMYTFGQGTKLE
IKRT.

In alternative embodiments, the ICM antagonist is a LAG-3 antagonist. LAG-3 is a 503 amino acid type I transmembrane protein, with four extracellular Ig-like domains. LAG-3 is expressed on activated T-cells, NK cells, B-cells, and plasmacytoid DCs. The representative mature amino acid sequence of human LAG-3 (UniProt accession no. P18627), is set out below:

[SEQ ID NO: 205]
LQPGAEVPVVWAQEGAPAQLPCSPTIPLQDLSLLR
RAGVTWQHQPDSGPPAAAPGHPLAPGPHPAAPSSW
GPRPRRYTVLSVGPGGLRSGRLPLQPRVQLDERGR
QRGDFSLWLRPARRADAGEYRAAVHLRDRALSCRL
RLRLGQASMTASPPGSLRASDWVILNCSFSRPDRP
ASVHWFRNRGQGRVPVRESPHHHLAESFLFLPQVS
PMDSGPWGCILTYRDGFNVSIMYNLTVLGLEPPTP
LTVYAGAGSRVGLPCRLPAGVGTRSFLTAKWTPPG
GGPDLLVTGDNGDFTLRLEDVSQAQAGTYTCHIHL
QEQQLNATVTLAIITVTPKSFGSPGSLGKLLCEVT
PVSGQERFVWSSLDTPSQRSFSGPWLEAQEAQLLS
QPWQCQLYQGERLLGAAVYFTELSSPGAQRSGRAP
GALPAGHLLLFLILGVLSLLLLVTGAFGFHLWRRQ
WRPRRFSALEQGIHPPQAQSKIEELEQEPEPEPEP
EPEPEPEPEPEQL.

In some embodiments, the LAG-3 antagonist is an anti-LAG-3 antigen-binding molecule. By way of an illustration, a suitable anti-LAG antigen-binding molecule is the anti-LAG3 humanized MAb, BMS-986016. Other anti-LAG-3 antibodies are described in U.S. Patent Publication No. 2011/0150892 and International PCT Publication Nos. WO2010/019570 and WO2014/008218, each of which is incorporated herein by reference in their entirety.

In some embodiments, the anti-LAG-3 antigen-binding molecules comprise the CDR sequences set forth in Table 15.

TABLE 15

| Heavy chain | | Light chain | |
|---|---|---|---|
| CDR1 | DYYWN | CDRI | RASQSISSYLA |
| | [SEQ ID NO: 206] | | [SEQ ID NO: 209] |
| CDR2 | EINHRGSTNSNPSLKS | CDR2 | DASNRAT |
| | [SEQ ID NO: 207] | | [SEQ ID NO: 210] |
| CDR3 | GYSDYEYNWFDP | CDR3 | QQRSNWPLT |
| | [SEQ ID NO: 208] | | [SEQ ID NO: 211] |

The anti-LAG-3 antigen-binding molecules suitably comprise the MAb BMS-986016 or an antigen-binding fragment thereof. More specifically, in some embodiments, the anti-LAG-3 antigen-binding molecule has the heavy chain amino acid sequence of BMS-986016 as set out for example below:

[SEQ ID NO: 212]
QVQLQQWGAGLLKPSETLSLTCAVYGGSFSDYYWN
WIRQPPGKGLEWIGEINHRGSTNSNPSLKSRVTLS
LDTSKNQFSLKLRSVTAADTAVYYCAFGYSDYEYN
WFDPWGQGTLVTVSSASTKGPSVFPLAPCSRSTSE
STAALGCLVKDYFPEPVTVSWNSGALTSGVHTFPA
VLQSSGLYSLSSVVTVPSSSLGTKTYTCNVDHKPS
NTKVDKRVESKYGPPCPPCPAPEFLGGPSVFLFPP
KPKDTLMISRTPEVTCVVVDVSQEDPEVQFNWYVD
GVEVHNAKTKPREEQFNSTYRVVSVLTVLHQDWLN
GKEYKCKVSNKGLPSSIEKTISKAKGQPREPQVYT
LPPSQEEMTKNQVSLTCLVKGFYPSDIAVEWESNG
QPENNYKTTPPVLDSDGSFFLYSRLTVDKSRWQEG
NVFSCSVMHEALHNHYTQKSLSLSLGK,

or an antigen-binding fragment thereof, a representative example of which comprises, consists or consists essentially of the amino acid sequence:

[SEQ ID NO: 213]
QVQLQQWGAGLLKPSETLSLTCAVYGGSFSDYYWN
WIRQPPGKGLEWIGEINHRGSTNSNPSLKSRVTLS
LDTSKNQFSLKLRSVTAADTAVYYCAFGYSDYEYN
WFDPWGQGTLVTVSS.

Similarly, the anti-LAG-3 antigen-binding molecules may comprise a light chain amino acid sequence of BMS-986016 as set forth in SEQ ID NO:45 and provided below, of an antigen-binging fragment thereof:

[SEQ ID NO: 214]
EIVLTQSPATLSLSPGERATLSCRASQSISSYLAWYQQKPGQAPRLLIYD
ASNRATGIPARFSGSGSGTDFTLTISSLEPEDFAVYYCQQRSNWPLTFGQ
GTNLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC

or an antigen-binding fragment thereof, a representative example of which comprises, consists or consists essentially of the amino acid sequence:

[SEQ ID NO: 215]
EIVLTQSPATLSLSPGERATLSCRASQSISSYLAWYQQKPGQAPRLLIYD
ASNRATGIPARFSGSGSGTDFTLTISSLEPEDFAVYYCQQRSNWPLTFGQ
GTNLEIK..

### 4. Multispecific Antigen-Binding Molecules

The present invention provides multispecific antigen-binding molecules formed from antigen-binding molecules with different specificities, which bind to RANKL or RANK and to at least one ICM. In certain embodiments, an antigen-binding molecule having a first antigen binding specificity can be functionally linked (e.g., by chemical coupling, genetic fusion, noncovalent association or otherwise) to one or more other molecular entities, such as another antigen-binding molecule having a second antigen-binding specificity to produce a bispecific antigen-binding molecule. Specific exemplary multispecific formats that can be used in the context of the present invention include, without limitation, single-chain diabody (scDb), tandem scDb (Tandab), linear dimeric scDb (LD-scDb), circular dimeric scDb (CD-scDb), bispecific T-cell engager (BiTE; tandem di-scFv), disulfide-stabilized Fv fragment (Brinkmann et al., Proc Natl Acad Sci USA. 1993; 90: 7538-7542), tandem tri-scFv, tribody, bispecific Fab2, di-miniantibody, tetrabody, scFv-Fc-scFv fusion, di-diabody, DVD-Ig, IgG-scFab, scFab-dsscFv, Fv2-Fc, IgG-scFv fusions, such as bsAb (scFv linked to C-terminus of light chain), Bs1Ab (scFv linked to N-terminus of light chain), Bs2Ab (scFv linked to N-terminus of heavy chain), Bs3Ab (scFv linked to C-terminus of heavy chain), Ts1Ab (scFv linked to N-terminus of both heavy chain and light chain), Ts2Ab (dsscFv linked to C-terminus of heavy chain), and Knob-into-Holes (KiHs) (bispecific IgGs prepared by the KiH technology) SEED technology (SEED-IgG) and DuoBodies (bispecific IgGs prepared by the DuoBody technology), a V_{H} and a V_{L} domain, each fused to one C-terminus of the two different heavy chains of a KiHs or DuoBody such that one functional Fv domain is formed. Particularly suitable for use herein is a single-chain diabody (scDb), in particular a bispecific monomeric scDb. For reviews discussing and presenting various multispecific constructs see, for example, Chan Carter, Nature Reviews Immunology 10 (2010) 301-316; Klein et al., MAbs 4(2012) 1-11; Schubert et al., Antibodies 1 (2012) 2-18; Byrne et al., Trends in Biotechnology 31 (2013) 621; Metz et al., Protein Engineering Design & Selection 25(2012) 571-580), and references cited therein.

In specific embodiments, the present invention provides bispecific antigen-binding molecules comprising a first antigen-binding molecule (e.g., an antibody or antigen-binding fragment) that binds specifically to RANK or RANKL, and a second antigen-binding molecule (e.g., an antibody or antigen-binding fragment) that binds specifically to an ICM. In specific embodiments, the ICM is other than CTLA-4.The bispecific antigen-binding molecules suitably comprise any of the antigen-binding molecules described in detail above and elsewhere herein.

By way of illustration, the first antigen-binding molecule may bind specifically to a region of human RANKL, and the second antigen-binding molecule may bind specifically to a region of human PD-1, and preferably to a region of the extracellular domain of human PD-1.

Non-limiting examples of these embodiments include the first antigen-binding molecule comprising CDR sequences as set forth in any one of Tables 1-3. In specific examples of this type, the first antigen-binding molecule may comprise at least an antigen-binding fragment of the MAb denosumab.

Suitably, the second antigen-binding molecule that binds specifically to PD-1 comprises the CDR sequences as set forth in any one of Tables 4-6. In specific examples of this type, the second antigen-binding molecule may comprises at least an antigen-binding fragment of any one of the MAbs selected from nivolumab, pembrolizumab, and pidilizumab.

In other embodiments, the second antigen-binding molecule binds specifically to a region of human PD-L1, and preferably to a region of the extracellular domain of human PD-L1. Thus, in some embodiments, the second antigen-binding molecule binds specifically to a region of PD-L1 and comprises the CDR sequences set forth in any one of Tables 5-9. In specific examples of this type, the second antigen-binding molecule may comprise at least an antigen-binding fragment of any one of the MAbs selected from durvalumab, atezolizumab, and avelumab.

In still other embodiments, the second antigen-binding molecule binds specifically to a region of human CTLA4. Thus, in some embodiments, the second antigen-binding molecule binds specifically to human CTLA4 and comprises the CDR sequences set forth in any one of Tables 10-11. In specific examples of this type, the second antigen-binding molecule may comprise at least an antigen-binding fragment of any one of the MAbs selected from ipilimumab and tremelimumab.

The present invention also provides multispecific constructs that comprise a RANK antagonist antigen-binding molecule that has specificity for RANKL or RANK and a plurality of ICM antagonist antigen-binding molecules that have specificity for two or more ICMs. In non-limiting examples, the plurality of ICM antagonist antigen-binding molecules have specificity for an ICM combination selected from (1) PD-1 and PD-L1, (2) PD-1 and CTLA4, (3) PD-L1 and CTLA4, and (4) PD-1, PD-L1 and CTLA4. The multispecific constructs may comprise any suitable antibody or antigen-binding fragment with specificity for a particular ICM combination, including the antibody or antigen-binding fragment disclosed herein.

Multispecific antigen-binding molecules of the present invention can be generated by any number of methods well known in the art. Suitable methods include biological methods (e.g., somatic hybridization), genetic methods (e.g., the expression of a non-native DNA sequence encoding the desired antibody structure in an organism), chemical methods (e.g., chemical conjugation of two antibodies), or a combination thereof (see, Kontermann R E (ed.), Bispecific Antibodies, Springer Heidelberg Dordrecht London New York, 1-28 (2011)).

4.1 Chemical methods of producing bispecific antigen-binding molecules.

Chemically conjugated bispecific antigen-binding molecules arise from the chemical coupling of two existing antibodies or antibody fragments, such as those described above and elsewhere herein. Typical couplings include cross-linking two different full-length antibodies, cross-linking two different Fab′ fragments to produce a bispecific F(ab′)₂, and cross-linking a F(ab′)₂ fragment with a different Fab′ fragment to produce a bispecific F(ab′)₃. For chemical conjugation, oxidative re-association strategies can be used. Current methodologies include the use of the homo- or heterobifunctional cross-linking reagents (Id.).

Heterobifunctional cross-linking reagents have reactivity toward two distinct reactive groups on, for example, antibody molecules. Examples of heterobifunctional cross-linking reagents include SPDP (N-succinimidyl-3-(2-pyridyldithio)propionate), SATA (succinimidyl acetylthioacetate), SMCC (succinimidγ1 trans-4-(maleimidylmethyl)cyclohexane-1-carboxylate), EDAC (1-ethyl-3-(3-dimethylaminopropyl) carbodiimide), PEAS (N-((2-pyridyldithio)ethyl)-4-azidosalicylamide), ATFB-SE (4-azido-2,3,5,6-tetrafluorobenzoic acid, succinimidγ1 ester), benzophenone-4-maleimide, benzophenone-4-isothiocyanate, 4-benzoylbenzoic acid, succinimidyl ester, iodoacetamide azide, iodoacetamide alkyne, Click-iT maleimide DIBO alkyne, azido (PEO)₄ propionic acid, succinimidγ1 ester, alkyne, succinimidγ1 ester, Click_iT succinimidγ1 ester DIBO alkyne, Sulfo-SBED (sulfo-N-hydroxysuccinimidyl-2-(6-[biotinamido]-2-(p-azido benzamido)-hexanoamido)ethyl-1,3′-dithioproprionate), photoreactive amino acids (e.g., L-photo-leucine and L-photo-methionine), NHS-haloacetγ1 crosslinkers (e.g., sulfo-SIAB), SIAB, SBAP, SIA, NHS-maleimide crosslinkers (e.g., sulfo-SMCC), SM(PEG)ₙ series cross-linkers, SMCC, LC-SMCC, sulfo-EMCS, sulfo-GMBS, GMBS, sulfo-KMUS, sulfo-MBS, MBS, Sulfo-SMPB, SMPB, AMAS, BMPS, SMPH, PEG12-SPDP, PEG4-SPDP, sulfo-LC-SPDP, LC-SPDP, SMPT, DCC (N,N′-Dicyclohexylcarbodiimide), EDC (1-Ethyl-3-(3-dimethylaminopropyl) carbodiimide), NHS (N-hydroxysuccinimide), sulfo-NHS (N-hydroxysulfosuccinimide), BMPH, EMCH, KMUH, MPBH, PDPH, and PMPI.

Homobifunctional cross-linking reagents have reactivity toward the same reactive group on a molecule, for example, an antibody. Examples of homobifunctional cross-linking reagents include DTNB (5,5′-dithiobis(2-nitrobenzoic acid), o-PDM (o-phenylenedimaleimide), DMA (dimethγ1 adipimidate), DMP (dimethγ1 pimelimidate), DMS (dimethyl suberimidate), DTBP (dithiobispropionimidate), BS(PEG)₅, BS(PEG)₉, BS³, BSOCOES, DSG, DSP, DSS, DST, DTSSP, EGS, sulfo-EGS, TSAT, DFDNB, BM(PEG)ₙ cross-linkers, BMB, BMDB, BMH, BMOE, DTME, and TMEA.

### 4.2 Biological Methods of Producing Bispecific Antigen-Binding Molecules

Somatic hybridization is the fusion of two distinct hybridoma (a fusion of B-cells that produce a specific antibody and myeloma cells) cell lines, producing a quadroma capable of generating two different antibody heavy chains (i.e., VHA and VHB) and light chains (i.e., VLA and V_{L}B). (Kontermann, supra). These heavy and light chains combine randomly within the cell, resulting in bispecific antigen-binding molecules (e.g., a V_{H}A chain combined with a V_{L}A chain and a V_{H}B chain combined with a V_{L}B chain), as well as some non-functional (e.g., two V_{H}A chains combined with two VLB chains) and monospecific (e.g., two VHA chains combined with two V_{H}A chains) antigen-binding molecules. The bispecific antigen-binding molecules can then be purified using well established methods, for example, using two different affinity chromatography columns.

Similar to monospecific antigen-binding molecules, bispecific antigen-binding molecules may also contain an Fc region that elicits Fc-mediated effects downstream of antigen binding. These effects may be reduced by, for example, proteolytically cleaving the Fc region from the bispecific antibody by pepsin digestion, resulting in bispecific F(ab′)₂ molecules (Id.).

4.3 Genetic methods of producing multispecific antigen-binding molecules

Multispecific antigen-binding molecules may also be generated by genetic means as well established in the art, e.g., in vitro expression of a plasmid containing a DNA sequence corresponding to the desired antibody structure (see, e.g., Kontermann, supra).

### 4.4 Diabodies

In some embodiments, the multispecific antigen-binding molecule is a diabody. Diabodies are composed of two separate polypeptide chains from, for example, antibodies that bind RANKL and an ICM, each chain bearing two variable domains (V_{H}A-V_{L}B and V_{H}B-V_{L}A or V_{L}A-V_{H}B and VLB-VHA). Typically, the polypeptide linkers joining the variable domains are short (i.e., from about 2, 3, 4, 5, 6, 7, 8, 9 or 10 amino acid residues). The short polypeptide linkers prevent the association of V_{H} and V_{L} domains on the same chain, and therefore promote the association of VH and V_{L} domains on different chains. Heterodimers that form are functional against both target antigens, (e.g., V_{H}A-V_{L}B with VHB-VLA or V_{L}A-V_{H}B with VLB-VHA). However, homodimers can also form (e.g., V_{H}A-V_{L}B with V_{H}A-V_{L}B, V_{H}B-V_{L}A with V_{H}B-V_{L}A, etc.), leading to non-functional molecules. Several strategies are known in the art for preventing homodimerization, including the introduction of disulphide bonds to covalently join the two polypeptide chains, modification of the polypeptide chains to include large amino acids on one chain and small amino acids on the other (knobs-into-holes structures, as discussed above and elsewhere herein), and addition of cysteine residues at C-terminal extensions. Another strategy is to join the two polypeptide chains by a polypeptide linker sequence, producing a single-chain diabody molecule (scDb) that exhibits a more compact structure than a taFv. ScDbs or diabodies can be also be fused to the IgG1 CH3 domain or the Fc region, producing di-diabodies. Examples of di-diabodies include, but are not limited to, scDb-Fc, Db-Fc, scDb-CH3, and Db-CH3. Additionally, scDbs can be used to make tetravalent bispecific molecules. By shortening the polypeptide linker sequence of scDbs from about 15 amino acids to about 5 amino acids, dimeric single-chain diabody molecules result, known as TandAbs (as described in Muller and Kontermann, in Bispecific Antibodies Kontermann R E (ed.), Springer Heidelberg Dordrecht London New York, 83-100 (2011)).

4.5 Other conjugation techniques for antigen-binding molecule generation

Another suitable strategy for generating multispecific antigen-binding molecules according to the present invention includes conjugating or otherwise linking heterodimerizing peptides to the C-terminus of the antibody molecules (e.g., scFvs or Fabs).

A non-limiting example of this strategy is the use of antibody fragments linked to jun-fos leucine zippers (e.g., scFv-Jun/Fos and Fab′-Jun/Fos).

An additional method for generating a bispecific antigen-binding molecules comprises derivatizing two antibodies with different antigen binding fragments with biotin and then linking the two antibodies via streptavidin, followed by purification and isolation of the resultant bispecific antibody.

Additional types of bispecific antigen-binding molecules according to the present invention include those that contain more than one antigen-binding site for each antigen. For example, additional V_{H} and V_{L} domains can be fused to the N-terminus of the V_{H} and V_{L} domains of an existing antibody, effectively arranging the antigen-binding sites in tandem. These types of antibodies are known as dual-variable-domain antibodies (DVD-Ig) (see, Tarcsa, E. et al., in Bispecific Antibodies. Kontermann, supra, pp. 171-185). Another method for producing antibodies that contain more than one antigen-binding site for an antigen is to fuse scFv fragments to the N-terminus of the heavy chain or the C-terminus of the light chain (discussed in more detail below).

The antibodies or antigen-binding fragments of a multispecific antigen-binding molecule complex or construct are independently selected from the group consisting of IgM, IgG, IgD, IgA, IgE, or fragments thereof, which are distinguished from each other by the amino acid sequence of the constant region of their heavy chains. Several of these Ig classes are further divided into subclasses, such as IgG1, IgG2, IgG3, and IgG4, IgAl and IgA2. The heavy chain constant regions that correspond to the different classes of antibodies are called α, δ, ε, γ and μ, respectively. The light chain constant regions (CL) which can be found in all five antibody classes are selected from K (kappa) and A (lambda). Antibody fragments that retain antigen recognition and binding capability that are Fab, Fab′, F(ab′)₂, and Fv fragments. Further, the first and second antigen binding fragments are connected either directly or by a linker (e.g., a polypeptide linker).

4.6 Generating Bispecific Antigen-Binding Molecules using an IgG Scaffold.

Constant immunoglobulin domains can suitably be used to promote heterodimerization of two polypeptide chains (e.g., IgG-like antibodies). Non-limiting examples of this strategy for producing bispecific antibodies include the introduction of knobs-into-holes structures into the two polypeptides and utilization of the naturally occurring heterodimerization of the C_{L} and C_{H}1 domains (see, Kontermann, supra, pp. 1 -28 (2011) Ridgway et al., Protein Eng. 1996 Jul;9(7):617-21; Atwell et al., J Mol Bio1.1997 Jul 4;270(1):26-35).

The majority of the recombinant antigen-binding molecules according to the present invention can be engineered to be IgG-like, meaning that they also include an Fc domain. Similar to diabodies that require heterodimerization of engineered polypeptide chains, IgG-like antigen-binding molecules also require heterodimerization to prevent the interaction of like heavy chains or heavy chains and light chains from two antibodies of different specificity (Jin, P. and Zhu, Z. In: Bispecific Antibodies. Kontermann RE (ed.), Springer Heidelberg Dordrecht London New York, pp. 151-169 (2011)).

Knobs-into-holes structures facilitate heterodimerization of polypeptide chains by introducing large amino acids (knobs) into one chain of a desired heterodimer and small amino acids (holes) into the other chain of the desired heterodimer. Steric interactions will favour the interaction of the knobs with holes, rather than knobs with knobs or holes with holes. In the context of bispecific IgG-like antibodies, like heavy chains can be prevented from homodimerizing by the introduction of knobs-into-holes (KiH) structures into the CH3 domain of the Fc region. Similarly, promoting the interaction of heavy chains and light chains specific to the same antigen can be accomplished by engineering KiH structures at the V_{H}-V_{L} interface. Specifically, in KiH methodology, large amino acid side chains are introduced into the CH3 domain of one of the heavy chains, which side chains fit into appropriately designed cavities in the CH3 domain of the other heavy chain (see, e.g., Ridgeway et al., Protein Eng. 9(1996), 617-621 and Atwell et al., J. Mol. Biol. 270(1997), 677-681, which are hereby incorporated by reference herein). Thus, heterodimers of the heavy chains tend to be more stable than either homodimer, and form a greater proportion of the expressed polypeptides. In addition, the association of the desired light-chain/heavy-chain pairings can be induced by modification of one Fab of the bispecific antibody (Fab region) to “swap” the constant or constant and variable regions between the light and heavy chains. Thus, in the modified Fab domain, the heavy chain would comprise, for example, CL-V_{H} or CL-V_{L} domains and the light chain would comprise CHI-V_{L} or CH_{I}-V_{H} domains, respectively. This prevents interaction of the heavy/light chain Fab portions of the modified chains (i.e., modified light or heavy chain) with and the heavy/light chain Fab portions of the standard/non-modified arm. By way of explanation, the heavy chain in the Fab domain of the modified arm, comprising a C_{L} domain, does not preferentially interact with the light chain of the non-modified arm/Fab domain, which also comprises a C_{L} domain (preventing “improper” or undesired pairings of heavy/light chains). This technique for preventing association of “improper” light/heavy chains is termed “CrossMAb” technology and, when combined with KiH technology, results in remarkably enhanced expression of the desired bispecific molecules (see, e.g., Schaefer et al. Proc Natl Acad Sci U S A. 2011; 108(27):11187-92; and U.S. Patent Publication No 2010/0159587, which are hereby incorporated by reference herein in their entirety). Other examples of KiH structures exist and the examples discussed above should not be construed to be limiting. Other methods to promote heterodimerization of Fc regions include engineering charge polarity into Fc domains (see, Gunasekaran et al., 2010) and SEED technology (SEED-IgG) (Davis et al., Protein Eng Des Sel. 2010 Apr;23(4):195-202, 2010).

In specific embodiments, the multispecific antigen-binding molecules are CrossMAbs, which are derived from independent parental antibodies in which antibody domain exchange is based on KiH methodology. Light chain mispairing is overcome using domain crossovers and heavy chains heterodimerized using the KIH method. For the domain crossovers either the variable domains or the constant domains are swapped between light and heavy chains to create two asymmetrical Fab arms to avoid light-chain mispairing while the “crossover” keeps the antigen-binding affinity. In comparison with natural antibodies, CrossMAbs show higher stability. There are several different CrossMAb formats, such as Fab, V_{H}-V_{L} and C_{H1}-C_{L} exchanged in different regions. In preferred embodiments, the multispecific antigen-binding molecules are based on the CrossMAbC_{H1}-C_{L} format, which exchanges the C_{H1} and C_{L} regions of the bispecific antibody.

Additional heterodimerized IgG-like antigen-binding molecules include, but are not limited to, heteroFc-scFvs, Fab-scFvs, IgG-scFv, and scFv-IgG. HeteroFc-scFvs link two distinct scFvs to heterodimerizable Fc domains while Fab-scFvs contain a Fab domain specific to one epitope linked to an scFv specific to a different epitope. IgG-scFv and scFv-IgG are Ig-like antibodies that have scFvs linked to their C-termini and N-termini, respectively (see, Kontermann R E (ed.), supra, pp. 151-169).

Representative CrossMAb embodiments are described in Section 5.4 herein, in which an engineered protuberance is created in the interface of a first IgG-like polypeptide by replacing at least one contact residue of that polypeptide within its CH3 domain. In particular, the contact residue to be replaced on the first polypeptide corresponds to an IgG residue at position 366 (residue numbering is according to Fc crystal structure (Deisenhofer, Biochem. 20:2361 [1981]) and wherein an engineered protuberance comprises replacing the nucleic acid encoding the original residue with nucleic acid encoding an import residue having a larger side chain volume than the original residue. Specifically, the threonine (T) residue at position 366 is mutated to tryptophan (W). In the second step, an engineered cavity is created in the interface of the second polypeptide by replacing at least one contact residue of the polypeptide within its C_{H}3 domain, wherein the engineered cavity comprises replacing the nucleic acid encoding an original residue with nucleic acid encoding an import residue having a smaller side chain volume than the original residue. Specifically, the contact residue to be replaced on the second polypeptide corresponds to an IgG residue at position 407. Specifically, the tyrosine (Y) residue at position 407 is mutated to alanine (A). This procedure can be engineered on different IgG subtypes, selected from the group consisting of IgG1, IgG2a, IgG2b, IgG3 and IgG4.

In another illustrative example of CrossMAb technology, the multispecific antigen-binding molecules can be based on the duobody platform /cFAE (GenMAb), as described for example in WO2008119353 and WO 2011131746 (each of which is hereby incorporated herein by reference in its entirety) in which the bispecific antibody is generated by separate expression of the component antibodies in two different host cells followed by purification and assembly into bi-specific heterodimeric antibodies through a controlled Fab-arm exchange between two monospecific antibodies. By introducing asymmetrical, matching mutations (e.g., F405L and K409R, according to EU numbering index) in the CH3 regions of two monospecific starting proteins, similar to the the Fab-arm exchange can be forced to become directional, thereby yielding stable heterodimeric pairs under reducing conditions (as described, for example by Labrijn et al., Proc Natl Acad Sci USA 2013;110(13):5145-5150; Gramer et al. MAbs 2013;5(6): 962-973; Labrijn et al. Nature Protocols 2014;9(10):2450-63, which are hereby incorporated by reference herein in their entirety). In practice, bispecific human IgG1 Abs can be produced from the two purified bivalent parental antibodies, each with the respective single complementary mutation: K409R or F405L. This same strategy can be performed on human IgG1, IgG2, IgG3 or IgG4 backbone (Labrijn 2013, supra).

### 4.7 Electrostatic Steering

In other embodiments, the multispecific antigen-binding molecules are based on electrostatic steering (Amgen, in which the charge complementarity at the CH3 domain is altered, through selected mutations, leading to enhanced antibody Fc heterodimer formation through electrostatic steering effects (Gunasekaran et al., J Biol Chem 2010;285(25):19637-46; WO 2009089004 A1, which are hereby incorporated herein by reference). This same strategy can be performed on human IgG1, IgG2, IgG3 or IgG4 backbone (WO 2009089004 A1).Linkers.

Linkers may be used to covalently link different antigen-binding molecules to form a chimeric molecule comprising at least two antigen-binding molecules. The linkage between antigen-binding molecules may provide a spatial relationship to permit binding of individual antigen-binding molecules to their corresponding cognate epitopes. In this context, an individual linker serves to join two distinct functional antigen-binding molecules. Types of linkers include, but are not limited to, chemical linkers and polypeptide linkers.

The linker may be chemical and include for example an alkylene chain, a polyethylene glycol (PEG) chain, polysuccinic anhydride, poly-L-glutamic acid, poly(ethyleneimine), an oligosaccharide, an amino acid chain, or any other suitable linkage. In certain embodiments, the linker itself can be stable under physiological conditions, such as an alkylene chain, or it can be cleavable under physiological conditions, such as by an enzyme (e.g., the linkage contains a peptide sequence that is a substrate for a peptidase), or by hydrolysis (e.g., the linkage contains a hydrolyzable group, such as an ester or thioester). The linker can be biologically inactive, such as a PEG, polyglycolic acid, or polylactic acid chain, or can be biologically active, such as an oligo- or polypeptide that, when cleaved from the moieties, binds a receptor, deactivates an enzyme, etc. The linker may be attached to the first and second antibodies or antigen-binding fragments by any suitable bond or functional group, including carbon-carbon bonds, esters, ethers, amides, amines, carbonates, carbamates, sulfonamides, etc.

In certain embodiments, the linker represents at least one (e.g., 1, 2, 3, 4, 5, 6, 7, 8, 9, 10, or more) derivatized or non-derivatized amino acid. In illustrative examples of this type, the linker is preferably non-immunogenic and flexible, such as those comprising serine and glycine sequences or repeats of Ala-Ala-Ala. Depending on the particular construct, the linkers may be long (e.g., greater than 12 amino acids in length) or short (e.g., 1, 2, 3, 4, 5, 6, 7, 8, 9, 10, 11, 12 amino acids in length). For example, to make a single chain diabody, the first and the third linkers are preferably about 3 to about 12 amino acids in length (and more preferably about 5 amino acids in length), and the second linker is preferably longer than 12 amino acids in length (and more preferably about 15 amino acids in length). Reducing the linker length to below three residues can force single chain antibody fragments into the present invention allowing the bispecific antibody to become bivalent, trivalent, or tetravalent, as desired.

Representative peptide linkers may be selected from: [AAA]ₙ, [SGGGG]ₙ, [GGGGS]ₙ, [GGGGG]ₙ, [GGGKGGGG]ₙ, [GGGNGGGG]ₙ, [GGGCGGGG]ₙ, wherein n is an integer from 1 to 10, suitably 1 to 5, more suitably 1 to 3.

### 5. Multispecific Antigen-Binding Constructs

One aspect of the present invention relates to chimeric constructs that comprise a plurality of antigen-binding molecules with different specificities that are fused to or otherwise conjugated together, either directly or via a linker.

### 5.1 Anti-RANKL-Anti-PD-1 Diabody

The present invention contemplates multispecific constructs which are bispecific and comprise an anti-RANKL antigen-binding molecule and an anti-PD-1 antigen-binding molecule, representative examples of which comprise, consist or consist essentially of a sequence selected from the following:

[SEQ ID NO: 216]
EVQLLESGGGLVQPGGSLRLSCAASGFTFSSYAMSWVRQAPGKGLEWVSG
ITGSGGSTYYADSVKGRFTISRDNSKNTLYLQMNSLRAEDTAVYYCAKDP
GTTVIMSWFDPWGQGTLVTVSS[SGGGG]ₙeivltqspatlslspgerat
lscrasasvssvlawyqqkpggaprlliydasnratgiparfsgsgsgtd
ftltisslepedfavyycqqssnwprtfgqgtkveik[SGGGG]ₙQVQLV
ESGGGVVQPGRSLRLDCKASGITFSNSGMHWVRQAPGKGLEWVAVIWYDG
SKRYYADSVKGRFTISRDNSKNTLFLQMNSLRAEDTAVYYCATNDDYWGQ
GTLVTVSS[SGGGG]ₙeivltqspgtlslspgeratlscrasqsvrgryl
awyqqkpgqaprlliygassratgipdrfsgsgsgtdftltisrlepedf
avfycqqygssprtfgqgtkveik

wherein:

Uppercase regular text corresponds to the variable heavy chain amino acid sequence of the anti-RANKL MAb denosumab,

Lowercase underlined text corresponds to the variable light chain amino acid sequence of the anti-PD-1 MAb nivolumab,

Uppercase underlined text corresponds to the variable heavy chain amino acid sequence of anti-PD-1 MAb nivolumab,

Lowercase regular text corresponds to the variable light chain amino acid sequence of anti-RANKL MAb denosumab,

Each occurrence of [SGGGG]ₙ is a flexible linker, wherein n=1, 2, 3, or 4, preferably n=1 for the first and third instances of the flexible linker, and n=3 for the second instance of the flexible linker.

[SEQ ID NO: 217]
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSS[SGGGG]ₙeivltaspatislspgeratlscrasqsv
rgrylawyqqkpgqaprlliygassratgipdrfsgsgsgtdftltisrl
epedfavfycqqygssprtfgqgtkveik[SGGGG]ₙEVQLLESGGGLVQ
PGGSLRLSCAASGFTFSSYAMSWVRQAPGKGLEWVSGITGSGGSTYYADS
VKGRFTISRDNSKNTLYLQMNSLRAEDTAVYYCAKDPGTTVIMSWFDPWG
QGTLVTVSS[SGGGG]ₙeivltaspatlslspgeratlscrasqsyssyl
awvqqkpgaarrlliydasnratgiparfsgsgsgtdftltisslepedf
avyycqqssnwprtfqqgtkveik

b)

wherein:

Uppercase underlined text corresponds to the variable heavy chain amino acid sequence of anti-PD-1 MAb nivolumab,

Lowercase regular text corresponds to the variable light chain amino acid sequence of anti-RANKL MAb denosumab,

Uppercase regular text corresponds to the variable heavy chain amino acid sequence of anti-RANKL MAb denosumab,

Lowercase underlined text corresponds to the variable light chain amino acid sequence of anti-PD-1 MAb nivolumab,

Each occurrence of [SGGGG]ₙ, is a flexible linker, wherein n=1, 2, 3, or 4, preferably n=1 for the first and third instances of the flexible linker, and n=3 for the second instance of the flexible linker. c)

[SEQ ID NO: 218]
QVQLVQSGAEVRKPGASVKVSCKASGYDFSNYAIHWVRQAPGQRLEWMGW
INAGNGNTKFSQKFQGRITVTRDTAASTAYMELRSLRSEDTAVYYCARDS
SNMVRGIIIAYYFDYWGQGTLVTVSS[SGGGG]ₙeivltqspatlslspg
eratlscrasqsvssvlawyqqkpgqaprlliydasnratgiparfsgsg
sgtpftltisslepedfavyycqqssnwprtfgqgtkveik[SGGGG]ₙQ
VQLVESGGGVVQPGRSLRLDCKASGITFSNSGMHWVRQAPGKGLEWVAVI
WYDGSKRYYADSVKGRFTISRDNSKNTLFLQMNSLRAEDTAVYYCATNDD
YWGQGTLVTVSS[SGGGG]ₙeivmtqspsslsasvgdrvtitcrasqsis
rylnwyqlkpgkaprlliygasslqsgvpsrfsgsgsgaeftltisslqp
ediatyycqhtrafgqgtkveik

wherein:

Uppercase regular text corresponds to the variable heavy chain amino acid sequence of another embodiment of an anti-RANKL antibody as disclosed in EP 1257648,

Lowercase underlined text corresponds to the variable light chain amino acid sequence of anti-PD-1 MAb nivolumab,

Uppercase underlined text corresponds to the variable heavy chain amino acid sequence of anti-PD-1 MAb nivolumab,

Lowercase regular text corresponds to the variable light chain amino acid sequence of another embodiment of an anti-RANKL antibody as disclosed in EP 1257648,

Each occurrence of [SGGGG]ₙ is a flexible linker, wherein n=1, 2, 3, or 4, preferably n=1 for the first and third instances of the flexible linker, and n=3 for the second instance of the flexible linker. d)

[SEQ ID NO: 219]
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSS[SGGGG]ₙeivmtqspsslsasvgdrvtitcrasqsi
srylnwyqlkpgkaprlliygasslqsgvpsrfsgsgsgaeftltisslq
pediatyycqhtrafgqgtkveik[SGGGG]ₙQVQLVQSGAEVRKPGASV
KVSCKASGYDFSNYAIHWVRQAPGQRLEWMGWINAGNGNTKFSQKFQGRI
TVTRDTAASTAYMELRSLRSEDTAVYYCARDSSNMVRGIIIAYYFDYWGQ
GTLVTVSS[SGGGG]ₙeivltqspatlslspqeratlscrasqsvssyla
wyqqkpqqaprlliydasnratqiparfsgsgsgtdftltisslepedfa
vyycqqssnwprtfgqgtkveik

wherein:

Uppercase underlined text corresponds to the variable heavy chain amino acid sequence of anti-PD-1 MAb nivolumab,

Lowercase regular text corresponds to the variable light chain amino acid sequence of another embodiment of an anti-RANKL antibody as disclosed in EP 1257648,

Uppercase regular text corresponds to the variable heavy chain amino acid sequence of another embodiment of an anti-RANKL antibody as disclosed in EP 1257648,

Lowercase underlined text corresponds to the variable light chain amino acid sequence of anti-PD-1 MAb nivolumab,

Each occurrence of [SGGGG]ₙ is a flexible linker, wherein n=1, 2, 3, or 4, preferably n=1 for the first and third instances of the flexible linker, and n=3 for the second instance of the flexible linker.

e)

[SEQ ID NO: 220]
EVQLLESGGGLVQPGGSLRLSCAASGFTFSSYAMSWVRQAPGKGLEWVSG
ITGSGGSTYYADSVKGRFTISRDNSKNTLYLQMNSLRAEDTAVYYCAKDP
GTTVIMSWFDPWGQGTLVTVSS[SGGGG]ₙeivltqspatlslspgerat
lscraskgvstsgysylhwyqqkpgqaprlliylasylesgvparfsgsg
sgtdftltisslepedfavyycqhsrdlpltfgggtkveik[SGGGG]ₙQ
VQLVQSGVEVKKPGASVKVSCKASGYTFTNYYMYWVRQAPGQGLEWMGGI
NPSNGGTNFNEKFKNRVTLTTDSSTTTAYMELKSLQFDDTAVYYCARRDY
RFDMGFDYWGQGTTVTVSS[SGGGG]ₙeivltqspgtlslspgeratlsc
rasqsvrgrylawyqqkpgqaprlliygassratgipdrfsgsgsgtdft
ltisrlepedfavfycqqygssprtfgqgtkveik

wherein:

Uppercase regular text corresponds to the variable heavy chain amino acid sequence of the anti-RANKL MAb denosumab,

Lowercase underlined text corresponds to the variable light chain amino acid sequence of the anti-PD-1 MAb pembrolizumab,

Uppercase underlined text corresponds to the variable heavy chain amino acid sequence of anti-PD-1 MAb pembrolizumab,

Lowercase regular text corresponds to the variable light chain amino acid sequence of anti-RANKL MAb denosumab,

Each occurrence of [SGGGG]ₙ is a flexible linker, wherein n=1, 2, 3, or 4, preferably n=1 for the first and third instances of the flexible linker, and n=3 for the second instance of the flexible linker.

f)

[SEQ ID NO: 221]
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSS[SGGGG]ₙeivltqspgtlslspgeratls
crasqsvrgrylawyqqkpgqaprlliygassratgipdrfsgsgsgtdf
tltisrlepedfavfycqqygssprtfgqgtkveik[SGGGG]ₙEVQLLE
SGGGLVQPGGSLRLSCAASGFTFSSYAMSWVRQAPGKGLEWVSGITGSGG
STYYADSVKGRFTISRDNSKNTLYLQMNSLRAEDTAVYYCAKDPGTTVIM
SWFDPWGQGTLVTVSS[SGGGG]ₙeivltqspatlslspgeratlscras
kgvstsgysylhwyqqkpqqaprlliylasylesgvparfsgsgsgtdft
ltisslepedfavyycqhsrdlpltfgggtkveik

wherein:

Uppercase underlined text corresponds to the variable heavy chain amino acid sequence of anti-PD-1 MAb pembrolizumab,

Lowercase regular text corresponds to the variable light chain amino acid sequence of anti-RANKL MAb denosumab,

Uppercase regular text corresponds to the variable heavy chain amino acid sequence of anti-RANKL MAb denosumab,

Lowercase underlined text corresponds to the variable light chain amino acid sequence of anti-PD-1 MAb pembrolizumab,

Each occurrence of [SGGGG]ₙ is a flexible linker, wherein n=1, 2, 3, or 4, preferably n=1 for the first and third instances of the flexible linker, and n=3 for the second instance of the flexible linker.

[SEQ ID NO: 222]
QVQLVQSGAEVRKPGASVKVSCKASGYDFSNYAIHWVRQAPGQRLEWMGW
INAGNGNTKFSQKFQGRITVTRDTAASTAYMELRSLRSEDTAVYYCARDS
SNMVRGIIIAYYFDYWGQGTLVTVSS[SGGGG]ₙeivltqspatlslspg
eratlscraskgvstsgysvlhwyqqkpgaaprlliylasylesgvparf
sgsgsgtdftltisslepedfavyycqhsrdlpltfgggtkveik[SGGG
G]ₙQVQLVQSGVEVKKPGASVKVSCKASGYTFTNYYMYWVRQAPGQGLEW
MGGINPSNGGTNFNEKFKNRVTLTTDSSTTTAYMELKSLOFDDTAVYYCA
RRDYRFDMGFDYWGOGTTVTVSS[SGGGG]ₙeivmtqspsslsasvgdrv
titcrasqsisrylnwyqlkpgkaprlliygasslqsgvpsrfsgsgsga
eftltisslqpediatyycqhtrafgqgtkveik

wherein:

Uppercase regular text corresponds to the variable heavy chain amino acid sequence of another embodiment of an anti-RANKL antibody as disclosed in EP 1257648,

Lowercase underlined text corresponds to the variable light chain amino acid sequence of anti-PD-1 MAb pembrolizumab,

Uppercase underlined text corresponds to the variable heavy chain amino acid sequence of anti-PD-1 MAb pembrolizumab,

Lowercase regular text corresponds to the variable light chain amino acid sequence of another embodiment of an anti-RANKL antibody as disclosed in EP 1257648,

Each occurrence of [SGGGG]ₙ is a flexible linker, wherein n=1, 2, 3, or 4, preferably n=1 for the first and third instances of the flexible linker, and n=3 for the second instance of the flexible linker.

[SEQ ID NO: 223]
QVQLVQSGVEVKKPGASVKVSCKASGYTFTNYYMYWVRQAPGQGLEWMGG
INPSNGGTNFNEKFKNRVTLTTDSSTTTAYMELKSLQFDDTAVYYCARRD
YRFDMGFDYWGQGTTVTVSS[SGGGG]ₙeivmtqspsslsasvgdrvtit
crasqsisrylnwyqlkpgkaprlliygasslqsgvpsrfsgsgsgaeft
ltisslqpediatyycqhtrafgqgtkveik[SGGGG]ₙQVQLVQSGAEV
RKPGASVKVSCKASGYDFSNYAIHWVRQAPGQRLEWMGWINAGNGNTKFS
QKFQGRITVTRDTAASTAYMELRSLRSEDTAVYYCARDSSNMVRGIIIAY
YFDYWGQGTLVTVSS[SGGGG]ₙeivltqspatlslspqeratlscrask
qvstsqvsylhwvqqkpqqaprlliylasylesqvparfsqsqsqtdftl
tisslepedfavyycqhsrdlpltfgggtkveik

wherein:

Uppercase underlined text corresponds to the variable heavy chain amino acid sequence of anti-PD-1 MAb pembrolizumab,

Lowercase regular text corresponds to the variable light chain amino acid sequence of another embodiment of an anti-RANKL antibody as disclosed in EP 1257648,

Uppercase regular text corresponds to the variable heavy chain amino acid sequence of another embodiment of an anti-RANKL antibody as disclosed in EP 1257648,

Lowercase underlined text corresponds to the variable light chain amino acid sequence of anti-PD-1 MAb pembrolizumab,

Each occurrence of [SGGGG]ₙ is a flexible linker, wherein n=1, 2, 3, or 4, preferably n=1 for the first and third instances of the flexible linker, and n=3 for the second instance of the flexible linker.

### 5.2 Anti-RANKL-anti-PD-L1 Diabody

Alternatively, the bispecific constructs comprise an anti-RANKL antigen-binding molecule and an anti-PD-L1 antigen-binding molecule, representative examples of which comprise, consist or consist essentially of a sequence selected from the following:

a)

[SEQ ID NO: 224]
EVQLLESGGGLVQPGGSLRLSCAASGFTFSSYAMSWVRQAPGKGLEWVSG
ITGSGGSTYYADSVKGRFTISRDNSKNTLYLQMNSLRAEDTAVYYCAKDP
GTTVIMSWFDPWGQGTLVTVSS[SGGGG]ₙeivltqspatlslspgerat
lscrasqrvsssvlawyqqkpgqaprlliydassratqipdrfsgsgsgt
dftltisrlepedfavyvcqqvgslpwtfgggtkveik[SGGGG]ₙVQLV
ESGGGLVQPGGSLRLSCAASGFTFSRYWMSWVRQAPGKGLEWVANIKQDG
SEKYYVDSVKGRFTISRDNAKNSLYLQMNSLRAEDTAVYYCAREGGWFGE
LAFDYWGQGTLVTVSS[SGGGG]ₙeivltqspgtlslspgeratlscras
qsvrgrylawyqqkpgqaprlliygassratgipdrfsgsgsgtdftlti
srlepedfavfycqqygssprtfgqgtkveik

wherein:

Uppercase regular text corresponds to the variable heavy chain amino acid sequence of the anti-RANKL MAb denosumab,

Lowercase underlined text corresponds to the variable light chain amino acid sequence of the anti-PD-L1 MAb durvalumab,

Uppercase underlined text corresponds to the variable heavy chain amino acid sequence of anti-PD-L1 MAb durvalumab,

Lowercase regular text corresponds to the variable light chain amino acid sequence of anti-RANKL MAb denosumab,

Each occurrence of [SGGGG]ₙ is a flexible linker, wherein n=1, 2, 3, or 4, preferably n=1 for the first and third instances of the flexible linker, and n=3 for the second instance of the flexible linker.

b) wherein:

[SEQ ID NO: 225]
VQLVESGGGLVQPGGSLRLSCAASGFTFSRYWMSWV
RQAPGKGLEWVANIKQDGSEKYYVDSVKGRFTISRD
NAKNSLYLQMNSLRAEDTAVYYCAREGGWFGELAFD
YWGQGTLVTVSS [SGGGG]ₙ eivltqspgtlsl
pgeratlscrasqsvrgrylawyqqkpgqaprlliy
gassratgipdrfsgsgsgtdftltisrlepedfav
fycqqygssprtfgqgtkveik[SGGGG]ₙ VQL
LESGGGLVQPGGSLRLSCAASGFTFSSYAMSWVRQA
PGKGLEWVSGITGSGGSTYYADSVKGRFTISRDNSK
NTLYLQMNSLRAEDTAVYYCAKDPGTTVIMSWFDPW
GQGTLVTVSS [SGGGG]ₙ eivltqspgtlslspge
ratlscrasqsvrgrylawyqqkpgqaprlliygas
sratgipdrfsgsgsgtdftlftltisrlepedfav
vvcqqyqslpwtfgqgtkveik

Uppercase underlined text corresponds to the variable heavy chain amino acid sequence of anti-PD-L1 MAb durvalumab,

Lowercase regular text corresponds to the variable light chain amino acid sequence of anti-RANKL MAb denosumab,

Uppercase regular text corresponds to the variable heavy chain amino acid sequence of anti-RANKL MAb denosumab,

Lowercase underlined text corresponds to the variable light chain amino acid sequence of anti-PD-L1 MAb durvalumab,

Each occurrence of [SGGGG]ₙ is a flexible linker, wherein n=1, 2, 3, or 4, preferably n=1 for the first and third instances of the flexible linker, and n=3 for the second instance of the flexible linker. c)

[SEQ ID NO: 226]
QVQLVQSGAEVRKPGASVKVSCKASGYDFSNYAIHWV
RQAPGQRLEWMGWINAGNGNTKFSQKFQGRITVTRDT
AASTAYMELRSLRSEDTAVYYCARDSSNMVRGIIIAY
YFDYWGQGTLVTVSS [SGGGG]ₙ eivltqsp
atlslspgeratlscrasarvsssvlawvaakpgqap
rilivdassrataipdrfsgsgsgtdftltisrlepe
dfavyycaavqslpwtfqgqtkveik [SGGGG]ₙ
VQLVESGGGLVQPGGSLRLSCAASGFTFSRYWMSWVR
QAPGKGLEWVANIKQDGSEKYYVDSVKGRFTISRDNA
KNSLYLQMNSLRAEDTAVYYCAREGGWFGELAFDYWG
QGTLVTVSS [SGGGG]ₙ eivmtqspsslsas
vgdrvtitcrasqsisrylnwyqlkpgkaprlliyga
sslqsgvpsrfsgsgsgaeftltisslqpediatyyc
qhtrafgqgtkveik

wherein:

Uppercase regular text corresponds to the variable heavy chain amino acid sequence of another embodiment of an anti-RANKL antibody as disclosed in EP 1257648,

Lowercase underlined text corresponds to the variable light chain amino acid sequence of anti-PD-L1 MAb durvalumab,

Uppercase underlined text corresponds to the variable heavy chain amino acid sequence of anti-PD-L1 MAb durvalumab,

Lowercase regular text corresponds to the variable light chain amino acid sequence of another embodiment of an anti-RANKL antibody as disclosed in EP 1257648,

Each occurrence of [SGGGG]ₙ is a flexible linker, wherein n=1, 2, 3, or 4, preferably n=1 for the first and third instances of the flexible linker, and n=3 for the second instance of the flexible linker.

d)

[SEQ ID NO: 227]
VQLVESGGGLVQPGGSLRLSCAASGFTFSRYWMSWVRQ
APGKGLEWVANIKQDGSEKYYVDSVKGRFTISRDNAKN
SLYLQMNSLRAEDTAVYYCAREGGWFGELAFDYWGQGT
LVTVSS[SGGGG]ₙ eivmtqspsslsasvgdr
vtitcrasqsisrylnwyqlkpgkaprlliygasslqs
gvpsrfsgsgsgaeftltisslqpediatyycqhtraf
gqgtkveik [SGGGG]ₙ QVQLVQSGAEVRKPG
ASVKVSCKASGYDFSNYAIHWVRQAPGQRLEWMGWINA
GNGNTKFSQKFQGRITVTRDTAASTAYMELRSLRSEDT
AVYYCARDSSNMVRGIIIAYYFDYWGQGTLVTVSS
[SGGGG]ₙ eivltqspgtlslspgeratlscra
sqrvsssylawyqqkpqgaprlliydassratgipdrfs
gsgsgtdftltisrlepedfavyycqqygslpwtfaaat
kveik

wherein:

Uppercase underlined text corresponds to the variable heavy chain amino acid sequence of anti-PD-L1 MAb durvalumab,

Lowercase regular text corresponds to the variable light chain amino acid sequence of another embodiment of an anti-RANKL antibody as disclosed in EP 1257648,

Uppercase regular text corresponds to the variable heavy chain amino acid sequence of another embodiment of an anti-RANKL antibody as disclosed in EP 1257648,

Lowercase underlined text corresponds to the variable light chain amino acid sequence of anti-PD-L1 MAb durvalumab,

Each occurrence of [SGGGG]ₙ is a flexible linker, wherein n=1, 2, 3, or 4, preferably n=1 for the first and third instances of the flexible linker, and n=3 for the second instance of the flexible linker.

e)

[SEQ ID NO: 228]
EVQLLESGGGLVQPGGSLRLSCAASGFTFSSYAMSWVRQ
APGKGLEWVSGITGSGGSTYYADSVKGRFTISRDNSKNT
LYLQMNSLRAEDTAVYYCAKDPGTTVIMSWFDPWGQGTL
VTVSS [SGGGG]ₙ diqmtqspsslsasvgdrvtit
crasqdvstavawyqqkpgkapklliysasflvsgvpsr
fsgsgsgtdftltisslqpedfatyycqqylyhpatfgq
gtkveik [SGGGG]ₙ EVQLVESGGGLVQPGGSL
RLSCAASGFTFSDSWIHWVRQAPGKGLEWVAWISPYGGS
TYYADSVKGRFTISADTSKNTAYLQMNSLRAEDTAVYYC
ARRHWPGGFDYWGQGTLVTVSS [SGGGG]ₙ eiv
ltqspgtlslspgeratlscrasqsvrgrylawyqqkpg
qaprlliygassratgipdrfsgsgsgtdftltisrlep
edfavfycqqygssprtfgqgtkveik

wherein:

Uppercase regular text corresponds to the variable heavy chain amino acid sequence of the anti-RANKL MAb denosumab,

Lowercase underlined text corresponds to the variable light chain amino acid sequence of the anti-PD-L1 MAb atezolizumab,

Uppercase underlined text corresponds to the variable heavy chain amino acid sequence of anti-PD-L1 MAb atezolizumab,

Lowercase regular text corresponds to the variable light chain amino acid sequence of anti-RANKL MAb denosumab,

Each occurrence of [SGGGG]ₙ is a flexible linker, wherein n=1, 2, 3, or 4, preferably n=1 for the first and third instances of the flexible linker, and n=3 for the second instance of the flexible linker.

f)

[SEQ ID NO: 229]
EVQLVESGGGLVQPGGSLRLSCAASGFTFSDSWIHWVRQ
APGKGLEWVAWISPYGGSTYYADSVKGRFTISADTSKNT
AYLQMNSLRAEDTAVYYCARRHWPGGFDYWGQGTLVTVS
S[SGGGG]ₙ eivltqspgtlslspgeratlscrasq
svrgrylawyqqkpgqaprlliygassratgipdrfsgs
gsgtdftltisrlepedfavfycqqygssprtfgqgtkve
ik [SGGGG]ₙ EVQLLESGGGLVQPGGSLRLSCAAS
GFTFSSYAMSWVRQAPGKGLEWVSGITGSGGSTYYADSVK
GRFTISRDNSKNTLYLQMNSLRAEDTAVYYCAKDPGTTVI
MSWFDPWGQGTLVTVSS [SGGGG]ₙ diqmtqspss
lsasvgdrvtitcrasqdvstavawyqqkpgkapklliys
asflysgvpsrfsgsgsgtdftltisslqpedfatyycqq
ylyhpatfgqgtkveik

wherein:

Uppercase underlined text corresponds to the variable heavy chain amino acid sequence of anti-PD-L1 MAb atezolizumab,

Lowercase regular text corresponds to the variable light chain amino acid sequence of anti-RANKL MAb denosumab,

Uppercase regular text corresponds to the variable heavy chain amino acid sequence of anti-RANKL MAb denosumab,

Lowercase underlined text corresponds to the variable light chain amino acid sequence of anti-PD-L1 MAb atezolizumab,

Each occurrence of [SGGGG]ₙ is a flexible linker, wherein n=1, 2, 3, or 4, preferably n=1 for the first and third instances of the flexible linker, and n=3 for the second instance of the flexible linker.

g)

[SEQ ID NO: 230]
QVQLVQSGAEVRKPGASVKVSCKASGYDFSNYAIHWVRQAP
GQRLEWMGWINAGNGNTKFSQKFQGRITVTRDTAASTAYME
LRSLRSEDTAVYYCARDSSNMVRGIIIAYYFDYWGQGTLVT
VSS [SGGGG]ₙ diqmtqspsslsasvgdrvtitcra
sqdvstavawvqqkpgkapklliysasflysqvpsrfsgsg
sgtdftltissIqpedfatyycqqylyhpatfgqgtkvei
k [SGGGG]ₙ EVQLVESGGGLVQPGGSLRLSCAASG
FTFSDSWIHWVRQAPGKGLEWVAWISPYGGSTYYADSVKGR
FTISADTSKNTAYLQMNSLRAEDTAVYYCARRHWPGGFDYW
GQGTLVTVSS [SGGGG]ₙ eivmtqspsslsasvgdrv
titcrasqsisrylnwyqlkpgkaprlliygasslqsgvps
rfsgsgsgaeftltisslqpediatyycqhtrafgqgtkve
ik

wherein:

Uppercase regular text corresponds to the variable heavy chain amino acid sequence of another embodiment of an anti-RANKL antibody as disclosed in EP 1257648,

Lowercase underlined text corresponds to the variable light chain amino acid sequence of anti-PD-L1 MAb atezolizumab,

Uppercase underlined text corresponds to the variable heavy chain amino acid sequence of anti-PD-L1 MAb atezolizumab,

Lowercase regular text corresponds to the variable light chain amino acid sequence of another embodiment of an anti-RANKL antibody as disclosed in EP 1257648,

Each occurrence of [SGGGG]ₙ is a flexible linker, wherein n=1, 2, 3, or 4, preferably n=1 for the first and third instances of the flexible linker, and n=3 for the second instance of the flexible linker.

h)

[SEQ ID NO: 231]
EVQLVESGGGLVQPGGSLRLSCAASGFTFSDSWIHWVRQ
APGKGLEWVAWISPYGGSTYYADSVKGRFTISADTSKNT
AYLQMNSLRAEDTAVYYCARRHWPGGFDYWGQGTLVTVS
S[SGGGG]ₙ eivmtqspsslsasvgdrvtitcra
sqsisrylnwyqlkpgkaprlliygasslqsgvpsrfsg
sgsgaeftltisslqpediatyycqhtrafgqgtkveik
[SGGGG]ₙ QVQLVQSGAEVRKPGASVKVSCKAS
GYDFSNYAIHWVRQAPGQRLEWMGWINAGNGNTKFSQKF
QGRITVTRDTAASTAYMELRSLRSEDTAVYYCARDSSNM
VRGIIIAYYFDYWGQGTLVTVSS [SGGGG]ₙ di
qmtqspsslsasvgdrvtitcrasqdvstavawyqqkpg
kapklliysasflysgvpsrfsgsgsgtdftltisslqp
edfatyycqqylyhpatfgqgtkveik

wherein:

Uppercase underlined text corresponds to the variable heavy chain amino acid sequence of anti-PD-L1 MAb atezolizumab,

Lowercase regular text corresponds to the variable light chain amino acid sequence of another embodiment of an anti-RANKL antibody as disclosed in EP 1257648,

Uppercase regular text corresponds to the variable heavy chain amino acid sequence of another embodiment of an anti-RANKL antibody as disclosed in EP 1257648,

Lowercase underlined text corresponds to the variable light chain amino acid sequence of anti-PD-L1 MAb atezolizumab,

Each occurrence of [SGGGG]ₙ is a flexible linker, wherein n=1, 2, 3, or 4, preferably n=1 for the first and third instances of the flexible linker, and n=3 for the second instance of the flexible linker.

### 5.3 Anti-RANKL-anti-CTLA4 Diabody

Alternatively, the bispecific constructs comprise an anti-RANKL antigen-binding molecule and an anti-CTLA4 antigen-binding molecule, representative examples of which comprise, consist or consist essentially a sequence selected from the following: a)

[SEQ ID NO: 232]
EVQLLESGGGLVQPGGSLRLSCAASGFTFSSYAMSWVRQ
APGKGLEWVSGITGSGGSTYYADSVKGRFTISRDNSKNT
LYLQMNSLRAEDTAVYYCAKDPGTTVIMSWFDPWGQGTL
VTVSS [SGGGG]ₙ eivltqspgtlslspgeratls
crasqsvgssylawyqqkpgqaprlliygafsratgipd
rfsgsgsgtdftltisrlepedfavyycqqygsspwtfg
qgtkveik [SGGGG]ₙ QVQLVESGGGVVQPGRS
LRLSCAASGFTFSSYTMHWVRQAPGKGLEWVTFISYDGN
NKYYADSVKGRFTISRDNSKNTLYLQMNSLRAEDTAIYY
CARTGWLGPFDYWGQGTLVTVSS [SGGGG]ₙ ei
vltqspgtlslspgeratlscrasqsvrgrylawyqqkp
gqaprlliygassratgipdrfsgsgsgtdftltisrle
pedfavfycqqygssprtfgqgtkveik

wherein:

Uppercase regular text corresponds to the variable heavy chain amino acid sequence of the anti-RANKL MAb denosumab,

Lowercase underlined text corresponds to the variable light chain amino acid sequence of the anti-CTLA4 MAb ipilimumab,

Uppercase underlined text corresponds to the variable heavy chain amino acid sequence of anti-CTLA4 MAb ipilimumab,

Lowercase regular text corresponds to the variable light chain amino acid sequence of anti-RANKL MAb denosumab,

Each occurrence of [SGGGG]ₙ is a flexible linker, wherein n=1, 2, 3, or 4, preferably n=1 for the first and third instances of the flexible linker, and n=3 for the second instance of the flexible linker.

b)

[SEQ ID NO: 233]
QVQLVESGGGVVQPGRSLRLSCAASGFTFSSYTMHWVRQ
APGKGLEWVTFISYDGNNKYYADSVKGRFTISRDNSKNT
LYLQMNSLRAEDTAIYYCARTGWLGPFDYWGQGTLVTVS
S [SGGGG]ₙ eivltqspgtlslspgeratlscr
asqsvrgrylawyqqkpgqaprlliygassratgipdrf
sgsgsgtdftltisrlepedfavfycqqygssprtfgqg
tkveik [SGGGG]ₙ EVQLLESGGGLVQPGGSLR
LSCAASGFTFSSYAMSWVRQAPGKGLEWVSGITGSGGST
YYADSVKGRFTISRDNSKNTLYLQMNSLRAEDTAVYYCA
KDPGTTVIMSWFDPWGQGTLVTVSS [SGGGG]ₙ e
ivltqspgtlslspgeratlscrasqsvgssvlawyqqk
pqgaprlliygafsratgipdrfsgsgsgtdftltisrl
epedfavyycqqyqsspwtfgqgtkveik

wherein:

Uppercase underlined text corresponds to the variable heavy chain amino acid sequence of anti-CTLA4 MAb ipilimumab,

Lowercase regular text corresponds to the variable light chain amino acid sequence of anti-RANKL MAb denosumab,

Uppercase regular text corresponds to the variable heavy chain amino acid sequence of anti-RANKL MAb denosumab,

Lowercase underlined text corresponds to the variable light chain amino acid sequence of anti-CTLA4 MAb ipilimumab,

Each occurrence of [SGGGG]ₙ is a flexible linker, wherein n=1, 2, 3, or 4, preferably n=1 for the first and third instances of the flexible linker, and n=3 for the second instance of the flexible linker. c)

[SEQ ID NO: 234]
QVQLVQSGAEVRKPGASVKVSCKASGYDFSNYAIHWVRQ
APGQRLEWMGWINAGNGNTKFSQKFQGRITVTRDTAAST
AYMELRSLRSEDTAVYYCARDSSNMVRGIIIAYYFDYWG
QGTLVTVSS [SGGGG]ₙ eivltqspgtlslspge
ratlscrasqsvgssylawvqqkpgqaprlliygafsra
tgipdrfsgsgsgtdftltisrlepedfavyycqqygss
pwtfqggtkveik [SGGGG]ₙ QVQLVESGGGVV
QPGRSLRLSCAASGFTFSSYTMHWVRQAPGKGLEWVTFI
SYDGNNKYYADSVKGRFTISRDNSKNTLYLQMNSLRAED
TAIYYCARTGWLGPFDYWGQGTLVTVSS [SGGGG]ₙ
eivmtqspsslsasvgdrvtitcrasqsisrylnwyqlk
pgkaprlliygasslqsgvpsrfsgsgsgaeftltissl
qpediatyycqhtrafgqgtkveik

wherein:

Uppercase regular text corresponds to the variable heavy chain amino acid sequence of another embodiment of an anti-RANKL antibody as disclosed in EP 1257648,

Lowercase underlined text corresponds to the variable light chain amino acid sequence of anti-CTLA4 MAb ipilimumab,

Uppercase underlined text corresponds to the variable heavy chain amino acid sequence of anti-CTLA4 MAb ipilimumab,

Lowercase regular text corresponds to the variable light chain amino acid sequence of another embodiment of an anti-RANKL antibody as disclosed in EP 1257648,

Each occurrence of [SGGGG]ₙ is a flexible linker, wherein n=1, 2, 3, or 4, preferably n=1 for the first and third instances of the flexible linker, and n=3 for the second instance of the flexible linker.

d)

[SEQ ID NO: 235]
QVQLVESGGGVVQPGRSLRLSCAASGFTFSSYTMHWVRQA
PGKGLEWVTFISYDGNNKYYADSVKGRFTISRDNSKNTLY
LQMNSLRAEDTAIYYCARTGWLGPFDYWGQGTLVTVS
S [SGGGG]ₙ eivmtqspsslsasvgdrvtitcra
sqsisrylnwyqlkpgkaprlliygasslqsgvpsrfsgs
gsgaeftltisslqpediatyycqhtrafgqgtkveik
[SGGGG]ₙ QVQLVQSGAEVRKPGASVKVSCKASGYD
FSNYAIHWVRQAPGQRLEWMGWINAGNGNTKFSQKFQGRI
TVTRDTAASTAYMELRSLRSEDTAVYYCARDSSNMVRGII
IAYYFDYWGQGTLVTVSS [SGGGG]ₙ eivltqspqtls
lspqeratlscrasasvqssvIawvaakpqgaprllivqa
fsratqipdrfsgsgsgtdftltisrlepedfavyycqqy
qsspwtfgqgtkveik

wherein:

Uppercase underlined text corresponds to the variable heavy chain amino acid sequence of anti-CTLA4 MAb ipilimumab,

Lowercase regular text corresponds to the variable light chain amino acid sequence of another embodiment of an anti-RANKL antibody as disclosed in EP 1257648,

Uppercase regular text corresponds to the variable heavy chain amino acid sequence of another embodiment of an anti-RANKL antibody as disclosed in EP 1257648,

Lowercase underlined text corresponds to the variable light chain amino acid sequence of anti-CTLA4 MAb ipilimumab,

Each occurrence of [SGGGG]ₙ is a flexible linker, wherein n=1, 2, 3, or 4, preferably n=1 for the first and third instances of the flexible linker, and n=3 for the second instance of the flexible linker.

e)

[SEQ ID NO: 236]
EVQLLESGGGLVQPGGSLRLSCAASGFTFSSYAMSWVRQA
PGKGLEWVSGITGSGGSTYYADSVKGRFTISRDNSKNTLY
LQMNSLRAEDTAVYYCAKDPGTTVIMSWFDPWGQGTLVTV
SS [SGGGG]ₙ diqmtqspsslsasvgdrvtitcrasqs
insyldwyqqkpgkapklliyaasslqsgvpsrfsgsgsg
tdftltisslqpedfatyycqqyystpftfgpgtkveik
[SGGGG]ₙ QVQLVESGGGVVQPGRSLRLSCAASGF
TFSSYTMHWVRQAPGKGLEWVTFISYDGNNKYYADSVKGR
FTISRDNSKNTLYLQMNSLRAEDTAIYYCARTGWLGPFDY
WGQGTLVTVSS [SGGGG]ₙ eivltqspgtlslsp
geratlscrasqsvrgrylawyqqkpgqaprlliygassr
atgipdrfsgsgsgtdftltisrlepedfavfycqqygss
prtfgqgtkveik

wherein:

Uppercase regular text corresponds to the variable heavy chain amino acid sequence of the anti-RANKL MAb denosumab,

Lowercase underlined text corresponds to the variable light chain amino acid sequence of the anti-CTLA4 MAb tremelimumab,

Uppercase underlined text corresponds to the variable heavy chain amino acid sequence of anti-CTLA4 MAb tremelimumab,

Lowercase regular text corresponds to the variable light chain amino acid sequence of anti-RANKL MAb denosumab,

Each occurrence of [SGGGG]ₙ is a flexible linker, wherein n=1, 2, 3, or 4, preferably n=1 for the first and third instances of the flexible linker, and n=3 for the second instance of the flexible linker.

g)

[SEQ ID NO: 237]
QVQLVESGGGVVQPGRSLRLSCAASGFTFSSYTMHWVRQA
PGKGLEWVTFISYDGNNKYYADSVKGRFTISRDNSKNTLY
LQMNSLRAEDTAIYYCARTGWLGPFDYWGQGTLVTVSS
[SGGGG]ₙ eivltqspgtlslspgeratlscrasq
svrgrylawyqqkpgqaprlliygassratgipdrfsgsg
sgtdftltisrlepedfavfycqqygssprtfgqgtkvei
k [SGGGG]ₙ EVQLLESGGGLVQPGGSLRLSCAAS
GFTFSSYAMSWVRQAPGKGLEWVSGITGSGGSTYYADSVK
GRFTISRDNSKNTLYLQMNSLRAEDTAVYYCAKDPGTTVI
MSWFDPWGQGTLVTVSS [SGGGG]ₙ diqmtqspssl
sasvgdrvtitcrasqsinsvldwvqqkpqkapklliyaa
sslqsgvpsrfsgsgsgtdftltisslqpedfatyycqqv
vstpftfqpqtkveik

wherein:

Uppercase underlined text corresponds to the variable heavy chain amino acid sequence of anti-CTLA4 MAb tremelimumab,

Lowercase regular text corresponds to the variable light chain amino acid sequence of anti-RANKL MAb denosumab,

Uppercase regular text corresponds to the variable heavy chain amino acid sequence of anti-RANKL MAb denosumab,

Lowercase underlined text corresponds to the variable light chain amino acid sequence of anti-CTLA4 MAb tremelimumab,

Each occurrence of [SGGGG]ₙ is a flexible linker, wherein n=1, 2, 3, or 4, preferably n=1 for the first and third instances of the flexible linker, and n=3 for the second instance of the flexible linker.

[SEQ ID NO: 238]
QVQLVQSGAEVRKPGASVKVSCKASGYDFSNYAIHWVRQA
PGQRLEWMGWINAGNGNTKFSQKFQGRITVTRDTAASTAY
MELRSLRSEDTAVYYCARDSSNMVRGIIIAYYFDYWGQGT
LVTVSS [SGGGG]ₙ diqmtaspsslsasvadrvt
itcrasasinsvldwvaakpakapkllivaasslasavps
rfsgsgsgtdftltisslapedfatyycqqvvstpftfqp
qtkveik [SGGGG]ₙ QVQLVESGGGVVQPGRSLR
LSCAASGFTFSSYTMHWVRQAPGKGLEWVTFISYDGNNKY
YADSVKGRFTISRDNSKNTLYLQMNSLRAEDTAIYYCART
GWLGPFDYWGQGTLVTVSS [SGGGG]ₙ eivmtqsps
slsasvgdrvtitcrasqsisrylnwyqlkpgkaprlliy
gasslqsgvpsrfsgsgsgaeftltisslqpediatyyc
qhtrafgqgtkveik

wherein:

Uppercase regular text corresponds to the variable heavy chain amino acid sequence of another embodiment of an anti-RANKL antibody as disclosed in EP 1257648,

Lowercase underlined text corresponds to the variable light chain amino acid sequence of anti-CTLA4 MAb tremelimumab,

Uppercase underlined text corresponds to the variable heavy chain amino acid sequence of anti-CTLA4 MAb tremelimumab,

Lowercase regular text corresponds to the variable light chain amino acid sequence of another embodiment of an anti-RANKL antibody as disclosed in EP 1257648,

Each occurrence of [SGGGG]ₙ is a flexible linker, wherein n=1, 2, 3, or 4, preferably n=1 for the first and third instances of the flexible linker, and n=3 for the second instance of the flexible linker.

h)

[SEQ ID NO: 239]
QVQLVESGGGVVQPGRSLRLSCAASGFTFSSYTMHWVRQAP
GKGLEWVTFISYDGNNKYYADSVKGRFTISRDNSKNTLYLQ
MNSLRAEDTAIYYCARTGWLGPFDYWGQGTLVTVSS
[SGGGG]ₙ eivmtqspsslsasvgdrvtitcrasqs
isrylnwyqlkpgkaprlliygasslqsgvpsrfsgsgsga
eftltisslqpediatyycqhtrafgqgtkveik [SGGGG]ₙ
QVQLVQSGAEVRKPGASVKVSCKASGYDFSNYAIHWVRQAP
GQRLEWMGWINAGNGNTKFSQKFQGRITVTRDTAASTAYME
LRSLRSEDTAVYYCARDSSNMVRGIIIAYYFDYWGQGTLVT
VSS [SGGGG]ₙ diqmtqspsslsasvgdrvtitcr
asqsinsvldwvqqkpqkapklliyaasslqsqvpsrfsgs
gsgtdftltisslqpedfatyycqqvvstpftfgpgtkvei
k

wherein:

Uppercase underlined text corresponds to the variable heavy chain amino acid sequence of anti-CTLA4 MAb tremelimumab,

Lowercase regular text corresponds to the variable light chain amino acid sequence of another embodiment of an anti-RANKL antibody as disclosed in EP 1257648,

Uppercase regular text corresponds to the variable heavy chain amino acid sequence of another embodiment of an anti-RANKL antibody as disclosed in EP 1257648,

Lowercase underlined text corresponds to the variable light chain amino acid sequence of anti-CTLA4 MAb tremelimumab,

Each occurrence of [SGGGG]ₙ is a flexible linker, wherein n=1, 2, 3, or 4, preferably n=1 for the first and third instances of the flexible linker, and n=3 for the second instance of the flexible linker.

### 5.4 Anti-RANKL-Anti-PD-1 CrossMAb Constructs

The present invention also contemplates CrossMAb multispecific antigen-binding molecules. In a first step of CrossMAb construction, an engineered protuberance is created in the interface of a first IgG-like polypeptide by replacing at least one contact residue of that polypeptide within its CH3 domain. Specifically, the contact residue to be replaced on the first polypeptide corresponds to an IgG residue at position 366 (residue numbering is according to Fc crystal structure (Deisenhofer, Biochem. 20:2361 [1981]) and wherein an engineered protuberance comprises replacing the nucleic acid encoding the original residue with nucleic acid encoding an import residue having a larger side chain volume than the original residue. Specifically, the threonine (T) residue at position 366 is mutated to tryptophan (W). In the second step, an engineered cavity is created in the interface of the second polypeptide by replacing at least one contact residue of the polypeptide within its C_{H}3 domain, wherein the engineered cavity comprises replacing the nucleic acid encoding an original residue with nucleic acid encoding an import residue having a smaller side chain volume than the original residue. Specifically, the contact residue to be replaced on the second polypeptide corresponds to an IgG residue at position 407. Specifically, the tyrosine (Y) residue at position 407 is mutated to alanine (A). This procedure can be engineered on different IgG subtypes, selected from the group consisting of IgG1, IgG2a, IgG2b, IgG3 and IgG4.

In a subsequent step, to promote the discrimination between the two light chain/heavy chain interactions possible in a heterodimeric bi-specific IgG, the association of the desired light-chain/heavy-chain pairings can be induced by modification of one Fab of the bispecific antibody (Fab region) to “swap” the constant or constant and variable regions between the light and heavy chains (see, e.g., Schaefer et al., 2011, supra). Thus, in the modified Fab domain, the heavy chain would comprise, for example, CL-V_{H} or CL-V_{L} domains and the light chain would comprise C_{H1}-V_{L} or C_{H1}-V_{H} domains, respectively. This prevents interaction of the heavy/light chain Fab portions of the modified chains (i.e., modified light or heavy chain) with and the heavy/light chain Fab portions of the standard/non-modified arm. By way of explanation, the heavy chain in the Fab domain of the modified arm, comprising a C_{L} domain, does not preferentially interact with the light chain of the non-modified arm/Fab domain, which also comprises a C_{L} domain (preventing “improper” or undesired pairings of heavy/light chains). This technique for preventing association of “improper” light/heavy chains is termed “CrossMAb” technology and, when combined with KiH technology, results in remarkably enhanced expression of the desired bispecific molecules (see, e.g., Schaefer et al., 2011, supra).

Production of the heterodimeric bi-specific IgG antibodies is achieved by first cloning each of the antibody genes encoding the 4 chains of the bi-specific IgG into mammalian expression vectors to enable secretory expression in mammalian cells (such as HEK293). Each of the antibody chain cDNAs is transfected together at equimolar ratios into HEK293 cells using 293fectin or similar techniques and antibody containing cell culture supernatants are harvested and antibodies are purified from supernatants using protein A Sepharose.

In some embodiments, a bi-specific heterodimeric IgG composed of both an anti-RANKL antigen-binding molecule and an anti-PD-1 antigen-binding molecule can be constructed using 2 heavy and 2 light chain constructs, in which one of the heavy chain CH3 domain is altered at position 366 (T366W), termed the “knob” and the other heavy chain CH3 domain is altered at position 407 (Y407A), termed the “hole” to promote KiH heterodimerization of the heavy chains.

### 5.4.1 Constructs for Denosumab CrossMAb-CHI-C_{L} Interchange

An illustrative denosumab CrossMAb may comprise heavy chain sequences derived from IgG2 and the desired light-chain/heavy-chain pairings can be induced by modification of the Fab domain of the anti-RANKL antigen-binding molecule, such that the C_{HL} and C_{L} domains are interchanged between Ig chains. The following four constructs are used for this construction.

Construct 1
Denosumab CrossMAb C_{H1}-C_{L} huIgG2 KNOB mutation,
heavy chain
[SEQ ID NO: 240]
MEFGLSWLFLVAILKGVOCEVQLLESGGGLVQPGGSLRLSCAASGFTFSS
YAMSWVRQAPGKGLEWVSGITGSGGSTYYADSVKGRFTISRDNSKNTLYL
QMNSLRAEOTAVYYCAKDPGTTVIMSWFOPWGQGTLVTVSSrtvaapsvf
ifppsdeqlksgtasvvcllnnfypreakvqwkvdnalqsgnsqesvteq
dskdstyslsstltlskadyekhkvyacevthqglsspvtksfnrgecer
kccvecppcpappvagpsvflfppkpkdtlmisrtpevtcvvvdvshedp
evqfnwyvdgvevhnaktkpreeqfnstfrvvsvltvvhqdwlngkeykc
kvsnkglpapiektisktkgqprepqvytlppsreemtknqvslWclvkg
fypsdiavewesngqpennykttppmldsdgsfflyskltvdksrwqqgn
vfscsvmhealhnhytqkslslspgk,

wherein:

IgG₂ signal peptide is in underlined uppercase text;

Denosumab V_{H} is in regular uppercase text;

Denosumab C_{L} domain is in bold lowercase text;

Hinge region is in underlined lowercase text;

Denosumab C_{H2}-C_{H3} domain is in regular lowercase text; and

T366W substitution is in bold uppercase text.

Construct 2
Denosumab CrossMAb C_{H1}-C_{L} light chain
[SEQ ID NO: 241]
METPAOLLFLLLLWLPDTTGEIVLTQSPGTLSLSPGERATLSCRASQSVR
GRYLAWYQQKPGQAPRLLIYGASSRATGIPDRFSGSGSGTDFTLTISRLE
PEDFAVFYCQQYGSSPRTFGQGTKVEIKastkgpsvfplapcsrstsest
aalgclvkdyfpepvtvswnsgaltsgvhtfpavlqssglyslssvvtvp
ssnfgtqtytcnvdhkpsntkvdktv,

wherein:

Kappa signal peptide is in underlined uppercase text;

Denosumab V_{L} is in regular uppercase text; and

Denosumab C_{H1} domain is in bold lowercase text.

Construct 3
Nivolumab IgG2 Hole mutation, heavy chain
[SEQ ID NO: 242]
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSSastkgpsvfplapcsrstsestaalgclvkdyfpepv
tvswnsgaltsgvhtfpavlqssglyslssvvtvpssslgtktytcnvdh
kpsntkvdkrverkccvecppcpappvagpsvflfppkpkdtlmisrtpe
vtcvvvdvshedpevqfnwyvdgvevhnaktkpreeqfnstfrvvsvltv
vhqdwlngkeykckvsnkglpapiektisktkgqprepqvytlppsreem
tknqvsltclvkgfypsdiavewesngqpennykttppmldsdgsfflAs
kltvdksrwqqgnvfscsvmhealhnhytqkslslspgk,

wherein:

Nivolumab V_{H} is in regular uppercase text;

Nivolumab C_{H1} domain is in bold lowercase text;

HuIgG2 Hinge region is in underlined lowercase text;

HuIgG2C_{H2}-C_{H3} domain is in regular lowercase text; and

Y407A substitution is in bold uppercase text.

Construct 4
Nivolumab light chain
[SEQ ID NO: 243]
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

5.4.2 Constructs for Denosumab CrossMAb-V_{H}-V_{L} Interchange

In another embodiment of a denosumab CrossMAb, the desired light-chain/heavy-chain pairings can be induced by modification of the Fab domain of the anti-RANKL antigen-binding molecule, such that the V_{H} and V_{L} domains are interchanged between Ig chains. In one embodiment, this comprises heavy chain sequences derived from IgG2 and heavy chain heterodimerization is promoted by KiH alterations. The following four constructs are used for this construction.

Construct l
Denosumab CrossMAb V_{H}-V_{L} huIgG2 KNOB mutation,
heavy chain
[SEQ ID NO: 244]
MEFGLSWLFLVAILKGVOCEIVLTQSPGTLSLSPGERATLSCRASQSVRG
RYLAWYQQKPGQAPRLLIYGASSRATGIPDRFSGSGSGTDFTLTISRLEP
EDFAVFYCQQYGSSPRTFGQGTKVEIKastkgpsvfplapcsrstsesta
algclvkdyfpepvtvswnsgaltsgvhtfpavlqssglyslssvvtvps
snfgtqtytcnvdhkpsntkvdktverkccvecppcpappvagpsvflfp
pkpkdtlmisrtpevtcvvvdvshedpevqfnwyvdqvevhnaktkpree
qfnstfrvvsvltvvhqdwlngkeykckvsnkglpapiektisktkgqpr
epqvytlppsreemtknqvslWclvkgfypsdiavewesngqpennyktt
ppmldsdgsfflyskltvdksrwqqgnvfscsvmhealhnhytqkslsls
pgk,

wherein: IgG₂ signal peptide is in underlined uppercase text;

Denosumab V_{L} is in regular uppercase text;

Denosumab C_{H1} domain is in bold lowercase text;

Hinge region is in underlined lowercase text;

Denosumab C_{H2}-C_{H3} domain is in regular lowercase text; and

T366W substitution is in bold uppercase text.

Construct 2
Denosumab CrossMAb V_{H}-V_{L} light chain
[SEQ ID NO: 245]
METPAOLLFLLLLWLPDTTGEVQLLESGGGLVQPGGSLRLSCAASGFTFS
SYAMSWVRQAPGKGLEWVSGITGSGGSTYYADSVKGRFTISRDNSKNTLY
LQMNSLRAEOTAVYYCAKDPGTTVIMSWFOPWGQGTLVTVSSrtvaapsv
fifppsdeqlksgtasvvcllnnfypreakvqwkvdnalqsgnsqesvte
qdskdstyslsstltlskadyekhkvyacevthqglsspvtksfnrgec,

wherein:

Kappa signal peptide is in underlined uppercase text;

Denosumab V_{H} is in regular uppercase text; and

Denosumab C_{L} domain is in bold lowercase text.

Construct 3
Nivolumab IgG2 Hole mutation, heavy chain
[SEQ ID NO: 246]
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSSastkgpsvfplapcsrstsestaalgclvkdyfpepv
tvswnsgaltsgvhtfpavlqssglyslssvvtvpssslgtktytcnvdh
kpsntkvdkrverkccvecppcpappvagpsvflfppkpkdtlmisrtpe
vtcvvvdvshedpevqfnwyvdgvevhnaktkpreeqfnstfrvvsvltv
vhqdwlngkeykckvsnkglpapiektisktkgqprepqvytlppsreem
tknqvsltclvkgfypsdiavewesngqpennykttppmldsdgsfflAs
kltvdksrwqqgnvfscsvmhealhnhytqkslslspgk,

wherein:

Nivolumab V_{H} is in regular uppercase text;

Nivolumab C_{H1} domain is in bold lowercase text;

HuIgG2 Hinge region is in underlined lowercase text;

HuIgG2C_{H2}-CH3 domain is in regular lowercase text; and

Y407A substitution is in bold uppercase text.

Construct 4
Nivolumab light chain
[SEQ ID NO: 247]
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

### 5.4.3 Constructs for Denosumab CrossMAb-Fab-Fab Interchange

In yet another embodiment of a denosumab CrossMAb, the desired light-chain/heavy-chain pairings can be induced by modification of the Fab domain of the anti-RANKL antigen-binding molecule, such that the Fab domains are interchanged between Ig chains. In this embodiment, this comprises heavy chain sequences derived from IgG2 and heavy chain heterodimerization is promoted by KIH alterations. The following four constructs are used for this construction.

Construct 1
Denosumab CrossMAb Fab huIgG2 KNOB
mutation, heavy chain
[SEQ ID NO: 248]
MEFGLSWLFLVAILKGVOCEIVLTQSPGTLSLSPG
ERATLSCRASQSVRGRYLAWYQQKPGQAPRLLIYG
ASSRATGIPDRFSGSGSGTDFTLTISRLEPEDFAV
FYCQQYGSSPRTFGQGTKVEIKrtvaapsvfifpp
sdeqlksgtasvvcllnnfypreakvqwkvdnalq
sgnsqesvteqdskdstyslsstltlskadyekhk
vyacevthqglsspvtksfnrgecerkccvecppc
pappvagpsvflfppkpkdtlmisrtpevtcwvdv
shedpevqfnwyvdgvevhnaktkpreeqfnstfr
vvsvltvvhqdwlngkeykckvsnkglpapiekti
SktkgqprepqvytlppsreemtknqvslWclvkg
fypsdiavewesngqpennykttppmldsdgsffl
yskltvdksrwqqgnvfscsvmhealhnhytqksl
slspgk,

wherein:

IgG₂ signal peptide is in underlined uppercase text;

Denosumab V_{L} is in regular uppercase text;

Denosumab C_{L} domain is in bold lowercase text;

Hinge region is in underlined lowercase text;

Denosumab C_{H2}-CH3 domain is in regular lowercase text; and

T366W substitution is in bold uppercase text.

Construct 2
Denosumab CrossMAb Fab light chain
[SEQ ID NO: 249]
METPAOLLFLLLLWLPDTTGEVQLLESGGGLVQPG
GSLRLSCAASGFTFSSYAMSWVRQAPGKGLEWVSG
ITGSGGSTYYADSVKGRFTISRDNSKNTLYLQMNS
LRAEOTAVYYCAKDPGTTVIMSWFOPWGQGTLVTV
SSastkgpsvfplapcsrstsestaalgclvkdyf
pepvtvswnsgaltsgvhtfpavlqssglyslssv
vtvpssnfgtqtytcnvdhkpsntkvdktv,

wherein:

Kappa signal peptide is in underlined uppercase text;

Denosumab V_{H} is in regular uppercase text; and

Denosumab C_{H1} domain is in bold lowercase text.

Construct 3
Nivolumab IgG2 Hole mutation, heavy chain
[SEQ ID NO: 250]
QVQLVESGGGVVQPGRSLRLDCKASGITFSNSGMH
WVRQAPGKGLEWVAVIWYDGSKRYYADSVKGRFTI
SRDNSKNTLFLQMNSLRAEDTAVYYCATNDDYWGQ
GTLVTVSSastkgpsvfplapcsrstsestaalgc
lvkdyfpepvtvswnsgaltsgvhtfpavlqssgl
yslssvvtvpssslgtktytcnvdhkpsntkvdkr
verkccvecppcpappvagpsvflfppkpkdtlmi
srtpevtcvvvdvshedpevqfnwyvdgvevhnak
tkpreeqfnstfrvvsvltvvhqdwlngkeykckv
snkglpapiektisktkgqprepqvytlppsreem
tknqvsltclvkgfypsdiavewesngqpennykt
tppmldsdgsfflAskltvdksrwqqgnvfscsvm
healhnhytqkslslspgk,

wherein:

Nivolumab V_{H} is in regular uppercase text;

Nivolumab C_{H1} domain is in bold lowercase text;

HuIgG2 Hinge region is in underlined lowercase text;

HuIgG2C_{H2}-CH3 domain is in regular lowercase text; and

Y407A substitution is in bold uppercase text.

Construct 4
Nivolumab light chain
[SEQ ID NO: 251]
EIVLTQSPATLSLSPGERATLSCRASQSVSSYLAW
YQQKPGQAPRLLIYDASNRATGIPARFSGSGSGTD
FTLTISSLEPEDFAVYYCQQSSNWPRTFGQGTKVE
IKRTVAAPSVFIFPPSDEQLKSGTASVVCLLNNFY
PREAKVQWKVDNALQSGNSQESVTEQDSKDSTYSL
SSTLTLSKADYEKHKVYACEVTHQGLSSPVTKSFN
RGEC.

5.4.4 Constructs for Denosumab CrossMAb—C_{HI}-C_{L} Interchange-IgG₄ C_{H}

In still another embodiment of a denosumab CrossMAb, the denosumab CrossMAb comprises heavy chain sequences derived from IgG₄. In this embodiment, the desired light-chain/heavy-chain pairings can be induced by modification of the Fab domain of the anti-RANKL antigen-binding molecule, such that the C_{H1} and C_{L} domains are interchanged between Ig chains. The following four constructs are used for this construction.

Construct 1
Denosumab CrossMAb C_{H1}-C_{L} huIgG4 KNOB
mutation, heavy chain
[SEQ ID NO: 252]
MEFGLSWLFLVAILKGVOCEVQLLESGGGLVQPGGS
LRLSCAASGFTFSSYAMSWVRQAPGKGLEWVSGITG
SGGSTYYADSVKGRFTISRDNSKNTLYLQMNSLRAE
OTAVYYCAKDPGTTVIMSWFOPWGQGTLVTVSSrtv
aapsvfifppsdeqlksgtasvvcIInnfypreakv
qwkvdnalqsgnsqesvteqdskdstyslsstltls
kadyekhkvyacevthqglsspvtksfnrgecesky
gppcpscpapeflggpsvflfppkpkdtlmisrtpe
vtcvvvdvsqedpevqfnwyvdgvevhnaktkpree
qfnstyrvvsvltvlhqdwlngkeykckvsnkglps
siektiskakgqprepqvytlppsqeemtknqvslW
clvkgfypsdiavewesngqpennykttppvldsdg
sfflysrltvdksrwqegnvfscsvmhealhnhytq
kslslslgk,

wherein:

IgG₂ signal peptide is in underlined uppercase text;

Denosumab V_{H} is in regular uppercase text;

Denosumab C_{L} domain is in bold lowercase text;

IgG₄ hinge region is in underlined lowercase text;

IgG₄ C_{H2}-CH3 domain is in regular lowercase text; and

T366W substitution is in bold uppercase text.

Construct 2
[SEQ ID NO: 253]
Denosumab CrossMAb C_{H1}-C_{L} light chain
METPAOLLFLLLLWLPDTTGEIVLTQSPGTLSLSPGER
ATLSCRASQSVRGRYLAWYQQKPGQAPRLLIYGASSRA
TGIPDRFSGSGSGTDFTLTISRLEPEDFAVFYCQQYGS
SPRTFGQGTKVEIKastkgpsvfplapcsrstsestaa
lgclvkdyfpepvtvswnsgaltsgvhtfpavlqssgl
yslssvvtvpssnfgtqtytcnvdhkpsntkvdktv,

wherein:

Kappa signal peptide is in underlined uppercase text;

Denosumab V_{L} is in regular uppercase text; and

Denosumab C_{HI}. domain is in bold lowercase text.

Construct 3
Nivolumab IgG₄ Hole mutation, heavy chain
[SEQ ID NO: 254]
QVQLVESGGGVVQPGRSLRLDCKASGITFSNSGMHWVRQA
PGKGLEWVAVIWYDGSKRYYADSVKGRFTISRDNSKNTLF
LQMNSLRAEDTAVYYCATNDDYWGQGTLVTVSSastkgps
vfplapcsrstsestaalaclvkdvfpepvtvswnsaalt
sgvhtfpavlassalvslssvvtvpssslatktvtcnvdh
kpsntkvdkrveskygppcppcpapeflggpsvflfppkp
kdtlmisrtpevtcvvvdvsqedpevqfnwyvdgvevhna
ktkpreeqfnstyrvvsvitvihqdwingkeykckvsnkg
lpssiektiskakgqprepqvytlppsqeemtknqvsItd
vkgfypsdiavewesngqpennykttppvIdsdgsfflAs
rltvdksrwqegnvfscsvmhealhnhytqkslslslgk,

wherein:

Nivolumab V_{H} is in regular uppercase text;

Nivolumab C_{H1} domain is in bold lowercase text;

IgG₄ hinge region is in underlined lowercase text;

IgG4 C_{H2}-C_{H3} domain is in regular lowercase text; and

Y407A substitution is in bold uppercase text.

Construct 4
Nivolumab light chain
[SEQ ID NO: 255]
EIVLTQSPATLSLSPGERATLSCRASQSVSSYLAWYQQK
PGQAPRLLIYDASNRATGIPARFSGSGSGTDFTLTISSL
EPEDFAVYYCQQSSNWPRTFGQGTKVEIKRTVAAPSVFI
FPPSDEQLKSGTASVVCLLNNFYPREAKVQWKVDNALQS
GNSQESVTEQDSKDSTYSLSSTLTLSKADYEKHKVYACE
VTHQGLSSPVTKSFNRGEC.

5.4.5 Constructs for Denosumab CrossMAb-V_{H}-V_{L} Interchange-IgG₄ CH

In a further embodiment of a denosumab CrossMAb, the desired light-chain/heavy-chain pairings can be induced by modification of the Fab domain of the anti-RANKL antigen-binding molecule, such that the V_{H} and V_{L} domains are interchanged between Ig chains. In this embodiment, this comprises heavy chain sequences derived from IgG₄ and heavy chain heterodimerization is promoted by KiH alterations. The following four constructs are used for this construction.

Construct 1
Denosumab CrossMAb V_{H}-V_{L} huIgG4
KNOB mutation, heavy chain
[SEQ ID NO: 256]
MEFGLSWLFLVAILKGVOCEIVLTQSPGTLSLSPGERAT
LSCRASQSVRGRYLAWYQQKPGQAPRLLIYGASSRATGI
PDRFSGSGSGTDFTLTISRLEPEDFAVFYCQQYGSSPRT
FGQGTKVEIKastkgpsvfplapcsrstsestaalgclv
kdyfpepvtvswnsgaltsgvhtfpavlqssglyslssv
vtvpssnfgtqtytcnvdhkpsntkvdktveskygppcp
scpapeflggpsvflfppkpkdtlmisrtpevtcvvvdv
sqedpevqfnwyvdgvevhnaktkpreeqfnstyrvvsv
ltvlhqdwlngkeykckvsnkglpssiektiskakgqpr
epqvytlppsqeemtknqvslWclvkgfypsdiavewes
ngqpennykttppvldsdgsfflysrltvdksrwqegnv
fscsvmhealhnhytqkslslslgk

wherein:

IgG2 signal peptide is in underlined uppercase text;

Denosumab V_{L} is in regular uppercase text;

Denosumab C_{H1} domain is in bold lowercase text;

IgG₄ hinge region is in underlined lowercase text;

IgG₄ C_{H2}-CH3 domain is in regular lowercase text; and

T366W substitution is in bold uppercase text.

Construct 2
Denosumab CrossMAb V_{H}-V_{L} light chain
[SEQ ID NO: 257]
METPAOLLFLLLLWLPDTTGEVQLLESGGGLVQPGGSLR
LSCAASGFTFSSYAMSWVRQAPGKGLEWVSGITGSGGST
YYADSVKGRFTISRDNSKNTLYLQMNSLRAEDTAVYYCA
KDPGTTVIMSWFQPWGQGTLVTVSSrtvaapsvfifpps
deqlksgtasvvcllnnfypreakvqwkvdnalqsgnsq
esvteqdskdstyslsstltlskadyekhkvyacevthq
glsspvtksfnrgec,

wherein:

Kappa signal peptide is in underlined uppercase text;

Denosumab V_{H} is in regular uppercase text; and

Denosumab C_{L} domain is in bold lowercase text.

Construct 3
Nivolumab IgG₄ Hole mutation, heavy chain
[SEQ ID NO: 258]
QVQLVESGGGVVQPGRSLRLDCKASGITFSNSGMHWVRQ
APGKGLEWVAVIWYDGSKRYYADSVKGRFTISRDNSKNT
LFLQMNSLRAEDTAVYYCATNDDYWGQGTLVTVSSastk
gpsvfplapcsrstsestaalgclvkdyfpepvtvswns
galtsgvhtfpavlqssglyslssvvtvpssslgtktyt
cnvdhkpsntkvdkrveskygppcppcpapeflggpsvf
lfppkpkdtlmisrtpevtcvvvdvsqedpevqfnwyvd
gvevhnaktkpreeqfnstyrvvsvitvihqdwingkey
kckvsnkglpssiektiskakgqprepqvytlppsqeem
tknqvsItdvkgfypsdiavewesngqpennykttppvl
dsdgsfflAsrltvdksrwqegnvfscsvmhealhnhyt
qkslslslgk,

wherein:

Nivolumab V_{H} is in regular uppercase text;

Nivolumab C_{H1} domain is in bold lowercase text;

IgG₄ hinge region is in underlined lowercase text;

IgG4 C_{H2}-CH3 domain is in regular lowercase text; and

Y407A substitution is in bold uppercase text.

Construct 4
Nivolumab light chain
[SEQ ID NO: 259]
EIVLTQSPATLSLSPGERATLSCRASQSVSSYLAWYQQK
PGQAPRLLIYDASNRATGIPARFSGSGSGTDFTLTISSL
EPEDFAVYYCQQSSNWPRTFGQGTKVEIKRTVAAPSVFI
FPPSDEQLKSGTASVVCLLNNFYPREAKVQWKVDNALQS
GNSQESVTEQDSKDSTYSLSSTLTLSKADYEKHKVYACE
VTHQGLSSPVTKSFNRGEC.

### 5.4.6 Constructs for Denosumab CrossMAb-Fab-Fab Interchange-IgG4 CH

In another embodiment of a denosumab CrossMAb, the desired light-chain/heavy-chain pairings can be induced by modification of the Fab domain of the anti-RANKL antigen-binding molecule, such that the Fab domains are interchanged between Ig chains. In this embodiment, this comprises heavy chain sequences derived from IgG₄ and heavy chain heterodimerization is promoted by KiH alterations. The following four constructs are used for this construction.

Construct 1
Denosumab CrossMAb Fab huIgG4 KNOB
mutation, heavy chain
[SEQ ID NO: 260]
MEFGLSWLFLVAILKGVOCEIVLTQSPGTLSLSPGERA
TLSCRASQSVRGRYLAWYQQKPGQAPRLLIYGASSRAT
GIPDRFSGSGSGTDFTLTISRLEPEDFAVFYCQQYGSS
PRTFGQGTKVEIKrtvaapsvfifppsdeqlksgtasv
vcllnnfypreakvqwkvdnalqsgnsqesvteqdskd
styslsstltlskadyekhkvyacevthqglsspvtks
fnrgeceskvappcpscpapeflggpsvflfppkpkd
tlmisrtpevtcvvvdvsqedpevqfnwyvdgvevhna
ktkpreeqfnstyrvvsvltvlhqdwlngkeykckvsn
kglpssiektiskakgqprepqvytlppsqeemtknqv
slWclvkgfypsdiavewesngqpennykttppvldsd
gsfflysrltvdksrwqegnvfscsvmhealhnhytqk
slslslgk

wherein:

IgG2 signal peptide is in underlined uppercase text;

Denosumab V_{L} is in regular uppercase text;

Denosumab C_{L} domain is in bold lowercase text;

IgG₄ hinge region is in underlined lowercase text;

IgG4 C_{H2}-CH3 domain is in regular lowercase text; and

T366W substitution is in bold uppercase text.

Construct 2
Denosumab CrossMAb Fab light chain
[SEQ ID NO: 261]
METPAOLLFLLLLWLPDTTGEVQLLESGGGLVQPGGSLR
LSCAASGFTFSSYAMSWVRQAPGKGLEWVSGITGSGGST
YYADSVKGRFTISRDNSKNTLYLQMNSLRAEOTAVYYCA
KDPGTTVIMSWFOPWGQGTLVTVSSastkgpsvfplapc
srstsestaalgclvkdyfpepvtvswnsgaltsgvhtf
pavlqssglyslssvvtvpssnfgtqtytcnvdhkpsnt
kvdktv,

wherein:

Kappa signal peptide is in underlined uppercase text;

Denosumab V_{H} is in regular uppercase text; and

Denosumab C_{H1} domain is in bold lowercase text.

Construct 3
Nivolumab IgG₄ Hole mutation, heavy chain
[SEQ ID NO: 262]
QVQLVESGGGVVQPGRSLRLDCKASGITFSNSGMHWVR
QAPGKGLEWVAVIWYDGSKRYYADSVKGRFTISRDNSK
NTLFLQMNSLRAEDTAVYYCATNDDYWGQGTLVTVSSr
stkgpsvfplapcsrstsestaalqclvkdvfpepvtv
swnsqaltSQvhtfpavlqssqlvslssvvtvpssslq
tktvtcnvdhkpsntkvdkrveskyqppcppcpapefl
ggpsvflfppkpkdtlmisrtpevtcvvvdvsqedpev
qfnwyvdgvevhnaktkpreeqfnstyrvvsvltvlhq
dwlngkeykckvsnkglpssiektiskakgqprepqvy
tlppsqeemtknqvsltclvkgfypsdiavewesngqp
ennykttppvldsdgsfflAsrltvdksrwqegnvfsc
svmhealhnhytqkslslslgk,

wherein:

Nivolumab V_{H} is in regular uppercase text;

Nivolumab C_{H1} domain is in bold lowercase text;

IgG₄ hinge region is in underlined lowercase text;

IgG4 C_{H2}-CH3 domain is in regular lowercase text; and

Y407A substitution is in bold uppercase text.

Construct 4
Nivolumab light chain
[SEQ ID NO: 263]
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

5.4.7 Constructs for Denosumab CrossMAb-CHI-C_{L} Interchange-IgG₁ C_{H}

Yet another embodiment of a denosumab CrossMAb comprises heavy chain sequences derived from IgG₁. In this embodiment, the desired light-chain/heavy-chain pairings can be induced by modification of the Fab domain of the anti-RANKL antigen-binding molecule, such that the C_{H1} and C_{L} domains are interchanged between Ig chains. The following four constructs are used for this construction.

Construct 1
Denosumab CrossMAb C_{H1}-C_{L} huIgG1 KNOB
mutation, heavy chain
[SEQ ID NO: 264]
MEFGLSWLFLVAILKGVOCEVQLLESGGGLVQPGGSLRLSCAASGFTFSS
YAMSWVRQAPGKGLEWVSGITGSGGSTYYADSVKGRFTISRDNSKNTLYL
QMNSLRAEOTAVYYCAKDPGTTVIMSWFOPWGQGTLVTVSSrtvaapsvf
ifppsdeqlksgtasvvcllnnfypreakvqwkvdnalqsgnsqesvteq
dskdstysisstltlskadyekhkvyacevthqglsspvtksfnrgecep
kscdkthtcppcpapellggpsvflfppkdkdtlmisrtpevtcvvvdvs
hedpevkfnwyvdgvevhnaktkpreeqynstyrvvsvltvlhqdwlngk
eykckvsnkalpapiektiskakgqprepqvytlppsrdeltknqvslWc
lvkgfypsdiavewesngqpennykttppvldsdgsfflyskltvdksrw
qqgnvfscsvmhealhnhytqkslslspgk

wherein:

IgG₂ signal peptide is in underlined uppercase text;

Denosumab V_{H} is in regular uppercase text;

Denosumab C_{L} domain is in bold lowercase text;

IgG₁ hinge region is in underlined lowercase text;

IgG₁ C_{H2}-C_{H3} domain is in regular lowercase text; and

T366W substitution is in bold uppercase text.

Construct 2
Denosumab CrossMAb C_{H1}-C_{L} light chain
[SEQ ID NO: 265]
METPAOLLFLLLLWLPDTTGEIVLTQSPGTLSLSPGERATLSCRASQSVR
GRYLAWYQQKPGQAPRLLIYGASSRATGIPDRFSGSGSGTDFTLTISRLE
PEDFAVFYCQQYGSSPRTFGQGTKVEIKastkgpsvfplapcsrstsest
aalgclvkdyfpepvtvswnsgaltsgvhtfpavlqssglyslssvvtvp
ssnfgtqtytcnvdhkpsntkvdktv,

wherein:

Kappa signal peptide is in underlined uppercase text;

Denosumab V_{L} is in regular uppercase text; and

Denosumab C_{H1} domain is in bold lowercase text.

Construct 3
Nivolumab IgG₁ Hole mutation, heavy chain
[SEQ ID NO: 266]
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSSastkgpsvfplapcsrstsestaalgclvkdyfpepv
tvswnsgaltsgvhtfpavlqssglyslssvvtvpssslgtktytcnvdh
kpsntkvdkrvepkscdkthtcppcpapellggpsvflfppkpkdtlmis
rtpevtcvvvdvshedpevkfnwyvdgvevhnaktkpreeqynstyrvvs
vltvlhqdwlngkeykckvsnkalpapiektiskakgqprepqvytlpps
rdeltknqvsltdvkgfypsdiavewesngqpennykttppvldsdgsff
lAskltvdksrwqqgnvfscsvmhealhnhytqkslslspgk,

wherein:

Nivolumab V_{H} is in regular uppercase text;

Nivolumab C_{H1} domain is in bold lowercase text;

IgG1 hinge region is in underlined lowercase text;

IgG₁ CH2⁻CH3 domain is in regular lowercase text; and

Y407A substitution is in bold uppercase text.

Construct 4
Nivolumab light chain
[SEQ ID NO: 267]
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

5.4.8 Constructs for Denosumab CrossMAb-V_{H}-V_{L} Interchange-IgG₁ CH

In another embodiment of a denosumab CrossMAb, the desired light-chain/heavy-chain pairings can be induced by modification of the Fab domain of the anti-RANKL antigen-binding molecule, such that the V_{H} and V_{L} domains are interchanged between Ig chains. In one embodiment, this comprises heavy chain sequences derived from IgG₁ and heavy chain heterodimerization is promoted by KiH alterations. The following four constructs are used for this construction.

Construct 1
Denosumab CrossMAb V_{H}-V_{L} huIgG1 KNOB
mutation, heavy chain
[SEQ ID NO: 268]
MEFGLSWLFLVAILKGVOCEIVLTQSPGTLSLSPGERATLSCRASQSVRG
RYLAWYQQKPGQAPRLLIYGASSRATGIPDRFSGSGSGTDFTLTISRLEP
EDFAVFYCQQYGSSPRTFGQGTKVEIKastkgpsvfplapcsrstsesta
algclvkdyfpepvtvswnsgaltsgvhtfpavlqssglyslssvvtvps
snfgtqtytcnvdhkpsntkvdktvepkscdkthtcppcapellggpsvf
lfppkpkdtlmisrtpevtcvvvdvshedpevkfnwyvdqvevhnaktkp
reeqvnstvrvvsvltvlhqdwlngkeykckvsnkalpapiektiskakg
qprepqvytlppsrdeltknqvslWclvkgfypsdiavewesngqpenny
kttppvldsdgsfflyskltvdksrwqqgnvfscsvmhealhnhytqksl
slspgk,

wherein:

IgG₂ signal peptide is in underlined uppercase text;

Denosumab V_{L} is in regular uppercase text;

Denosumab C_{H1} domain is in bold lowercase text;

IgG₁ hinge region is in underlined lowercase text;

IgG₁ C_{H2}-CH3 domain is in regular lowercase text; and

T366W substitution is in bold uppercase text.

Construct 2
Denosumab CrossMAb V_{H}-V_{L} light chain
[SEQ ID NO: 269]
METPAOLLFLLLLWLPDTTGEVQLLESGGGLVQPGGSLRLSCAASGFTFS
SYAMSWVRQAPGKGLEWVSGITGSGGSTYYADSVKGRFTISRDNSKNTLY
LQMNSLRAEOTAVYYCAKDPGTTVIMSWFOPWGQGTLVTVSSrtvaapsv
fifppsdeqlksgtasvvcllnnfypreakvqwkvdnalqsgnsqesvte
qdskdstyslsstltlskadyekhkvyacevthqglsspvtksfnrgec,

wherein:

Kappa signal peptide is in underlined uppercase text;

Denosumab V_{H} is in regular uppercase text; and

Denosumab C_{L} domain is in bold lowercase text.

Construct 3
Nivolumab IgG₁ Hole mutation, heavy chain
[SEQ ID NO: 270]
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSSastkgpsvfplapcsrstsestaalgclvkdyfpepv
tvswnsgaltsgvhtfpavlqssglyslssvvtvpssslgtktytcnvdh
kpsntkvdkrvepkscdkthtcppcapellqapsvflfppkpkdtlmisr
tpevtcvvvdvshedpevkfnwyvdgvevhnaktkpreeqynstvrvvsv
ltvlhadwlngkeykckvsnkalpapiektiskakgqprepqvytlppsr
deltknqvsltdvkgfypsdiavewesngqpennykttppvldsdgsffl
Askltvdksrwqqgnvfscsvmhealhnhytqkslslspgk,

wherein:

Nivolumab V_{H} is in regular uppercase text;

Nivolumab C_{H1} domain is in bold lowercase text;

IgG₁ hinge region is in underlined lowercase text;

IgG₁ C_{H2}-C_{H3} domain is in regular lowercase text; and

Y407A substitution is in bold uppercase text.

Construct 4
Nivolumab light chain
[SEQ ID NO: 271]
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

5.4.9 Constructs for Denosumab CrossMAb-Fab-Fab Interchange-IgG₁ C_{H}

In another embodiment of a denosumab CrossMAb, the desired light-chain/heavy-chain pairings can be induced by modification of the Fab domain of the anti-RANKL antigen-binding molecule, such that the Fab domains are interchanged between Ig chains. In one embodiment, this comprises heavy chain sequences derived from IgG1 and heavy chain heterodimerization is promoted by KiH alterations. The following four constructs are used for this construction.

Construct 1
Denosumab CrossMAb Fab huIgG1 KNOB
mutation, heavy chain
[SEQ ID NO: 272]
MEFGLSWLFLVAILKGVOCEIVLTQSPGTLSLSPGERATLSCRASQSVRG
RYLAWYQQKPGQAPRLLIYGASSRATGIPDRFSGSGSGTDFTLTISRLEP
EDFAVFYCQQYGSSPRTFGQGTKVEIKrtvaapsvfifppsdeqlksgta
svvcllnnfypreakvqwkvdnalqsgnsqesvteqdskdstyslsstlt
lskadyekhkvyacevthqglsspvtksfnrgecepkscdkthtcppcpa
pellggpsvflfppkpkdtlmisrtpevtcvvvdvshedpevkfnwyvdg
vevhnaktkpreeqynstyrvvsvItvIhqdwIngkeykckvsnkalpap
iektiskakgqprepqvytlppsrdeltknqvslWclvkgfypsdiavew
esngqpennykttppvldsdgsfflyskltvdksrwqqgnvfscsvmhea
lhnhytqkslslspgk,

wherein:

IgG2 signal peptide is in underlined uppercase text;

Denosumab V_{L} is in regular uppercase text;

Denosumab C_{L} domain is in bold lowercase text;

IgG₁ hinge region is in underlined lowercase text;

IgG₁ C_{H2}-C_{H3} domain is in regular lowercase text; and

T366W substitution is in bold uppercase text.

Construct 2
Denosumab CrossMAb Fab light chain
[SEQ ID NO: 273]
METPAOLLFLLLLWLPDTTGEVQLLESGGGLVQPGGSLRLSCAASGFTFS
SYAMSWVRQAPGKGLEWVSGITGSGGSTYYADSVKGRFTISRDNSKNTLY
LQMNSLRAEOTAVYYCAKDPGTTVIMSWFOPWGQGTLVTVSSastkgpsv
fplapcsrstsestaalgclvkdyfpepvtvswnsgaltsgvhtfpavlq
ssglyslssvvtvpssnfgtqtytcnvdhkpsntkvdktv,

wherein:

Kappa signal peptide is in underlined uppercase text;

Denosumab V_{H} is in regular uppercase text; and

Denosumab C_{HI}. domain is in bold lowercase text.

Construct 3
Nivolumab IgG₁ Hole mutation, heavy chain
[SEQ ID NO: 274]
QVQLVESGGGVVQPGRSLRLDCKASGITFSNSGMHWVRQAPGKGLEWVAV
IWYDGSKRYYADSVKGRFTISRDNSKNTLFLQMNSLRAEDTAVYYCATND
DYWGQGTLVTVSSastkgpsvfplapcsrstsestaalgclvkdyfpepv
tvswnsgaltsgvhtfpavlqssglyslssvvtvpsssigtktytcnvdh
kpsntkvdkrvepkscdkthtcppcpapellggpsvflfppkpkdtlmis
rtpevtcvvvdvshedpevkfnwyvdgvevhnaktkpreeqynstyrvvs
vltvlhqdwlngkeykckvsnkalpapiektiskakgqprepqvytlpps
rdeltknqvsltdvkgfypsdiavewesngqpennykttppvldsdgsff
lAskltvdksrwqqgnvfscsvmhealhnhytqkslslspgk,

wherein:

Nivolumab V_{H} is in regular uppercase text;

Nivolumab C_{H1} domain is in bold lowercase text;

IgG₁ hinge region is in underlined lowercase text;

IgG1 C_{H2}-CH3 domain is in regular lowercase text; and

Y407A substitution is in bold uppercase text.

Construct 4
Nivolumab light chain
[SEQ ID NO: 275]
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC.

Following production and purification of the monospecific bivalent parental antibodies bi-specific heterodimeric antibodies can be assembled through a controlled Fab-arm exchange between two monospecific antibodies, as described (Labrijn et al. Nature Protocols 2014;9(10):2450-63).

In various embodiments, the anti-RANKL antigen-binding molecule comprises an Fab domain of the anti-RANKL antigen-binding molecule, such that the Fab domains are interchanged between Ig chains, comprised of SEQ ID NO:272 (denosumab CrossMAb Fab huIgG1 KNOB mutation, heavy chain) and SEQ ID NO:273 (denosumab CrossMAb Fab light chain).

In other embodiments, the Fab domain of the anti-RANKL antigen-binding molecule is modified such that the V_{H} and V_{L} domains are interchanged between Ig chains, comprised of SEQ ID NO:268 (denosumab CrossMAb V_{H}-V_{L} huIgG1 KNOB mutation, heavy chain) and SEQ ID NO:269 (denosumab CrossMAb V_{H}-V_{L} light chain).

6. Pharmaceutical compositions

The pharmaceutical compositions of the present invention generally comprise a therapeutic combination or multispecific antigen-binding molecule as described above and elsewhere herein, formulated with one or more pharmaceutically-acceptable carriers. Optionally, the pharmaceutical composition comprises one or more other compounds, drugs, ingredients and/or materials. Regardless of the route of administration selected, the therapeutic combinations or multispecific antigen-binding molecules of the present invention are formulated into pharmaceutically-acceptable dosage forms by conventional methods known to those of skill in the art (see, e.g., Remington, The Science and Practice of Pharmacy (21st Edition, Lippincott Williams and Wilkins, Philadelphia, Pa.)).

A pharmaceutical composition of the present invention may be administered to a subject in any desired and effective manner. For example, the pharmaceutic compositions may be formulated for oral ingestion, or as an ointment or drop for local administration to the eyes, or for parenteral or other administration in any appropriate manner such as intraperitoneal, subcutaneous, topical, intradermal, inhalation, intrapulmonary, rectal, vaginal, sublingual, intramuscular, intravenous, intraatrial, intrathecal, or intralymphatic. Further, a pharmaceutical composition of the present invention may be administered in conjunction with one or more ancillary treatment, as described in detail below. A pharmaceutical composition of the present invention may be encapsulated or otherwise protected against gastric or other secretions, if desired.

The pharmaceutical compositions of the invention may comprise one or more active ingredients in admixture with one or more pharmaceutically-acceptable carriers and, optionally, one or more other compounds, drugs, ingredients and/or materials. Regardless of the route of administration selected, the bispecific antibodies of the present invention are formulated into pharmaceutically-acceptable dosage forms by conventional methods known to those of skill in the art (see, e.g., Remington, The Science and Practice of Pharmacy (21St Edition, Lippincott Williams and Wilkins, Philadelphia, Pa.)).

Pharmaceutically acceptable carriers are well known in the art (see, e.g., Remington, The Science and Practice of Pharmacy (21st Edition, Lippincott Williams and Wilkins, Philadelphia, Pa.) and The National Formulary (American Pharmaceutical Association, Washington, D.C.)) and include sugars (e.g., lactose, sucrose, mannitol, and sorbitol), starches, cellulose preparations, calcium phosphates (e.g., dicalcium phosphate, tricalcium phosphate and calcium hydrogen phosphate), sodium citrate, water, aqueous solutions (e.g., saline, sodium chloride injection, Ringer's injection, dextrose injection, dextrose and sodium chloride injection, lactated Ringer's injection), alcohols (e.g., ethγ1 alcohol, propγ1 alcohol, and benzγ1 alcohol), polyols (e.g., glycerol, propylene glycol, and polyethylene glycol), organic esters (e.g., ethγ1 oleate and triglycerides), biodegradable polymers (e.g., polylactide-polyglycolide, poly(orthoesters), and poly(anhydrides)), elastomeric matrices, liposomes, microspheres, oils (e.g., corn, germ, olive, castor, sesame, cottonseed, and groundnut), cocoa butter, waxes (e.g., suppository waxes), paraffins, silicones, talc, silicylate, etc. Each pharmaceutically acceptable carrier used in a pharmaceutical composition of the invention must be “acceptable” in the sense of being compatible with the other ingredients of the formulation and not injurious to the subject. Carriers suitable for a selected dosage form and intended route of administration are well known in the art, and acceptable carriers for a chosen dosage form and method of administration can be determined using ordinary skill in the art.

The pharmaceutical compositions of the invention optionally contain additional ingredients and/or materials commonly used in pharmaceutical compositions, including therapeutic antigen-binding molecule preparations. These ingredients and materials are well known in the art and include (1) fillers or extenders, such as starches, lactose, sucrose, glucose, mannitol, and silicic acid; (2) binders, such as carboxymethylcellulose, alginates, gelatin, polyvinγ1 pyrrolidone, hydroxypropylmethγ1 cellulose, sucrose and acacia; (3) humectants, such as glycerol; (4) disintegrating agents, such as agar-agar, calcium carbonate, potato or tapioca starch, alginic acid, certain silicates, sodium starch glycolate, cross-linked sodium carboxymethγ1 cellulose and sodium carbonate; (5) solution retarding agents, such as paraffin; (6) absorption accelerators, such as quaternary ammonium compounds; (7) wetting agents, such as cetγ1 alcohol and glycerol monostearate; (8) absorbents, such as kaolin and bentonite clay; (9) lubricants, such as talc, calcium stearate, magnesium stearate, solid polyethylene glycols, and sodium laurγ1 sulfate; (10) suspending agents, such as ethoxylated isostearγ1 alcohols, polyoxyethylene sorbitol and sorbitan esters, microcrystalline cellulose, aluminum metahydroxide, bentonite, agar-agar and tragacanth; (11) buffering agents; (12) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (13) inert diluents, such as water or other solvents; (14) preservatives; (15) surface-active agents; (16) dispersing agents; (17) control-release or absorption-delaying agents, such as hydroxypropylmethγ1 cellulose, other polymer matrices, biodegradable polymers, liposomes, microspheres, aluminum monostearate, gelatin, and waxes; (18) opacifying agents; (19) adjuvants; (20) wetting agents; (21) emulsifying and suspending agents; (22), solubilizing agents and emulsifiers, such as ethγ1 alcohol, isopropyl alcohol, ethγ1 carbonate, ethγ1 acetate, benzγ1 alcohol, benzγ1 benzoate, propylene glycol, 1,3-butylene glycol, oils (in particular, cottonseed, groundnut, corn, germ, olive, castor and sesame oils), glycerol, tetrahydrofurγ1 alcohol, polyethylene glycols and fatty acid esters of sorbitan; (23) propellants, such as chlorofluorohydrocarbons and volatile unsubstituted hydrocarbons, such as butane and propane; (24) antioxidants; (25) agents which render the formulation isotonic with the blood of the intended recipient, such as sugars and sodium chloride; (26) thickening agents; (27) coating materials, such as lecithin; and (28) sweetening, flavoring, coloring, perfuming and preservative agents. Each such ingredient or material must be “acceptable” in the sense of being compatible with the other ingredients of the formulation and not injurious to the subject. Ingredients and materials suitable for a selected dosage form and intended route of administration are well known in the art, and acceptable ingredients and materials for a chosen dosage form and method of administration may be determined using ordinary skill in the art.

Pharmaceutical compositions of the present invention suitable for oral administration may be in the form of capsules, cachets, pills, tablets, powders, granules, a solution or a suspension in an aqueous or non-aqueous liquid, an oil-in-water or water-in-oil liquid emulsion, an elixir or syrup, a pastille, a bolus, an electuary or a paste. These formulations may be prepared by methods known in the art, e.g., by means of conventional pan-coating, mixing, granulation or lyophilization processes.

Solid dosage forms for oral administration (capsules, tablets, pills, dragees, powders, granules and the like) may be prepared, e.g., by mixing the active ingredient(s) with one or more pharmaceutically-acceptable carriers and, optionally, one or more fillers, extenders, binders, humectants, disintegrating agents, solution retarding agents, absorption accelerators, wetting agents, absorbents, lubricants, and/or coloring agents. Solid compositions of a similar type may be employed as fillers in soft and hard-filled gelatin capsules using a suitable excipient. A tablet may be made by compression or molding, optionally with one or more accessory ingredients. Compressed tablets may be prepared using a suitable binder, lubricant, inert diluent, preservative, disintegrant, surface-active or dispersing agent. Molded tablets may be made by molding in a suitable machine. The tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient therein. They may be sterilized by, for example, filtration through a bacteria-retaining filter. These compositions may also optionally contain opacifying agents and may be of a composition such that they release the active ingredient only, or preferentially, in a certain portion of the gastrointestinal tract, optionally, in a delayed manner. The active ingredient can also be in microencapsulated form.

Pharmaceutical compositions of the present invention for rectal or vaginal administration may be presented as a suppository, which may be prepared by mixing one or more active ingredient(s) with one or more suitable non-irritating carriers which are solid at room temperature, but liquid at body temperature and, therefore, will melt in the rectum or vaginal cavity and release the active compound. Pharmaceutical compositions of the present invention which are suitable for vaginal administration also include pessaries, tampons, creams, gels, pastes, foams or spray formulations containing such pharmaceutically-acceptable carriers as are known in the art to be appropriate.

Liquid dosage forms for oral administration include pharmaceutically-acceptable emulsions, microemulsions, solutions, suspensions, syrups and elixirs. The liquid dosage forms may contain suitable inert diluents commonly used in the art. Besides inert diluents, the oral compositions may also include adjuvants, such as wetting agents, emulsifying and suspending agents, sweetening, flavouring, colouring, perfuming and preservative agents. Suspensions may contain suspending agents.

Pharmaceutical compositions of the present invention suitable for parenteral administrations comprise one or more agent(s)/compound(s)/antigen-binding molecules in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain suitable antioxidants, buffers, solutes which render the formulation isotonic with the blood of the intended recipient, or suspending or thickening agents. Proper fluidity can be maintained, for example, by the use of coating materials, by the maintenance of the required particle size in the case of dispersions, and by the use of surfactants. These compositions may also contain suitable adjuvants, such as wetting agents, emulsifying agents and dispersing agents. It may also be desirable to include isotonic agents. In addition, prolonged absorption of the injectable pharmaceutical form may be brought about by the inclusion of agents which delay absorption.

Dosage forms for the topical or transdermal administration include powders, sprays, ointments, pastes, creams, lotions, gels, solutions, patches, drops and inhalants. The active agent (e.g., therapeutic combination or multispecific antigen-binding molecule) may be mixed under sterile conditions with a suitable pharmaceutically-acceptable carrier. The ointments, pastes, creams and gels may contain excipients. Powders and sprays may contain excipients and propellants.

In some cases, in order to prolong the effect of a pharmaceutical composition, it is desirable to slow its absorption from subcutaneous or intramuscular injection. This may be accomplished by the inclusion of a liquid suspension of crystalline or amorphous material having poor water solubility.

The rate of absorption of the active agent (e.g., therapeutic combination or multispecific antigen-binding molecule) then depends upon its rate of dissolution which, in turn, may depend upon crystal size and crystalline form. Alternatively, delayed absorption of a parenterally-administered agent or antibody may be accomplished by dissolving or suspending the active agent or antibody in an oil vehicle. Injectable depot forms may be made by forming microencapsulated matrices of the active ingredient in biodegradable polymers. Depending on the ratio of the active ingredient to polymer, and the nature of the particular polymer employed, the rate of active ingredient release can be controlled. Depot injectable formulations are also prepared by entrapping the drug in liposomes or microemulsions which are compatible with body tissue. The injectable materials can be sterilized for example, by filtration through a bacterial-retaining filter.

The formulations may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

### 6.1 Ancillary Treatments

The therapeutic combinations, multispecific antigen-binding molecules, and pharmaceutical compositions disclosed above and elsewhere herein, may be co-administered with one or more additional therapeutic agents (e.g., anti-cancer agents, cytotoxic or cytostatic agents, hormone treatment, vaccines, and/or other immunotherapies). Alternatively or in addition, the therapeutic agents, bispecific antibodies, and pharmaceutical compositions are administered in combination with other therapeutic treatment modalities, including surgery, radiation, cryosurgery, and/or thermotherapy. Such combination therapies may advantageously utilize lower dosages of the administered therapeutic agents, thus avoiding possible toxicities or complications.

For example, the combination therapies disclosed herein can also be combined with a standard cancer treatment. For example, PD-1 monotherapy is known to be effectively combined with chemotherapeutic regimes. In these instances, it may be possible to reduce the dose of chemotherapeutic reagent administered (Mokyr, M. et al. (1998) Cancer Research 58: 5301-5304). In certain embodiments, the methods and compositions described herein are administered in combination with one or more other antibody molecules, chemotherapy, other anti-cancer therapy (e.g., targeted anti-cancer therapies, or oncolytic drugs), cytotoxic agents, immune-based therapies (e.g., cytokines), surgical and/or radiation procedures. Exemplary cytotoxic agents that can be administered in combination with include antimicrotubule agents, topoisomerase inhibitors, anti-metabolites, mitotic inhibitors, alkylating agents, anthracyclines, vinca alkaloids, intercalating agents, agents capable of interfering with a signal transduction pathway, agents that promote apoptosis, proteasome inhibitors, and radiation (e.g., local or whole body irradiation).

In some embodiments, the therapeutic combination or multispecific antigen-binding molecule is used in combination with a chemotherapeutic agent that is already routinely used as standard in the treatment of the subject. Suitable chemotherapeutic agents include, but are not limited to, anastrozole (ARIMIDEX), bicalutamide (CASODEX), bleomycin sulfate (BLENOXANE), busulfan (MYLERAN), busulfan injection (BUSULFEX), capecitabine (XELODA), N4-pentoxycarbonyl-5-deoxy-5-fluorocytidine, carboplatin (PARAPLATIN), carmustine (BICNU), chlorambucil (LEUKERAN), cisplatin (PLATINOL), cladribine (LEUSTATIN), cyclophosphamide (CYTOXAN or NEOSAR), cytarabine, cytosine arabinoside (CYTOSAR-U), cytarabine liposome injection (DEPOCYT), dacarbazine (DTIC-DOME), dactinomycin (actinomycin D, Cosmegan), daunorubicin hydrochloride (CERUBIDINE), daunorubicin citrate liposome injection (DAUNOXOME), dexamethasone, docetaxel (TAXOTERE), doxorubicin hydrochloride (ADRIAMYCIN, RUBEX), etoposide (VEPESID), fludarabine phosphate (FLUDARA), 5-fluorouracil (ADRUCIL, EFUDEX), flutamide (EULEXIN), tezacitibine, gemcitabine (GEMZAR), hydroxyurea (HYDREA), idarubicin (IDAMYCIN), ifosfamide (IFEX), irinotecan (CAMPTOSAR), L-asparaginase (ELSPAR), leucovorin calcium, melphalan (ALKERAN), 6-mercaptopurine (PURINETHOL), methotrexate (FOLEX), mitoxantrone (NOVANTRONE), mylotarg, paclitaxel (TAXOL), nab-paclitaxel (ABRAXANE), phoenix (Yttrium90/MX-DTPA), pentostatin, polifeprosan 20 with carmustine implant (GLIADEL wafer), tamoxifen citrate (NOLVADEX), teniposide (VUMON), 6-thioguanine, thiotepa, tirapazamine (TIRAZONE), topotecan hydrochloride for injection (HYCAMPTIN), vinblastine (VELBAN), vincristine (ONCOVIN), and vinorelbine (NAVELBINE).

Exemplary alkylating agents include nitrogen mustards, ethylenimine derivatives, alkγ1 sulfonates, nitrosoureas and triazenes): uracil mustard (AMINOURACIL MUSTARD, CHLORETHAMINACIL, DEMETHYLDOPAN, DESMETHYLDOPAN, HAEMANTHAMINE, NORDOPAN, URACIL NITROGEN MUSTARD, URACILLOST, URACILMOSTAZA, URAMUSTIN, URAMUSTINE), chlormethine (MUSTARGEN), cyclophosphamide (CYTOXAN, NEOSAR, CLAFEN, ENDOXAN, PROCYTOX, REVIMMUNE), dacarbazine (DTIC-DOME), ifosfamide (MITOXANA), melphalan (ALKERAN), chlorambucil (LEUKERAN), pipobroman (AMEDEL, VERCYTE), triethylenemelamine (HEMEL, HEXALEN, HEXASTAT), triethylenethiophosphoramine, Temozolomide (TEMODAR and TEMODAL), thiotepa (THIOPLEX), busulfan (BUSILVEX, MYLERAN), carmustine (BICNU), lomustine (CCNUCEENU), streptozocin (ZANOSAR), oxaliplatin (ELOXATIN); dactinomycin (also known as actinomycin-D, COSMEGEN); melphalan (L-PAM, L-sarcolysin, phenylalanine mustard, ALKERAN), altretamine (hexamethylmelamine (HMM), HEXALEN), bendamustine (TREANDA), busulfan (BUSULFEX and MYLERAN), carboplatin (PARAPLATIN), cisplatin (CDDP, PLATINOL and PLATINOL-AQ), chlorambucil (LEUKERAN), dacarbazine (DTIC, DIC and imidazole carboxamide, DTIC-DOME), altretamine (hexamethylmelamine (HMM), HEXALEN), ifosfamide (IFEX), prednumustine, procarbazine (MATULANE), and thiotepa (thiophosphoamide, TESPA and TSPA, THIOPLEX).

Exemplary anthracyclines include, e.g., doxorubicin (ADRIAMYCIN and RUBEX), bleomycin (LENOXANE), daunorubicin (dauorubicin hydrochloride, daunomycin, rubidomycin hydrochloride, and CERUBIDINE), daunorubicin liposomal (daunorubicin citrate liposome, and DAUNOXOME), mitoxantrone (DHAD and NOVANTRONE), epirubicin (ELLENCE), idarubicin (IDAMYCIN and IDAMYCIN PFS), mitomycin C (MUTAMYCIN), geldanamycin, herbimycin, ravidomycin, and desacetylravidomycin.

Exemplary vinca alkaloids that can be used in combination with the agents, antibodies and methods discloses above and elsewhere herein include, but are not limited to, vinorelbine tartrate (NAVELBINE), vincristine (ONCOVIN), vindesine (ELDISINE), and vinblastine (vinblastine sulfate, vincaleukoblastine, VLB, ALKABAN-AQ and VELBAN).

Exemplary proteasome inhibitors that can be used with the present invention include, but are not limited to, bortezomib (VELCADE), carfilzomib (PX-171-007), marizomib (NPI-0052), ixazomib citrate (MLN-9708), delanzomib (CEP-18770), O-Methyl-N-[(2-methyl-5-thiazolyl)carbonyl]-L-seryl-O-methyl-N-[(1S)-2-[(2R)-2-methyl-2-oxiranyl]-2-oxo-1-(phenylmethyl)ethyl]-L-serinamide (ONX-0912); danoprevir (RG7227, CAS 850876-88-9), ixazomib (MLN2238, CAS 1072833-77-2), and (S)-N-[(phenylmethoxy)carbonyl]-L-leucyl-N-(1-formyl-3-methylbutyl)-L-Leucinamide (MG-132, CAS 133407-82-6).

In some embodiments, the agents (e.g., therapeutic combinations or multispecific antigen-binding molecules) may be used in combination with a tyrosine kinase inhibitor (e.g., a receptor tyrosine kinase (RTK) inhibitor). Exemplary tyrosine kinase inhibitors include, but are not limited to, an epidermal growth factor (EGF) pathway inhibitor (e.g., an epidermal growth factor receptor (EGFR) inhibitor), a vascular endothelial growth factor (VEGF) pathway inhibitor (e.g., a vascular endothelial growth factor receptor (VEGFR) inhibitor (e.g., a VEGFR-1 inhibitor, a VEGFR-2 inhibitor, a VEGFR-3 inhibitor)), a platelet derived growth factor (PDGF) pathway inhibitor (e.g., a platelet derived growth factor receptor (PDGFR) inhibitor (e.g., a PDGFR-13 inhibitor)), a RAF-1 inhibitor, a KIT inhibitor and a RET inhibitor.

In some embodiments, the compositions of the present invention are formulated with a hedgehog pathway inhibitor. Suitable hedgehog inhibitors known to be effective in the treatment of cancer include, but are not limited to, axitinib (AG013736), bosutinib (SKI-606), cediranib (RECENTIN, AZD2171), dasatinib (SPRYCEL, BMS-354825), erlotinib (TARCEVA), gefitinib (IRESSA), imatinib (GLEEVEC, CGP57148B, STI-571), lapatinib (TYKERB, TYVERB), lestaurtinib (CEP-701), neratinib (HKI-272), nilotinib (TASIGNA), semaxanib (semaxinib, SU5416), sunitinib (SUTENT, SU11248), toceranib (PALLADIA), vandetanib (ZACTIMA, ZD6474), vatalanib (PTK787, PTK/ZK), trastuzumab (HERCEPTIN), bevacizumab (AVASTIN), rituximab (RITUXAN), cetuximab (ERBITUX), panitumumab (VECTIBIX), ranibizumab (Lucentis), nilotinib (TASIGNA), sorafenib (NEXAVAR), alemtuzumab (CAMPATH), gemtuzumab ozogamicin (MYLOTARG), ENMD-2076, PCI-32765, AC220, dovitinib lactate (TKI258, CHIR-258), BIBW 2992 (TOVOKTM), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, XL228, AEE788, AG-490, AST-6, BMS-599626, CUDC-101, PD153035, pelitinib (EκB-569), vandetanib (zactima), WZ3146, WZ4002, WZ8040, ABT-869 (linifanib), AEE788, AP24534 (ponatinib), AV-951(tivozanib), axitinib, BAY 73-4506 (regorafenib), brivanib alaninate (BMS-582664), brivanib (BMS-540215), cediranib (AZD2171), CHIR-258 (dovitinib), CP 673451, CYC116, E7080, Ki8751, masitinib (AB1010), MGCD-265, motesanib diphosphate (AMG-706), MP-470, OSI-930, pazopanib hydrochloride, PD173074, Sorafenib Tosylate(Bay 43-9006), SU 5402, TSU-68(SU6668), vatalanib, XL880 (GSK1363089, EXEL-2880), vismodegib (2-chloro-N-[4-chloro-3-(2-pyridinyl)phenyl]-4-(methylsulfonyl)-benzamide, GDC-0449 (as disclosed in PCT Publication No. WO 06/028958), 1-(4-Chloro-3-(trifluoromethyl)phenyl)-3-((3-(4-fluorophenyl)-3,4-dihydro-4-oxo-2-quinazolinyl)methyl)-urea (CAS 330796-24-2), N-[(2S,3R,3′R,3aS,4′aR,6S,6′aR,6′bS,7aR,12′aS,12′bS)-2′,3′,3a,4,4′,4′a,5,5′,6,6′,6′a,6′b,7,7′,7a,8′,10′,12′,12′a,12′b-Eicosahydro-3,6,11′,12′b-tetramethylspiro[furo[3,2-b]pyridine-[2(3H),9′(1⁺ H)-naphth[2,1-a]azulen]-3′-yl1-methanesulfonamide (IPI926, CAS 1037210-93-7), 4-Fluoro-N-methyl-N4144-(1-methyl-1H-pyrazol-5-yl)-1-phthalazinyl]-4-piperidinyl]-2-(trifluoromethyl)-benzamide (LY2940680, CAS 1258861-20-9), erismodegib (LDE225).

In certain embodiments, the compositions of the present invention are formulated with a vascular endothelial growth factor (VEGF) receptor inhibitors, including but not limited to, bevacizumab (AVASTIN), axitinib (INLYTA), brivanib alaninate (BMS-582664, (S)-((R)-1-(4-(4-Fluoro-2-methyl-1H-indo1-5-yloxy)-5-methylpyrrolo[2,14][1,2,4]triazin-6-yloxy)propan-2-γ1)₂-aminopropanoate), sorafenib (NEXAVAR), pazopanib (VOTRIENT), sunitinib malate (SUTENT), cediranib (AZD2171, CAS 288383-20-1), vargatef (BIBF1120, CAS 928326-83-4), foretinib (GSK1363089), telatinib (BAY57-9352, CAS 332012-40-5), apatinib (YN968D1, CAS 811803-05-1), imatinib (GLEEVEC), ponatinib (AP24534, CAS 943319-70-8), tivozanib (AV951, CAS 475108-18-0), regorafenib (BAY73-4506, CAS 755037-03-7), vatalanib dihydrochloride (PTK787, CAS 212141-51-0), brivanib (BMS-540215, CAS 649735-46-6), vandetanib (CAPRELSA or AZD6474), motesanib diphosphate (AMG706, CAS 857876-30-3, N-(2,3-dihydro-3,3-dimethyl-1H-indo1-6-yl)-2-[(4-pyridinylmethyl)amino]-3-pyridinecarboxamide, described in the International PCT Publication No. WO 02/066470), dovitinib dilactic acid (TKI258, CAS 852433-84-2), linfanib (ABT869, CAS 796967-16-3), cabozantinib (XL184, CAS 849217-68-1), lestaurtinib (CAS 111358-88-4), N-[5-[[[5-(1,1-Dimethylethyl)-2-oxazolyl]methyl]thio]-2-thiazolyl]-4-piperidinecarboxamide (BMS38703, CAS 345627-80-7), (3R,4R)-4-Amino-1-((4-((3-methoxyphenyl)amino)pyrrolo[2,1-f][1,2,4]triazin-5-yl)methyl)piperidin-3-ol (BMS690514), N-(3,4-Dichloro-2-fluorophenyl)-6-methoxy-7-[[(3aα,5β, 6aα)-octahydro-2-methylcyclopenta[c]pyrrol-5-yl]methoxy]-4-quinazolinamine (XL647, CAS 781613-23-8), 4-Methyl-3-[[1-methyl-6-(3-pyridinyl)-1H-pyrazolo[3,4-d]pyrimidin-4-yl]amino]-N43-(trifluoromethyl)phenylFbenzamide (BHG712, CAS 940310-85-0), and aflibercept (EYLEA).

In some embodiments, the compositions of the present invention are formulated with a PI3K inhibitor. In one embodiment, the PI3K inhibitor is an inhibitor of delta and gamma isoforms of PI3K. Exemplary PI3K inhibitors that can be used in combination are described in, e.g., WO2010/036380, WO2010/006086, WO09/114870, WO05/113556, the contents of which are incorporated herein by reference. Suitably, PI3K inhibitors include 4-[2-(1H-Indazol-4-yl)-[6-[[[4-(methylsulfonyl)piperazin-1-yl]methyl]thieno[3,2-d]pyrimidin-4-yl]morpholine (also known as GDC-0941 (as described in International PCT Publication Nos. WO 09/036082 and WO 09/055730), 2-Methyl-24443-methyl-2-oxo-8-(quinolin-3-yl)-2,3-dihydroimidazo[4,5-c]quinolin-1-yl]phenyl]propionitrile (BEZ235 or NVP-BEZ 235, as described in International PCT Publication No. WO06/122806); 4-(trifluoromethyl)-5-(2,6-dimorpholinopyrimidin-4-yl)pyridin-2-amine (BKM120 or NVP-BKM120, described in International PCT Publication No. WO2007/084786), tozasertib (VX680 or MK-0457, CAS 639089-54-[6); (5Z)-54[4-(4-pyridinyl)-6-quinolinyl]methylene]-2,4-thiazolidinedione (GSK1059615, CAS 958852-01-2); (1E,4S, 4aR, 5R, 6aS, 9aR)-5-(Acetyloxy)-1-[(di-2-propenylamino)methylene]-4,4a,5,6,6a,8,9,9a-octahydro-11-hydroxy-4-(methoxymethyl)-4a,6a-dimethyl-cyclopenta[5,6]naphtho[1,2-c]pyran-2,7,10(1H)-trione (PX866, CAS 502632-66-8); 8-phenyl-2-(morpholin-4-yl)-chromen-4-one (LY294002, CAS 154447-36-6), 2-amino-8-ethyl-4-methyl-6-(1H-pyrazol-5-yl)pyrido[2,3-d]pyrimidin-7(8H)-one (SAR 245409 or XL 765), 1,3-dihydro-8-(6-methoxy-3-pyridinyl)-3-methyl-1-[4-(1-piperazinyl)-3-(trifluoromethyl)phenyl]-2H-imidazo[4,5-c]quinolin-2-one, (2Z)-2-butenedioate (1:1) (BGT 226), 5-fluoro-[3-phenyl-2-[(1S)-1-(9H-purin-6-ylamino)ethyl]-4(3H)-quinazolinone (CAL101), 2-amino-N434N-[3-[(2-chloro-5-methoxyphenyl)amino]quinoxalin-2-yl][sulfamoyl]phenyl]-2-methylpropanamide (SAR 245408 or XL 147), and (S)-pyrrolidine-1,2-dicarboxylic acid 2-amide 1-({4-methyl-542-(2,2,2-trifluoro-1,1-dimethyl-ethyl)-pyridin-4-yl)thiazol-2-yl}-amide) (BYL719).

In some embodiments, the compositions disclosed herein are formulated with a mTOR inhibitor, for example, one or more mTOR inhibitors chosen from one or more of rapamycin, temsirolimus (TORISEL), AZD8055, BEZ235, BGT226, XL765, PF-4691502, GDC0980, SF1126, OSI-027, GSK1059615, KU-0063794, WYE-354, Palomid 529 (P529), PF-04691502, or PKI-587, ridaforolimus (formally known as deferolimus, (1R,2R,4S)-4-[(2R)-2 [(1R,9S,12S,15R,16E,18R,19R,21R, 23S,24E,26E,28Z,30S,32S,35R)-1,18-dihydroxy-19,30-dimethoxy-15,17,21,23, 29,35-hexamethyl-2,3,10,14,20-pentaoxo-11,36-dioxa-4-azatricyclo[30.3.1.0^{4.9} ]hexatriaconta-16,24,26,28-tetraen-12-yl]propyl]-2-methoxycyclohexyl dimethylphosphinate, also known as AP23573 and MK8669, and those described in PCT Publication No. WO03/064383), everolimus (ARINITOR or RAD001), rapamycin (AY22989, SIROLIMUS), simapimod (CAS 164301-51-3), emsirolimus, (5-{2,4-Bis[(3S)-3-methylmorpholin-4-yl]pyrido[2,3-d]pyrimidin-7-yl}-2-methoxyphenyl)methanol (AZD8055), 2-Amino-8-[trans-4-(2-hydroxyethoxy)cyclohexyl]-6-(6-methoxy-3-pyridinyl)-4-methyl-pyrido[2,3-d]pyrimidin-7(8H)-one (PF04691502, CAS 1013101-36-4), and N²-[1,4-dioxo-44[4-(4-oxo-8-phenyl-4H-1-benzopyran-2-yl)morpholinium-4-yl]methoxy]butyl)-arginylglycyl-L-a-aspartylL-serine-, inner salt (SF1126, CAS 936487-67-1), (1r,4-O-4-(4-amino-5-(7-methoxy-1H-indo1-2-yl)imidazo[1,54][1,2,4]triazin-7-γ1)cyclohexanecarboxylic acid (OSI-027); and XL765.

In some embodiments, the compositions of the present invention can be used in combination with a BRAF inhibitor, for example, GSK2118436, RG7204, PLX4032, GDC-0879, PLX4720, and sorafenib tosylate (Bay 43-9006). In further embodiments, a BRAF inhibitor includes, but is not limited to, regorafenib (BAY73-4506, CAS 755037-03-7), tuvizanib (AV951, CAS 475108-18-0), vemurafenib (ZELBORAF, PLX-4032, CAS 918504-65-1), encorafenib (also known as LGX818), [1-Methyl-54-2]-5-(trifluoromethyl)-[1H-imidazol-2-yl]-[4-pyridinyl]oxy-N44-(trifluoromethyl)phenyl-1H-benzimidazol-2-amine (RAF265, CAS 927880-90-8), 541-(2-Hydroxyethyl)-3-(pyridin-4-yl)-1[H-pyrazol-4-yl]-2,3-dihydroinden-1-one oxime (GDC-0879, CAS 905281-76-7), 5-[2-[[2-(Dimethylamino)ethoxy]phenyl]-5-(4-pyridinyl)-1H-imidazol-4-yl]-2,3-dihydro-1H-Inden-1-one oxime (GSK2118436 or SB590885), (+/−)-Methγ1 (5-(2-(5-chloro-2-methylphenyl)-1-hydroxy-3-oxo-2,3-dihydro-1H-isoindo1-1-yl)-1H-benzimidazol-2-yl)carbamate (also known as XL-281 and BMS908662), and N-(3-(5-chloro-1H-pyrrolo[2,3-b]pyridine-3-carbonyl)-2,4-difluorophenyl)propane-l-sulfonamide (also known as PLX4720).

The compositions of the present invention can also be used in combination with a MEK inhibitor. Any MEK inhibitor can be used in combination including, but not limited to, selumetinib (5-[(4-bromo-2-chlorophenyl)amino]-4-fluoro-N-(2-hydroxyethoxy)-1-methyl-1H-benzimidazole-6-carboxamide (AZD6244 or ARRY 142886, described in PCT Publication No. WO2003/077914), trametinib dimethγ1 sulfoxide (GSK-1120212, CAS 1204531-25-80), RDEA436, N43,4-Difluoro-2-[(2-fluoro-4-iodophenyl)amino]-6-[methoxyphenyl]-1-[(2R)-2,3-dihydroxypropyl]-cyclopropanesulfonamide (RDEA119 or BAY869766, described in PCT Publication No. WO2007/014011), AS703026, BIX 02188, BIX 02189, 2-[(2-Chloro-4-iodophenyl)amino]-N-(cyclopropylmethoxy)-3,4-difluoro-benzamide (also known as CI-1040 or PD184352, described in PCT Publication No. WO2000/035436), N-[(2R)-2,3-Dihydroxypropoxy]-3,4-difluoro-2-[(2-fluoro-4-iodophenyl)amino]-benzamide (PD0325901 and described in PCT Publication No. WO2002/006213), 2′-amino-3′-methoxyflavone (PD98059), 2,3-bis[amino[(2-aminophenyl)thio]methylene]-butanedinitrile (U0126 and described in U.S. Pat. No. 2,779,780), XL-518 (GDC-0973, Cas No. 1029872-29-4), G-38963, and G02443714 (also known as AS703206), or a pharmaceutically acceptable salt or solvate thereof. Other MEK inhibitors are disclosed in WO2013/019906, WO03/077914, WO2005/121142, WO2007/04415, WO2008/024725 and WO2009/085983, the contents of which are incorporated herein by reference. Further examples of MEK inhibitors include, but are not limited to, benimetinib (6-(4-bromo-2-fluorophenylamino)-7-fluoro-3-methyl-3H-benzoimidazole-5-carboxylic acid (2-hydroxyethyoxy)-amide (MEK162, CAS 1073666-70-2, described in PCT Publication No. WO2003/077914), 2,3-Bis[amino[(2-aminophenyl)thio]methylene]-butanedinitrile (U0126 and described in U.S. Pat. No. 2,779,780), (3S, 4R, 5Z, 8S, 9S, 11E)-14-(Ethylamino)-8,9,16-trihydroxy-3,4-dimethyl-3,4,9,19-tetrahydro-1H-2-benzoxacyclotetradecine-1,7(8H)-[dione] (E6201, described in PCT Publication No. WO2003/076424), vemurafenib (PLX-4032, CAS 918504-65-1), (R)-3-(2,3-Dihydroxypropyl)-6-fluoro-5-(2-fluoro-4-iodophenylamino)-8-methylpyrido[2,3-d]pyrimidine-4,7(3H,8H)-dione (TAK-733, CAS 1035555-63-5), pimasertib (AS-703026, CAS 1204531-26-9), 2-(2-Fluoro-4-iodophenylamino)-N-(2-hydroxyethoxy)-1,5-dimethyl-6-oxo-1,6-dihydropyridine-3-carboxamide (AZD 8330), and 3,4-Difluoro-2-[(2-fluoro-4-iodophenyl)amino]-N-(2-hydroxyethoxy)-5-[(3-oxo-[1,2]oxazinan-2-yl)methyl]benzamide (CH 4987655 or Ro 4987655).

In some embodiments, the compositions of the present invention are administered with a JAK2 inhibitor, for example, CEP-701, INCB18424, CP-690550 (tasocitinib). Exemplary JAK inhibitors include, but are not limited to, ruxolitinib (JAKAFI), tofacitinib (CP690550), axitinib (AG013736, CAS 319460-85-0), 5-Chloro-N2-[(1S)-1-(5-fluoro-2-pyrimidinylpethyl]-N4-(5-methyl-1H-pyrazol-3-y)-12,4-pyrimidinediamine (AZD1480, CAS 935666-88-9), (9E)-15-[2-(1-Pyrrolidinyl)ethoxy]-7,12,26-trioxa-19,21,24-triazatetracyclo[18.3.1.12,5.114,18]-hexacosa-1(24),2,4,9,14,16,18(25),20,22-nonaene (SB-1578, CAS 937273-04-6), momelotinib (CYT 387), baricitinib (INCB-028050 or LY-3009104), pacritinib (SB1518), (16E)-14-Methyl-20-oxa-5,7,14,27-tetraazatetracyclo[19.3.1.12,6.18,12]heptacosa-1(25),2,4,6(27),8,10,12(26),16,21,23-decaene (SB 1317), gandotinib (LY 2784544), and N,N-cicyclopropyl-4-[(1,5-dimethyl-1H-pyrazol-3-yl)amino]-6-ethyl-1,6-dihydro-l-methyl-imidazo[4,5-d]pyrrolo[2,3-b]pyridine-7-carboxamide (BMS 911543).

In yet other embodiments, the compositions of the present invention are administered in combination with a vaccine, e.g., a dendritic cell renal carcinoma (DC-RCC) vaccine. In certain embodiments, the combination of pharmaceutical compositions and the DC-RCC vaccine is used to treat a cancer, e.g., a cancer as described herein (e.g., a renal carcinoma, e.g., metastatic renal cell carcinoma (RCC) or clear cell renal cell carcinoma (CCRCC).

In yet other embodiments, the pharmaceutical compositions described herein may be administered in combination with chemotherapy, and/or immunotherapy. For example, the compositions can be used to treat a myeloma, alone or in combination with one or more of: chemotherapy or other anti-cancer agents (e.g., thalidomide analogs, e.g., lenalidomide), an anti-TIM3 antibody, tumor antigen-pulsed dendritic cells, fusions (e.g., electrofusions) of tumor cells and dendritic cells, or vaccination with immunoglobulin idiotype produced by malignant plasma cells. In one embodiment, the compositions may be used in combination with an anti-TIM-3 antibody to treat a myeloma, e.g., a multiple myeloma.

In some embodiment, the pharmaceutical compositions of the present invention are used in combination with chemotherapy to treat a lung cancer, e.g., non-small cell lung cancer. In some embodiments, the pharmaceutical compositions are used with platinum doublet therapy to treat lung cancer.

In yet another embodiment, the pharmaceutical compositions disclosed herein may be used to treat a renal cancer, e.g., renal cell carcinoma (RCC) (e.g., clear cell renal cell carcinoma (CCRCC) or metastatic RCC. The anti-PD-1 or PD-L1 antibody molecule can be administered in combination with one or more of: an immune-based strategy (e.g., interleukin-2 or interferon-y), a targeted agent (e.g., a VEGF inhibitor such as a monoclonal antibody to VEGF); a VEGF tyrosine kinase inhibitor such as sunitinib, sorafenib, axitinib and pazopanib; an RNAi inhibitor), or an inhibitor of a downstream mediator of VEGF signaling, e.g., an inhibitor of the mammalian target of rapamycin (mTOR), e.g., everolimus and temsirolimus.

An example of suitable ancillary therapeutics for use in combination for treatment of pancreatic cancer includes, but is not limited to, a chemotherapeutic agent, for example, paclitaxel or a paclitaxel agent (e.g., a paclitaxel formulation such as TAXOL, an albumin-stabilized nanoparticle paclitaxel formulation (e.g., ABRAXANE) or a liposomal paclitaxel formulation), gemcitabine (e.g., gemcitabine alone or in combination with AXP107-11), other chemotherapeutic agents such as oxaliplatin, 5-fluorouracil, capecitabine, rubitecan, epirubicin hydrochloride, NC-6004, cisplatin, docetaxel (e.g., TAXOTERE), mitomycin C, ifosfamide, interferon, tyrosine kinase inhibitor (e.g., EGFR inhibitor (e.g., erlotinib, panitumumab, cetuximab, nimotuzumab), HER2/neu receptor inhibitor (e.g., trastuzumab), dual kinase inhibitor (e.g., bosutinib, saracatinib, lapatinib, vandetanib), multikinase inhibitor (e.g., sorafenib, sunitinib, XL184, pazopanib), VEGF inhibitor (e.g., bevacizumab, AV-951, brivanib), radioimmunotherapy (e.g., XR303), cancer vaccine (e.g., GVAX, survivin peptide), COX-2 inhibitor (e.g., celecoxib), IGF-1 receptor inhibitor (e.g., AMG 479, MK-0646), mTOR inhibitor (e.g., everolimus, temsirolimus), IL-6 inhibitor (e.g., CNTO 328), cyclin-dependent kinase inhibitor (e.g., P276-00, UCN-01), altered energy metabolism-directed (AEMD) compound (e.g., CPI-613), HDAC inhibitor (e.g., vorinostat), TRAIL receptor 2 (TR-2) agonist (e.g., conatumumab), MEK inhibitor (e.g., AS703026, selumetinib, GSK1120212), Raf/MEK dual kinase inhibitor (e.g., R05126766), notch signaling inhibitor (e.g., MK0752), monoclonal antibody-antibody fusion protein (e.g., L19IL2), curcumin, HSP90 inhibitor (e.g., tanespimycin, STA-9090), rIL-2;, denileukin diftitox, topoisomerase 1 inhibitor (e.g., irinotecan, PEP02), statin (e.g., simvastatin), Factor VIIa inhibitor (e.g., PCI-27483), AKT inhibitor (e.g., RX-0201), hypoxia-activated prodrug (e.g., TH-302), metformin hydrochloride, gamma-secretase inhibitor (e.g., R04929097), ribonucleotide reductase inhibitor (e.g., 3-AP), immunotoxin (e.g., HuC242-DM4), PARP inhibitor (e.g., KU-0059436, veliparib), CTLA-4 inhbitor (e.g., CP-675,206, ipilimumab), AdV-tk therapy, proteasome inhibitor (e.g., bortezomib (Velcade), NPI-0052), thiazolidinedione (e.g., pioglitazone), NPC-1C, Aurora kinase inhibitor (e.g., R763/AS703569), CTGF inhibitor (e.g., FG-3019), siG12D LODER, and radiation therapy (e.g., tomotherapy, stereotactic radiation, proton therapy), surgery, and a combination thereof. In certain embodiments, a combination of paclitaxel or a paclitaxel agent, and gemcitabine can be used with the pharmaceutical compositions described herein.

An example of suitable therapeutics for use in combination for treatment of small cell lung cancer includes, but is not limited to, a chemotherapeutic agent, e.g., etoposide, carboplatin, cisplatin, irinotecan, topotecan, gemcitabine, liposomal SN-38, bendamustine, temozolomide, belotecan, NK012, FR901228, flavopiridol), tyrosine kinase inhibitor (e.g., EGFR inhibitor (e.g., erlotinib, gefitinib, cetuximab, panitumumab), multikinase inhibitor (e.g., sorafenib, sunitinib), VEGF inhibitor (e.g., bevacizumab, vandetanib), cancer vaccine (e.g., GVAX), Bcl-2 inhibitor (e.g., oblimersen sodium, ABT-263), proteasome inhibitor (e.g., bortezomib (Velcade), NPI-0052), paclitaxel or a paclitaxel agent, docetaxel, IGF-1 receptor inhibitor (e.g., AMG 479), HGF/SF inhibitor (e.g., AMG 102, MK-0646), chloroquine, Aurora kinase inhibitor (e.g., MLN8237), radioimmunotherapy (e.g., TF2), HSP90 inhibitor (e.g., tanespimycin, STA-9090), mTOR inhibitor (e.g., everolimus), Ep-CAM-/CD3-bispecific antibody (e.g., MT110), CK-2 inhibitor (e.g., CX-4945), HDAC inhibitor (e.g., belinostat), SMO antagonist (e.g., BMS 833923), peptide cancer vaccine, and radiation therapy (e.g., intensity-modulated radiation therapy (IMRT), hypofractionated radiotherapy, hypoxia-guided radiotherapy), surgery, and/or any combination thereof.

An example of suitable therapeutics for use in combination for treatment of non-small cell lung cancer includes, but is not limited to, a chemotherapeutic agent, e.g., vinorelbine, cisplatin, docetaxel, pemetrexed disodium, etoposide, gemcitabine, carboplatin, liposomal SN-38, TLK286, temozolomide, topotecan, pemetrexed disodium, azacitidine, irinotecan, tegafur-gimeracil-oteracil potassium, sapacitabine), tyrosine kinase inhibitor (e.g., EGFR inhibitor (e.g., erlotinib, gefitinib, cetuximab, panitumumab, necitumumab, PF-00299804, nimotuzumab, R05083945), MET inhibitor (e.g., PF-02341066, ARQ 197), PI3K kinase inhibitor (e.g., XL147, GDC-0941), Raf/MEK dual kinase inhibitor (e.g., R05126766), PI3K/mTOR dual kinase inhibitor (e.g., XL765), SRC inhibitor (e.g., dasatinib), dual inhibitor (e.g., BIBW 2992, GSK1363089, ZD6474, AZD0530, AG-013736, lapatinib, MEHD7945A, linifanib), multikinase inhibitor (e.g., sorafenib, sunitinib, pazopanib, AMG 706, XL184, MGCD265, BMS-690514, R935788), VEGF inhibitor (e.g., endostar, endostatin, bevacizumab, cediranib, BIBF 1120, axitinib, tivozanib, AZD2171), cancer vaccine (e.g., BLP25 liposome vaccine , GVAX, recombinant DNA and adenovirus expressing L523S protein), Bcl-2 inhibitor (e.g., oblimersen sodium), proteasome inhibitor (e.g., bortezomib, carfilzomib, NPI-0052, MLN9708), paclitaxel or a paclitaxel agent, docetaxel, IGF-1 receptor inhibitor (e.g., cixutumumab, MK-0646, OSI 906, CP-751,871, BIIB022), hydroxychloroquine, HSP90 inhibitor (e.g., tanespimycin, STA-9090, AUY922, XL888), mTOR inhibitor (e.g., everolimus, temsirolimus, ridaforolimus), Ep-CAM-/CD3-bispecific antibody (e.g., MT110), CK-2 inhibitor (e.g., CX-4945), HDAC inhibitor (e.g., MS 275, LBH589, vorinostat, valproic acid, FR901228), DHFR inhibitor (e.g., pralatrexate), retinoid (e.g., bexarotene, tretinoin), antibody-drug conjugate (e.g., SGN-15), bisphosphonate (e.g., zoledronic acid), cancer vaccine (e.g., belagenpumatucel-L), low molecular weight heparin (LMWH) (e.g., tinzaparin, enoxaparin), GSK1572932A, melatonin, talactoferrin, dimesna, topoisomerase inhibitor (e.g., amrubicin, etoposide, karenitecin), nelfinavir, cilengitide, ErbB3 inhibitor (e.g., MM-121, U3-1287), survivin inhibitor (e.g., YM155, LY2181308), eribulin mesylate, COX-2 inhibitor (e.g., celecoxib), pegfilgrastim, Polo-like kinase 1 inhibitor (e.g., BI 6727), TRAIL receptor 2 (TR-2) agonist (e.g., CS-1008), CNGRC peptide-TNF alpha conjugate, dichloroacetate (DCA), HGF inhibitor (e.g., SCH 900105), SAR240550, PPAR-gamma agonist (e.g., CS-7017), gamma-secretase inhibitor (e.g., R04929097), epigenetic therapy (e.g., 5-azacitidine), nitroglycerin, MEK inhibitor (e.g., AZD6244), cyclin-dependent kinase inhibitor (e.g., UCN-01), cholesterol-Fust, antitubulin agent (e.g., E7389), farnesyl-OH-transferase inhibitor (e.g., lonafarnib), immunotoxin (e.g., BB-10901, SS1 (dsFv) PE38), fondaparinux, vascular-disrupting agent (e.g., AVE8062), PD-L1 inhibitor (e.g., MDX-1105, MDX-1106), beta-glucan, NGR-hTNF, EMD 521873, MEK inhibitor (e.g., GSK1120212), epothilone analog (e.g., ixabepilone), kinesin-spindle inhibitor (e.g., 4SC-205), telomere targeting agent (e.g., KML-001), P70 pathway inhibitor (e.g., LY2584702), AKT inhibitor (e.g., MK-2206), angiogenesis inhibitor (e.g., lenalidomide), Notch signaling inhibitor (e.g., OMP-21M18), radiation therapy, surgery, and combinations thereof.

An example of suitable therapeutics for use in combination for treatment of ovarian cancer includes, but is not limited to, a chemotherapeutic agent (e.g., paclitaxel or a paclitaxel agent, docetaxel, carboplatin, gemcitabine, doxorubicin, topotecan, cisplatin, irinotecan, TLK286, ifosfamide, olaparib, oxaliplatin, melphalan, pemetrexed disodium, SJG-136, cyclophosphamide, etoposide, decitabine), ghrelin antagonist (e.g., AEZS-130), immunotherapy (e.g., APC8024, oregovomab, OPT-821), tyrosine kinase inhibitor (e.g., EGFR inhibitor (e.g., erlotinib), dual inhibitor (e.g., E7080), multikinase inhibitor (e.g., AZD0530, JI-101, sorafenib, sunitinib, pazopanib), ON 01910.Na), VEGF inhibitor (e.g., bevacizumab, BIBF 1120, cediranib, AZD2171), PDGFR inhibitor (e.g., IMC-3G3), paclitaxel, topoisomerase inhibitor (e.g., karenitecin, Irinotecan), HDAC inhibitor (e.g., valproate, vorinostat), folate receptor inhibitor (e.g., farletuzumab), angiopoietin inhibitor (e.g., AMG 386), epothilone analog (e.g., ixabepilone), proteasome inhibitor (e.g., carfilzomib), IGF-1 receptor inhibitor (e.g., OSI 906, AMG 479), PARP inhibitor (e.g., veliparib, AG014699, iniparib, MK-4827), Aurora kinase inhibitor (e.g., MLN8237, ENMD-2076), angiogenesis inhibitor (e.g., lenalidomide), DHFR inhibitor (e.g., pralatrexate), radioimmunotherapeutic agnet (e.g., Hu3S193), statin (e.g., lovastatin), topoisomerase 1 inhibitor (e.g., NKTR-102), cancer vaccine (e.g., p53 synthetic long peptides vaccine, autologous OC-DC vaccine), mTOR inhibitor (e.g., temsirolimus, everolimus), BCR/ABL inhibitor (e.g., imatinib), ET-A receptor antagonist (e.g., ZD4054), TRAIL receptor 2 (TR-2) agonist (e.g., CS-1008), HGF/SF inhibitor (e.g., AMG 102), EGEN-001, Polo-like kinase 1 inhibitor (e.g., BI 6727), gamma-secretase inhibitor (e.g., R04929097), Wee-1 inhibitor (e.g., MK-1775), antitubulin agent (e.g., vinorelbine, E7389), immunotoxin (e.g., denileukin diftitox), SB-485232, vascular-disrupting agent (e.g., AVE8062), integrin inhibitor (e.g., EMD 525797), kinesin-spindle inhibitor (e.g., 4SC-205), revlimid, HER2 inhibitor (e.g., MGAH22), ErrB3 inhibitor (e.g., MM-121), radiation therapy, and combinations thereof.

An example of suitable therapeutics for use in combination to treat a myeloma, alone or in combination with one or more of: chemotherapy or other anti-cancer agents (e.g., thalidomide analogs, e.g., lenalidomide), HSCT (Cook, R. (2008) J Manag Care Pharm. 14(7 Suppl):19-25), an anti-TIM3 antibody (Hallett, WHD et al. (2011) J of American Society for Blood and Marrow Transplantation 17(8):1133-145), tumor antigen-pulsed dendritic cells, fusions (e.g., electrofusions) of tumor cells and dendritic cells, or vaccination with immunoglobulin idiotype produced by malignant plasma cells (reviewed in Yi, Q. (2009) Cancer J. 15(6):502-10).

Examples of suitable therapeutics for use with the compositions of the present invention for treatment of chronic lymphocytic leukemia (CLL) include, but are not limited to, a chemotherapeutic agent (e.g., fludarabine, cyclophosphamide, doxorubicin, vincristine, chlorambucil, bendamustine, chlorambucil, busulfan, gemcitabine, melphalan, pentostatin, mitoxantrone, 5-azacytidine, pemetrexed disodium), tyrosine kinase inhibitor (e.g., EGFR inhibitor (e.g., erlotinib), BTK inhibitor (e.g., PCI-32765), multikinase inhibitor (e.g., MGCD265, RGB-286638), CD-20 targtting agent (e.g., rituximab, ofatumumab, 805072759, LFB-R603), CD52 targeting agent (e.g., alemtuzumab), prednisolone, darbepoetin alfa, lenalidomide, Bcl-2 inhibitor (e.g., ABT-263), immunotherapy (e.g., allogeneic CD4+memory Th1-like T cells/microparticle-bound anti-CD3/anti-CD28, autologous cytokine induced killer cells (CIK)), HDAC inhibitor (e.g., vorinostat, valproic acid, LBH589, JNJ-26481585, AR-42), XIAP inhibitor (e.g., AEG35156), CD-74 targeting agent (e.g., milatuzumab), mTOR inhibitor (e.g., everolimus), AT-101, immunotoxin (e.g., CAT-8015, anti-Tac(Fv)-PE38 (LMB-2)), CD37 targeting agent (e.g., TRU-016), radioimmunotherapy (e.g., 131-tositumomab), hydroxychloroquine, perifosine, SRC inhibitor (e.g., dasatinib), thalidomide, PI3K delta inhibitor (e.g., CAL-101), retinoid (e.g., fenretinide), MDM2 antagonist (e.g., R05045337), plerixafor, Aurora kinase inhibitor (e.g., MLN8237, TAK-901), proteasome inhibitor (e.g., bortezomib), CD-19 targeting agent (e.g., MEDI-551, MOR208), MEK inhibitor (e.g., ABT-348), JAK-2 inhibitor (e.g., INCB018424), hypoxia-activated prodrug (e.g., TH-302), paclitaxel or a paclitaxel agent, HSP90 inhibitor, AKT inhibitor (e.g., MK2206), HMG-CoA inhibitor (e.g., simvastatin), GNKG186, radiation therapy, bone marrow transplantation, stem cell transplantation, and a combination thereof.

An example of suitable therapeutics for use in combination for treatment of acute lymphocytic leukemia (ALL) includes, but is not limited to, a chemotherapeutic agent (e.g., prednisolone, dexamethasone, vincristine, asparaginase, daunorubicin, cyclophosphamide, cytarabine, etoposide, thioguanine, mercaptopurine, clofarabine, liposomal annamycin, busulfan, etoposide, capecitabine, decitabine, azacitidine, topotecan, temozolomide), tyrosine kinase inhibitor (e.g., BCR/ABL inhibitor (e.g., imatinib, nilotinib), ON 01910.Na, multikinase inhibitor (e.g., sorafenib)), CD-20 targeting agent (e.g., rituximab), CD52 targeting agent (e.g., alemtuzumab), HSP90 inhibitor (e.g., STA-9090), mTOR inhibitor (e.g., everolimus, rapamycin), JAK-2 inhibitor (e.g., INCB018424), HER2/neu receptor inhibitor (e.g., trastuzumab), proteasome inhibitor (e.g., bortezomib), methotrexate, asparaginase, CD-22 targeting agent (e.g., epratuzumab, inotuzumab), immunotherapy (e.g., autologous cytokine induced killer cells (CIK), AHN-12), blinatumomab, cyclin-dependent kinase inhibitor (e.g., UCN-01), CD45 targeting agent (e.g., BC8), MDM2 antagonist (e.g., R05045337), immunotoxin (e.g., CAT-8015, DT2219ARL), HDAC inhibitor (e.g., JNJ-26481585), JVRS-100, paclitaxel or a paclitaxel agent, STAT3 inhibitor (e.g., OPB-31121), PARP inhibitor (e.g., veliparib), EZN-2285, radiation therapy, steroid, bone marrow transplantation, stem cell transplantation, or a combination thereof.

An example of suitable therapeutics for use in combination for treatment of acute myeloid leukemia (AML) includes, but is not limited to, a chemotherapeutic agent (e.g., cytarabine, daunorubicin, idarubicin, clofarabine, decitabine, vosaroxin, azacitidine, clofarabine, ribavirin, CPX-351, treosulfan, elacytarabine, azacitidine), tyrosine kinase inhibitor (e.g., BCR/ABL inhibitor (e.g., imatinib, nilotinib), ON 01910.Na, multikinase inhibitor (e.g., midostaurin, SU 11248, quizartinib, sorafinib)), immunotoxin (e.g., gemtuzumab ozogamicin), DT388IL3 fusion protein, HDAC inhibitor (e.g., vorinostat, LBH589), plerixafor, mTOR inhibitor (e.g., everolimus), SRC inhibitor (e.g., dasatinib), HSP90 inhbitor (e.g., STA-9090), retinoid (e.g., bexarotene, Aurora kinase inhibitor (e.g., BI 811283), JAK-2 inhibitor (e.g., INCB018424), Polo-like kinase inhibitor (e.g., BI 6727), cenersen, CD45 targeting agent (e.g., BC8), cyclin-dependent kinase inhibitor (e.g., UCN-01), MDM2 antagonist (e.g., R05045337), mTOR inhibitor (e.g., everolimus), LY573636-sodium, ZRx-101, MLN4924, lenalidomide, immunotherapy (e.g., AHN-12), histamine dihydrochloride, radiation therapy, bone marrow transplantation, stem cell transplantation, and a combination thereof.

Examples of suitable therapeutics for use with the compositions of the present invention for treatment of multiple myeloma (MM) includes, but is not limited to, a chemotherapeutic agent (e.g., melphalan, amifostine, cyclophosphamide, doxorubicin, clofarabine, bendamustine, fludarabine, adriamycin, SyB L-0501), thalidomide, lenalidomide, dexamethasone, prednisone, pomalidomide, proteasome inhibitor (e.g., bortezomib, carfilzomib, MLN9708), cancer vaccine (e.g., GVAX), CD-40 targeting agent (e.g., SGN-40, CHIR-12.12), perifosine, zoledronic acid, Immunotherapy (e.g., MAGE-A3, NY-ESO-1 , HuMax-CD38), HDAC inhibitor (e.g., vorinostat, LBH589, AR-42), aplidin, cycline-dependent kinase inhibitor (e.g., PD-0332991, dinaciclib), arsenic trioxide, CB3304, HSP90 inhibitor (e.g., KW-2478), tyrosine kinase inhibitor (e.g., EGFR inhibitor (e.g., cetuximab), multikinase inhibitor (e.g., AT9283)), VEGF inhibitor (e.g., bevacizumab), plerixafor, MEK inhibitor (e.g., AZD6244), IPH2101, atorvastatin, immunotoxin (e.g., BB-10901), NPI-0052, radioimmunotherapeutic (e.g., yttrium Y 90 ibritumomab tiuxetan), STAT3 inhibitor (e.g., OPB-31121), MLN4924, Aurora kinase inhibitor (e.g., ENMD-2076), IMGN901, ACE-041, CK-2 inhibitor (e.g., CX-4945), radiation therapy, bone marrow transplantation, stem cell transplantation, and a combination thereof.

Examples of suitable therapeutics for use with the compositions of the present invention for treatment of prostate cancer includes, but is not limited to, a chemotherapeutic agent (e.g., docetaxel, carboplatin, fludarabine), abiraterone, hormonal therapy (e.g., flutamide, bicalutamide, nilutamide, cyproterone acetate, ketoconazole, aminoglutethimide, abarelix, degarelix, leuprolide, goserelin, triptorelin, buserelin), tyrosine kinase inhibitor (e.g., dual kinase inhibitor (e.g., lapatanib), multikinase inhibitor (e.g., sorafenib, sunitinib)), VEGF inhibitor (e.g., bevacizumab), TAK-700, cancer vaccine (e.g., BPX-101, PEP223), lenalidomide, TOK-001, IGF-1 receptor inhibitor (e.g., cixutumumab), TRC105, Aurora A kinase inhibitor (e.g., MLN8237), proteasome inhibitor (e.g., bortezomib), OGX-011, radioimmunotherapy (e.g., HuJ591-GS), HDAC inhibitor (e.g., valproic acid, SB939, LBH589), hydroxychloroquine, mTOR inhibitor (e.g., everolimus), dovitinib lactate, diindolylmethane, efavirenz, OGX-427, genistein, IMC-3G3, bafetinib, CP-675,206, radiation therapy, surgery, or a combination thereof.

The combination therapies can be administered in combination with one or more of the existing modalities for treating cancers, including, but not limited to: surgery; radiation therapy (e.g., external-beam therapy which involves three dimensional, conformal radiation therapy where the field of radiation is designed, local radiation (e.g., radiation directed to a preselected target or organ), or focused radiation). Focused radiation can be selected from the group consisting of stereotactic radiosurgery, fractionated stereotactic radiosurgery, and intensity-modulated radiation therapy. The focused radiation can have a radiation source selected from the group consisting of a particle beam (proton), cobalt-60 (photon), and a linear accelerator (x-ray), e.g., as described in WO201²/₁77624, which is incorporated herein by reference in its entirety.

Radiation therapy can be administered through one of several methods, or a combination of methods, including external-beam therapy, internal radiation therapy, implant radiation, stereotactic radiosurgery, systemic radiation therapy, radiotherapy and permanent or temporary interstitial brachytherapy. The term “brachytherapy,” refers to radiation therapy delivered by a spatially confined radioactive material inserted into the body at or near a tumor or other proliferative tissue disease site. The term is intended without limitation to include exposure to radioactive isotopes (e.g., At-211, 1-131, 1-125, Y-90, Re-186, Re-188, Sm-153, Bi-212, P-32, and radioactive isotopes of Lu). Suitable radiation sources for use as a cell conditioner of the present disclosure include both solids and liquids. By way of non-limiting example, the radiation source can be a radionuclide, such as 1-125, 1-131, Yb-169, Ir-192 as a solid source, 1-125 as a solid source, or other radionuclides that emit photons, beta particles, gamma radiation, or other therapeutic rays. The radioactive material can also be a fluid made from any solution of radionuclide(s), e.g., a solution of 1-125 or 1-131, or a radioactive fluid can be produced using a slurry of a suitable fluid containing small particles of solid radionuclides, such as Au-198, Y-90. Moreover, the radionuclide(s) can be embodied in gels or radioactive microspheres.

### 7. Methods of Treatment

Also encapsulated by the present invention is a method of treating cancer in a subject. The method comprises administering to the subject an effect amount of an agent (e.g., therapeutic combination or multispecific antigen-binding molecule) as broadly described above and elsewhere herein.

In accordance with the present invention, it is proposed that the agents of the present invention (e.g., therapeutic combinations and multispecific antigen-binding molecules) that antagonize RANKL and antagonize at least one ICM may be used therapeutically after a cancer or tumor is diagnosed, or may be used prophylactically before the subject develops a cancer or tumor. The present invention therefore provides a therapeutic combination, multispecific antigen-binding molecule, and pharmaceutical composition that antagonizes both RANKL and at least one ICM for use in (a) treating cancer, (b) delaying progression of cancer, c) prolonging the survival of a patient suffering from cancer, or (d) stimulating a cell mediated immune response to the cancer. Accordingly, the present invention also provides methods for (a) treating cancer, (b) delaying progression of cancer, (c) prolonging the survival of a patient suffering from cancer, or (d) stimulating a cell mediated immune response to the cancer. Cancers which can be suitably treated in accordance with the practices of this invention include melanoma, breast cancer, colon cancer, ovarian cancer, endometrial and uterine carcinoma, gastric or stomach cancer, pancreatic cancer, prostate cancer, salivary gland cancer, lung cancer, hepatocellular cancer, glioblastoma, cervical cancer, liver cancer, bladder cancer, hepatoma, rectal cancer, colorectal cancer, kidney cancer, vulval cancer, thyroid cancer, hepatic carcinoma, anal carcinoma, penile carcinoma, testicular cancer, oesophageal cancer, tumours of the biliary tract, head and neck cancer, and squamous cell carcinoma.

Specific concurrent and/or sequential dosing regimens for any given subject may be established based upon the specific disease for which the patient has been diagnosed, or in conjunction with the stage of the patient's disease. For example, if a patient is diagnosed with a less-aggressive cancer, or a cancer that is in its early stages, the patient may have an increased likelihood of achieving a clinical benefit and/or immune-related response to a concurrent administration of an anti-RANKL agent followed by an anti-ICM agent and/or a sequential administration of an anti-RANKL agent followed by an anti-ICM agent. Alternatively, if a patient is diagnosed with a more-aggressive cancer, or a cancer that is in its later stages, the patient may have a decreased likelihood of achieving a clinical benefit and/or immune-related response to said concurrent and/or sequential administration, and thus may suggest that either higher doses of said anti-RANKL agent and/or said anti-ICM agent therapy should be administered or more aggressive dosing regimens or either agent or combination therapy may be warranted. In one aspect, an increased dosing level of an anti-RANKL antigen-binding molecule, such as denosumab, would be about 10, 20, 30, 40, 50, 60, 70, 80, 90, or 95% more than the typical anti-RANKL agent dose for a particular indication or individual (e.g., about 0.3 mg/kg, about 1 mg/kg, about 3 mg/kg, about 10 mg/kg, about 15 mg/kg, about 20 mg kg, about 25 mg/kg, about 30 mg/kg), or about 1.5, 2, 2.5, 3, 3.5, 4, 4.5, 5, 6, 7, 8, 9, or 10 times more anti-RANKL agent than the typical dose for a particular indication or for individual. In another aspect, an increased dosing level of an anti-ICM agent would be about 10, 20, 30, 40, 50, 60, 70, 80, 90, or 95% more than the typical anti-PD-1 agent dose for a particular indication or individual (e.g., about 0.03 mg/kg, 0.1 mg/kg, 0.3 mg/kg, about 3 mg/kg, about 10 mg/kg, about 15 mg/kg, about 20 mg/kg, about 25 mg/kg, about 30 mg/kg; or about 3 mg, about 4 mg, about 5 mg, about 6 mg, about 7 mg, about 8 mg, about 9 mg, about 10 mg, about 11 mg, about 12 mg, about 13 mg, about 14 mg, about 15 mg or about 16 mg), or about 1.5, 2, 2.5, 3, 3.5, 4, 4.5, 5, 6, 7, 8, 9, or 10 times more anti-ICM agent than the typical dose for a particular indication.

A therapeutically effective amount of an anti-RANKL agent and/or an anti-ICM agent, will preferably be injected into the subject, for example, if it is a biologic agent. The actual dosage employed can be varied depending upon the requirements of the patient and the severity of the condition being treated. Determination of the proper starting dosage for a particular situation is within the repertoire of a skilled person in the art, though the assignment of a treatment regimen will benefit from taking into consideration the indication and the stage of the disease. Nonetheless, it will be understood that the specific dose level and frequency of dosing for any particular subject can be varied and will depend upon a variety of factors including the activity of the specific compound employed, the metabolic stability and length of action of that compound, the species, age, body weight, general health, sex and diet of the patient, the mode and time of administration, rate of excretion, drug combination, and severity of the particular condition. Preferred subjects for treatment include animals, most preferably mammalian species such as humans, and domestic animals such as dogs, cats, and the like, patient to cancer.

### 8. Kits

A further embodiment of the present invention is a kit for treating a cancer in a subject. This kit comprises any pharmaceutical composition as disclosed herein.

For use in the kits of the invention, pharmaceutical compositions comprising suitable therapeutic combinations and/or multispecific antigen-binding molecules, and optionally with instructions for cancer treatment. The kits may also include suitable storage containers (e.g., ampules, vials, tubes, etc.), for each pharmaceutical composition and other included reagents (e.g., buffers, balanced salt solutions, etc.), for use in administering the pharmaceutical compositions to subjects. The pharmaceutical compositions and other reagents may be present in the kits in any convenient form, such as, e.g., in a solution of in a powder pharmaceutical compositions. The kits may further include a packaging container, optionally having one or more partitions for housing the pharmaceutical composition and other optional reagents.

In order that the invention may be readily understood and put into practical effect, particular preferred embodiments will now be described by way of the following non-limiting examples.

### EXAMPLES

### Example 1

### Suppression of Lung Metastasis by Co-Blockade of CTLA4 And RANKL is Dependent on Nk Cells and IFN-Gamma

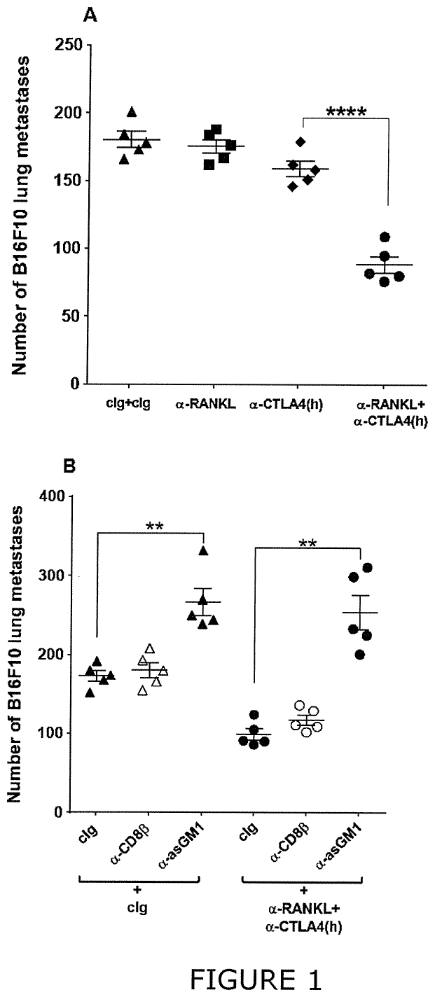

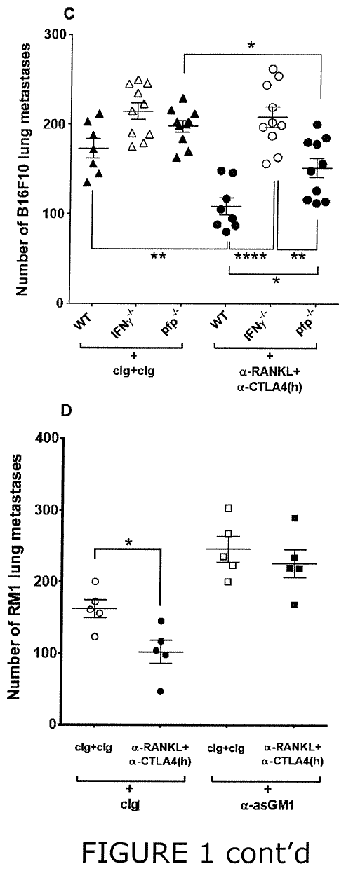

In mice bearing experimental B16F10 melanoma lung metastases, wild-type (WT) mice treated with the combination of hamster anti-CTLA4 (UC10-4F10) and rat anti-RANKL (IK22/5) MAbs showed superior resistance to metastases compared with mice treated with either antibody alone or control immunoglobulin (cIg) (FIG. 1A). The mechanism of action of anti-CTLA4 and anti-RANKL combination therapy was determined in wild-type mice depleted of CD8⁺ or NK cells or mice deficient for perforin or IFNy. As shown in FIG. 1B, the efficacy of the combination relied on the presence of NK cells, but not CD8⁺ T-cells, and IFNy was critical and to a lesser extent, perforin (FIG. 1C). A similar dependence on NK cells was demonstrated for the effective control of prostate carcinoma RM-1 experimental lung metastases following treatment the same with anti-CTLA4 and anti-RANKL combination therapy (FIG. 1D).

### Example 2

### Anti-RANKL Optimally Synergizes with Ctla4 Antibodies of the Igg2A Isotype

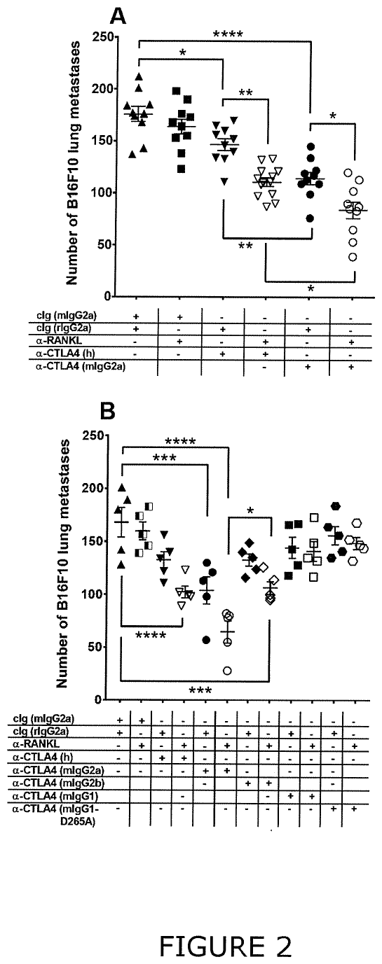

Given that the immunoglobulin constant region of anti-CTLA4 has been reported to influence antitumor activity (Selby et al., 2013, Cancer Immunol. Res., 1(1):32-[2]), the inventors next assessed the impact of how different anti-CTLA4 antibody isotypes synergized with anti-RANKL in suppressing experimental B16F10 lung metastases (FIG. 2). 9D9 is an anti-CTLA4 clone which has been produced as a number of isotypes, including mouse IgG1, IgG2a and IgG2b; while another isotype (IgG1-D265A) contained a mutation which eliminated binding to all Fcy receptors (FcγR)(Selby et al., 2013, supra). As shown in FIG. 2A, the IgG2a isotype of the anti-CTLA4 (filled circles) alone resulted in greater suppression of lung metastases compared with the hamster clone of anti-CTLA4 (inverted filled triangles), and this suppression was further increased with the addition of anti-RANKL to either anti-CTLA4 clones. Similarly, significant suppression of RM-1 and LWT1 lung metastases were also seen with the mouse IgG2a anti-CTLA4 and anti-RANKL combination therapy (FIG. 2C).

Interestingly, the other three anti-CTLA4 isotypes alone (IgG2b (filled diamonds), IgG1(filled squares) or IgG1-D265A (filled hexagons)) were not as effective in suppressing lung metastases compared to the anti-CTLA4-IgG2a isotype (filled circles) as they did not result in significant suppression of metastases compared with the cIg treated group (filled triangles)(FIG. 2B). However, the addition of anti-RANKL to the hamster (open inverted triangles) or mouse IgG2b (open diamonds) isotypes of anti-CTLA4 resulted in significant suppression of lung metastases compared with cIg (filled triangles). Nevertheless the group treated with anti-RANKL and the anti-CTLA4-IgG2a clone (open circles) was significantly superior to the combination anti-RANKL with anti-CTLA4-IgG2b (FIG. 2B). Overall, anti-RANKL treatment alone did not significantly suppress metastasis, although it significantly improved the control of metastases when used in combination with specific anti-CTLA4 isotypes, most notably that of the IgG2a (FIGS. 2A, B).

### Example 3

### Anti-RANKL and Anti-CTLA4 Suppress Subcutaneous B16F10 Melanoma Growth

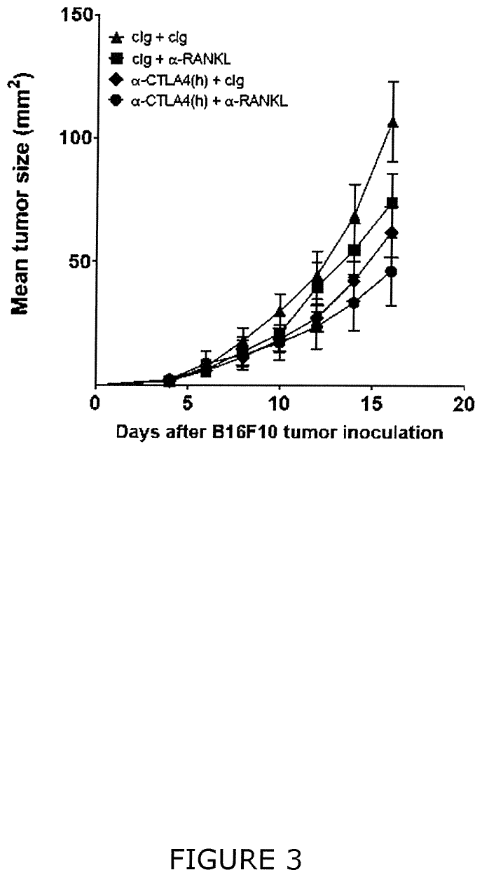

Next, the efficacy of dual blockade of RANKL and CTLA4 was assessed in mice bearing subcutaneous B16F10 melanoma, which is generally poorly immunogenic (FIG. 3). Similar to the lung metastasis models, the combination therapy again demonstrated a greater suppression of growth compared with monotherapies, although the combination effect with the anti-CTLA4 hamster isotype was not significant.

### Example 4

### Anti-RANKL and Anti-CTLA4 Suppress Subcutaneous B16F10 Melanoma Growth

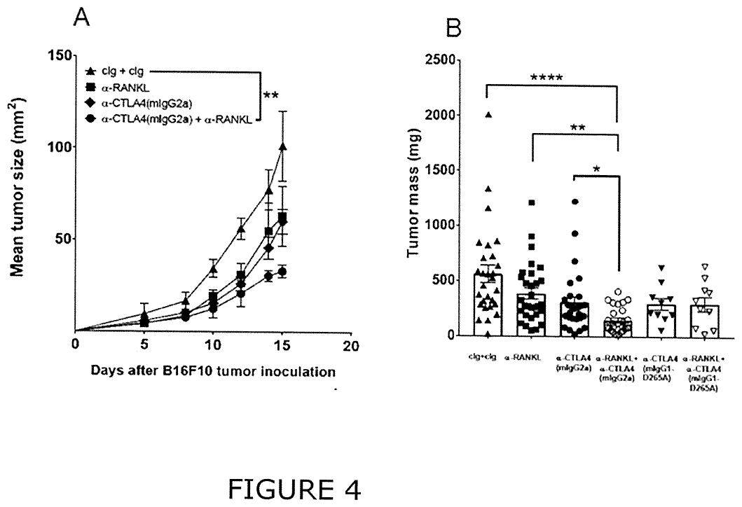

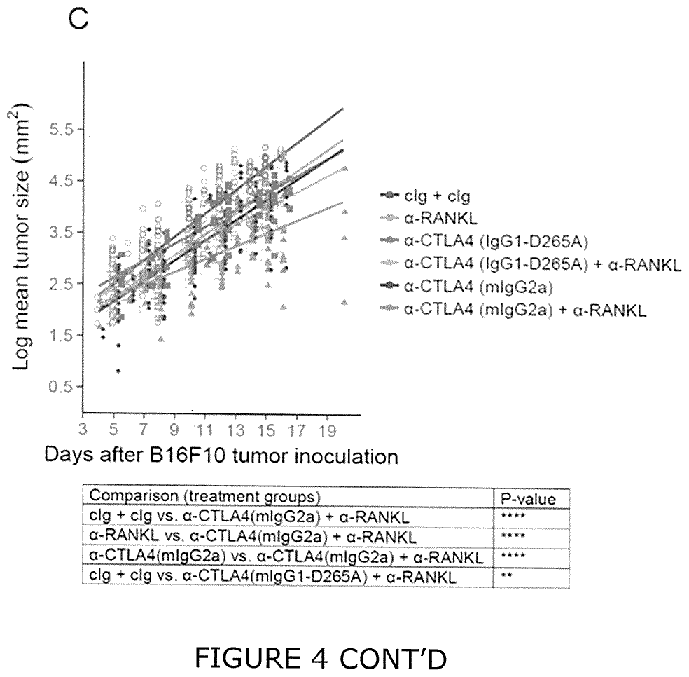

Similar to the lung metastasis models, the combination therapy was again dependent on antibody isotype, with significant suppression of growth observed when the anti-CTLA4-IgG2a isotype (FIG. 4A), rather than the hamster isotype (FIG. 3). A longitudinal analysis of seven independent pooled experiments was performed, comparing anti-CTLA4-IgG2a or the FcR-non-engaging clone of anti-CTLA4 (IgG1-D265A) and/or anti-RANKL with control Ig (FIG. 4C). Overall the data demonstrated the combination of anti-CTLA4-IgG2a with anti-RANKL significantly suppressed tumor growth compared with monotherapy or cIg (FIG. 4C). By contrast, the combination of anti-CTLA4-IgG1-D265A and anti-RANKL was superior to cIg treated groups but was not superior to either treatment as a monotherapy (FIG. 4C). Similarly, tumor mass at endpoint of mice treated with the combination therapy containing the anti-CTLA4-IgG2a isotype was also significantly decreased compared to the respective monotherapy treated groups. However this benefit was not observed in the groups treated with the combination therapy containing the anti-CTLA4-IgG1-D265A isotype compared with mice treated with anti-CTLA4-IgG1-D265A alone (FIG. 4B).

### Example 5

### RANKL And Rank Expression in the Tumor Microenvironment

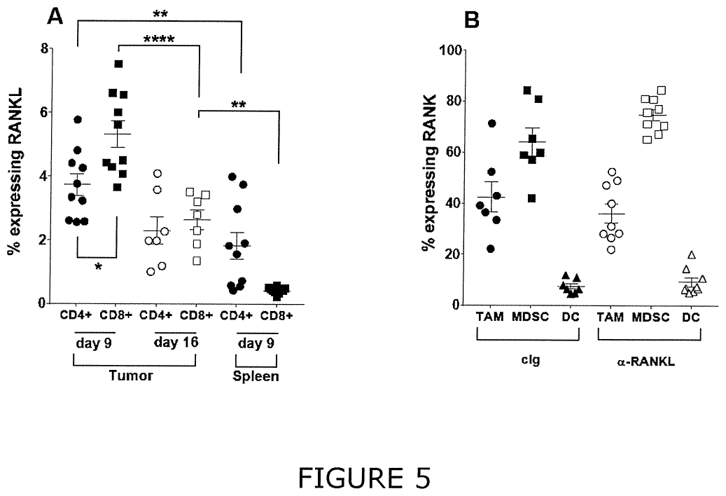

Expression of RANKL and RANK in the B16F10 tumor microenvironment (TME) was next defined (FIG. 5). The majority of intratumor RANKL was expressed by a small fraction of T cells, with expression higher at an earlier time point (day 9) and higher in tumor than in spleen, with more CD8⁺ T cell compared to CD4⁺ T cells expressing RANKL in the tumor (FIG. 5A). Overall, about 20% of tumor-infiltrating leukocytes (TILs) expressed RANK (although the range can be quite large) with greater than 90% staining for CD11b (data not shown) suggesting intratumor RANK was expressed almost exclusively by tumor-infiltrating myeloid cells. About 40% of tumor-infiltrating macrophages (TAM), 60% of MDSCs, and a low but variable proportion of DCs (5-20%), expressed RANK (FIG. 4B). Treatment with anti-RANKL did not significantly alter myeloid RANK expression on these cell types (FIG. 5B). Recently it was reported in a B16 melanoma model that Lγ6C^{low}MHCII^{high} intratumor macrophages had an RNA expression profile consistent with an inflammatory M1 subtype, while those with MHCII^{low/negative} expression were thought to be have an immunosuppressive M2 phenotype (De Henau et al., 2016, Nature, 539(7629):443-7). Notably, in the inventor's B16F10 model, a higher proportion of Lγ6C/Lγ6G (GR-1)^{low} TAMs expressing RANK had negative or low MHCII expression compared with those not expressing RANK, suggesting that the RANK-expressing TAM population may be more suppressive than those TAMs not expressing RANK (data not shown). However, anti-RANKL treatment did not alter the proportion of CD11b⁺ myeloid TILs, the proportion of TAMs expressing CD206 (an M2 marker) in either B16F10 or RM-1 subcutaneous tumors (data not shown). Less than 1% of RANKL- or RANK-expressing cells are CD45.2 negative (indicating a negligible level of intratumor expression of either in vivo), and additionally all tumor cell lines used in this study when assessed by flow cytometry were negative for RANKL or RANK expression (data not shown).

### Example 6

### Anti-Tumor Efficacy Of Anti-RANKL And Anti-ctla4-IGG2A Combination Therapy is FcrRIV Receptor, IFNγ AND CD8⁺ T Cell-Dependent

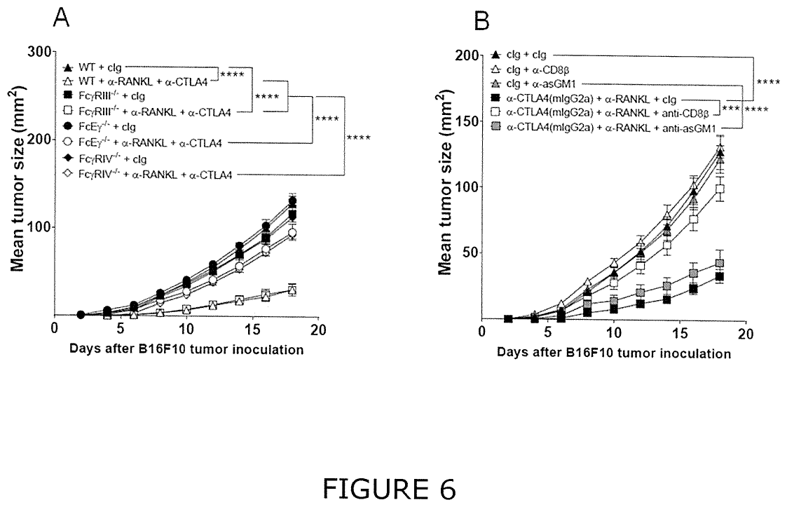

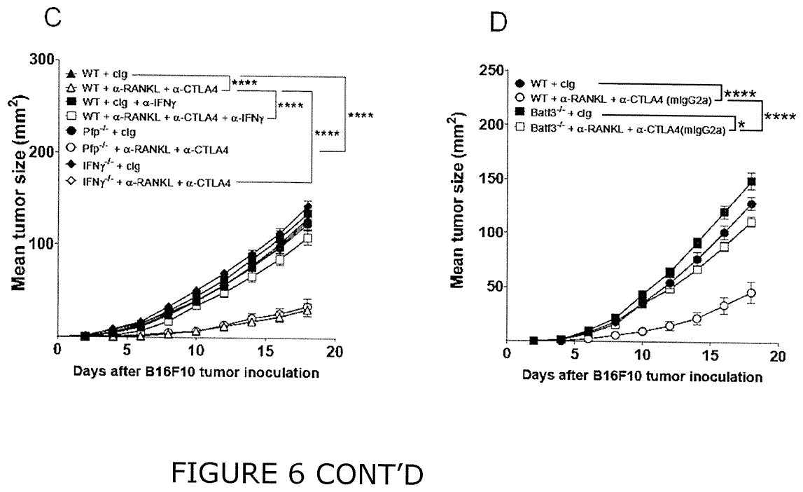

Next, the present inventors assessed the reliance of the combinatorial efficacy of anti-RANKL with anti-CTLA4-IgG2a on Fc receptors as well as the presence and function of effector lymphocytes in the subcutaneous B16F10 tumor model (FIG. 6). Consistent with the known mechanism of action of anti-CTLA4-IgG2a, its combination activity with anti-RANKL against B16F10 was abrogated in mice lacking FcγRIV or FcEyR, but not FcγRIII (FIG. 6A). Although anti-CTLA4 MAb was FcγRIV-dependent, it was not clear whether blockade of RANKL had a similar requirement. Next, the role of CD8⁺ T cells and NK cells in the control of B16F10 tumor growth by the anti-CTLA4-mIgG2a and anti-RANKL was assessed by the selective depletion of each subset (FIG. 6B). When CD8⁺ T cells were depleted, the anti-tumor efficacy of the combination therapy was almost completely abrogated. By contrast NK-cell depletion was without effect, demonstrating the reliance of this combination therapy on CD8⁺ T cells (FIG. 6B). Similar to results observed in the metastatic setting, this combination therapy was IFNγ⁻, but not perforin-, dependent (FIG. 6C). The essential role for cross-presenting CD8α⁺ conventional DCs in this combination therapy was also revealed by the use of mice deficient in the transcription factor Batf3; the efficacy of the combination therapy was abrogated in these mice compared with WT treated mice (FIG. 6D).

### Example 7

### CDS+T Cell Influx into Tumor Post CTLA4 And RANKL Blockade

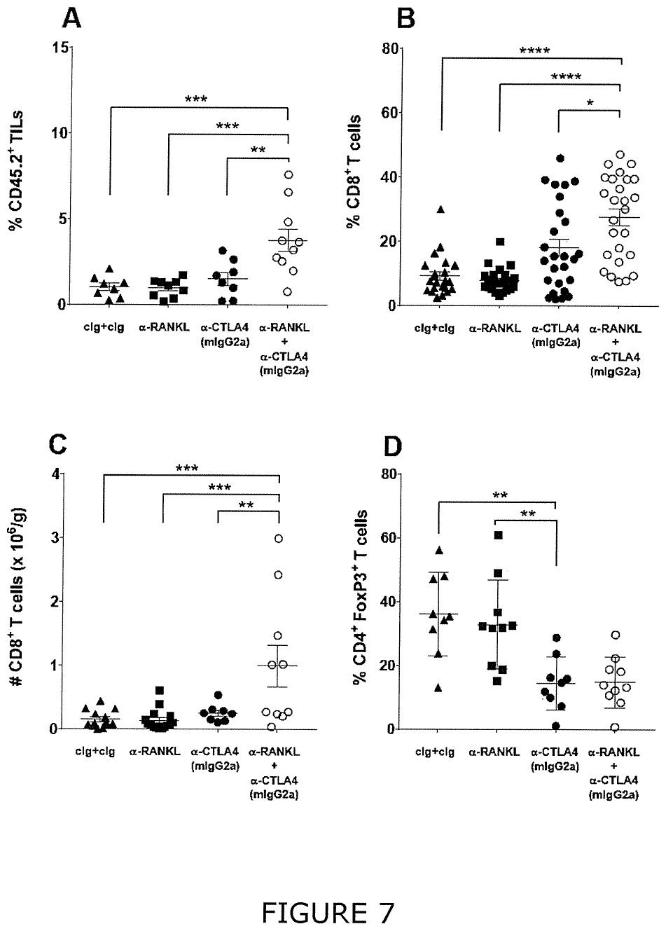

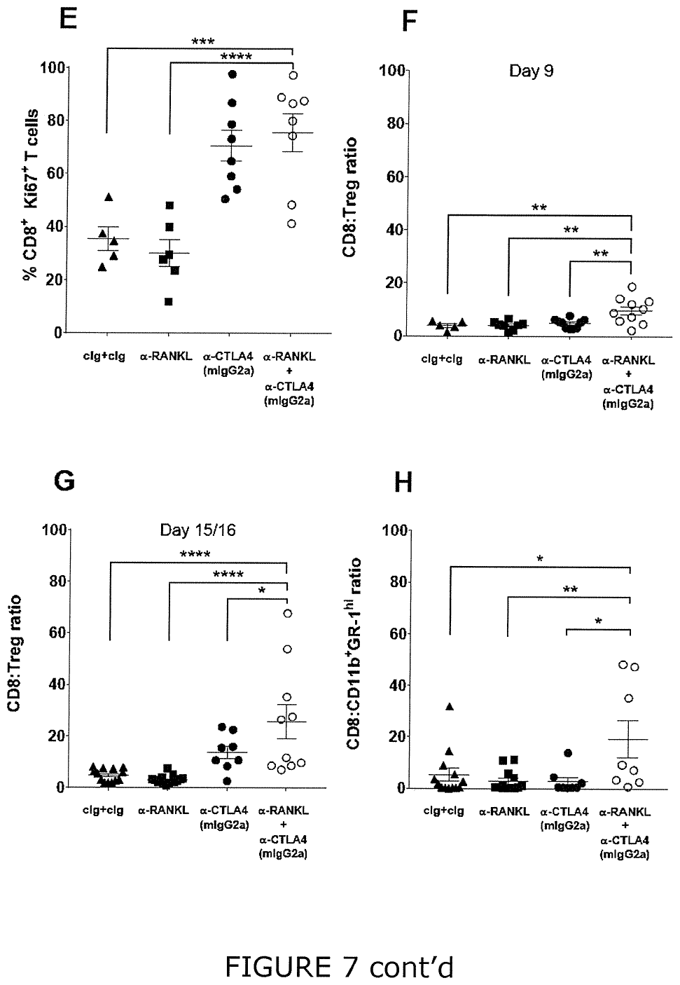

To further understand the mechanism of the combination therapy and the role of CD8⁺ T cells, the composition of TILs was assessed in subcutaneous B16F10 tumors that had been treated with the optimal combination therapy of anti-CTLA4 (IgG2a) and anti-RANKL (FIG. 7). When assessed at tumor end-point, the proportion of CD45⁺ TILs was significantly increased in the combination therapy compared to cIg or monotherapy treated groups (FIG. 7A). In contrast, this increase was not observed in mice treated with the combination therapy containing the anti-CTLA4-IgGl-D265A isotype (data not shown). The increase in CD45⁺ TILs in the combination therapy treated group was largely accounted for by a marked increase in CD8⁺ T cells, both in proportion (FIG. 7B) and absolute numbers (FIG. 7C). Again, these changes were not seen with the combination therapy containing the 9D9-IgG1-D265A isotype (data not shown).

The proportion of Tregs (CD45⁺ Foxp3⁺), as a percentage of CD4⁺ T cells in the tumor, was reduced with anti-CTLA4-IgG2a monotherapy (consistent with the reported mechanism of action of this isotype [Selby et al., 2013, Cancer Immunol. Res., 1(1):32-42]) but was not further reduced with the addition of anti-RANKL antibody (FIG. 7D). In addition the FcγR-IV expression on CD11b⁺ cells was not further increased with the combination therapy in the tumor (data not shown), suggesting that enhanced Treg depletion in the TME does not explain the mechanism of action of this combination. In the spleen, no significant changes in Treg proportion or number were detected between treatment groups (data not shown). Overall, the inventors concluded that the mechanism of action of the combination therapies in these models does not appear to be due to more efficient Treg depletion.

Another potential mechanism of action of anti-RANKL could be the enhancement of T cell proliferation. However, the inventors did not observe any further increase in Ki-67 expressing CD8⁺ T cells in the combination treated groups compared to anti-CTLA4 monotherapy (FIG. 7E). This suggests that the additional CD8⁺ T cells observed in the tumor post combination treatment might be a result of selective CD8⁺ T cell recruitment. Thus influx of CD8⁺ T cells with the combination therapy, combined with a lack of increase in suppressive immune cells such as Tregs or myeloid cells, may change the TME to favour anti-tumor activity. Indeed, significant increase in CD8⁺ -to-Treg ratio were noted when measured at an early time-point (day 9) (FIG. 7F) or at tumor end-point (FIG. 7G). In addition, the ratio of CD8⁺ T cells to MDSCs was also significantly increased with the combination therapy (FIG. 7H). Importantly these changes observed was specific for the tumor as no significant changes in the proportions of leukocyte subsets were observed in the spleen of these tumor-bearing mice (data not shown).

### Example 8

Anti-RANKL and Anti-CTLA4 Therapy Increases T-Cell Cytokine Production and Polyfunctionality

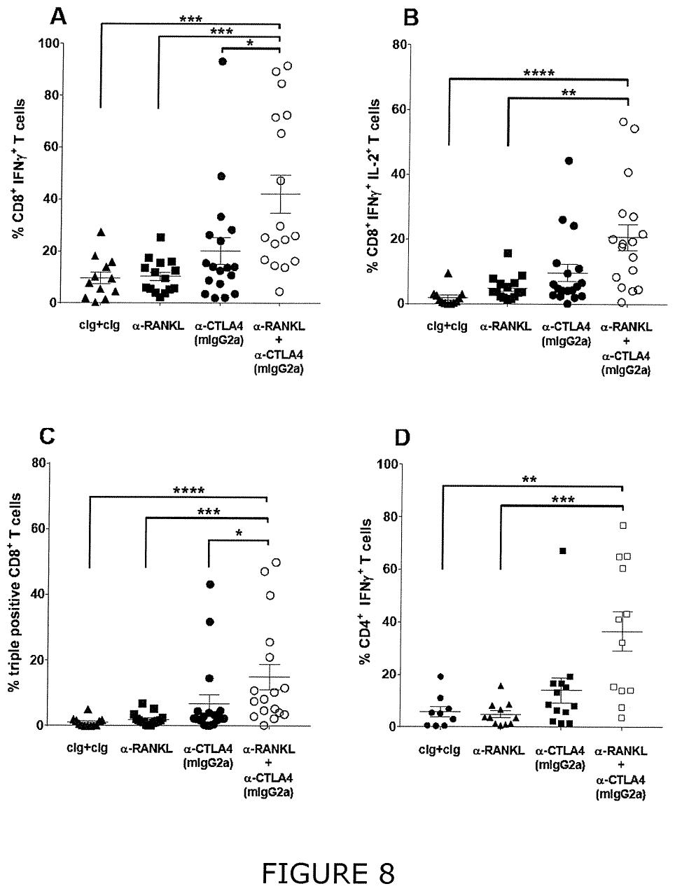

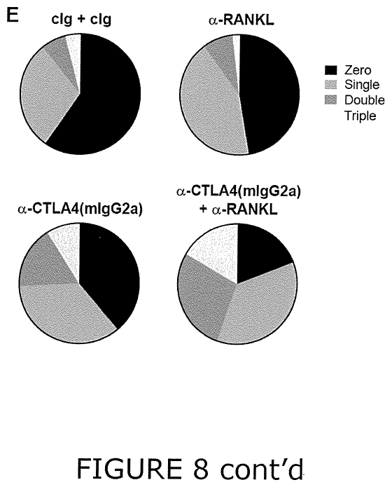

The inventors also assessed how this combination immunotherapy impacted on Thl cytokine production (IFNy, TNF, IL-2) from CD8 and CD4⁺ T cells in B16F10 tumor at experimental endpoint (day 16)(FIG. 8). TNFa was the most commonly produced cytokine ex vivo after stimulation, but significant differences were noted in the production of IFNy by CD8⁺ T cells (FIG. 8A) and IL-2 (data not shown) following combination therapy compared to cIg or monotherapy alone. Furthermore, CD8⁺ T cells co-expressing IFNy and IL-2 (FIG. 8B) or IFNy, IL-2 and TNFa (FIG. 8C) were also increased with the combination therapy. Similar findings were seen with CD4⁺ T cells, particularly in the proportion that produced IFNy (FIG. 8D). The majority of CD8⁺ T cell from the cIg treated group produced no cytokines after stimulation, while the combination therapy generated T cells with the most polyfunctionality, with monotherapy treatment groups displaying intermediate phenotypes (FIG. 8E). The effect of the combination therapy on cytokine polyfunctionality was tumor-specific, as these differences were not observed in the splenic T cells of tumor-bearing mice (data not shown).

### Materials and Methods for Examples 1-8

Cell culture

Mouse melanoma cell line B16F10 (ATCC) and LWT1 and prostate carcinoma cell line RM-1 were maintained, injected, and monitored as previously described (Ferrari de Andrade et al., 2014, Cancer Res, 74:7298-7308). Fibrosarcoma cell line MCA1956 (derived from MCA inoculated C57BL/6 wild-type mouse) was kindly provided by Robert Schreiber (Washington University School of Medicine, St Louis, MO, USA). Prostate cancer cell line Tramp-C1 was maintained as described (Dalezis et al., 2012, In Vivo, 26:75-86) but without dehydroisoandrosterone. All cell lines were routinely tested negative for Mycoplasma, but cell line authentication was not routinely performed.

Mice

C57BL/6 wild-type (WT) mice were bred-in-house or purchased from the Walter and Eliza Hall Institute for Medical Research. C57BL/6 perforin-deficient (pfp⁻/⁻), interferon-deficient (IFNγ^{-/-}), Fc receptor deficient (FcγRIII, FcγRIV and FcεγR), Batf3 transcription factor deficient (Batf3⁻'⁻)(as described in Hildner et al., 2008, Science, 322:1097-1100) and FoxP3-DTR (as described in Teng et al., 2010, Cancer Res, 70:7800-7809) mice were bred in-house at the QIMR Berghofer Medical Research Institute (QIMRB). All mice were used between the ages of 6 to 16 weeks. Groups of 5 to 13 mice per experiment were used for experimental tumor metastasis assays and subcutaneous (s.c.) tumor growth. All experiments were approved by the QIMRB Animal Ethics Committee.

Antibodies

Purified anti-mouse anti-RANKL (IK22/5; rat IgG2a, as described in Kamijo S. et al., 2006, Biochem Biophys Resh Commun, 347:124-132), anti-CTLA4 (UC10-4F10, hamster IgG) and control antibodies (hamster Ig, 1-1 or rat IgG2a, 2A3) were produced in house or purchased from BioXcell (West Lebanon, NH). Anti-CTLA4 clone 9D9 (various isotypes as indicated), and control antibody 1D12 (mouse IgG2a), were supplied by Bristol-Myers Squibb (San Francisco, CA). Antibodies to deplete NK cells (anti-asialoGM1, Wako) or anti-CD813 (53.5.8, BioXcell) were administered as indicated.

Subcutaneous Tumor Models

For B16F10 (1×10⁵), RM-1 (5×10⁴), MCA1956 (1×10⁶) or TRAMP-C1 (1 x 10⁶) tumor formation, cells were inoculated s.c. into the abdominal flank of female (B16F10, MCA1956) or male (RM-1, TRAMP-C1) mice. Therapeutic antibody treatment commenced as indicated on day 3-12 after tumor inoculation and was given every 2-4 days up to a maximum of 4 doses. Tumors were measured in two dimensions with digital callipers and tumor sizes are presented as mean±SEM.

Experimental Lung Metastasis Models

Single-cell suspensions of B16F10, RM-1 or LWT1 were injected i.v. into the tail vein of the indicated strains of mice. Lungs were harvested on day 14, and surface tumor nodules were counted under a dissection microscope. Antibody treatments were as indicated, with anti-CTLA4 and/or anti-RANKL MAbs administered on days -1, 0 and 2 relative to tumor inoculation. Antibodies to deplete CD8⁺ T cells or NK cells were administered where indicated on days −1, 0 and 7 relative to tumor inoculation.

Flow Cytometry

Tumor-bearing mice were sacrificed at two time points: day 9, or at end-point (when the experiment was terminated due to tumors reaching ethical endpoint size). Tumor, draining lymph node (inguinal) and spleen were collected and wet weight was recorded. Single-cell suspensions were generated from indicated organs as previously described (Teng et al., 2010, supra).

The following antibodies (from Biolegend, eBioscience, BD) were used: CD4-BV605 (RM4-5), CD8-BV711 (53-6.7), CD11b-BV650 (M1/70), CD11b-PE (M1/70), CD11c-PE (N418), purified CD16.2 (9E9) followed by goat-anti-hamster FITC, CD206-AF647 and CD206-PECγ7 (C068C2), and Zombie Aqua live/dead dye; TCR13-PerCP-Cγ5.5 (H57-597), CD45.2-A780 (104), Lγ6C/Lγ6G (GR-1)-EF450 (RB6-8C5), MHCII-APC (M5/114.15.2), CD265 (RANK)-PE (R12-31); RANKL-AF647 (IK22/5). For intracellular cytokine staining (ICS), cells were stimulated for 4 hours with Cell Stimulation Cocktail (1/1000) (eBioscience). Cells were then surface stained as described above before being fixed/permeabilized with Cytofix/Cytoperm (BD) and stained with IFNγ-AF488 (Biolegend), TNFα-PE (BD), and IL-2-Pacific Blue (Biolegend).

For intracellular transcription factor staining, cells were surface stained as described above before being fixed and permeabilized using the Foxp3/Transcription Factor Staining Buffer Set (eBioscience), according to the manufacturer's protocol and stained with FoxP3-EF450 or FoxP3-AF488 (FJK-16s) and Ki67-EF450 (So1185) (eBioscience). All immune cell analysis was first gated on live, single CD45.2⁺. T cells were defined as TCRβ⁺NK1.1⁻. NK cells were defined as TCRβ⁻NK1⁺. DCs were defined as CD11c⁺MHCII^{high}cells. Tumor-associated macrophages (TAM) were defined as CD11b⁺F4/80⁺ non-DC cells. MDSCs were defined as CD11b⁺, Lγ6C/Lγ6G (GR-1)^{hi}, non-TAM, non-DC cells. To determine absolute counts in samples, liquid-counting beads (BD Biosciences) were added immediately before samples were ran on a flow cytometer. All data were collected on a Fortessa 4 (BD) flow cytometer and analyzed with FlowJo v10 software (Tree Star, Inc.).

Statistical analysis

GraphPad Prism software was used for statistical analysis. For column analyses, Brown-Forsythe test was used to assess equal variances. If non-significant, one-way ANOVA with Dunn's multiple comparisons was used. In the event of unequal variances between groups, Kruskal-Wallace analysis with Sidak's or Dunnett's multiple comparisons were employed as appropriate. For longitudinal tumor growth analysis, treatment group random effects models were employed, for within-experiment mice only. Data were considered to be statistically significant where the P value was less than 0.05.

### Example 9

### Suppression of Lung Metastases by Co-Blockade of RANKL and PD-1-PD-L1 Interactions

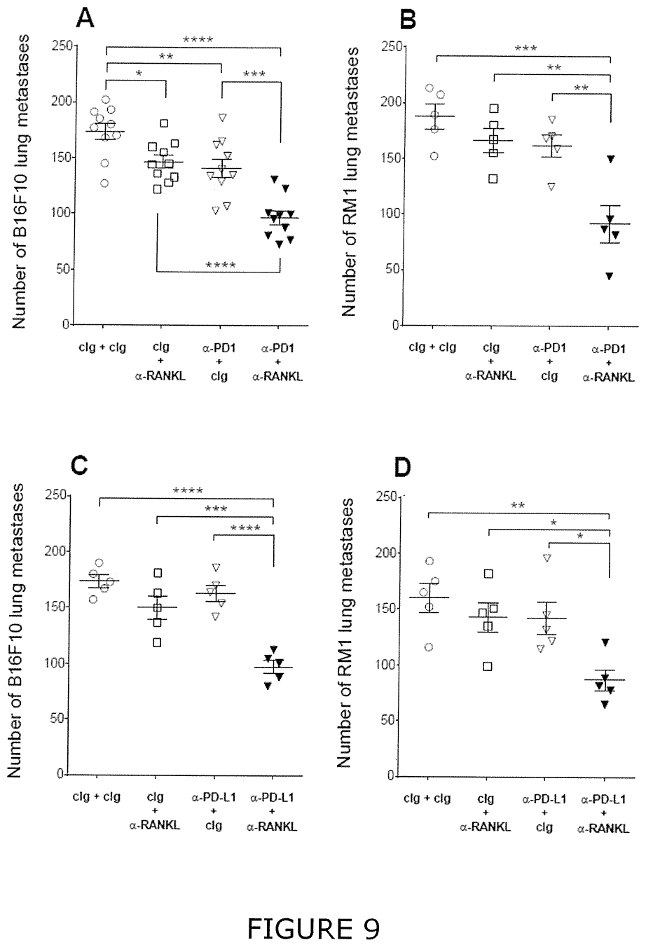

The combination of anti-RANKL and anti-PD-1 (FIG. 9A-B) or anti-RANKL and anti-PD-L1 (FIG. 9C-D) results in superior resistance to metastasis in lung metastasis models of melanoma (B16F10) or prostate cancer (RM1).

### Example 10

### Suppression of Subcutaneous Tumor Growth by Co-blockade of RANKL And Pd-1

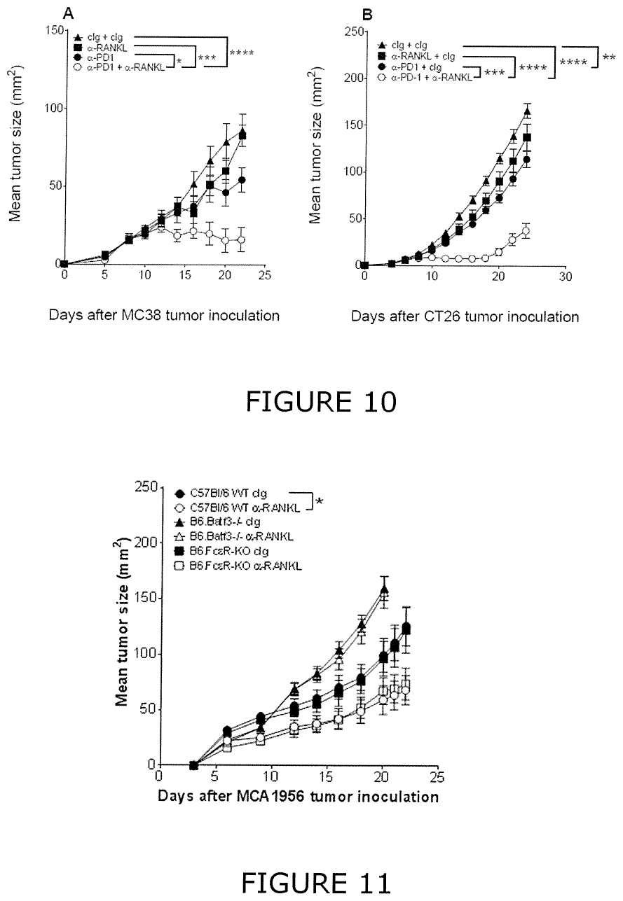

To extend these results from experimental metastasis models, the efficacy of dual blockade of RANKL and PD-1 in mice bearing subcutaneous tumors was next assessed. In the PD-1-sensitive cell line MC38 and the PD-1-intermediate-response cell line CT26 (both colon cancer models), the addition of anti-RANKL enhanced anti-PD-1 efficacy (FIG. 10A-B). Combinatorial efficacy against CT26 was maintained when the therapy was commenced at a later time point against more established tumors (data not shown).

### Example 11

### The Ability of Anti-RANKL to Suppress Subcutaneous Tumor Growth is Dependent on Batf3, but is not Dependent on FC Receptor Expression

The efficacy of some immunomodulatory antibodies includes the depletion of antigen-expressing cells by antibody dependent cytotoxicity (Dahan et al., 2015, Cancer Cell, 28(3):285-295) or, alternatively, the agonistic activity of targeted antigens (Dahan et al., 2016, Cancer Cell, 29(6):820-831). Both of these processes require engagement of Fc receptors within the tumor microenvironment. Additionally, the anti-tumor efficacy of certain antibodies (e.g., anti-CD137, anti-PD-L1) requires the presence of CD103⁺ Batf3-dependent dendritic cells (Sanchez-Paulete et al., 2016, Cancer Discov. 6(1):71-9). Therefore, to understand the mechanism of action of anti-RANKL, the reliance of anti-RANKL efficacy on Fc receptors or BatF3-dpendent dendritic cells was tested in gene-targeted mice. Groups of C57BI/6 or gene-targeted mice were injected subcutaneously with MCA1956 fibrosarcoma cells (1×10⁶). Mice were treated on days 3, 7, 11 and 15 relative to tumor inoculation with anti-RANKL (IK22/5, 200 μg i.p.) or cIg (1-1, 200 μg i.p.). The anti-RANKL MAb IK22/5 demonstrated efficacy as a monotherapy in the MCA1956 subcutaneous tumor (FIG. 11). The anti-tumor efficacy of anti-RANKL was preserved in mice lacking FcERy. This is consistent with a mechanism of action of anti-RANKL which occurs via blockade of RANKL to its receptor, RANK, and does not act via the depletion of RANKL-expressing cells. In contrast, the anti-tumor efficacy of anti-RANKL IK22/5 was abrogated in mice lacking BatF3, suggesting that an essential role for CD103⁺ DC-mediated cross-presentation. These data are consistent with a mechanism of action whereby anti-RANKL disrupts an immunosuppressive or tolerogenic axis in the tumor microenvironment between RANK-expressing myeloid cells (e.g., dendritic cells, MDSC or macrophages) and RANKL-expressing cells, such as lymphocytes, lymph node cells or other stromal components.

### Example 12

### CO-Expression of Rank And Pd-L1 In Infiltrating Myeloid Cells From Tumors

Given the mechanistic data of anti-RANKL in MCA1956 tumor described above, the potential role of RANKL in the tumor microenvironment is via action on BatF3-dependent dendritic cells which may express the RANKL receptor, RANK. For a bi-specific antibody blocking two immunosuppressive pathways, co-expression of the target antigens on the same cell type would be favoured as functionality would be target cell intrinsic. Co-expression of target antigens on a single cell type may also proscribe a more cell- or tissue-specific action of the bispecific modality within the tumor microenvironment and less peripheral toxicity, by virtue of greater cell selectivity within the tumor. Alternatively, or in addition, a bi-specific antibody blocking two immunosuppressive pathways expressed on two distinct cells in trans may also be advantageous, as two distinct immunosuppressive mechanisms may be inhibited simultaneously.

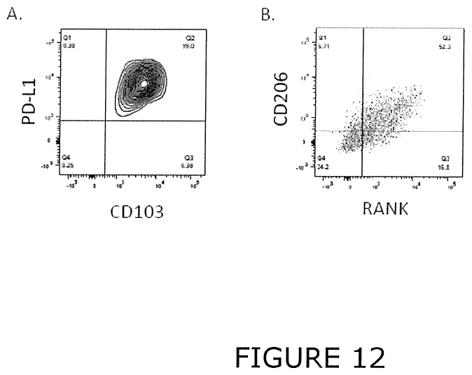

Therefore as a rationale for bi-specific targeting of RANK and PD-L1 or other antigens on the myeloid compartment, the expression of these factors were analysed on tumor-infiltrating myeloid cells by flow cytometry. MCA1956 cells (1×10⁶ cells /mouse) were injected subcutaneously into WT C57BL/6 mice. Tumors were allowed to grow for 22 days without any treatment until they reached approximately 50mm³. Tumors were collected and single-cell suspensions were generated and flow cytometry was done as described above. Flow cytometry analysis of CD11c+/MHCII+dendritic cells (DC) indicated that 100% of RANK-positive DC also expressed PD-L1 and CD103 (FIG. 12A). A similar analysis was performed in tumor-infiltrating macrophages (gated on CD11b+, F4/80+) isolated from MCA1956 subcutaneous tumors. This analysis indicated that 52% of tumor infiltrating CD11b+/F480+cells co-expressed RANK and CD206, while only 7% of RANK-negative CD11b+/F480+expressed CD206 (FIG. 12B).

To summarize, the anti-tumor efficacy of anti-RANKL IK22/5 MAb was abrogated in mice lacking BatF3, suggesting that an essential role for CD103⁺ dendritic cell (DC)-mediated cross-presentation. These data are consistent with a mechanism of action whereby anti-RANKL disrupts an immunosuppressive or tolerogenic axis in the tumor microenvironment between RANK-expressing myeloid cells (e.g., dendritic cells or macrophages) and RANKL-expressing cells, such as lymphocytes, lymph node cells or other stromal components. Flow cytometry analysis of CD11c⁺/MHCII⁺DC from tumors indicated that 100% of RANK-positive DC also expressed PDL-1 and CD103. A similar analysis indicated a significant enrichment for CD206 expression on RANK-positive tumor-infiltrating macrophages. For a bi-specific antibody blocking two immunosuppressive pathways, co-expression of the target antigens on the same cell type would be favored as functionality would be target cell intrinsic. Co-expression of target antigens on a single cell type may also proscribe a more cell- or tissue-specific action of the bispecific modality within the tumor microenvironment and less peripheral toxicity, by virtue of greater cell selectivity within the tumor. Alternatively, or in addition, a bi-specific antibody blocking two immunosuppressive pathways expressed on two distinct cells in trans may also be advantageous, as two distinct immunosuppressive mechanisms may be inhibited simultaneously.

Altogether, these observations demonstrate a remarkable enrichment for PD-L1 expression on RANK-positive DC in the tumor and provide an additional rationale for bispecific targeting of these two antigens in cis. Modalities that target PD-L1 are well validated as checkpoint inhibitor therapies in oncology and provide a rational partner for an anti-RANK bispecific. Additionally, the high degree of co-expression of CD103 and CD206 with RANK suggests additional antigen partners for a bispecific modality targeting RANK.

### Example 13

### Anti-RANKL and Anti-PD-1 Diabodies

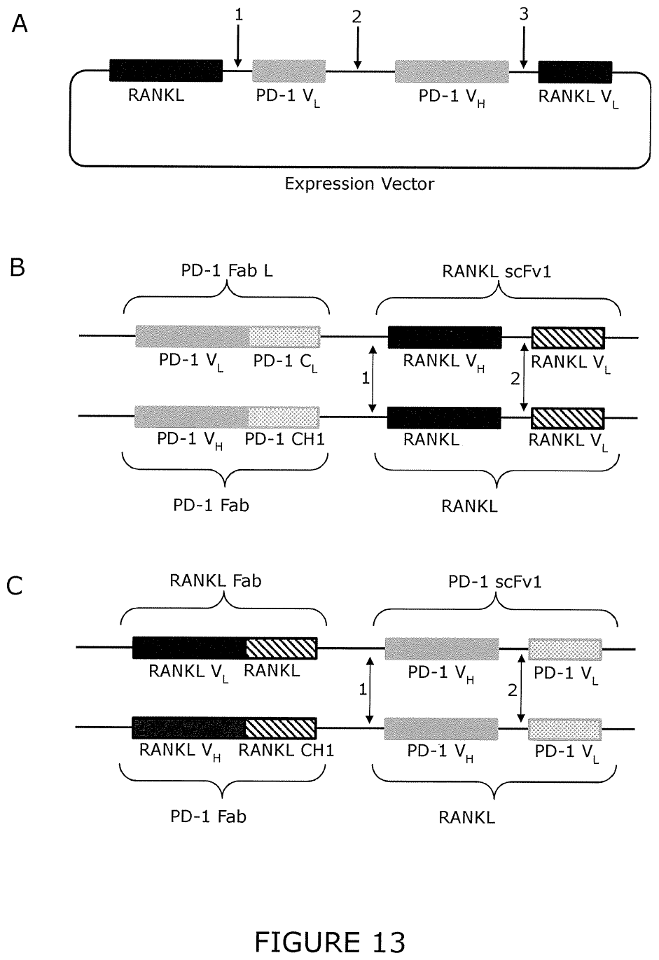

DNA encoding bispecific single chain diabodies are constructed as follows and as illustrated in FIG. 13A. Specifically, the variable heavy chain of an anti-RANKL antibody (e.g., denosumab) is linked via a 5-amino acid linker to the variable light chain of an anti-PD-1 antibody, which, in turn, is linked via a 15-amino acid linker to the variable heavy chain of the anti-PD-1 antibody, which is linked via another 5-amino acid linker to the variable light chain of the anti-RANKL antibody.

In an exemplary construct of a bispecific single chain diabody, the variable heavy chain of a RANKL antibody (denosumab V_{H} having the amino acid sequence: EVQLLESGGGLVQPGGSLRLSCAASGFTFSSYAMSWVRQAPGKGLEWVSGITGSGGSTYYADSVKGRFTISRD NSKNTLYLQMNSLRAEDTAVYYCAKDPGTTVIMSWFDPWGQGTLVTVSS [SEQ ID NO:3]) is linked via a first linker (5G₄), to the variable light chain of an anti-PD-1 antibody (nivolumab V_{L} having the amino acid sequence: EIVLTQSPATLSLSPGERATLSCRASQSVSSYLAWYQQKPGQAPRLLIYDASNRATGIPARFSGSGSGTDFTLTIS SLEPEDFAVYYCQQSSNWPRTFGQGTKVEIK [SEQ ID NO:91]), which in turn is linked via a second linker, (5G₄)₃, to the variable heavy chain of the anti-PD-1 antibody (nivolumab V_{H} having the sequence: QVQLVESGGGVVQPGRSLRLDCKASGITFSNSGMHWVRQAPGKGLEWVAVIWYDGSKRYYADSVKGRFTISR DNSKNTLFLQMNSLRAEDTAVYYCATNDDYWGQGTLVTVSS [SEQ ID NO:88]), followed by a third linker (5G₄) and the variable light chain of anti-RANKL antibody (denosumab V_{L} having the amino acid sequence:

[SEQ ID NO: 4]
EIVLTQSPGTLSLSPGERATLSCRASQSVRGRYLAWYQQKPGQAPRLLIY
GASSRATGIPDRFSGSGSGTDFTLTISRLEPEDFAVFYCQQYGSSPRTFG
QGTKVEIK).

DNA encoding the bispecific diabody is cloned into an expression vector (for example, pSecTag2/HygroA, Invitrogen). The resulting plasmid encoding the bispecific antibody is then amplified, extracted, and purified using standard protocols.

The expression plasmid is transiently transfected into human kidney cell line 293T with LipfectAMINE-plus (Invitrogen) and cultured. The supernatant is sterilized with a 0.22 pm PVDF filter, and concentrated using 40% PEG20,000 solution. The concentrated supernatant is purified using a HiTrap Chelating HP column (GE Healthcare).

### Example 14

### Anti-RANKL and Anti-PD-1 Tribodies

A pair of plasmids is required for the production of the bispecific tribodies as shown in FIG. 13B. Specifically, a first construct is prepared that encodes a single chain polypeptide that comprises the variable light chain of an anti-PD-1 antibody fused to the constant region of a human κ light chain, which is linked via an amino acid linker to a variable heavy chain of an anti-RANKL antibody. For ease of purification, a tag (e.g., his-tag (His6)) may be added to the C-terminus of the single chain polypeptide. A second construct is also prepared encoding a single chain polypeptide that comprises the variable heavy chain of an anti-PD-1 antibody fused to constant region 1 of a human IgG2, which is linked via a first amino acid linker to a variable heavy chain of an anti-RANKL antibody, which in turn is linked via a second amino acid linker to the variable light chain of an anti-RANKL antibody. For ease of purification, a tag (e.g., his-tag (His6)) may also be added to the C-terminus of the single chain polypeptide.

The two constructs are cloned into two separate expression vectors, which are typically in the form of plasmids e.g., pCAGGS (De Sutter et al., 1992, Gene 113, 223-230). The resulting plasmid pair encoding the bispecific tribody, pCAGGS-FabL-scFv-His6 and pCAGGS-FabFd-scFv-His6, are then amplified, extracted, and purified.

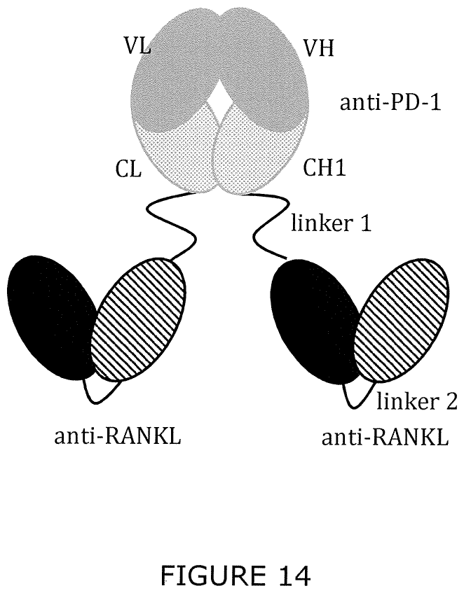

An alternative plasmid pair is shown in FIG. 13C. In this embodiment, a first construct is prepared that encodes a single chain polypeptide comprising the variable light chain of an anti-RANKL antibody fused to the constant region of a human κ light chain, which is linked via an amino acid linker to a variable heavy chain of an anti-PD-1 antibody. For ease of purification, a tag (e.g., his-tag (His6)) may be added to the C-terminus of the single chain polypeptide. A second construct is prepared encoding a single chain polypeptide that comprises the variable heavy chain of an anti-RANKL antibody fused to constant region 1 of a human IgG2, which is linked via a first amino acid linker to a variable heavy chain of an anti-PD-1 antibody, which in turn is linked via a second amino acid linker to the variable light chain of an anti-PD-1 antibody. An exemplary tribody of this type is depicted in FIG. 14.

### Example 15

### Assays for Determining Antagonist Activity

The RANKL and RANK ligand-receptor pairs are cross-reactive between human and mouse proteins, with equivalent binding and functional activity detected (Bossen et al., 2006, J Biol Chem., 281(20):13964-71). There are a variety of recombinant forms of RANKL and RANK available commercially for antigen and assay preparation. Both RANKL and anti-RANK antibodies will selectively bind CRD2 and CRD3 of RANK or full-length RANK (i.e., comprising CRDs1, 2, 3, 4).

TNF superfamily ligand/receptor interactions can be assessed by ELISA (see, for example, Schneider et al., 2014, Methods Enzymol., 545:103-25; Kostenuik et al., 2009, J. Bone Miner Res., 24(2):182-95) providing a straightforward screen for RANKL or RANK antagonists. Biological activity of RANKL and RANKL inhibitors can be monitored using osteoclastogenesis (and generation of TRAP5b in conditioned media by ELISA) in cultures of murine RAW 264.7 macrophages that served as osteoclast precursors, as previously described (Kostenuik et al., 2009, supra; Xu et al., 2000, J. Bone Miner Res., 15(11):2178-86). These assays are amenable to medium-throughput (e.g., 384-well) screening assays.

Cross-reactivity of anti-RANK antibodies can be screened against other related members of TNFR superfamily. Flow-cytometry or ELISA based screens can be used in this regard.

In determining antagonist activity for RANKL or RANK antagonists it is preferable to eliminate any RANKL or RANK antagonists which may also have agonistic activity on the RANK receptor. In vitro screens for agonistic activity of anti-RANK antibodies can be performed using bivalent or monovalent antibody forms in the RANK-Fas Jurkat assay, as described for example by Schneider et al. (2014, supra) and Chypre et al. (2016, Immunol. Lett., 171:5-14). Analysis of antibodies against the related TNFR member, EDAR, indicated that the correlation with agnostic activity was not the affinities of antibodies but their ability to detach slowly once bound (small koff) (Kowalczyk-Quintas et al., 2011, 3. Biol. Chem., 286(35):30769-79). Similarly, analysis of antibodies against the TNFR member FAS has demonstrated an inverse correlation between receptor affinity and (agonist) potency (Chodorge et al., 2012, Cell Death Differ., 19(7):1187-95). Of note, phage screens have identified scFvs with agonistic activity against other TNFR members (e.g., TRAILR) (Dobson et al., 2009, MAbs, 1(6):552-62).

One can also test anti-RANK binders in the context of a second target (e.g., PDL-1) in a bi-specific format, to verify that binding and functional RANKL antagonism blockade are retained. For example, a cell line may be engineered to express RANK (e.g., as a RANK-Fas chimera [Schneider et al., 2014, supra]) along with human PD-L1 and RANK antagonist activity could be confirmed. Alternatively, the expression of PD-L1 has been demonstrated on RANK-positive osteoclast precursors (An et al., 2016, Blood, 128(12):1590-603) and an in vitro functional assay can be developed to address functionality of RANK- and PD-L1 binders. Osteoclast formation would address RANK blockade, while PD-1 binding or T-cell suppression could be used to address PD-L1 blockade. In support of the latter, An et al. 2016, supra) demonstrated that anti-PD-L1 increases CTL activity from osteoclast progenitor cultures.

In vivo activity of anti-RANK or anti-RANKL antibodies (RANKL antagonists) can be performed using bivalent or monovalent antibody forms to address osteoclast antagonistic function in mouse studies. Analysis of bone density (using X-ray or DEXA) in mice challenged with anti-RANK or anti-RANKL antibodies can also be performed. Alternatively, the effects of anti-RANK or anti-RANKL antibodies on hypercalcemia in normal mice challenged with subcutaneous injections of human RANKL may be assessed using analysis of blood ionized calcium or serum TRAP5b monitored daily for 4 days. Anti-RANK or anti-RANKL antibodies (RANKL antagonists) could also be tested in the MCA1956 tumor model (as shown in FIG. 9 herein for the positive control anti-RANKL MAb) for tumor response. Positive controls for RANKL stimulation (e.g., recombinant forms of RANKL) and inhibition (e.g., recombinant OPG-Fc) are readily available for in vitro and in vivo studies (Lacey et al., 2012, Nat Rev Drug Discov., 11(5):401-19).

### Example 16

### Anti-Tumor Efficacy of Anti-RANKL Mab does not Require T-Regulatory Cells (Tregs)

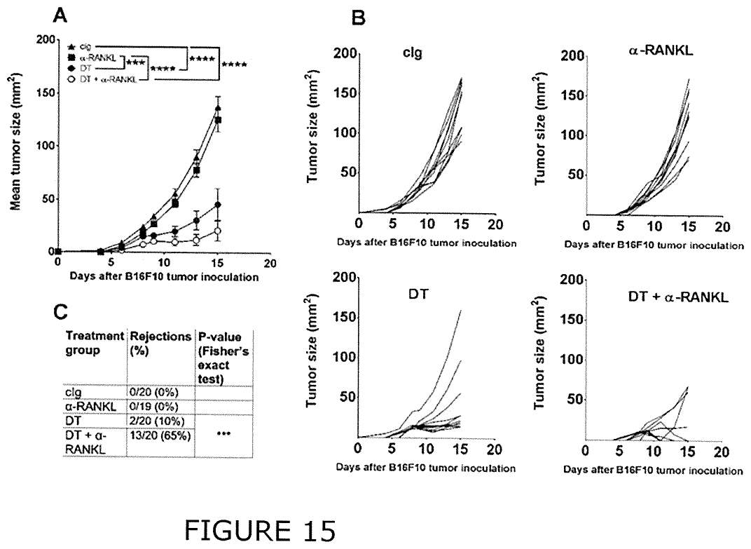

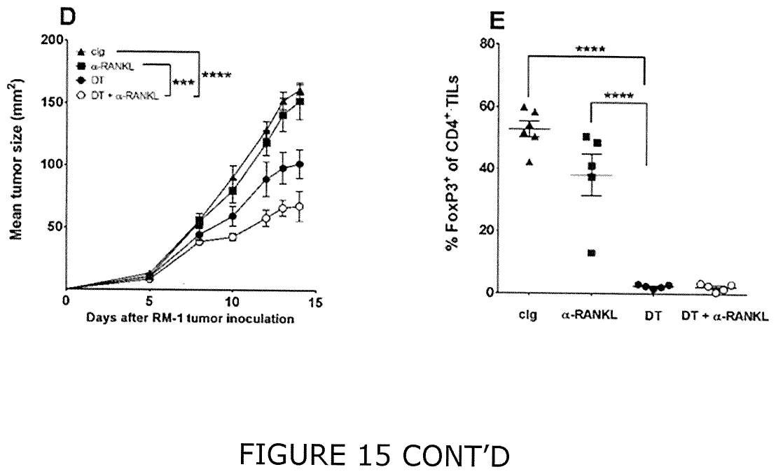

Previous publications have reported a role for RANKL-expressing Tregs in the promotion of metastasis in a mouse model of RANK-expressing mammary carcinoma (Tan et al., Nature 470 (2011), 548-553), or the role of systemic Treg control via cutaneous RANKL-RANK interactions in the restraint of UV-induced cutaneous inflammation (Loser et al., Nat. Med 12(2006), 1372-1379). In order to further assess any essential role for Tregs in combinatorial efficacy of anti-RANKL in immunotherapy, the FoxP3-DTR mouse model was employed. In these mice, the diphtheria toxin receptor (DTR) is expressed under the control of the foxp3 locus, allowing for the conditional and near-complete depletion of Tregs through administration of diphtheria toxin (DT), resulting in enhanced anti-tumor immunity (Teng et al., 2010, supra). A trend to greater B16F10 melanoma subcutaneous tumor growth suppression was seen (FIG. 15A-C) and a higher proportion of mice were cured when anti-RANKL (IK22.5) therapy was given in combination with DT compared with DT alone (FIG. 15A-C). Additionally, a similar trend of enhanced growth suppression of subcutaneous RM-1 prostate carcinoma was also seen in FoxP3-DTR mice treated with DT and anti-RANKL (FIG. 15D). FACS analysis of RM-1 tumors at endpoint revealed near-complete Treg depletion by DT alone, with no additional depletion of Tregs noted when anti-RANKL was combined with DT (FIG. 15E). Taken together, the mechanism of action of the combination therapies in these models does not appear to be due to more efficient Treg depletion, and the efficacy of anti-RANKL mAb is intact in settings of near-complete intratumoral Treg depletion. Indeed, additional efficacy of combination anti-RANKL with DT-induced Treg depletion was seen in FoxP3-DTR mice compared with DT alone, where both DT-containing arms showed >95% Treg depletion compared with cIg at endpoint; thus, indicating that the mechanism of action of anti-RANKL was not directly on Tregs.

Example 17

### Distinct Co-Expression of RANKL And PD-1 Compared with Co-Expression of RANKL And CTLA4 in Tumor Infiltrating Lymphocytes

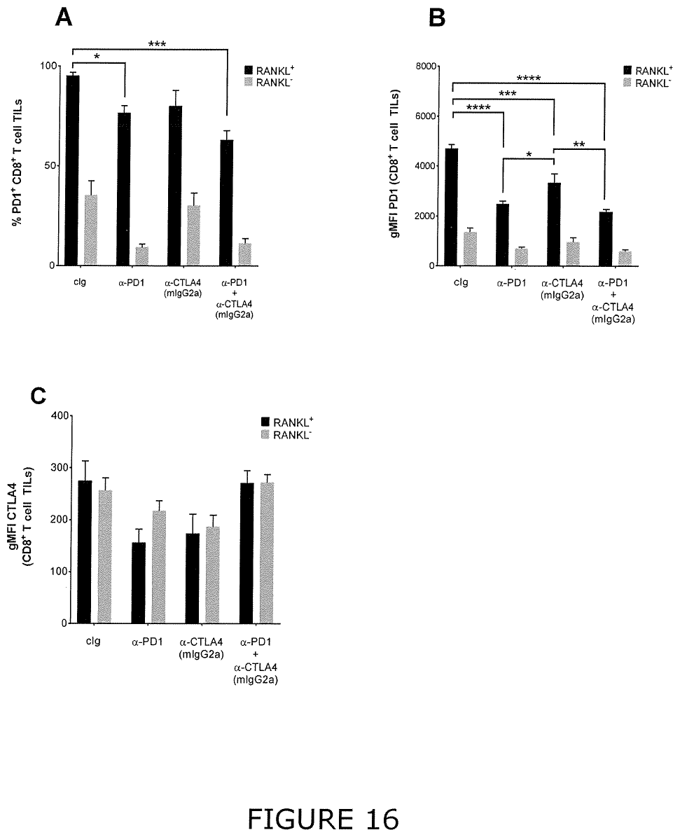

While preclinical results have demonstrated that anti-RANKL blockade increases the anti-tumor efficacy of either anti-CTLA4 mAb or blocking PD-1/PD-L1, there is evidence that this occurs through distinct mechanisms, consistent with the described non-overlapping mechanisms of action for CTLA4 vs. PD-1 blockade. For instance, in T-cells isolated from CT26 tumors in mice, almost all CD8⁺ RANKL⁺ T cell TILs (>90%) co-expressed PD-1; in comparison, less than 40% CD8+RANKL-T cell TILs were PD-1 positive (FIG. 16A). Furthermore, the MFI of PD-1 was at least 3 fold higher on CD8⁺ RANKL⁺ compared to CD8⁺ RANKL-T cells identifying the former as PD-1^{hi} cells (FIG. 16B). Expression analysis indicated CTLA4 expression was not significantly higher in RANKL⁺ CD8⁺ T cells compared with their RANKL-counterparts (FIG. 16C). Despite an enrichment for PD-1 co-expression, the characteristics of RANKL⁺ CD8⁺ T cell TILs in the CT26 model is more consistent with an activated rather than exhausted phenotype, given that RANKL-expressing cells generally were more proliferative and had low expression of another immune checkpoint, CTLA4.

Example 18

### Triple Combination Therapy of Anti-PD-1, Anti-CTLA4 and Anti-RANKL Antibodies Improves Anti-Tumor Response and T Cell Effector Function in Tumor-bearing Mice

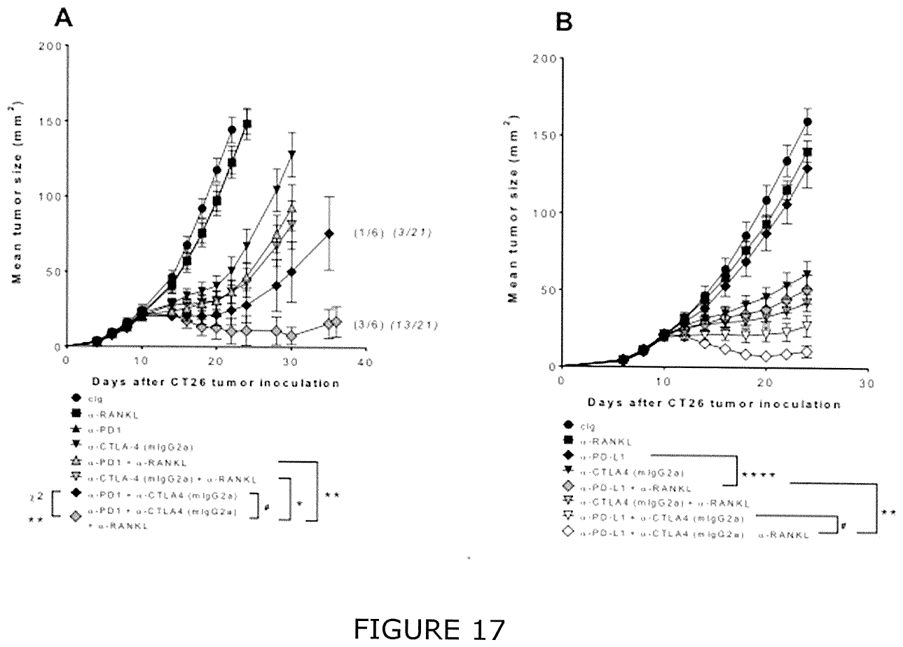

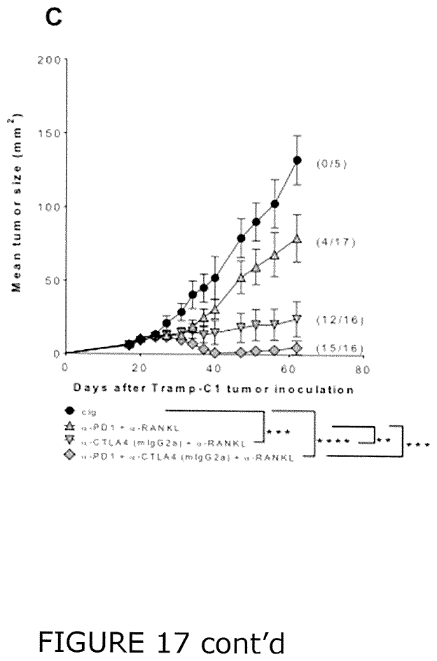

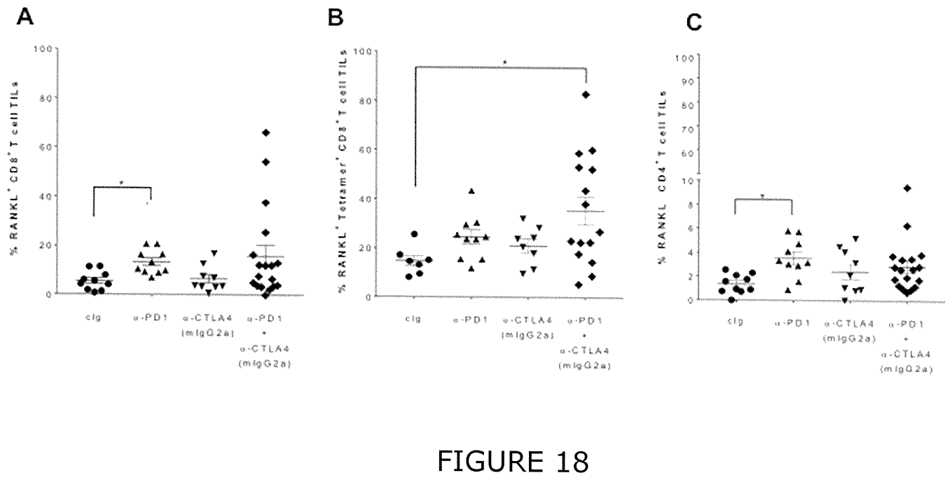

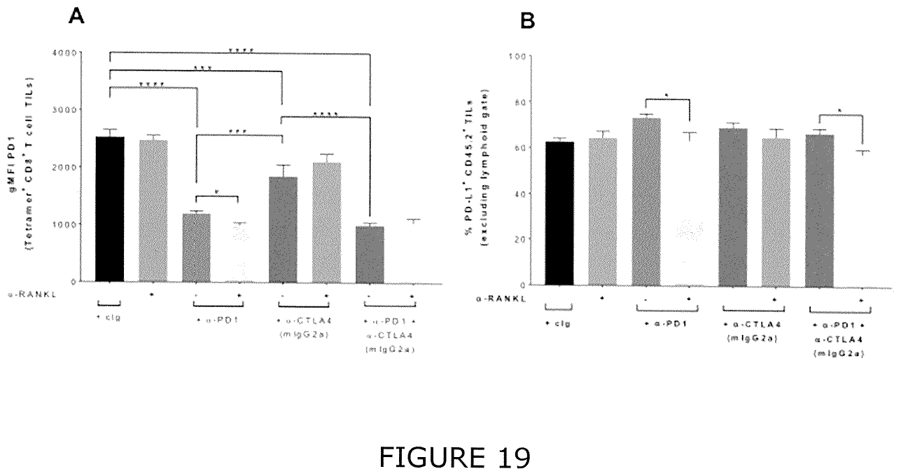

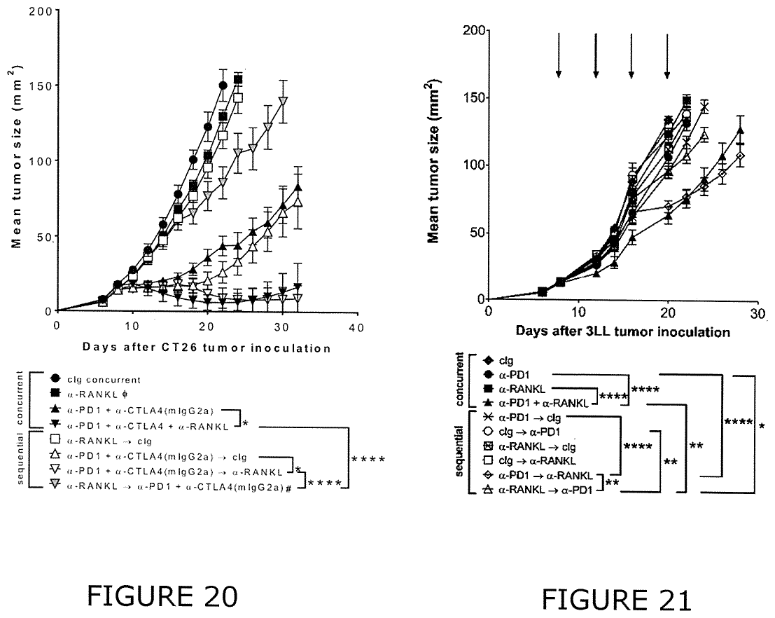

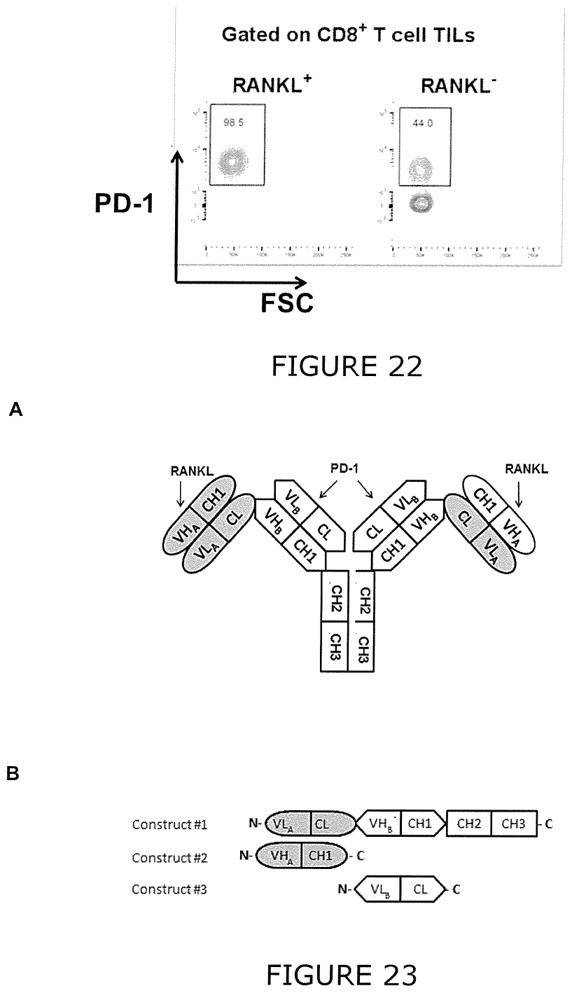

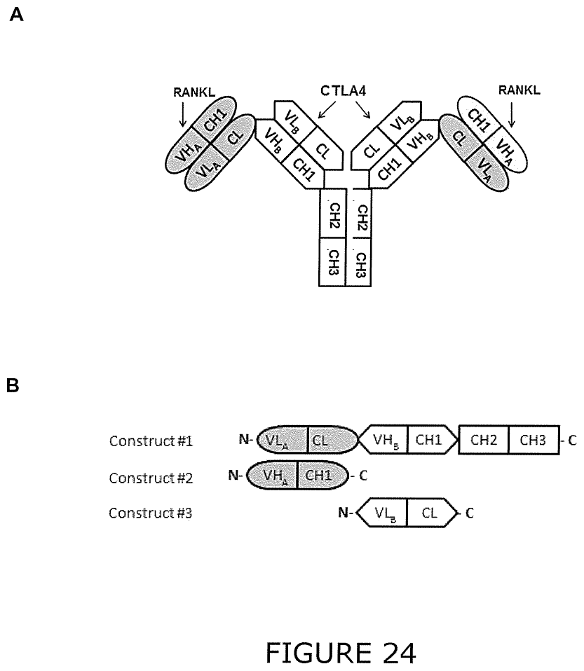

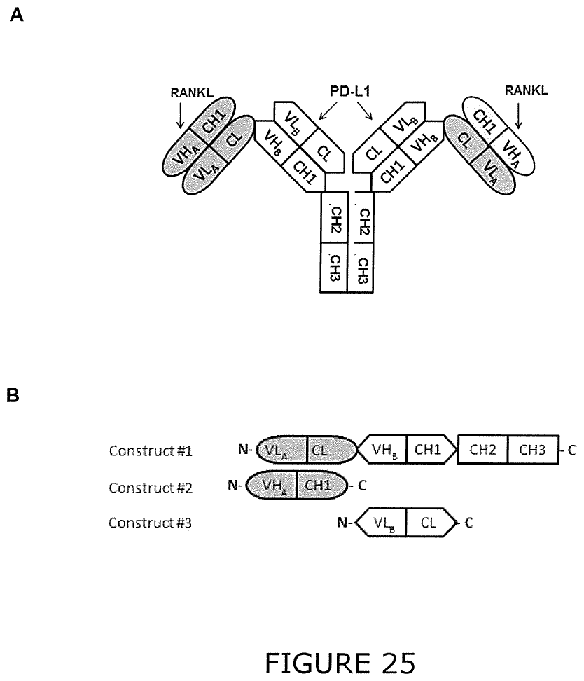

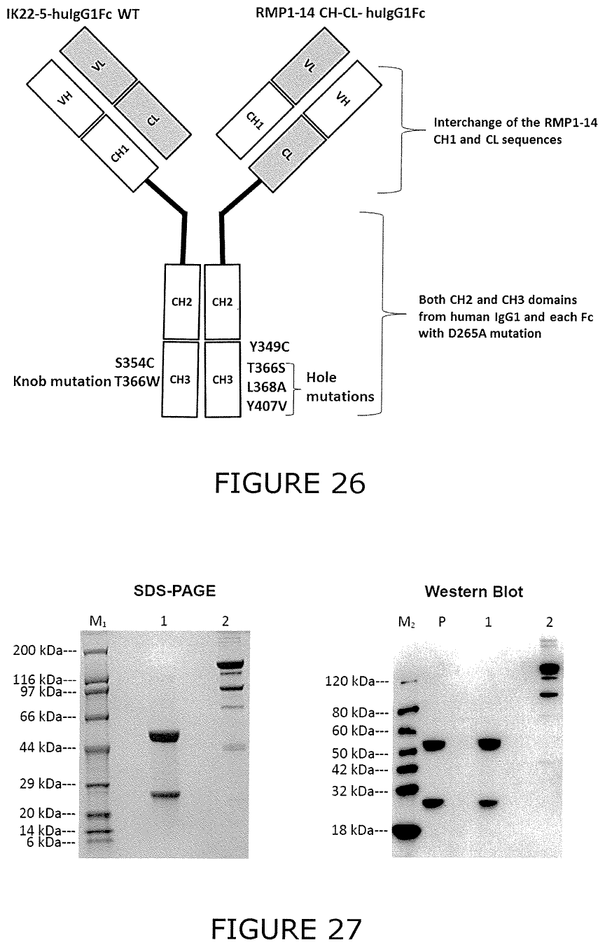

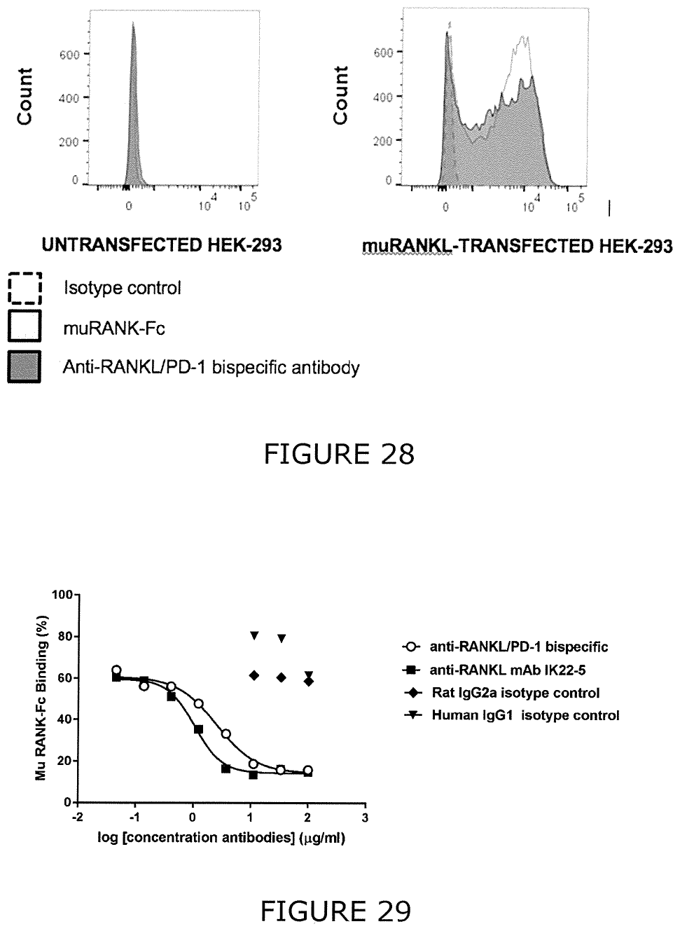

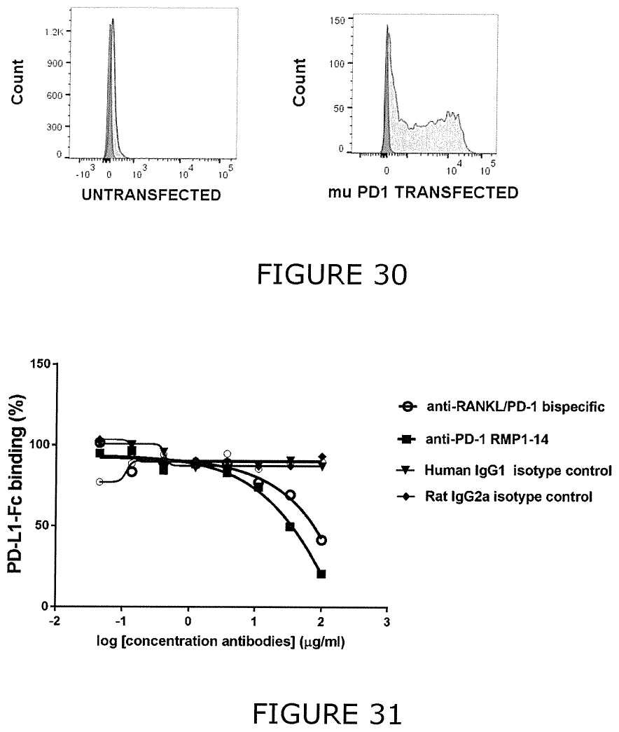

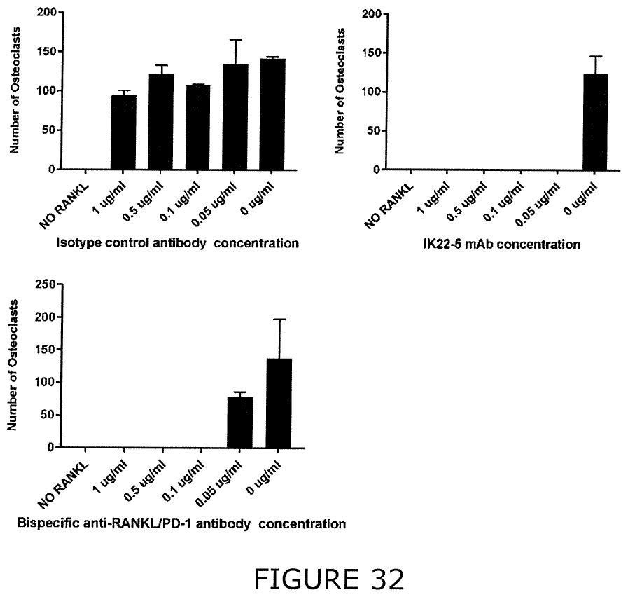

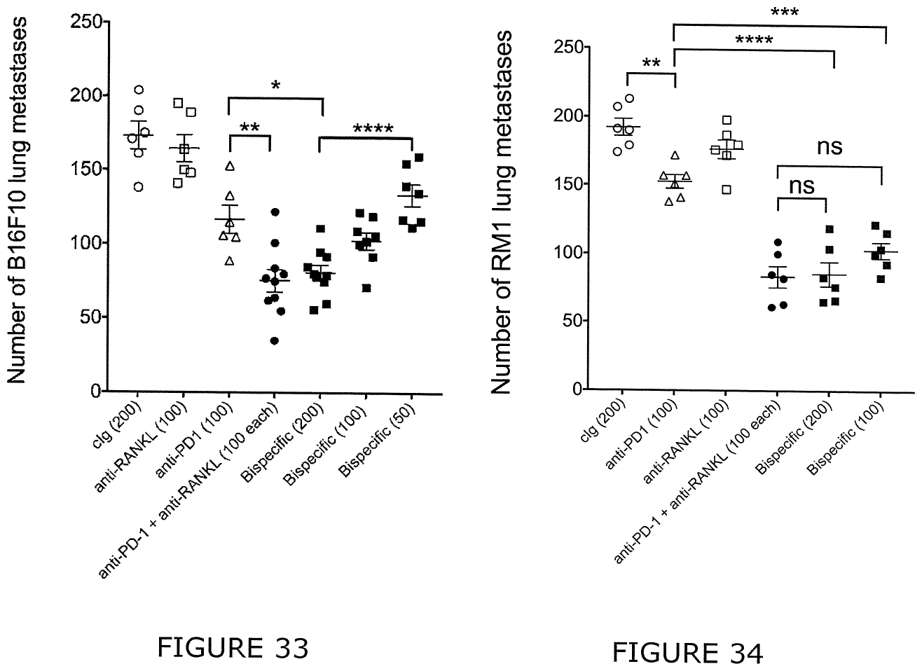

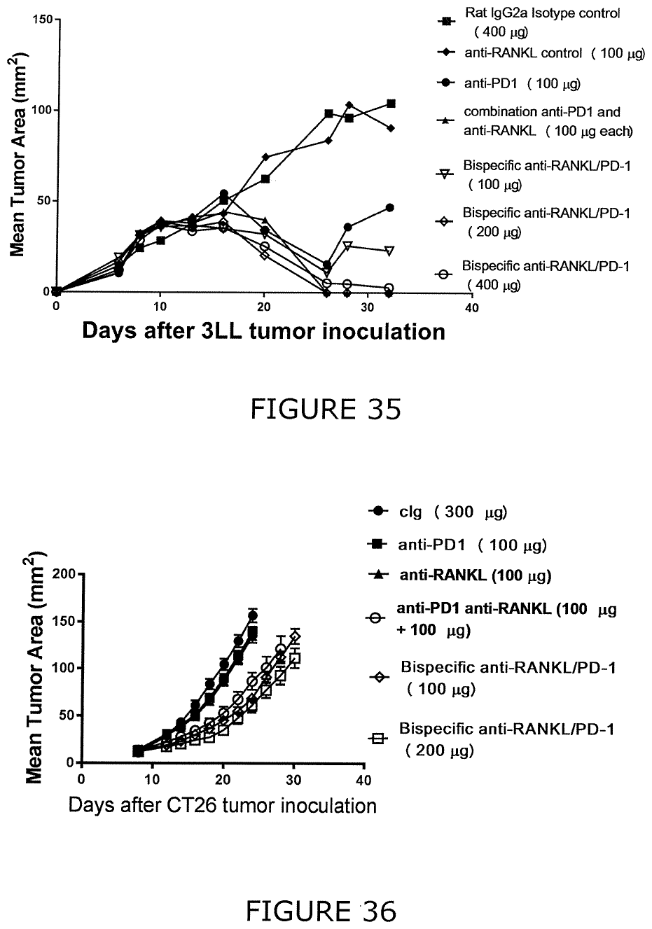

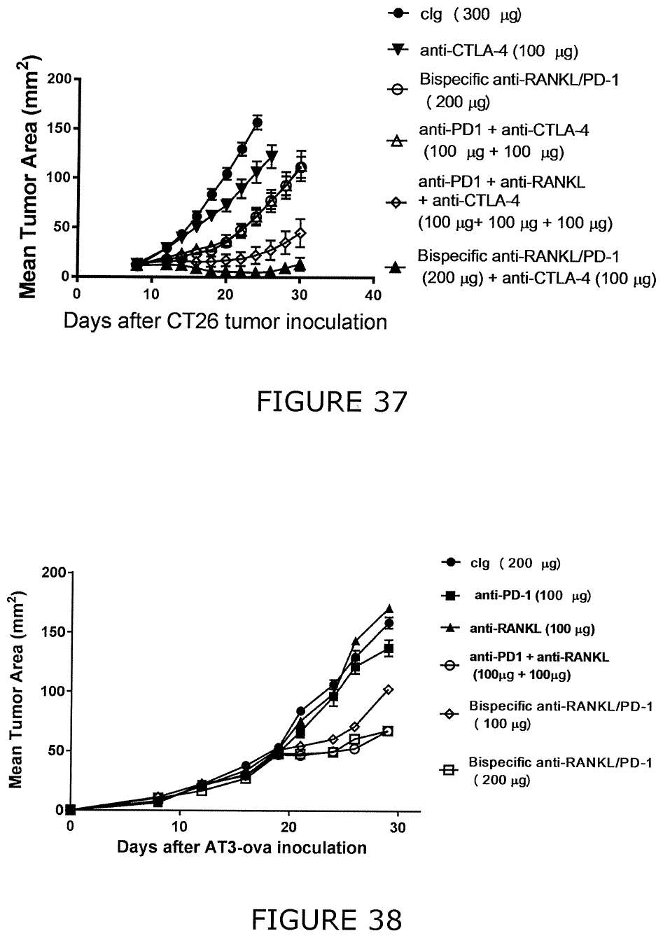

Given that combination immune checkpoint blockade (ICB) of PD-1 and CTLA4 is an emerging standard of care in certain clinical contexts such as advanced melanoma (Larkin et al. 2015. N Engl J Med;373:23-3), whether the addition of anti-RANKL could further improve the anti-tumor efficacy of anti-CTLA4 and anti-PD-¹/ₐnti-PD-L1 combination therapy was assessed (FIG. 17). Anti-RANKL was first assessed in combination using lower doses of anti-PD-1 (100 μg) in the suppression of WT mice bearing established CT26 tumors (FIG. 17A). The addition of anti-RANKL to anti-PD-1 significantly suppressed tumor growth, but triple combination therapy significantly suppressed growth of CT26 tumor-bearing mice compared to any dual combination therapy, and importantly, addition of anti-RANKL to combined anti-CTLA4 plus anti-PD-1 improved the tumor rejection rate (FIG. 17A). Next, the efficacy of anti-PD-L1 in combination with anti-RANKL with or without anti-CTLA4 in the suppression of CT26 s.c. tumor growth was assessed (FIG. 17B). Compared with anti-PD-L1 alone, which (similarly to anti-RANKL and anti-PD-1 monotherapies) has minimal efficacy, combination anti-PD-L1 and anti-RANKL significantly suppressed tumor growth (FIG. 17B). Additionally, triple combination of anti-PD-L1 and anti-RANKL with anti-CTLA4 was the most efficacious in suppression of CT26 s.c. growth; when this triple combination was specifically compared with dual ICB (anti-PD-L1 and anti-CTLA4), a small but significant difference was evident (FIG. 17B). Finally, the ability of triple combination therapy (anti-PD-1+anti-CTLA4+anti-RANKL) to control tumor growth was also assessed in the autochthonous TRAMP transgenic mice, bearing subcutaneous Tramp-C1 prostate carcinoma. In this setting where endogenous tumor-specific T cells may be tolerized, triple combination therapy was again most efficacious in controlling subcutaneous tumor growth with 15 out of 16 mice completely rejecting their tumors compared with select dual therapies and cIg (FIG. 17C). The increase in tumor control observed with triple combination therapy was correlated with a significant increase in Th1-type cytokine polyfunctionality in tumor-infiltrating CD8⁺ and CD4⁺ T cells as reflected in their co-expression of IFN-γ and TNFa compared to anti-CTLA4 plus anti-PD-1 dual combination therapy. This increase in TIL effector function was only observed in the tumor and not the spleen of triple combination therapy-treated mice.

### Example 19

### Unique Alterations in the TME Which may Distinctly Cross-modulate Anti-rankl and Anti-PD-1/PD-L1 Combination Therapy vs. Anti-RANKL And Anti-CTLA4 Combination Therapy

To address mechanisms by which anti-RANKL was improving immune checkpoint blockade (ICB) therapy in the CT26 model, the proportion of CD8⁺ T cells that expressed RANKL was assessed (FIG. 18A). In cIg-treated mice, approximately 5% expressed RANKL but this increased to over 10% following anti-PD-1 monotherapy. Furthermore, RANKL expression was enriched amongst the subset of gp70-reactive CD8+T cell TILs (almost 15% in cIg-treated mice), and was significantly increased in tumors which had received dual ICB as compared with anti-PD-1 monotherapy, anti-CTLA4 monotherapy or cIg (FIG. 18B). Although a lower proportion of CD4⁺ T cell TILs express RANKL compared with CD8⁺ T cell TILs, anti-PD-1 similarly increased RANKL expression (FIG. 18C). Through upregulation of the main intratumor source of RANKL expression, administration of anti-PD-1 possibly primed the TME to respond to RANKL blockade (FIG. 18A-C), while anti-CTLA4 monotherapy did not significantly alter RANKL levels in CD4⁺ or CD8⁺ T cell TILs. The observation that anti-PD-1 alone (or combined anti-PD-1+anti-CTLA4) can itself increase RANKL expression by tumor-infiltrating T cells, while the RANKL increase is not observed in TILs after anti-CTLA4 alone suggests that the anti-tumor efficacy achieved through combination of anti-RANKL plus anti-PD-1/PD-L1 occurs through a distinct mechanism compared with combination of anti-RANKL plus anti-CTLA4. It is unclear why anti-CTLA4 treatment alone did not also modify RANKL expression levels in this study. One explanation relies on the observation that RANKL is generally upregulated early after activation of T cells, particularly in a tolerogenic context (Hochweller et al., 2005. Eur J Immunol 35:1086-96).

Additional distinct changes to the TME observed after addition of anti-RANKL to anti-PD-1 vs. addition of anti-RANKL to anti-CTLA4 therapies were observed when the phenotype of infiltrating T-cells were examined. Previous reports indicated that tumor infiltrating PD-1^{hi} T cells were insensitive to anti-PD-1 therapy and displayed an exhausted phenotype. However, some immunotherapies such as anti-CD40 can lower the levels of PD-1 on PD-1^{hi} T cells to re-sensitize them to PD-1 blockade (Ngiow et al., 2015, Cancer Res 75:3800-11). Consistent with this, although anti-PD-1 monotherapy significantly attenuated PD-1 expression compared with cIg, administration of anti-PD-1 plus anti-RANKL further decreased PD-1 expression in gp70-specific CD8⁺ T cell TILs (FIG. 19A), as well as unselected CD8⁺ T cell TILs. Importantly, addition of anti-CTLA4 either alone or in combination to anti-RANKL did not significantly alter PD-1 levels in CD8⁺ T cell TILs, indicating that the modulation of PD-1 expression was uniquely observed with anti-PD-1 monotherapy and in the anti-RANKL plus anti-PD-1 combination but not with anti-CTLA4 therapy. Interestingly, PD-1 expression by gp70-specific CD8⁺ T cell TILs was not further reduced with the further addition of anti-CTLA4 to anti-PD-1 and anti-RANKL.

In addition, expression of PD-L1 (a ligand for PD-1) in the non-lymphoid CD45.2+components of the tumor was assessed in the CT26 model (FIG. 19B). In keeping with adaptive immune resistance secondary to ICB, it was noted that the proportion of such cells expressing PD-L1 was slightly increased after a single dose of anti-PD-1, but this was mitigated when anti-RANKL was given with anti-PD-1 (FIG. 19B). The addition of anti-CTLA4 did not affect expression of PD-L1 (FIG. 19A-B). These results imply that anti-RANKL improves anti-PD-1 or anti-PD-1+anti-CTLA4 therapy through modulating expression of immunosuppressive PD-L1 in non-lymphoid TILs and this mechanism was distinct from combination anti-RANKL and anti-CTLA4.

Altogether, these data suggest that the mechanisms by which anti-RANKL enhance anti-PD-1/PD-L1 efficacy are distinct from the enhancement of anti-CTLA4 efficacy with anti-RANKL blockade. First, these data demonstrated that the anti-tumor efficacy of anti-PD-1 and anti-CTLA4 could be further improved by the addition of RANKL blockade, and that the anti-tumor efficacy of this triple combination therapy was superior to any dual combination. Secondly, the unique mechanistic interaction of anti-RANKL with different combination therapies, might be explained by alterations of the TME upon RANKL inhibition, which would uniquely cross-modulate with certain combination therapies. Treatment with anti-CTLA4 did not alter RANKL level on T-cell TILs. Therefore, in tumors which usually have a low expression of RANKL (e.g., melanoma, etc.), the cross-modulation hypothesis would predict that administration of anti-PD-1 could result in upregulation of RANKL in the TME, thereby resulting in increased RANK signaling, and priming the tumor for response to concurrent or subsequent RANKL blockade. It has been previously demonstrated in preclinical models that PD-1 expression by T cell TILs above a threshold level can result in resistance to anti-PD-1 antibodies, and strategies (such as combination with alternative immunotherapies) to reduce expression below this level results in therapeutic benefit (Selby et al., 2013. Cancer Immunol Res 1:32-42; Ngiow et al., 2015, supra). In the described analysis of CT26 tumors, therapeutic sensitivity of dual anti-RANKL and anti-PD-1 treatment was associated with favorable changes to the tumor microenvironment including both reduction of PD-1 expression by T cells, and reduction in PD-L1 expression. These changes to the tumor microenvironment were not observed upon treatment with anti-CTLA4 mAb alone, indicating that CTLA-4 and PD-1 regulate non-overlapping mechanisms of action and suggest that concurrent combination therapy with anti-RANKL occurs through distinct inhibitory pathways and distinct mechanisms.

### Example 20

### ANTI-RANKL Mab is Optimally Administered Concurrently with or following Immune Checkpoint Blockade

The optimal sequence of anti-RANKL antibody therapy relative to dual immune checkpoint blockade (ICB) therapy (combined anti-PD-1 and anti-CTLA4 mAb treatment) was assessed. Concurrent antibody therapy (antibody treatment on days 8, 12, 16, 20 after tumor inoculation) was compared with sequential therapy (equivalent total dose of antibody on days 8, 12 or 16, 20) in s.c. growth suppression of colon carcinoma CT26. Significantly superior growth suppression was achieved when anti-RANKL mAb was administered concurrently with, or following, dual ICB therapy (FIG. 20). Compared with concurrent anti-RANKL monotherapy, sequential anti-RANKL followed by dual ICB significantly suppressed tumor growth; however this sequence was less efficacious than concurrent dual ICB alone (FIG. 20).

The anti-tumor efficacy of combination anti-RANKL and anti-PD-1 in the mouse 3LL lung adenocarcinoma model was also tested in order to address the optimal sequence of mAb therapy. Tumor growth suppressive activity of concurrent mAb therapy was compared with equivalent total dose of mAbs given as sequential therapy. Preclinical data show combination anti-RANKL and anti-PD-1 mAbs were superior in efficacy to either monotherapy or control Ig, irrespective of whether therapies were given concurrently or sequentially (FIG. 21). Sequencing anti-PD-1 therapy prior to anti-RANKL treatment led to a significantly greater reduction in tumor volume as compared with anti-RANKL treatment prior to anti-PD-1 therapy (p<0.01) (FIG. 21). Sequential anti-RANKL followed by anti-PD-1 significantly suppressed tumor growth; however this sequence was less efficacious than concurrent treatment of anti-RANKL and anti-PD-1.

Taken together, these data indicate that anti-tumor efficacy observed in preclinical models was enhanced upon combination of RANKL blockade and anti-PD-1 antibody (compared with each antibody alone) irrespective of sequence. However, the data did indicate that sequential anti-RANKL followed by dual ICB (anti-PD-1 and anti-CTLA4) or anti-PD-1 alone was significantly less efficacious than concurrent treatment. Therefore, these data suggest that administration of anti-RANKL therapy should occur concurrently (or after) combination treatment with anti-PD-1/PD-L1 or anti-CTLA4 (or both). Moreover, the data demonstrating that concurrent treatment with anti-RANKL in combination with anti-PD-1/PD-L1 or anti-CTLA4 (or both) achieves superior anti-tumor response than sequentially later treatment with anti-RANKL supports the potential activity of multi-specific (e.g., bispecific) antibodies which would simultaneously block RANKL and PD-1, PD-L1 and/or CTLA4.

### Example 21

### Co-Expression of RANKL And PD-1 on Cells as Rationale for Co-Targeting RANKL and PD-1 using a Multi-Specific Antagonist

For a bi-specific antibody blocking two immunosuppressive pathways, co-expression of the target antigens on the same cell type would be favored as function would be target cell intrinsic. Co-expression of target antigens on a single cell type may also proscribe a more cell- or tissue-specific action of the bispecific modality within the tumor microenvironment and less peripheral toxicity, by virtue of greater cell selectivity within the tumor. Alternatively, or in addition, a bi-specific antibody blocking two immunosuppressive pathways expressed on two distinct cells in trans may also be advantageous, as two distinct immunosuppressive mechanisms may be inhibited simultaneously.

Therefore as a rationale for multi-specific antagonist targeting of RANKL and PD-1 in the TME, the co-expression of these factors on tumor-infiltrating immune cells should be considered. Anti-tumor efficacy and mechanistic data of anti-RANKL combined with anti-PD-1 in the CT26 tumor model has been described and the co-expression of RANKL and PD-1 on T cell TILs was characterized in this model, as described above. For instance, in T cells isolated from CT26 tumors in mice, almost all CD8⁺ RANKL⁺ T cell TILs (>90%) co-expressed PD-1; in comparison, less than 40% CD8+RANKL⁻T cell TILs were PD-1 positive (FIG. 16). Furthermore, the MFI of PD-1 was at least 3 fold higher on CD8⁺ RANKL⁻ compared to CD8⁺ RANKL⁻T cells identifying the former as PD1^{hi} cells (FIG. 16). As an example, 98.5% of tumor infiltrating CD8⁺ RANKL⁺ T cells expresses PD-1, while 44% of CD8+RANKL⁻T cells express PD-1 (FIG. 22), demonstrating a very high level of RANKL/PD-1 co-expression in TILs.

### Example 22

### Design of Tetravalent Anti-RANKL/PD-1 Fit-IG Construct (Denosumab+Nivolumab)

One example of a multi-specific antibody that antagonizes RANKL and at least one ICM can be constructed as a multi-specific FIT-Ig antibody constructed from two antibodies, one which binds RANKL (mAb A) and one which binds PD-1 (mAb B). By way of illustration, the first antigen-binding molecule can bind specifically to a region of human RANKL, and the second antigen-binding molecule can bind specifically to a region of human PD-1, and preferably to a region of the extracellular domain of human PD-1. One such anti-RANKL mAb that is suitable for use with the present invention is denosumab. Accordingly, in some embodiments, the anti-RANKL antigen-binding molecule comprises the fully human IgG2 mAb denosumab, or an antigen-binding fragment thereof. In some of the same embodiments and other embodiments, the anti-RANKL antigen-binding molecule comprises the CDR sequences as set forth in Table 1 herein. In specific examples of the multi-specific FIT-Ig antibody, the second antigen-binding molecule may comprise at least an antigen-binding fragment of any one of the mAbs selected from nivolumab, pembrolizumab, and pidilizumab. One such anti-PD-1 mAb that is suitable for use with the present invention is nivolumab.

To construct a tetravalent, multi-specific FIT-Ig molecule which binds and antagonizes both RANKL and PD-1, the light chain (V_{L}-CL) domains of denosumab is directly fused in tandem with the heavy chain (V_{H}-C_{H1}-C_{H}2-C_{H}3) of nivolumab at the NH2-terminus. The second construct is V_{H}-C_{H1} of denosumab and the third construct is V_{L}-C_{L} of nivolumab. The schematic diagram of the anti-RANKL/PD-1 FIT-Ig molecule is depicted in FIG. 23A and the DNA construct design for the anti-RANKL/PD-1 FIT-Ig molecule is depicted in FIG. 23B. All three DNA constructs are subcloned into a mammalian expression vector and protein production can be achieved upon transient transfection of all three DNA constructs subcloned into mammalian expression vectors into HEK-293 cells. Purification of RANKL/PD-1 FIT-Ig molecule can be achieved by Protein A purification.

Amino acid seouence of RANKL/PD-1 FIT-Ig
construct #1: VL (denosumab)-CL
(denosumab)- V_{H}-C_{H1}-C_{H2}-C_{H3} (nivolumab)
(655 aa):
[SEQ ID NO: 276]
EIVLTQSPGTLSLSPGERATLSCRASQSVRGRYLAWYQQKPGQAPRLLIY
GASSRATGIPDRFSGSGSGTDFTLTISRLEPEDFAVFYCQQYGSSPRTFG
QGTKVEIKrtvaapsvfifppsdeqlksgtasvvcllnnfypreakvqwk
vdnalqsgnsqesvteqdskdstyslsstltlskadyekhkvyacevthq
glsspvtksfnrgecQVQLVESGGGVVQPGRSLRLDCKASGITFSNSGMH
WVRQAPGKGLEWVAVIWYDGSKRYYADSVKGRFTISRDNSKNTLFLQMNS
LRAEDTAVYYCATNDDYWGQGTLVTVSSASTKGPSVFPLAPCSRSTSEST
AALGCLVKDYFPEPVTVSWNSGALTSGVHTFPAVLQSSGLYSLSSVVTVP
SSSLGTKTYTCNVDHKPSNTKVDKRVESKYGPPCPPCPAPEFLGGPSVFL
FPPKPKDTLMISRTPEVTCVVVDVSQEDPEVQFNWYVDGVEVHNAKTKPR
EEQFNSTYRVVSVLTVLHQDWLNGKEYKCKVSNKGLPSSIEKTISKAKGQ
PREPQVYTLPPSQEEMTKNQVSLTCLVKGFYPSDIAVEWESNGQPENNYK
TTPPVLDSDGSFFLYSRLTVDKSRWQEGNVFSCSVMHEALHNHYTQKSLS
LSLGK,

wherein the mature amino acid sequence of the anti-RANKL antibody (denosumab) light chain (US 7,364,736 B2) variable region (V_{L}) is shown in capital letters, and the constant region (C_{L}) is shown in lowercase letters; the anti-PD-1 antibody (nivolumab, WO2006/121 168) heavy chain (V_{H}-C_{H1}-C_{H2}-C_{H3}) is shown in bold, capital letters.

Amino acid sequence of RANKL/PD-1 FIT-Ig
construct #2: V_{H}-C_{H1} denosumab) (218 aa):
[SEQ ID NO: 277]
EVQLLESGGGLVQPGGSLRLSCAASGFTFSSYAMSWVRQAPGKGLEWVSG
ITGSGGSTYYADSVKGRFTISRDNSKNTLYLQMNSLRAEOTAVYYCAKDP
GTTVIMSWFOPWGQGTLVTVSSastkgpsvfplapcsrstsestaalgcl
vkdyfpepvtvswnsgaltsgvhtfpavlqssglyslssvvtvpssnfgt
qtytcnvdhkpsntkvdktv,

wherein the mature amino acid sequence of the anti-RANKL antibody (denosumab) heavy chain (U.S. Pat. No. 7,364,736 B2) variable region (VH) is shown in capital letters, and the constant region (C_{H1}) is shown in lowercase letters.

Amino acid sequence of RANKL/PD-1 FIT-Ig
construct #3: V_{L}-C_{L} (nivolumab) (214 aa}:
[SEQ ID NO: 278]
EIVLTQSPATLSLSPGERATLSCRASQSVSSYLAWYQQKPGQAPRLLIYD
ASNRATGIPARFSGSGSGTDFTLTISSLEPEDFAVYYCQQSSNWPRTFGQ
GTKVEIKRTVaapsvfifppsdeqlksgtasvvclinnfypreakvqwkv
dnalqsgnsqesvteqdskdstyslsstltlskadyekhkvyacevthqg
lsspvtksfnrgec,

wherein the mature amino acid sequence of the anti-PD-1 antibody light chain (nivolumab, WO2006/121 168) variable region (VL) is shown in capital letters, and the constant region (C_{L}) is shown in lowercase letters.

### Example 23

### Design of Tetravalent Anti-RANKL/CTLA4 FIT-IG Construct (denosumab+Ipilimumab)

One example of a multi-specific antibody that antagonizes RANKL and at least one ICM can be constructed as a multi-specific FIT-Ig antibody constructed from two antibodies, one which binds RANKL (mAb A) and one which binds CTLA4 (mAb B). By way of illustration, the first antigen-binding molecule can bind specifically to a region of human RANKL, and the second antigen-binding molecule can bind specifically to a region of human CTLA4, and preferably to a region of the extracellular domain of human CTLA4. One such anti-RANKL mAb that is suitable for use with the present invention is denosumab. Accordingly, in some embodiments, the anti-RANKL antigen-binding molecule comprises the fully human IgG2 mAb denosumab, or an antigen-binding fragment thereof. In some of the same embodiments and other embodiments, the anti-RANKL antigen-binding molecule comprises the CDR sequences as set forth in Table 1 herein. In specific examples of the multi-specific FIT-Ig antibody, the second antigen-binding molecule comprises at least an antigen-binding fragment of any one of the MAbs selected from ipilimumab and tremelimumab. One such anti-CTLA4 mAb that is suitable for use with the present invention is ipilimumab.

To construct a tetravalent, multi-specific FIT-Ig molecule which binds and antagonizes both RANKL and CTLA4, the light chain (V_{L}-C_{L}) domains of denosumab are directly fused in tandem with the heavy chain (V_{H}-C_{H2}-C_{H2}-CHs) of ipilimumab at the NH2-terminus. The second construct is V_{H}-C_{H1} of denosumab and the third construct is V_{L}-C_{L} of ipilimumab. The schematic diagram of the anti-RANKL/CTLA4 FIT-Ig molecule is depicted in FIG. 24A and the DNA construct design for the anti-RANKL/CTLA4 FIT-Ig molecule is depicted in FIG. 24B. All three DNA constructs are subcloned into a mammalian expression vector and protein production can be achieved upon transient transfection of all three DNA constructs subcloned into mammalian expression vectors into HEK-293 cells. Purification of RANKL/CTLA4 FIT-Ig molecule can be achieved by Protein A purification.

Amino acid sequence of RANKL/CTLA4 FIT-Ig construct #1: V_{L} (denosumab)-C_{L} (denosumab)-V_{H}-CH1⁻C_{H2}-CH3 (ipilimumab) (663 aa):

[SEQ ID NO: 279]
EIVLTQSPGTLSLSPGERATLSCRASQSVRGRYLAWYQQKPGQAPRLLIY
GASSRATGIPDRFSGSGSGTDFTLTISRLEPEDFAVFYCQQYGSSPRTFG
QGTKVEIKrtvaapsvfifppsdeqlksgtasvvcllnnfypreakvqwk
vdnalqsgnsqesvteqdskdstyslsstltlskadyekhkvyacevthq
glsspvtksfnrgecQVQLVESGGGVVQPGRSLRLSCAASGFTFSSYTMH
WVRQAPGKGLEWVTFISYDGNNKYYADSVKGRFTISRDNSKNTLYLQMNS
LRAEDTAIYYCARTGWLGPFDYWGQGTLVTVSSASTKGPSVFPLAPSSKS
TSGGTAALGCLVKDYFPEPVTVSWNSGALTSGVHTFPAVLQSSGLYSLSS
VVTVPSSSLGTQTYICNVNHKPSNTKVDKRVEPKSCDKTHTCPPCPAPEL
LGGPSVFLFPPKPKDTLMISRTPEVTCVVVDVSHEDPEVKFNWYVDGVEV
HNAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWESN
GQPENNYKTTPPVLDSDGSFFLYSKLTVDKSRWQQGNVFSCSVMHEALHN
HYTQKSLSLSPGK,

wherein the mature amino acid sequence of the anti-RANKL antibody (denosumab) light chain (U.S. Pat. No. 7,364,736 B2) variable region (V_{L}) is shown in capital letters, and the constant region (C_{L}) is in lowercase letters; the anti-CTLA4 antibody (ipilimumab, US20150283234) heavy chain (V_{H}-C_{H1}-C_{H2}-C_{H3}) is shown in bold, capital letters.

Amino acid sequence of RANKL/CTLA4 FIT-Ig construct #2: V_{H}-C_{Hl}denosumab) (214 aa):

The sequence is the same as V_{H}-C_{H1} denosumab construct used for RANKL/PD-1 FIT-Ig construct #2 above [SEQ ID NO:277].

Amino acid sequence of RANKL/CTLA4 FIT-Ig construct #3: V_{L}-C_{L} (ipilimumab) (215 aa):

[SEQ ID NO: 280]
EIVLTQSPGTLSLSPGERATLSCRASQSVGSSYLAWYQQKPGQAPRLLIY
GAFSRATGIPDRFSGSGSGTDFTLTISRLEPEDFAVYYCQQYGSSPWTFG
QGTKVEIKRTVaapsvfifppsdeqlksgtasvvcllnnfypreakvqwk
vdnalqsgnsqesvteqdskdstyslsstltlskadyekhkvyacevthq
glsspvtksfnrgec,

wherein the mature amino acid sequence of the anti-CTLA4 antibody light chain (ipilimumab, US20150283234) variable region (VL) is shown in capital letters, and the constant region (C_{L}) is shown in lowercase letters.

### Example 24

### Design of Tetravalent Anti-RANKL/PD-L1 FIT-IG Construct (Denosumab+Atezolizumab)

One example of a multi-specific antibody that antagonizes RANKL and at least one ICM can be constructed as a multi-specific FIT-Ig antibody constructed from two antibodies, one which binds RANKL (mAb A) and one which binds PD-L1 (mAb B). By way of illustration, the first antigen-binding molecule may bind specifically to a region of human RANKL, and the second antigen-binding molecule may bind specifically to a region of human PD-L1, and preferably to a region of the extracellular domain of human PD-L1. One such anti-RANKL MAb that is suitable for use with the present invention is denosumab. Accordingly, in some embodiments, the anti-RANKL antigen-binding molecule comprises the fully human IgG2 mAb denosumab, or an antigen-binding fragment thereof. In some of the same embodiments and other embodiments, the anti-RANKL antigen-binding molecule comprises the CDR sequences as set forth in Table 1 herein. In specific examples of the multi-specific FIT-Ig antibody, the second antigen-binding molecule comprises at least an antigen-binding fragment of any one of the mAbs selected from durvalumab (MEDI4736), atezolizumab (Tecentriq), avelumab, BMS-936559/MDX-1105, MSB0010718C, LY3300054, CA-170, GNS-1480 and MPDL3280A, or antigen-binding fragments thereof. One such anti-PD-L1 mAb that is suitable for use with the present invention is atezolizumab.

To construct a tetravalent, multi-specific FIT-Ig molecule which binds and antagonizes both RANKL and PD-L1, the light chain (V_{L}-C_{L}) domains of denosumab are directly fused in tandem with the heavy chain (V_{H}-C_{H1}-C_{H}2-C_{H}3) of atezolizumab at the NH2-terminus. The second construct is V_{H}-C_{H1} of denosumab and the third construct is V_{L}-C_{L} of atezolizumab. The schematic diagram of the anti-RANKL/PD-L1 FIT-Ig molecule is depicted in FIG. 25A and the DNA construct design for the anti-RANKL/PD-L1 FIT-Ig molecule is depicted in FIG. 25B. All three DNA constructs are subcloned into a mammalian expression vector and protein production can be achieved upon transient transfection of all three DNA constructs subcloned into mammalian expression vectors into HEK-293 cells. Purification of anti-RANKL/PD-L1 FIT-Ig molecule can be achieved by Protein A purification.

### Amino acid sequence of RANKL/PD-L1 FIT-Ig construct #1: V_{L} (denosumab)-C_{L} (denosumab)-V_{H}-C_{H1}-C_{H2}-C_{H3} (Atezolizumab) (663 aa):

[SEQ ID NO: 281]
EIVLTQSPGTLSLSPGERATLSCRASQSVRGRYLAWYQQKPGQAPRLLIY
GASSRATGIPDRFSGSGSGTDFTLTISRLEPEDFAVFYCQQYGSSPRTFG
QGTKVEIKrtvaapsvfifppsdeqlksgtasvvcllnnfypreakvqwk
vdnalqsgnsqesvteqdskdstyslsstltlskadyekhkvyacevthq
glsspvtksfnrgecEVQLVESGGGLVQPGGSLRLSCAASGFTFSDSWIH
WVRQAPGKGLEWVAWISPYGGSTYYADSVKGRFTISADTSKNTAYLQMNS
LRAEDTAVYYCARRHWPGGFDYWGQGTLVTVSSASTKGPSVFPLAPSSKS
TSGGTAALGCLVKDYFPEPVTVSWNSGALTSGVHTFPAVLQSSGLYSLSS
VVTVPSSSLGTQTYICNVNHKPSNTKVDKKVEPKSCDKTHTCPPCPAPEL
LGGPSVFLFPPKPKDTLMISRTPEVTCVVVDVSHEDPEVKFNWYVDGVEV
HNAKTKPREEQYASTYRVVSVLTVLHQDWLNGKEYKCKVSNKALPAPIEK
TISKAKGQPREPQVYTLPPSREEMTKNQVSLTCLVKGFYPSDIAVEWESN
GQPENNYKTTPPVLDSDGSFFLYSKLTVDKSRWQQGNVFSCSVMHEALHN
HYTQKSLSLSPGK,

wherein the mature amino acid sequence of the anti-RANKL antibody (denosumab) light chain (US 7,364,736 B2) variable region (V_{L}) is shown in capital letters, and the constant region (C_{L}) is shown in lowercase letters; the anti-PD-L1 antibody (atezolizumab, U.S. Patent No. 8,217148) heavy chain (V_{H}-C_{H1}-C_{H2}-C_{H3}) is shown in bold, capital letters.

### Amino Acid Sequence of RANKL/PD-L1 FIT-IG Construct #2: V_{H}-C_{H1} Denosumab):

The sequence is the same as V_{H}-C_{H1} denosumab construct used for RANKL/PD-1 FIT-Ig construct #2 above [SEQ ID NO:277].

Amino acid sequence of RANKL/PD-L1 FIT-Ig construct #3: V_{L}-C_{L} (atezolizumab) (214 aa):

[SEQ ID NO: 282]
DIQMTQSPSSLSASVGDRVTITCRASQDVSTAVAWYQQKPGKAPKLLIYS
ASFLYSGVPSRFSGSGSGTDFTLTISSLQPEDFATYYCQQYLYHPATFGQ
GTKVEIKRTVAAPsVfifppsdeqlksgtasvvcllnnfypreakvqwkv
dnalqsgnsqesvteqdskdstyslsstltlskadyekhkvyacevthqg
lsspvtksfnrgec,

wherein the mature amino acid sequence of the anti-PD-L1 antibody light chain (atezolizumab, U.S. Patent No. 8,217148) variable region (VL) is shown in capital letters, and the constant region (C_{L}) is shown in lowercase letters.

### Example 25

### Construction of Bispecific Anti-RANKL/PD-1 Antibody (ik22-5/rmp1-14)

A heterodimeric (bispecific) antibody was generated which binds to both mouse RANKL and mouse PD-1 by fusing the Fab-encoding sequences onto a human IgG1 backbone. Assembly of heterodimeric bi-specific IgG antibodies was achieved by first introduction of complementary KIH mutations into the CH3 domain of IgG heavy chains. The association of the desired light-chain/heavy-chain pairings was promoted by the “CrossMab” approach (see, e.g., Schaefer et al., 2011. Proc Natl Acad Sci U S A 108: 11187-11192), in which modification of one Fab of the bispecific antibody (Fab region) to “swap” the constant or constant and variable regions between the light and heavy chains. The D265A mutation was also introduced into the human IgG1 Fc domain to reduce binding to Fc receptors and reduce effector function. Using these techniques, a bispecific anti-RANKL/PD-1 antibody (also called RMP1-14 C_{H}-C_{L} X IK22/5 WT bispecific) was constructed, produced and purified by standard techniques. Furthermore, the bispecific anti-RANKL/PD-1 antibody is capable of binding both targets and has antagonistic activity against RANKL and PD-1 in vitro and in vivo.

The mAb cDNA sequences were obtained from rat hybridomas encoding anti-RANKL IK22-5 (Kamijo et al., 2006. Biochem Biophys Res Commun. 347(1):124-32) and anti-PD-1 RMP1-14 (Curran et al., 2010. Proc Natl Acad Sci U S A 107(9):4275-80). Total RNA was isolated from the hybridoma cells following the technical manual of TRIzol® Reagent (Ambion, Cat. No. : 15596-026). Total RNA was then reverse transcribed into cDNA using isotype-specific anti-sense primers or universal primers following the technical manual of PrimeScriptTM 1st Strand cDNA Synthesis Kit (Takara, Cat. No. 6110A). The antibody fragments of V_{H} and V_{L} were amplified according to the standard operating procedure (SOP) of rapid amplification of cDNA ends (RACE), according to standardized techniques. Amplified antibody fragments were cloned into a standard cloning vector separately and DNA sequencing performed. The amino acid sequences for variable domain and leader sequence of anti-RANKL mAb IK22-5 and anti-PD-1 mAb RMP1-14 are provided (see below). Sequence analysis of immunoglobulin variable regions and determination of framework (FR) and CDRs were achieved using NCBI Nucleotide BLAST, IMGT/V Quest program and NCBI IgBLAST algorithms.

Amino acid sequences for variable domain of rat monoclonal antibody anti-RANKL mAb IK22-5:

Heavy chain IK22-5: Amino acids sequence (135 aa):
Leader sequence-FR1-CDR1-FR2-CDR2-FR3-CDR3-FR4
[SEQ ID NO: 283]
MDVLVLWLCLLTFSSCVLSQVQLKESGPGLVLSSATLSLTCTVSGFSLTN
YDVSWIRHLPGKGLEWMGGVWLSGNTEYNSDFKSRLSISRDISKSQVFLK
MSNLKIEDTGTYYCARDIGTTSDYWGQGVTVTVSS
Light chain IK22-5: Amino acids sequence (126 aa):
Leader seauence-FR1-CDR1-FR2-CDR2-FR3-CDR3-FR4
[SEQ ID NO: 284]
MMAPVQLLGLLLLWLPALRCDIQVTQSPSFLSASVGDRVTFNCKTSQNIN
KYLAWYQAKFGEGPKLLIFNADSLQSGIPPRFSGSGSGTDFTLTISGLQP
EDFATYFCLQYNSWPTFGSGTKLEIK

Amino acid sequences for variable domain of rat monoclonal antibody anti-PD-1 mAb RMPI-14:

Heavy chain RMP1-14: Amino acids
sequence (138 aa)
Leader sequence-FR1-CDR1-FR2-
CDR2-FR3-CDR3-FR4
[SEQ ID NO: 285]
MRMLVLLYLLTALPGILSEVQLQESGPGLVKPSQSLSLTCSVTGYSITSS
YRWNWIRKFPGNRLEWMGYINSAGISNYNPSLKRRISITRDTSKNQFFLQ
VNSVTTEDAATYYCARSDNMGTTPFTYWGQGTLVTVSS
Light chain RMP1-14: Amino acids
sequence (131 aa)
Leader seouence-FR1-CDR1-FR2-
CDR2-FR3-CDR3-FR4
[SEQ ID NO: 286]
MRCSLQFLGLLVLWIPGLNGDIVMTQGTLPNPVPSGESVSITCRSSKSLL
YSDGKTYLNWYLQRPGQSPQLLIYWMSTRASGVSDRFSGSGSGTDFTLKI
SGVEAEDVGIYYCQQGLEFPTFGGGTKLELK

### Example 26

### Bispecific Anti-RANKL/PD-1 Antibody Construction, Production And Purification

In order to generate a multi-specific antigen-binding molecule with ability to bind RANKL and PD-1 (bispecific anti-RANKL/PD-1 antibody, also called RMP1-14 C_{H}-C_{L} X IK22/5 WT bispecific), the “CrossMAb” technology was employed, in which the desired light-chain/heavy-chain pairings can be induced by modification of one Fab of the bispecific antibody (Fab region) to “swap” the constant or constant and variable regions between the light and heavy chains. Secondly, to produce the specific pair of heterodimer of heavy chain, “knob-in-hole” (KiH) mutations in Fc domain of two heavy chains was utilized. The DNAs encoding the rat monoclonal antibody variable regions (from IK22-5 and RMP1-14) were synthesized as a fusion with the human IgG1 Fc domain. This technique for preventing association of “improper” light/heavy chains is termed “CrossMab” technology and, when combined with KiH technology, results in remarkably enhanced expression of the desired bispecific molecules (see, e.g., Schaefer et al., 2011. Proc Natl Acad Sci U S A 108: 11187-11192).

In order to generate the CrossMab form of a bispecific anti-RANKL/PD-1 antibody, the RMP1-14 (anti-PD-1 antibody) sequence was engineered as a “CrossMabC_{Hl}-C_{L}”, in which the C_{H1} and C_{L} sequences were interchanged (termed RMP1-14 CH-CL-huIgGlFc). The Fab region of the anti-RANKL antibody (IK22-5) was unchanged (termed IK22-5-huIgGlFc WT). Heterodimerization of polypeptide chains was facilitated by introducing large amino acids (knobs) into one chain of a desired heterodimer and small amino acids (holes) into the other chain of the desired heterodimer, also called “Knobs-into-holes” (KIH) structures (see, e.g., Ridgeway et al., Protein Eng. 9(1996), 617-621 and Atwell et al., J. Mol. Biol. 270(1997), 677-681). Specifically, the “knob” mutation (T366W) was introduced into the CH3 domain of IK22-5-huIgGlFc WT, and three “hole” mutations (T366S, L368A, and Y407V) were introduced into the heavy chain of RMP1-14 C_{H}-C_{L}-huIgGlFc. In addition, two Cys residues were introduced (S354C on the “knob” and Y349C on the “hole” side) in order form a stabilizing disulfide bridge and further enhance heterodimerization. Furthermore, a D265A mutation was also introduced into the human IgG1 Fc domain of both IK22-5-huIgGlFc WT and RMP1-14 CH-CL-huIgGlFc. A schematic representation of the bispecific anti-RANKL/PD-1 antibody is shown in FIG. 26.

Amino acid sequences for four antibody chains for bispecific CrossMab anti-RANKL/PD-1 antibody

IK22-5-huIgG1Fc WT Heavy chain (465 aa);
Leader sequence-FR1-CDR1-FR2-CDR2-FR3-CDR3-FR4-C_{H1}-_{CH2}-C_{H3} (Heavy 1)
[SEQ ID NO: 287]
MGWSCIILFLVATATGVHSQVQLKESGPGLVLSSATLSLTCTVSGFSLTNYDVSWIR
HLPGKGLEWMGGVWLSGNTEYNSDFKSRLSISRDISKSQVFLKMSNLKIEDTGTYYCARDIGTTSDYW
GQGVTVTVSSASTKGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKKVEPKSCDKTHTCPPCPAPELLGGPSVFLFPPKPKDTLMISRT
PEVTCVVVAVSHEDPEVKFNWYVDGVEVHNAKTKPREEQYNSTYRVVSVLTVLHQDWLNGKEYKCKVSNKALP
APIEKTISKAKGQPREPQV [symbol] TLPPCRDELTKNQVSL [symbol] VKGFYPSDIAVEWESNGQPENNYKTTPPVLDSDGS
FFL [symbol] SKLTVDKSRWQQGNVFSCSVLHEALHNHYTQKSLSLSPGK
IK22-5-huIgG1Fc WT Light chain (232 aa):
Leader seguence-FR1-CDR1-FR2-CDR2-FR3-CDR3-FR4-C_{L}
[SEQ ID NO: 288]
MGWSCIILFLVATATGVHSDIQVTQSPSFLSASVGDRVTFNCKISQNINKYLAWYQA
KFGEGPKLLIFNADSLQSGIPPRFSGSGSGTDFTLTISGLQPEDFATYFCLQYNSWPTFGSGTKLEIKRTV
AAPSVFIFPPSDEQLKSGTASVVCLLNNFYPREAKVQWKVDNALQSGNSQESVTEQDSKDSTYSLSSTLTLSKA
DYEKHKVYACEVTHQGLSSPVTKSFNRGEC
RMP1-14 C_{H}-C_{L}- huIgG1Fc Heavy chain (473 aa):
Leader sequence- FR1-CDR1-FR2-CDR2-FR3-CDR3-FR4-CL-C_{H2}-C_{H3}
[SEQ ID NO: 289]
MGWSCIILFLVATATGVHSEVQLQESGPGLVKPSQSLSLTCSVTGYSITSSYRWNWI
RKFPGNRLEWMGYINSAGISNYNPSLKRRISITRDTSKNQFFLQVNSVTTEDAATYYCARSDNMGTTPFT
YWGQGTLVTVSSRTVAAPSVFIFPPSDEQLKSGTASVVCLLNNFYPREAKVQWKVDNALQSGNSQESVTEQD
SKDSTYSLSSTLTLSKADYEKHKVYACEVTHQGLSSPVTKSFNRGECDKTHTCPPCPAPELLGGPSVFLFPPKPKD
TLMISRTPEVTCVVV [symbol] VSHEDPEVKFNWYVDGVEVHNAKTKPREEQYNSTYRVVSVLTVLHQDWLNGKEYKCK
VSNKALPAPIEKTISKAKGQPREPQV [symbol] TLPP [symbol] RDELTKNQVSL [symbol] C [symbol] VKGFYPSDIAVEWESNGQPENNYKTTPPV
LDSDGSFFL [symbol] SKLTVDKSRWQQGNVFSCSVLHEALHNHYTQKSLSLSPGK
RMP1-14 C_{H}-C_{L} - huIgG1Fc Light chain (233 aa):
[SEQ ID NO: 290]
Leader seauence-FR1-CDR1-FR2-CDR2-FR3-CDR3-FR4-C_{H1}
MGWSCIILFLVATATGVHSDIVMTQGTLPNPVPSGESVSITCRSSKSLLYSDGKTYLNWYLQRPGQSPQLL
IYWMSTRASGVSDRFSGSGSGTDFTLKISGVEAEDVGIYYCQQGLEFPTFGGGTKLELKASTKGPSVFPLA
PSSKSTSGGTAALGCLVKDYFPEPVTVSWNSGALTSGVHTFPAVLQSSGLYSLSSVVTVPSSSLGTQTYICNVNH
KPSNTKVDKKVEPKSC

In order to produce recombinant bispecific antibody, cDNAs encoding each of the four chains were subcloned into the mammalian expression vector pcDNA3.4 and transfection grade plasmids were maxi-prepared according to standard techniques. The bispecific antibody was produced by transient expression in ExpiCHO-S suspension cells grown in serum-free ExpiCHO Expression Medium (Thermo Fisher Scientific) with four expression plasmids (encoding heavy and light chains for RMP1-14 CH-CL-huIgGlFc and IK22-5-huIgGlFc WT) at equimolar ratios. The cells (1L culture volume) were maintained in Erlenmeyer Flasks (Corning Inc.) at 37° C. with 8% CO2 on an orbital shaker and the cell culture supernatant collected on day 14 post-transfection was used for purification. Antibody titers were in the range of transient expression titers of conventional IgG1 antibodies. Cell culture broth was centrifuged followed by filtration. Filtered supernatant was loaded onto a Monofinity A Resin Prepacked Column 1 mL (GenScript, Cat.No.L00433-11) at 1.0 mL/min. After washing and elution with appropriate buffers, the eluted fractions of the antibody were pooled and buffer exchanged to PBS, pH 7.2. Protein was sterilized via a 0.22 pm filter, packaged aseptically and stored at −80° C.

To determine the molecular weight, yield and purity, the purified proteins were subsequently analyzed by SDS-PAGE, Western blot and HPLC using standard protocols. Based on SDS-PAGE and Western blot analysis under non-reducing conditions, target protein was detected with estimated molecular weight of —80 kDa, —100kDa and 150 kDa (Calculated M.W. 145 kDa) (as shown in FIG. 27). From SDS-PAGE and Western blot analysis as shown, the heavy and light chains of the antibody were detected with the estimated molecular weights of —55 kDa and —25 kDa. Purity of the bispecific anti-RANKL/PD-1 antibody was 85.86%, estimated by SEC-HPLC and concentration was 3.69mg/mL, determined by A280 (Extinction coefficients: 1.494).

The bispecific anti-RANKL/PD-1 antibody was obtained in high purity via standard protein A affinity chromatography after expression in suspension ExpiCHO-S cell culture for additional in vitro validation and testing in vivo.

### Example 27

### In Vitro Characterization of Bispecific Anti-RANKL/PD-1 Antibody

Bispecific anti-RANKL/PD-1 antibody (IK22-5/RMP1-14) binding to muRANKL on HEK-293

To characterize the ability of the bispecific anti-RANKL/PD-1 antibody to bind RANKL expressed on cells, flow cytometry analysis was performed. The specificity of the interaction was determined by comparing the signal intensity measured on HEK-293 cells transiently transfected with a cDNA encoding muRANKL compared with the signal intensity with that obtained with the untransfected HEK-293 cells. The bispecific anti-RANKL/PD-1 antibody failed to recognize untransfected HEK-293 cells but bound the muRANKL-expressing cells (FIG. 28). The binding of the bispecific anti-RANKL/PD-1 antibody to muRANKL was very similar to that observed with the positive control, muRANK-Fc (FIG. 28). Therefore, the RANKL/PD-1 antibody specifically recognized the extra-cellular domain of muRANKL expressed on the surface of cells with high affinity.

### Example 28

### Bispecific Anti-RANKL/PD-1 Antibody (ik22-5/rmp1-14) Competition With Rank-FC Binding

The ability of bispecific anti-RANKL/PD-1 antibody to block ligand binding was tested in a competition assay with recombinant muRANK-Fc, the high affinity receptor of RANKL. HEK-293 cells were transiently transfected with muRANKL and binding of RANK-Fc was tested in the presence of isotype control antibodies (rat IgG2a and huIgG1), positive control anti-muRANKL antibody (IK22-5 rat IgG2a) and bispecific anti-RANKL/PD-1. The anti-RANKL/PD-1 bispecific antibody was able to fully block RANK-Fc binding to muRANKL, as did the positive control anti-RANKL antibody IK22-5 (FIG. 29). The anti-RANKL/PD-1 bispecific antibody demonstrated antagonistic activity in blocking RANK-Fc binding to RANKL with an _{ICso} of 2.6 μg/mL, comparable to that observed with the control anti-RANKL mAb IK22-5 (IC₅₀ of 1.1 μg/mL). Neither the rat IgG2a nor human IgG1 isotype controls blocked RANK-Fc binding to RANKL.

### Example 29

### Bispecific Anti-RANKL/PD-1 (IK22-5/RMP1-14) Binding To Ectopically Expressed PD-1 on HEK-293 Cells

To characterize the ability of the bispecific anti-RANKL/PD-1 antibody to bind PD-1 expressed on cells, flow cytometry analysis was performed. The bispecific anti-RAN KL/PD-1 bispecific antibody specifically bound to muPD-1-transfected HEK-293 cells, but not to untransfected HEK-293 cells (FIG. 30). Therefore, the RANKL/PD-1 antibody specifically recognized the extra-cellular domain of muPD-1 expressed on the surface of cells with high affinity.

### Example 30

### Bispecific Anti-RANKL/PD-1 Antibody (IK22-5/RMP1-14) Competition with PD-L1-FC Binding

The ability of bispecific anti-RANKL/PD-1 antibody to block ligand binding was tested in a competition assay with recombinant muPD-L1-Fc, the high affinity ligand of PD-1. HEK-293 cells were transiently transfected with muPD-1 and binding of PD-L1-Fc was tested in the presence of isotype control antibodies (rat IgG2a and huIgG1), positive control anti-muPD-1 antibody (RMP1-14 rat IgG2a) and bispecific anti-RANKL/PD-1 antibody. The anti-RANKL/PD-1 bispecific antibody was able to block PD-L1-Fc binding to muPD-1, as did the positive control anti-PD-1 antibody RMP1-14 (FIG. 31). The anti-RANKL/PD-1 bispecific antibody demonstrated antagonistic activity in blocking PD-L1-Fc binding to PD-1 comparable to that observed with the control anti-PD-1 mAb RMP1-14. Neither the rat IgG2a nor human IgG1 isotype controls blocked PD-L1-Fc binding to PD-1.

### Example 31

### Antagonistic Activity Of Anti-RANKL/PD-1 Bispecific Antibody in Cell-Based Functional Assay

To evaluate the functional inhibitory effect of the bispecific anti-RANKL/PD-1 antibody in a cell-based functional assay, the effect of this antibody on in vitro osteoclastogenesis was tested. The methods for the in vitro TRAP⁺ osteoclast assays were essentially as described (Simonet et al., 1997. Cell 89(2): 309-19). Bone marrow (BM) cells from normal BL/6 mice were seeded in a 96 well flat bottom plate at a density of 25000 cells/well in a total volume of 200 pL/well of complete DMEM (10% FCS+PS+Glu) supplemented with 50 ng/mL of human recombinant CSF-1 (Preprotech). After culture for 48 hr, media is replaced with complete DMEM supplemented with 50 ng/mL of human recombinant CSF-1 and 200 ng/mL of soluble muRANKL (Miltenyi). Cells are cultured with CSF-1 and RANKL for 4 days (with and without antibody inhibitors) and then TRAP⁺ multinucleated (more than three nuclei) osteoclast cells were counted. The generated osteoclasts were evaluated by TRAP cytochemical staining as previously described (Simonet et al., 1997, supra). Similar to the effect of the positive control antibody IK22-5, the addition of the anti-RANKL/PD-1 bispecific antibody, but not the addition of control human IgG, inhibited the formation of TRAP⁺ multinucleated cells in a dose-dependent manner (FIG. 32). At a concentration of 100 ng/mL both the anti-RANKL mAb IK22-5 and the bispecific anti-RANKL/PD-1 antibody completely blocked osteoclast formation. These results indicated that the anti-RANKL/PD-1 bispecific antibody retains an antagonistic activity against RANKL and the differentiation of osteoclasts in vitro.

### Example 32

### In Vivo Testing of Bispecific Anti-RANKL/PD-1 Antibody in Tumor Models

Co-targeting of RANKL and PD-1 with a bispecific anti-RANKL/PD-1 antibody is superior to monotherapy anti-RANKL or anti-PD-1 in suppressing experimental metastasis to lung

In order to test the effect of bispecific anti-RANKL/PD-1 antibody to control metastases, wild type (WT) mice bearing experimental B16F10 melanoma lung metastases were used. Mice were treated on days -1, 0 and 2 (relative to tumor inoculation) with cIg (200 μg i.p., recombinant Mac4-human IgG1 D265A), anti-RANKL (100 μg i.p., recombinant IK22.5-human IgG1 D265A), anti-PD-1 (100 μg i.p., recombinant RMP1-14-human IgG1 D265A), anti-RANKL +anti-PD-1 (100 μg i.p. each), and a dose titration of the anti-RANKL/PD-1 bispecific (50 to 200 μg i.p., human IgG1 D265A) as indicated. The anti-RANKL or anti-PD-1 alone displayed modest efficacy compared with the control immunoglobulin (cIg)-treated group, while combined treatment with the 2 antibodies (anti-RANKL and anti-PD-1) or treatment with the bispecific anti-RANKL/PD-1 antibody significantly improved metastatic control (FIG. 33).

It is expected that treatment with the bispecific antibody has an in vivo inhibitory effect on lung metastases that is greater than either antibody alone or a combination of anti-PD-1 antibody and anti-RANKL antibody. The bispecific anti-RANKL/PD-1 antibody demonstrated a dose-dependent reduction in lung metastatic burden, with the 100 and 200 μg dose groups resulting in a superior reduction in lung metastases compared with anti-PD-1 alone (*p<0.05,***p<0.001, respectively). Compared with the combination treatments of anti-PD-1 antibody and anti-RANKL antibody dosed at 100 μg of each antibody (i.e., 200 μg of total antibody), treatment with the bispecific anti-RANKL/PD-1 antibody with an equivalent antibody dose (200 μg bispecific anti-RANKL/PD-1) achieved at least an equivalent improvement in metastatic control (FIG. 33).

A similar effect for the bispecific anti-RANKL/PD-1 antibody was seen in WT mice bearing experimental RM1 prostate cancer lung metastases (FIG. 34). Mice were treated on days -1, 0 and 2 (relative to tumor inoculation) with cIg (200 μg i.p., human IgG1 D265A), anti-RANKL (100 μg i.p., IK22.5 human IgG1 D265A), anti-PD-1 (100 μg i.p., human IgG1 D265A), anti-RANKL +anti-PD-1 (100 μg i.p. each), anti-RANKL-PD-1 bispecific (100 or 200 μg i.p., human IgG1 D265A) as indicated. The anti-RANKL or anti-PD-1 alone displayed modest efficacy compared with the control immunoglobulin (cIg)-treated group, while combined treatment with the 2 antibodies (anti-RANKL and anti-PD-1) or treatment with the bispecific anti-RANKL/PD-1 antibody significantly improved metastatic control (FIG. 34).

It is expected that treatment with the bispecific antibody has an in vivo inhibitory effect on lung metastases that is greater than either antibody alone or a combination of anti-PD-1 antibody and anti-RANKL antibody. The bispecific anti-RANKL/PD-1 antibody demonstrated a dose-dependent reduction in lung metastatic burden, with the 200 μg dose group resulting in a superior reduction in lung metastases compared with anti-PD-1 alone (*****p<0.0001). Compared with the combination treatments of anti-PD-1 antibody and anti-RANKL antibody dosed at 100 μg of each antibody (i.e., 200 μg of total antibody), treatment with the bispecific anti-RANKL/PD-1 antibody with an equivalent overall antibody dose (200 μg bispecific anti-RANKL/PD-1) achieved an equivalent improvement in metastatic control (FIG. 34). These results demonstrate that the bispecific anti-RANKL/PD-1 antibody achieved equivalent metastatic control compared with the groups treated with an equivalent dose of combination of anti-PD-1 and anti-RANKL MAbs, and indicate that the bispecific anti-RANKL/PD-1 has superior efficacy.

### Example 33

### Co-Targeting of RANKL and PD-1 with Bispecific Anti-RANKL/PD-1 Suppresses Subcutaneous Tumor Growth of a Lung Cancer Cell Line 3LL

In order to test the activity of the anti-RANKL/PD-1 bispecific antibody on growth of a subcutaneous tumor, the mouse 3LL lung adenocarcinoma model was utilized. Mice were treated on days 8, 12, 16 and 20 (relative to tumor inoculation) with cIg (400 μg i.p., rat IgG2a), anti-RANKL (100 μg i.p., IK22-5 rat IgG2a), anti-PD-1 (100 μg i.p., RMP1-14 rat IgG2a), anti-RANKL+anti-PD-1 (100 μg i.p. each IK22-5 and RMP1-14), and a dose titration of the anti-RANKL/PD-1 bispecific (100 to 400 μg i.p., human IgG1 D265A) as indicated. Treatment with the anti-RANKL mAb IK22-5 alone had no effect on 3LL s.c. tumor growth, while anti-PD-1 alone displayed modest efficacy compared with the control immunoglobulin (cIg)-treated group. All doses of the bispecific anti-RANKL/PD-1 antibody clearly had activity to reduce s.c. tumor growth of 3LL compared with cIg or control ant-RANKL treatment alone. The anti-tumor effect of the 200 μg dose of the anti-RANKL/PD-1 antibody was similar to that observed with an equivalent total dose (200 μg) of combined treatment with the 2 antibodies (anti-RANKL and anti-PD-1, 100 μg each) (FIG. 35). These data confirm the in vivo efficacy of the bispecific anti-RANKL/PD-1 antibody in a s.c. tumor model.

### Example 34

### Co-Targeting of RANKL and PD-1 with Bispecific Anti-RANKL/PD-1 Suppresses Subcutaneous Tumor Growth of a Colon Carcinoma Cell Line CT26

The efficacy of the bispecific anti-RANKL/PD-1 antibody was compared with the combination treatment with anti-RANKL and anti-PD-1 antibodies in mice bearing s.c. CT26 colon tumors (FIG. 36). In CT26 tumor-bearing mice, either anti-RANKL or anti-PD-1 (100 μg) had minimal effect as monotherapies, but when combined therapies (anti-RANKL plus anti-PD-1, 100 μgeach) were used, a suppression of established tumor growth was observed (FIG. 2A). The 100 μgand 200 μg doses of the bispecific anti-RANKL/PD-1 antibody clearly reduced s.c. tumor growth of CT26 compared with cIg, anti-PD-1 treatment alone or control anti-RANKL treatment alone. The lack of response to anti-PD-1 monotherapy indicates this tumor demonstrates some resistance to this immunotherapy and treatment with the single agent, bispecific anti-RANKL/PD-1 antibody overcomes this resistance. It is expected that treatment with the bispecific antibody has an in vivo inhibitory effect on CT26 tumor control that is greater than either antibody alone or a combination of anti-PD-1 antibody and anti-RANKL antibody. The anti-tumor effect of the 200 μg dose of the bispecific anti-RANKL/PD-1 antibody was similar to that observed with an equivalent total dose (200 μg of combined treatment with the 2 antibodies (anti-RANKL and anti-PD-1, 100 μg each) (FIG. 36). These data confirm the in vivo efficacy of the bispecific anti-RANKL/PD-1 antibody in a s.c. tumor model.

### Example 35

### Co-Targeting of RANKL and PD-1 with Bispecific Anti-RANKL/PD-1 Enhances the Anti-Tumor Efficacy of Anti-CTLA4 Treatment in the Ct26 Tumor Model

The results presented herein demonstrate that the anti-tumor efficacy of combined anti-PD-1/PD-L1 and anti-CTLA4 therapy, monotherapy anti-PD-1/PD-L1 or monotherapy anti-CTLA4 could be further improved by the addition of RANKL blockade. Furthermore, the anti-tumor efficacy of this triple combination therapy (anti-RANKL plus anti-PD-1 plus anti-CTLA4) was superior to any dual combination. These data suggest that the mechanisms by which anti-RANKL enhance anti-PD-1/PD-L1 efficacy are distinct from the mechanisms enhancement of anti-CTLA4 efficacy with anti-RANKL blockade.

To address whether the anti-RANKL/PD-1 bispecific antibody (as a single drug treatment) could enhance the anti-tumor efficacy of anti-CTLA4 mAb, the efficacy of the bispecific anti-RANKL/PD-1 antibody was compared (either alone or in combination with anti-CTLA4) with anti-CTLA4 treatment alone, the combination treatment of anti-CTLA4 plus anti-PD-1, or the combination treatment of anti-RANKL plus anti-PD-1 plus anti-CTLA4 (triple treatment therapy) in mice bearing s.c. CT26 colon tumors. In this model, treatment with anti-CTLA4 resulted in a moderate reduction in tumor growth, which was improved upon addition of anti-PD-1 (anti-CTLA4 plus anti-PD-1 combination) (FIG. 37). The addition of anti-RANKL mAb to the anti-CTLA4 plus anti-PD-1 combination (triple treatment therapy) further improved tumor control. Addition of the anti-RANKL/PD-1 bispecific antibody to anti-CTLA4 mAb reduced tumor growth certainly to a greater extent to that observed with either bispecific anti-RANKL/PD-1 antibody or anti-CTLA4 treatments alone, and improved tumor control compared with the triple therapy (anti-RANKL plus anti-PD-1 plus anti-CTLA4) (FIG. 37). These data indicate the ability of the anti-RANKL/PD-1 (as single drug treatment) to enhance the anti-tumor efficacy of anti-CTLA4 in a s.c.

### Example 36

### Co-Targeting of RANKL and PD-1 with Bispecific Anti-RANKL/PD-1 Suppresses Subcutaneous Tumor Growth of a Breast Cancer Cell Line AT3OVA

The efficacy of the bispecific anti-RANKL/PD-1 antibody was compared with the combination treatment with anti-RANKL and anti-PD-1 antibodies in mice bearing s.c. AT3OVA breast tumors (FIG. 38). In AT3OVA tumor-bearing mice, either anti-RANKL or anti-PD1 (100 □g) had minimal effect as monotherapies, but when combined therapies (anti-RANKL plus anti-PD1, 100 □g each) were used, a suppression of established tumor growth was observed (FIG. 38). The 100 □g and 200 □g doses of the bispecific anti-RANKL/PD-1 antibody clearly reduced s.c. tumor growth of AT3OVA compared with cIg, anti-PD-1 treatment alone or control anti-RANKL treatment alone. The lack of response to anti-PD-1 monotherapy indicates this tumor demonstrates some resistance to this immunotherapy and treatment with the single agent, bispecific anti-RANKL/PD-1 antibody overcomes this resistance. It is expected that treatment with the bispecific antibody has an in vivo inhibitory effect on AT3OVA tumor control that is greater than either antibody alone or a combination of anti-PD-1 antibody and anti-RANKL antibody. The anti-tumor effect of the 200 μg dose of the bispecific anti-RANKL/PD-1 antibody was similar to that observed with an equivalent total dose (200 μg of combined treatment with the 2 antibodies (anti-RANKL and anti-PD1, 100 μg each) (FIG. 38). These data confirm the in vivo efficacy of the bispecific anti-RANKL/PD-1 antibody in a s.c. breast tumor model.

The disclosure of every patent, patent application, and publication cited herein is hereby incorporated herein by reference in its entirety.

The citation of any reference herein should not be construed as an admission that such reference is available as “Prior Art” to the instant application.

Throughout the specification the aim has been to describe the preferred embodiments of the invention without limiting the invention to any one embodiment or specific collection of features. Those of skill in the art will therefore appreciate that, in light of the instant disclosure, various modifications and changes can be made in the particular embodiments exemplified without departing from the scope of the present invention. All such modifications and changes are intended to be included within the scope of the appended claims.

1. A method for inhibiting the development, progression or recurrence of a metastatic cancer in a subject, the method comprising administering to the subject an effective amount of a multispecific antigen-binding molecule comprising an antibody or antigen-binding fragment thereof that binds specifically to receptor activator of NF-κB ligand (RANKL) or to receptor activator of NF-κB (RANK) and at least one antibody or antigen-binding fragment thereof that binds specifically to an immune checkpoint molecule (ICM), to thereby inhibit the development, progression or recurrence of the metastatic cancer in the subject.

2. A method for treating a metastatic cancer in a subject, the method comprising administering to the subject an effective amount of a multispecific antigen-binding molecule comprising an antibody or antigen-binding fragment thereof that binds specifically to RANKL or to RANK and at least one antibody or antigen-binding fragment thereof that binds specifically to an ICM, to thereby treat the metastatic cancer in the subject.

3. The method of claim 1 or claim 2, wherein the ICM is selected from the group consisting of: programmed death 1 receptor (PD-1), programmed death ligand 1 (PD-L1), programmed death ligand 2 (PD-L2), cytotoxic T-lymphocyte-associated antigen 4 (CTLA-4), A2A adenosine receptor (AZAR), A2B adenosine receptor (A2BR), B7-H3 (CD276), V-set domain-containing T-cell activation inhibitor 1 (VTCN1), B- and T-lymphocyte attenuator (BTLA), indoleamine 2,3-dioxygenase (IDO), killer-cell immunoglobulin-like receptor (KIR), lymphocyte activation gene-3 (LAG3), T cell immunoglobulin domain and mucin domain 3 (TIM-3), V-domain Ig suppressor of T cell activation (VISTA), 5′-nucleotidase (CD73), tactile (CD96), poliovirus receptor (CD155), DNAX Accessory Molecule-1 (DNAM-1), poliovirus receptor-related 2 (CD112), cytotoxic and regulatory T-cell molecule (CRTAM), tumor necrosis factor receptor superfamily member 4 (TNFRS4; OX40; CD134), tumor necrosis factor (ligand) superfamily, member 4 (TNFSF4; OX40 ligand (OX4OL), natural killer cell receptor 2B4 (CD244), CD160, glucocorticoid-induced TNFR-related protein (GITR), glucocorticoid-induced TNFR-related protein ligand (GITRL), inducible costimulator (ICOS), galectin 9 (GAL-9), 4-1BB ligand (4-1BBL; CD137L), 4-1BB (4-1BB; CD137), CD70 (CD27 ligand (CD27L)), CD28, B7-1 (CD80), B7-2 (CD86), signal-regulatory protein (SIRP-1), integrin associated protein (IAP; CD47); B-lymphocyte activation marker (BLAST-1; CD48), natural killer cell receptor 2B4 (CD244); CD40, CD40 ligand (CD4OL), herpesvirus entry mediator (HVEM), transmembrane and immunoglobulin domain containing 2 (TMIGD2), HERV-H LTR-associating 2 (HHLA2), vascular endothelial growth inhibitor (VEGI), tumor necrosis factor receptor superfamily member 25 (TNFRS25), inducible T-cell co-stimulator ligand (ICOLG; B7RP1) and T cell immunoreceptor with Ig and ITIM (immunoreceptor tyrosine-based inhibition motif) domains (TIGIT).

4. The method of claim 1 or claim 2, wherein the ICM is selected from PD-1, PD-L1 and CTLA4.

5-10. (canceled)

11. The method of claim 1, wherein the anti-RANKL antibody or antibody fragment binds specifically to a region of RANKL that comprises the amino acid sequence TEYLQLMVY [SEQ ID NO:1] (i.e., residues 233-241 of the native RANKL sequence set forth in SEQ ID NO:2).

12. The method of claim 1, wherein the anti-RANKL antibody or antibody fragment is the MAb denosumab or an antigen-binding fragment thereof. 13-15. (Canceled)

16. The method of claim 1, wherein the anti-RANK antibody or antibody fragment binds specifically to at least a portion of a cysteine-rich domain (CRD) of RANK selected from CDR2 (i.e., residues 44-85) and CRD3 (i.e., residues 86-123), or wherein the anti-RANK antibody or antibody fragment binds specifically to one or more amino acids of the amino acid sequence: VSKTEIEEDSFRQMPTEDEYMDRPSQPTDQLLFLTEPGSKSTPP FSEPLEVGENDSLSQCFTGTQSTVGSESCNC TEPLCRTDWTPMS [SEQ ID NO:7] (i.e., residues 330-417 of the native RANK sequence set forth in SEQ ID NO:8).

17-19. (canceled)

20. The method of claim 1, wherein the anti-RANK antibody or antibody fragment competes with MAbs 64C1385, N-1H8 or N-2610 for binding to RANK.

21. (canceled)

22. The method of claim 1, wherein the anti-PD-1 antibody or antibody fragment is a monoclonal antibody (MAb) (e.g., nivolumab, pembrolizumab, pidilizumab, and MEDI-0680 (AMP-514), AMP-224, 3S001-PD-1, SHR-1210, Gendor PD-1, PDR001, CT-011, REGN2810, and BGB-317), or an antigen-binding fragment thereof; or wherein the anti-PD-L1 antibody or antibody fragment is a MAb (e.g., durvalumab (MEDI4736), atezolizumab (Tecentriq), avelumab, BMS-936559/MDX-1105, MSB0010718C, LY3300054, CA-170, GNS-1480 and MPDL3280A), or an antigen-binding fragment thereof; or
wherein the anti-CTLA4 antibody or antibody fragment is a MAb, (e.g., ipilimumab and tremelimumab), or an antigen-binding fragment thereof.

23-31. (canceled)

32. The method of any procccding claim 1, wherein one or both of the anti-RANKL or anti-RANK antibody or antibody fragment and the at least one anti-ICM antibody or antibody fragment is linked to an immunoglobulin constant chain (e.g., an IgG1, IgG2a, IgG2b, IgG3, or IgG4 constant chain).

33. (canceled)

34. The method of any procccding claim 1, wherein the multispecific antigen-binding molecule comprises an anti-RANKL antibody or antibody fragment and two or more different anti-ICM antibodies or antibody fragments.

35-44. (canceled)

45. The method of claim 1 or claim 2, wherein the multispecific antigen-binding molecule is in a FIT-Ig format which antagonizes RANKL and PD-1, the multispecific antigen-binding molecule comprising a first polypeptide, a second polypeptide and a third polypeptide, the first polypeptide comprising an amino acid sequence corresponding to SEQ ID NO:276, the second polypeptide comprising an amino acid sequence corresponding to SEQ ID NO:277, and the third polypeptide comprising an amino acid sequence corresponding to SEQ ID NO:278.

46. The method of claim 1, wherein the multispecific antigen-binding molecule is in a FIT-Ig format which antagonizes RANKL and CTLA4, the multispecific antigen-binding molecule comprising a first polypeptide, a second polypeptide and a third polypeptide, the first polypeptide comprising an amino acid sequence corresponding to SEQ ID NO:279, the second polypeptide comprising an amino acid sequence corresponding to SEQ ID NO:277, and the third polypeptide comprising an amino acid sequence corresponding to SEQ ID NO:280.

47. The method of claim 1, wherein the multispecific antigen-binding molecule of claim 54, which is in a FIT-Ig format and which antagonizes RANKL and PD-L1, the multispecific antigen-binding molecule comprising a first polypeptide, a second polypeptide and a third polypeptide, the first polypeptide comprising an amino acid sequence corresponding to SEQ ID NO:281, the second polypeptide comprising an amino acid sequence corresponding to SEQ ID NO:277, and the third polypeptide comprising an amino acid sequence corresponding to SEQ ID NO:282.

48. The method of claim 1, wherein the metastatic cancer is selected from metastatic melanoma, metastatic breast cancer, metastatic colon cancer, metastatic ovarian cancer, metastatic endometrial metastatic uterine carcinoma, metastatic gastric or stomach cancer, metastatic pancreatic cancer, metastatic prostate cancer, metastatic salivary gland cancer, metastatic lung cancer, metastatic hepatocellular cancer, metastatic glioblastoma, metastatic cervical cancer, metastatic liver cancer, metastatic bladder cancer, metastatic hepatoma, metastatic rectal cancer, metastatic colorectal cancer, metastatic kidney cancer, metastatic vulval cancer, metastatic thyroid cancer, metastatic hepatic carcinoma, metastatic anal carcinoma, metastatic penile carcinoma, metastatic testicular cancer, metastatic esophageal cancer, metastatic tumors of the biliary tract, metastatic head and neck cancer, and metastatic squamous cell carcinoma.

49. The method of claim 1, wherein the subject is resistant or has reduced or impaired responsiveness to an immunomodulatory agent, and preferably wherein the immunomodulatory agent is anti-ICM antigen-binding molecule (e.g., an anti-PD-1 or anti-PD-L1 antigen-binding molecule).

50-98. (canceled)

99. The method of claim 1, wherein the multispecific antigen-binding molecule is a complex of two or more polypeptides.

100. The method of claim 1, wherein the multispecific antigen-binding molecule is a single chain polypeptide.

101-103. (canceled)

104. The method of claim 1, wherein the RANKL antagonist and the ICM antagonist are connected by an intervening linker (e.g., a polypeptide linker).

105. (canceled)

106. The method of claim 1, wherein the antibodies or antigen-binding molecules are chimeric, humanized or human antibodies or antigen-binding fragments.

107. (canceled)


SEQUENCE LISTING (362 sequences)

SEQ ID NO: 1 - skipped

SEQ ID NO: 2 - protein, 317 aa, Homo sapiens
MRRASRDYTKYLRGSEEMGGGPGAPHEGPLHAPPPPAPHQPPAASRSMFVALLGLGLGQVVCSVALFFYFRAQMDPNRISEDGTHCIYRILRLHENADFQDTTLESQDTKLIPDSCRRIKQAFQGAVQKELQHIVGSQHIRAEKAMVDGSWLDLAKRSKLEAQPFAHLTINATDIPSGSHKVSLSSWYHDRGWAKISNMTFSNGKLIVNQDGFYYLYANICFRHHETSGDLATEYLQLMVYVTKTSIKIPSSHTLMKGGSTKYWSGNSEFHFYSINVGGFFKLRSGEEISIEVSNPSLLDPDQDATYFGAFKVRDID

SEQ ID NO: 3 - protein, 452 aa, Homo sapiens
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - protein, 215 aa, Homo sapiens
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 5 - protein, 11 aa, Artificial Sequence
  RANK CDR3 mimetic antagonist
YCWNSDCECCY

SEQ ID NO: 6 - skipped

SEQ ID NO: 7 - protein, 88 aa, Homo sapiens
VSKTEIEEDSFRQMPTEDEYMDRPSQPTDQLLFLTEPGSKSTPPFSEPLEVGENDSLSQCFTGTQSTVGSESCNCTEPLCRTDWTPMS

SEQ ID NO: 8 - protein, 616 aa, Homo sapiens
MAPRARRRRPLFALLLLCALLARLQVALQIAPPCTSEKHYEHLGRCCNKCEPGKYMSSKCTTTSDSVCLPCGPDEYLDSWNEEDKCLLHKVCDTGKALVAVVAGNSTTPRRCACTAGYHWSQDCECCRRNTECAPGLGAQHPLQLNKDTVCKPCLAGYFSDAFSSTDKCRPWTNCTFLGKRVEHHGTEKSDAVCSSSLPARKPPNEPHVYLPGLIILLLFASVALVAAIIFGVCYRKKGKALTANLWHWINEACGRLSGDKESSGDSCVSTHTANFGQQGACEGVLLLTLEEKTFPEDMCYPDQGGVCQGTCVGGGPYAQGEDARMLSLVSKTEIEEDSFRQMPTEDEYMDRPSQPTDQLLFLTEPGSKSTPPFSEPLEVGENDSLSQCFTGTQSTVGSESCNCTEPLCRTDWTPMSSENYLQKEVDSGHCPHWAASPSPNWADVCTGCRNPPGEDCEPLVGSPKRGPLPQCAYGMGLPPEEEASRTEARDQPEDGADGRLPSSARAGAGSGSSPGGQSPASGNVTGNSNSTFISSGQVMNFKGDIIVVYVSQTSQEGAAAAAEPMGRPVQEETLARRDSFAGNGPRFPDPCGGPEGLREPEKASRPVQEQGGAKA

SEQ ID NO: 9 - protein, 25 aa, Homo sapiens
SFVLNWYRMSPSNQTDKLAAFPEDR

SEQ ID NO: 10 - protein, 268 aa, Homo sapiens
PGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 11 - protein, 19 aa, Homo sapiens
SGTYLCGAISLAPKAQIKE

SEQ ID NO: 12 - protein, 32 aa, Homo sapiens
NWYRMSPSNQTDKLAAFPEDRSQPGQDCRFRV

SEQ ID NO: 13 - protein, 12 aa, Homo sapiens
SKKQSDTHLEET

SEQ ID NO: 14 - protein, 290 aa, Homo sapiens
MRIFAVFIFMTYWHLLNAFTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPELPLAHPPNERTHLVILGAILLCLGVALTFIFRLRKGRMMDVKKCGIQDTNSKKQSDTHLEET

SEQ ID NO: 15 - protein, 18 aa, Homo sapiens
YASPGKATEVRVTVLRQA

SEQ ID NO: 16 - protein, 188 aa, Homo sapiens
KAMHVAQPAVVLASSRGIASFVCEYASPGKATEVRVTVLRQADSQVTEVCAATYMMGNELTFLDDSICTGTSSGNQVNLTIQGLRAMDTGLYICKVELMYPPPYYLGIGNGTQIYVIDPEPCPDSDFLLWILAAVSSGLFFYSFLLTAVSLSKMLKKRSPLTTGVYVKMPPTEPECEKQFQPYFIPIN

SEQ ID NO: 17 - protein, 23 aa, Homo sapiens
DSQVTEVCAATYMMGNELTFLDD

SEQ ID NO: 18 - protein, 14 aa, Homo sapiens
VELMYPPPYYLGIG

SEQ ID NO: 19 - skipped

SEQ ID NO: 20 - protein, 17 aa, Homo sapiens
GITGSGGSTYYADSVKG

SEQ ID NO: 21 - protein, 13 aa, Homo sapiens
DPGTTVIMSWFDP

SEQ ID NO: 22 - protein, 12 aa, Homo sapiens
RASQSVRGRYLA

SEQ ID NO: 23-24 - skipped (2 entries)

SEQ ID NO: 25 - protein, 122 aa, Homo sapiens
EVQLLESGGGLVQPGGSLRLSCAASGFTFSSYAMSWVRQAPGKGLEWVSGITGSGGSTYYADSVKGRFTISRDNSKNTLYLQMNSLRAEDTAVYYCAKDPGTTVIMSWFDPWGQGTLVTVSS

SEQ ID NO: 26 - protein, 108 aa, Homo sapiens
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIK

SEQ ID NO: 27 - protein, 467 aa, Homo sapiens
  MISC_FEATURE (18)..(18): X is O
  MISC_FEATURE (109)..(109): X is O
  MISC_FEATURE (129)..(129): X is O
MEFGLSWLFLVAILKGVXCEVQLLESGGGLVQPGGSLRLSCAASGFTFSSYAMSWVRQAPGKGLEWVSGITGSGGSTYYADSVKGRFTISRDNSKNTLYLQMNSLRAEXTAVYYCAKDPGTTVIMSWFXPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 28 - protein, 235 aa, Homo sapiens
  MISC_FEATURE (6)..(6): X is O
METPAXLLFLLLLWLPDTTGEIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 29 - skipped

SEQ ID NO: 30 - protein, 17 aa, Homo sapiens
WINAGNGNTKFSQKFQG

SEQ ID NO: 31 - protein, 17 aa, Homo sapiens
DSSNMVRGIIIAYYFDY

SEQ ID NO: 32 - protein, 11 aa, Homo sapiens
RASQSISRYLN

SEQ ID NO: 33-34 - skipped (2 entries)

SEQ ID NO: 35 - protein, 230 aa, Homo sapiens
AQVQLVQSGAEVRKPGASVKVSCKASGYDFSNYAIHWVRQAPGQRLEWMGWINAGNGNTKFSQKFQGRITVTRDTAASTAYMELRSLRSEDTAVYYCARDSSNMVRGIIIAYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 36 - protein, 126 aa, Homo sapiens
QVQLVQSGAEVRKPGASVKVSCKASGYDFSNYAIHWVRQAPGQRLEWMGWINAGNGNTKFSQKFQGRITVTRDTAASTAYMELRSLRSEDTAVYYCARDSSNMVRGIIIAYYFDYWGQGTLVTVSS

SEQ ID NO: 37 - protein, 215 aa, Homo sapiens
SHSALEIVMTQSPSSLSASVGDRVTITCRASQSISRYLNWYQLKPGKAPRLLIYGASSLQSGVPSRFSGSGSGAEFTLTISSLQPEDIATYYCQHTRAFGQGTKVEIKRTVAAPSVFIFPPSDEQLKSGTASVVCLLNNFYPREAKVQWKVDNALQSGNSQESATEQDSKDSTYSLSSTLTLSKADYEKHKVYACEVTHQGLSSPVTKSFNRGEC

SEQ ID NO: 38 - protein, 103 aa, Homo sapiens
EIVMTQSPSSLSASVGDRVTITCRASQSISRYLNWYQLKPGKAPRLLIYGASSLQSGVPSRFSGSGSGAEFTLTISSLQPEDIATYYCQHTRAFGQGTKVEIK

SEQ ID NO: 39 - skipped

SEQ ID NO: 40 - protein, 17 aa, Homo sapiens
WINAGNGNTKFSQKFQG

SEQ ID NO: 41 - protein, 17 aa, Homo sapiens
DSSNMVRGIIIAYYFDY

SEQ ID NO: 42 - protein, 11 aa, Homo sapiens
RASQSVGSYLA

SEQ ID NO: 43-44 - skipped (2 entries)

SEQ ID NO: 45 - protein, 230 aa, Homo sapiens
AEVQLVQSGAEVRKPGASVKVSCKASGYDFSNYAIHWVRQAPGQRLEWMGWINAGNGNTKFSQKFQGRITVTRDTAASTAYMELRSLRSEDTAVYYCARDSSNMVRGIIIAYYFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 46 - protein, 126 aa, Homo sapiens
EVQLVQSGAEVRKPGASVKVSCKASGYDFSNYAIHWVRQAPGQRLEWMGWINAGNGNTKFSQKFQGRITVTRDTAASTAYMELRSLRSEDTAVYYCARDSSNMVRGIIIAYYFDYWGQGTLVTVSS

SEQ ID NO: 47 - protein, 215 aa, Homo sapiens
SHSALEIVLTQSPATLSFSPGERATLSCRASQSVGSYLAWYQQRPGQAPRPLIYDATNRATGIPTRFSGSGSGTDFTLTISSLEPEDFATYYCQHRRTFGRGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 48 - protein, 103 aa, Homo sapiens
EIVLTQSPATLSFSPGERATLSCRASQSVGSYLAWYQQRPGQAPRPLIYDATNRATGIPTRFSGSGSGTDFTLTISSLEPEDFATYYCQHRRTFGRGTKLEIK

SEQ ID NO: 49 - protein, 11 aa, Homo sapiens
RASQSISRYLN

SEQ ID NO: 50 - protein, 11 aa, Homo sapiens
RASQSVGSYLA

SEQ ID NO: 51 - protein, 12 aa, Homo sapiens
RASQSVSSSSLA

SEQ ID NO: 52-59 - skipped (8 entries)

SEQ ID NO: 60 - protein, 11 aa, Homo sapiens
QSTDSSGTYVV

SEQ ID NO: 61-63 - skipped (3 entries)

SEQ ID NO: 64 - protein, 17 aa, Homo sapiens
WINAGNGNTKFSQKFQG

SEQ ID NO: 65 - protein, 17 aa, Homo sapiens
VISYDGNNKYYADSVKG

SEQ ID NO: 66 - protein, 17 aa, Homo sapiens
  MISC_FEATURE (17)..(17): X is O
GISMNSGRIGYADSVKX

SEQ ID NO: 67 - protein, 17 aa, Homo sapiens
DSSNMVRGIIIAYYFDY

SEQ ID NO: 68 - skipped

SEQ ID NO: 69 - protein, 14 aa, Homo sapiens
GGSTSARYSSGWYY

SEQ ID NO: 70-72 - skipped (3 entries)

SEQ ID NO: 73 - protein, 11 aa, Artificial Sequence
  Newa et al. anti-RANKL antibody CDR1 (VL)
RASQSISSYLN

SEQ ID NO: 74-75 - skipped (2 entries)

SEQ ID NO: 76 - protein, 115 aa, Artificial Sequence
  Newa et al. anti-RANKL antibody VH
MAEVQLLESGGGLVQPGGSLRLSCAASGFTFSSYAMSWVRQAPGKGLEWVSAISGDGYYTDYADSVGRFTISRDNSKNTLYLQNSLRAEDTAVYYCAKNAYSFDYWGQGTLVTVS

SEQ ID NO: 77 - protein, 116 aa, Artificial Sequence
  Newa et al. anti-RANKL antibody VH
MAEVQLLESGGGLVQPGGSLRLSCAASGFTFSSYAMSWVRQAPGKGLEWVSAISGDGYYTDYADSVGRFTISRDNSKNTLYLQMNSLRAEDTAVYYCAKNAYSFDYWGQGTLVTVS

SEQ ID NO: 78 - protein, 113 aa, Artificial Sequence
  Antigen-binding fragment of SEQ ID NO:76
EVQLLESGGGLVQPGGSLRLSCAASGFTFSSYAMSWVRQAPGKGLEWVSAISGDGYYTDYADSVGRFTISRDNSKNTLYLQNSLRAEDTAVYYCAKNAYSFDYWGQGTLVTVS

SEQ ID NO: 79 - protein, 114 aa, Artificial Sequence
  Antigen-binding fragment of SEQ ID NO:77
EVQLLESGGGLVQPGGSLRLSCAASGFTFSSYAMSWVRQAPGKGLEWVSAISGDGYYTDYADSVGRFTISRDNSKNTLYLQMNSLRAEDTAVYYCAKNAYSFDYWGQGTLVTVS

SEQ ID NO: 80 - protein, 111 aa, Artificial Sequence
  Newa et al. anti-RANKL antibody VL
DIQMTQSPSSLSASVGDRVTITCRASQSISSYLNWYQQKPGKAPKLLIYYASSLQSGVPSRFSGSGSGTDFTLTISSLQPEDFATYYCQQGSSSPNTFGQGTKVEIKRAAA

SEQ ID NO: 81 - protein, 107 aa, Artificial Sequence
  Antigen-binding fragment of SEQ ID NO:80
DIQMTQSPSSLSASVGDRVTITCRASQSISSYLNWYQQKPGKAPKLLIYYASSLQSGVPSRFSGSGSGTDFTLTISSLQPEDFATYYCQQGSSSPNTFGQGTKVEIK

SEQ ID NO: 82 - skipped

SEQ ID NO: 83 - protein, 17 aa, Artificial Sequence
  Nivolumab CDR2 (VH)
VIWYDGSKRYYADSVKG

SEQ ID NO: 84 - skipped

SEQ ID NO: 85 - protein, 11 aa, Artificial Sequence
  Nivolumab CDR1 (VL)
RASQSVSSYLA

SEQ ID NO: 86-87 - skipped (2 entries)

SEQ ID NO: 88 - protein, 440 aa, Artificial Sequence
  Nivolumab heavy chain
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 89 - protein, 113 aa, Artificial Sequence [sequence omitted]

SEQ ID NO: 90 - protein, 214 aa, Artificial Sequence
  Nivolumab light chain
EIVLTQSPATLSLSPGERATLSCRASQSVSSYLAWYQQKPGQAPRLLIYDASNRATGIPARFSGSGSGTDFTLTISSLEPEDFAVYYCQQSSNWPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 91 - protein, 107 aa, Artificial Sequence [sequence omitted]

SEQ ID NO: 92 - skipped

SEQ ID NO: 93 - protein, 17 aa, Artificial Sequence
  Pembrolizumab CDR2 (VH)
GINPSNGGTNFNEKFKN

SEQ ID NO: 94 - protein, 11 aa, Artificial Sequence
  Pembrolizumab CDR3 (VH)
RDYRFDMGFDY

SEQ ID NO: 95 - protein, 15 aa, Artificial Sequence
  Pembrolizumab CDR1 (VL)
RASKGVSTSGYSYLH

SEQ ID NO: 96-97 - skipped (2 entries)

SEQ ID NO: 98 - protein, 447 aa, Artificial Sequence
  Pembrolizumab heavy chain
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 99 - protein, 120 aa, Artificial Sequence [sequence omitted]

SEQ ID NO: 100 - protein, 218 aa, Artificial Sequence
  Pembrolizumab light chain
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 101 - protein, 111 aa, Artificial Sequence [sequence omitted]

SEQ ID NO: 102 - skipped

SEQ ID NO: 103 - protein, 17 aa, Artificial Sequence
  Pidilizumab CDR2 (VH)
WINTDSGESTYAEEFKG

SEQ ID NO: 104 - skipped

SEQ ID NO: 105 - protein, 10 aa, Artificial Sequence
  Pidilizumab CDR1 (VL)
SARSSVSYMH

SEQ ID NO: 106-107 - skipped (2 entries)

SEQ ID NO: 108 - protein, 447 aa, Artificial Sequence
  Pidilizumab heavy chain
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 117 aa, Artificial Sequence
  Pidilizumab VH
QVQLVQSGSELKKPGASVKISCKASGYTFTNYGMNWVRQAPGQGLQWMGWINTDSGESTYAEEFKGRFVFSLDTSVNTAYLQITSLTAEDTGMYFCVRVGYDALDYWGQGTLVTVSS

SEQ ID NO: 110 - protein, 213 aa, Artificial Sequence
  Pidilizumab light chain
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 111 - protein, 106 aa, Artificial Sequence
  Pidilizumab VL
EIVLTQSPSSLSASVGDRVTITCSARSSVSYMHWFQQKPGKAPKLWIYRTSNLASGVPSRFSGSGSGTSYCLTINSLQPEDFATYYCQQRSSFPLTFGGGTKLEIK

SEQ ID NO: 112 - protein, 15 aa, Artificial Sequence
  WO2015/026634 anti-PD-1 CDR1 (VL)
RASKSVSTSGFSYLH

SEQ ID NO: 113-115 - skipped (3 entries)

SEQ ID NO: 116 - protein, 17 aa, Artificial Sequence
  WO2015/026634 anti-PD-1 CDR2 (VH)
GVNPSNGGTNFSEKFKS

SEQ ID NO: 117 - protein, 11 aa, Artificial Sequence
  WO2015/026634 anti-PD-1 CDR3 (VH)
RDSNYDGGFDY

SEQ ID NO: 118 - protein, 15 aa, Artificial Sequence
  WO2015/026634 anti-PD-1 CDR1 (VL)
RASKGVSTSGYSYLH

SEQ ID NO: 119-121 - skipped (3 entries)

SEQ ID NO: 122 - protein, 17 aa, Artificial Sequence
  WO2015/026634 anti-PD-1 CDR2 (VH)
GINPSNGGTNFNEKFKN

SEQ ID NO: 123 - protein, 11 aa, Artificial Sequence
  WO2015/026634 anti-PD-1 CDR3 (VH)
RDYRFDMGFDY

SEQ ID NO: 124 - protein, 120 aa, Artificial Sequence [sequence omitted]

SEQ ID NO: 125 - protein, 111 aa, Artificial Sequence [sequence omitted]

SEQ ID NO: 126 - protein, 110 aa, Artificial Sequence
  WO2015/026634 anti-PD-1 VL
IVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIK

SEQ ID NO: 127 - protein, 111 aa, Artificial Sequence
  WO2015/026634 anti-PD-1 VL
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIK

SEQ ID NO: 128 - protein, 447 aa, Artificial Sequence; identical to SEQ ID NO: 98
  WO2015/026634 anti-PD-1 heavy chain

SEQ ID NO: 129 - protein, 218 aa, Artificial Sequence; identical to SEQ ID NO: 100
  WO2015/026634 anti-PD-1 light chain

SEQ ID NO: 130 - protein, 218 aa, Artificial Sequence
  WO2015/026634 anti-PD-1 light chain
EIVLTQSPLSLPVTPGEPASISCRASKGVSTSGYSYLHWYLQKPGQSPQLLIYLASYLESGVPDRFSGSGSGTDFTLKISRVEAEDVGVYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 218 aa, Artificial Sequence
  WO2015/026634 anti-PD-1 light chain
DIVMTQTPLSLPVTPGEPASISCRASKGVSTSGYSYLHWYLQKPGQSPQLLIYLASYLESGVPDRFSGSGSGTAFTLKISRVEAEDVGLYYCQHSRDLPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - skipped

SEQ ID NO: 133 - protein, 16 aa, Artificial Sequence
  Durvalumab CDR2 (VH)
NIKQDGSEKYYVDSVK

SEQ ID NO: 134 - protein, 12 aa, Artificial Sequence
  Durvalumab CDR3 (VH)
EGGWFGELAFDY

SEQ ID NO: 135 - protein, 12 aa, Artificial Sequence
  Durvalumab CDR1 (VL)
RASQRVSSSYLA

SEQ ID NO: 136 - protein, 11 aa, Artificial Sequence
  Durvalumab CDR2 (VL)
DASSRATGIPD

SEQ ID NO: 137 - skipped

SEQ ID NO: 138 - protein, 449 aa, Artificial Sequence
  Durvalumab heavy chain
VQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 139 - protein, 120 aa, Artificial Sequence
  Durvalumab VH
VQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 140 - protein, 215 aa, Artificial Sequence
  Durvalumab light chain
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 108 aa, Artificial Sequence
  Durvalumab VL
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 142 - protein, 10 aa, Artificial Sequence
  Atezolizumab CDR1 (VH)
  MISC_FEATURE (6)..(6): X is D or G
GFTFSXSWIH

SEQ ID NO: 143 - protein, 18 aa, Artificial Sequence
  Atezolizumab CDR2 (VH)
  MISC_FEATURE (4)..(4): X is S or L
  MISC_FEATURE (10)..(10): X is T or S
AWIXPYGGSXYYADSVKG

SEQ ID NO: 144 - skipped

SEQ ID NO: 145 - protein, 11 aa, Artificial Sequence
  Atezolizumab CDR1 (VL)
  MISC_FEATURE (5)..(5): X is D or V
  MISC_FEATURE (6)..(6): X is V or I
  MISC_FEATURE (7)..(7): X is S or N
  MISC_FEATURE (9)..(9): X is A or F
  MISC_FEATURE (10)..(10): X is V or L
RASQXXXTXXA

SEQ ID NO: 146-147 - skipped (2 entries)

SEQ ID NO: 148 - protein, 448 aa, Artificial Sequence
  Atezolizumab heavy chain
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 149 - protein, 118 aa, Artificial Sequence [sequence omitted]

SEQ ID NO: 150 - protein, 214 aa, Artificial Sequence
  Atezolizumab light chain
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 151 - protein, 107 aa, Artificial Sequence [sequence omitted]

SEQ ID NO: 152 - skipped

SEQ ID NO: 153 - protein, 17 aa, Artificial Sequence
  Avelumab CDR2 (VH)
  MISC_FEATURE (8)..(8): X is F or I
  MISC_FEATURE (14)..(14): X is S or T
SIYPSGGXTFYADXVKG

SEQ ID NO: 154 - protein, 11 aa, Artificial Sequence
  Avelumab CDR3 (VH)
  MISC_FEATURE (10)..(10): X is E or D
IKLGTVTTVXY

SEQ ID NO: 155 - protein, 14 aa, Artificial Sequence
  Avelumab CDR1 (VL)
  MISC_FEATURE (4)..(4): X is N or S
  MISC_FEATURE (5)..(5): X is T, R, or S
  MISC_FEATURE (9)..(9): X is A or G
TGTXXDVGXYNYVS

SEQ ID NO: 156-157 - skipped (2 entries)

SEQ ID NO: 158 - protein, 450 aa, Artificial Sequence
  Avelumab heavy chain
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 159 - protein, 120 aa, Artificial Sequence
  Avelumab VH
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 160 - protein, 216 aa, Artificial Sequence
  Avelumab light chain
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 161 - protein, 110 aa, Artificial Sequence
  Avelumab VL
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 162 - skipped

SEQ ID NO: 163 - protein, 17 aa, Homo sapiens
FISYDGNNKYYADSVKG

SEQ ID NO: 164 - skipped

SEQ ID NO: 165 - protein, 12 aa, Homo sapiens
RASQSVGSSYLA

SEQ ID NO: 166-167 - skipped (2 entries)

SEQ ID NO: 168 - protein, 448 aa, Homo sapiens
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 169 - protein, 118 aa, Homo sapiens
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 170 - protein, 215 aa, Homo sapiens
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 171 - protein, 108 aa, Homo sapiens
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 172 - protein, 10 aa, Homo sapiens
GFTFSSYGMH

SEQ ID NO: 173 - protein, 15 aa, Homo sapiens
VIWYDGSNKYYADSV

SEQ ID NO: 174 - protein, 16 aa, Homo sapiens
DPRGATLYYYYYGMDV

SEQ ID NO: 175 - protein, 11 aa, Homo sapiens
RASQSINSYLD

SEQ ID NO: 176-177 - skipped (2 entries)

SEQ ID NO: 178 - protein, 451 aa, Homo sapiens
QVQLVESGGGVVQPGRSLRLSCAASGFTFSSYGMHWVRQAPGKGLEWVAVIWYDGSNKYYADSVKGRFTISRDNSKNTLYIQMNSLRAEDTAVYYCARDPRGATLYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 179 - protein, 118 aa, Homo sapiens; identical to SEQ ID NO: 169

SEQ ID NO: 180 - protein, 214 aa, Homo sapiens
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 181 - protein, 107 aa, Homo sapiens
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIK

SEQ ID NO: 182 - protein, 534 aa, Homo sapiens
MLRRRGSPGMGVHVGAALGALWFCLTGALEVQVPEDPVVALVGTDATLCCSFSPEPGFSLAQLNLIWQLTDTKQLVHSFAEGQDQGSAYANRTALFPDLLAQGNASLRLQRVRVADEGSFTCFVSIRDFGSAAVSLQVAAPYSKPSMTLEPNKDLRPGDTVTITCSSYQGYPEAEVFWQDGQGVPLTGNVTTSQMANEQGLFDVHSILRVVLGANGTYSCLVRNPVLQQDAHSSVTITPQRSPTGAVEVQVPEDPVVALVGTDATLRCSFSPEPGFSLAQLNLIWQLTDTKQLVHSFTEGRDQGSAYANRTALFPDLLAQGNASLRLQRVRVADEGSFTCFVSIRDFGSAAVSLQVAAPYSKPSMTLEPNKDLRPGDTVTITCSSYRGYPEAEVFWQDGQGVPLTGNVTTSQMANEQGLFDVHSVLRVVLGANGTYSCLVRNPVLQQDAHGSVTITGQPMTFPPEALWVTVGLSVCLIALLVALAFVCWRKIKQSCEEENAGAEDQDGEGEGSKTALQPLKHSDSKEDDGQEIA

SEQ ID NO: 183 - skipped

SEQ ID NO: 184 - protein, 16 aa, Artificial Sequence
  Enoblituzumab CDR2 (VH)
YISSDSSAIYYADTVK

SEQ ID NO: 185 - protein, 13 aa, Artificial Sequence
  Enoblituzumab CDR3 (VH)
GRENIYYGSRLDY

SEQ ID NO: 186 - protein, 11 aa, Artificial Sequence
  Enoblituzumab CDR1 (VL)
KASQNVDTNVA

SEQ ID NO: 187-188 - skipped (2 entries)

SEQ ID NO: 189 - protein, 451 aa, Artificial Sequence
  Enoblituzumab heavy chain
VQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELVGGPSVFLLPPKPKDTLMISRTPEVTCVVVDVSHEDPEVKFNWYVDGVEVHNAKTKPPEEQYNSTLRVVSVLTVLHQDWLNGKEYKCKVSNKALPAPIEKTISKAKGQPREPQVYTLPPSREEMTKNQVSLTCLVKGFYPSDIAVEWESNGQPENNYKTTPLVLDSDGSFFLYSKLTVDKSRWQQGNVFSCSVMHEALHNHYTQKSLSLSPGK

SEQ ID NO: 190 - protein, 121 aa, Artificial Sequence
  Enoblituzumab VH
VQLVESGGGLVQPGGSLRLSCAASGFTFSSFGMHWVRQAPGKGLEWVAYISSDSSAIYYADTVKGRFTISRDNAKNSLYLQMNSLRDEDTAVYYCGRGRENIYYGSRLDYWGQGTTVTVSS

SEQ ID NO: 191 - protein, 214 aa, Artificial Sequence
  Enoblituzumab light chain
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 192 - protein, 107 aa, Artificial Sequence
  Enoblituzumab VL
DIQLTQSPSFLSASVGDRVTITCKASQNVDTNVAWYQQKPGKAPKALIYSASYRYSGVPSRFSGSGSGTDFTLTISSLQPEDFATYYCQQYNNYPFTFGQGTKLEIK

SEQ ID NO: 193 - protein, 403 aa, Homo sapiens [sequence omitted]

SEQ ID NO: 194 - protein, 327 aa, Homo sapiens
HEGVHRKPSLLAHPGPLVKSEETVILQCWSDVMFEHFLLHREGMFNDTLRLIGEHHDGVSKANFSISRMTQDLAGTYRCYGSVTHSPYQVSAPSDPLDIVIIGLYEKPSLSAQPGPTVLAGENVTLSCSSRSSYDMYHLSREGEAHERRLPAGPKVNGTFQADFPLGPATHGGTYRCFGSFHDSPYEWSKSSDPLLVSVTGNPSNSWPSPTEPSSKTGNPRHLHILIGTSVVIILFILLFFLLHRWCSNKKNAAVMDQESAGNRTANSEDSDEQDPQEVTYTQLNHCVFTQRKITRPSQRPKTPPTDIIVYTELPNAESRSKVVSCP

SEQ ID NO: 195 - skipped

SEQ ID NO: 196 - protein, 16 aa, Artificial Sequence
  Lirilumab CDR2 (VH)
GFIPIFGAANYAQKFQ

SEQ ID NO: 197 - protein, 14 aa, Artificial Sequence
  Lirilumab CDR3 (VH)
IPSGSYYYDYDMDV

SEQ ID NO: 198 - protein, 11 aa, Artificial Sequence
  Lirilumab CDR1 (VL)
RASQSVSSYLA

SEQ ID NO: 199-200 - skipped (2 entries)

SEQ ID NO: 201 - protein, 450 aa, Artificial Sequence
  Lirilumab heavy chain
QVQLVQSGAEVKKPGSSVKVSCKASGGTFSFYAISWVRQAPGQGLEWMGGFIPIFGAANYAQKFQGRVTITADESTSTAYMELSSLRSDDTAVYYCARIPSGSYYYDYDMDVWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 202 - protein, 123 aa, Artificial Sequence
  Lirilumab VH
QVQLVQSGAEVKKPGSSVKVSCKASGGTFSFYAISWVRQAPGQGLEWMGGFIPIFGAANYAQKFQGRVTITADESTSTAYMELSSLRSDDTAVYYCARIPSGSYYYDYDMDVWGQGTTVTVSS

SEQ ID NO: 203 - protein, 214 aa, Artificial Sequence
  Lirilumab light chain
EIVLTQSPVTLSLSPGERATLSCRASQSVSSYLAWYQQKPGQAPRLLIYDASNRATGIPARFSGSGSGTDFTLTISSLEPEDFAVYYCQQRSNWMYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 204 - protein, 109 aa, Artificial Sequence
  Lirilumab VL
EIVLTQSPVTLSLSPGERATLSCRASQSVSSYLAWYQQKPGQAPRLLIYDASNRATGIPARFSGSGSGTDFTLTISSLEPEDFAVYYCQQRSNWMYTFGQGTKLEIKRT

SEQ ID NO: 205 - protein, 503 aa, Homo sapiens
LQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 206 - skipped

SEQ ID NO: 207 - protein, 16 aa, Artificial Sequence
  BMS-986016 CDR2 (VH)
EINHRGSTNSNPSLKS

SEQ ID NO: 208 - protein, 12 aa, Artificial Sequence
  BMS-986016 CDR3 (VH)
GYSDYEYNWFDP

SEQ ID NO: 209 - protein, 11 aa, Artificial Sequence
  BMS-986016 CDR1 (VL)
RASQSISSYLA

SEQ ID NO: 210-211 - skipped (2 entries)

SEQ ID NO: 212 - protein, 447 aa, Artificial Sequence
  BMS-986016 heavy chain
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 213 - protein, 120 aa, Artificial Sequence
  BMS-986016 VH
QVQLQQWGAGLLKPSETLSLTCAVYGGSFSDYYWNWIRQPPGKGLEWIGEINHRGSTNSNPSLKSRVTLSLDTSKNQFSLKLRSVTAADTAVYYCAFGYSDYEYNWFDPWGQGTLVTVSS

SEQ ID NO: 214 - protein, 214 aa, Artificial Sequence
  BMS-986016 light chain
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 215 - protein, 107 aa, Artificial Sequence
  BMS-986016 VL
EIVLTQSPATLSLSPGERATLSCRASQSISSYLAWYQQKPGQAPRLLIYDASNRATGIPARFSGSGSGTDFTLTISSLEPEDFAVYYCQQRSNWPLTFGQGTNLEIK

SEQ ID NO: 216 - protein, 122 aa, Artificial Sequence; identical to SEQ ID NO: 25
  Denosumab VH

SEQ ID NO: 217 - protein, 113 aa, Artificial Sequence [sequence omitted]

SEQ ID NO: 218 - protein, 126 aa, Artificial Sequence; identical to SEQ ID NO: 36
  VH of anti-RANKL antibody disclosed in EP 1257648

SEQ ID NO: 219 - protein, 113 aa, Artificial Sequence [sequence omitted]

SEQ ID NO: 220 - protein, 122 aa, Artificial Sequence; identical to SEQ ID NO: 25
  Denosumab VH

SEQ ID NO: 221 - protein, 120 aa, Artificial Sequence [sequence omitted]

SEQ ID NO: 222 - protein, 126 aa, Artificial Sequence; identical to SEQ ID NO: 36
  VH of anti-RANKL antibody disclosed in EP 1257648

SEQ ID NO: 223 - protein, 120 aa, Artificial Sequence [sequence omitted]

SEQ ID NO: 224 - protein, 122 aa, Artificial Sequence; identical to SEQ ID NO: 25
  Denosumab VH

SEQ ID NO: 225 - protein, 120 aa, Artificial Sequence; identical to SEQ ID NO: 139
  Durvalumab VH

SEQ ID NO: 226 - protein, 126 aa, Artificial Sequence; identical to SEQ ID NO: 36
  VH of anti-RANKL antibody disclosed in EP 1257648

SEQ ID NO: 227 - protein, 120 aa, Artificial Sequence; identical to SEQ ID NO: 139
  Durvalumab VH

SEQ ID NO: 228 - protein, 122 aa, Artificial Sequence; identical to SEQ ID NO: 25
  Denosumab VH

SEQ ID NO: 229 - protein, 118 aa, Artificial Sequence [sequence omitted]

SEQ ID NO: 230 - protein, 126 aa, Artificial Sequence; identical to SEQ ID NO: 36
  VH of anti-RANKL antibody disclosed in EP 1257648

SEQ ID NO: 231 - protein, 118 aa, Artificial Sequence [sequence omitted]

SEQ ID NO: 232 - protein, 122 aa, Artificial Sequence; identical to SEQ ID NO: 25
  Denosumab VH

SEQ ID NO: 233 - protein, 118 aa, Artificial Sequence; identical to SEQ ID NO: 169
  Ipilimumab VH

SEQ ID NO: 234 - protein, 126 aa, Artificial Sequence; identical to SEQ ID NO: 36
  VH of anti-RANKL antibody disclosed in EP 1257648

SEQ ID NO: 235 - protein, 118 aa, Artificial Sequence; identical to SEQ ID NO: 169
  Ipilimumab VH

SEQ ID NO: 236 - protein, 122 aa, Artificial Sequence; identical to SEQ ID NO: 25
  Denosumab VH

SEQ ID NO: 237 - protein, 118 aa, Artificial Sequence; identical to SEQ ID NO: 169
  Tremelimumab VH

SEQ ID NO: 238 - protein, 126 aa, Artificial Sequence; identical to SEQ ID NO: 36
  VH of anti-RANKL antibody disclosed in EP 1257648

SEQ ID NO: 239 - protein, 118 aa, Artificial Sequence; identical to SEQ ID NO: 169
  Tremelimumab VH

SEQ ID NO: 240 - protein, 476 aa, Artificial Sequence
  Denosumab CrossMAb CH1-CL huIgG2 KNOB mutation, heavy chain
  MISC_FEATURE (18)..(18): X is O
  MISC_FEATURE (109)..(109): X is O
  MISC_FEATURE (129)..(129): X is O
MEFGLSWLFLVAILKGVXCEVQLLESGGGLVQPGGSLRLSCAASGFTFSSYAMSWVRQAPGKGLEWVSGITGSGGSTYYADSVKGRFTISRDNSKNTLYLQMNSLRAEXTAVYYCAKDPGTTVIMSWFXPWGQGTLVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLWCLVKGFYPSDIAVEWESNGQPENNYKTTPPMLDSDGSFFLYSKLTVDKSRWQQGNVFSCSVMHEALHNHYTQKSLSLSPGK

SEQ ID NO: 241 - protein, 226 aa, Artificial Sequence
  Denosumab CrossMAb CH1-CL light chain
  MISC_FEATURE (6)..(6): X is O
METPAXLLFLLLLWLPDTTGEIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 242 - protein, 439 aa, Homo sapiens
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLASKLTVDKSRWQQGNVFSCSVMHEALHNHYTQKSLSLSPGK

SEQ ID NO: 243 - protein, 214 aa, Homo sapiens; identical to SEQ ID NO: 90

SEQ ID NO: 244 - protein, 453 aa, Artificial Sequence
  Denosumab CrossMAb VH-VL huIgG2 KNOB mutation, heavy chain
  MISC_FEATURE (18)..(18): X is O
MEFGLSWLFLVAILKGVXCEIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLWCLVKGFYPSDIAVEWESNGQPENNYKTTPPMLDSDGSFFLYSKLTVDKSRWQQGNVFSCSVMHEALHNHYTQKSLSLSPGK

SEQ ID NO: 245 - protein, 249 aa, Artificial Sequence
  Denosumab CrossMAb VH-VL light chain
  MISC_FEATURE (6)..(6): X is O
  MISC_FEATURE (110)..(110): X is O
  MISC_FEATURE (130)..(130): X is O
METPAXLLFLLLLWLPDTTGEVQLLESGGGLVQPGGSLRLSCAASGFTFSSYAMSWVRQAPGKGLEWVSGITGSGGSTYYADSVKGRFTISRDNSKNTLYLQMNSLRAEXTAVYYCAKDPGTTVIMSWFXPWGQGTLVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 246 - protein, 439 aa, Artificial Sequence; identical to SEQ ID NO: 242
  Nivolumab IgG2 Hole mutation, heavy chain

SEQ ID NO: 247 - protein, 214 aa, Homo sapiens; identical to SEQ ID NO: 90

SEQ ID NO: 248 - protein, 462 aa, Artificial Sequence
  Denosumab CrossMAb Fab huIgG2 KNOB mutation, heavy chain
  MISC_FEATURE (18)..(18): X is O
MEFGLSWLFLVAILKGVXCEIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLWCLVKGFYPSDIAVEWESNGQPENNYKTTPPMLDSDGSFFLYSKLTVDKSRWQQGNVFSCSVMHEALHNHYTQKSLSLSPGK

SEQ ID NO: 249 - protein, 240 aa, Artificial Sequence
  Denosumab CrossMAb Fab light chain
  MISC_FEATURE (6)..(6): X is O
  MISC_FEATURE (110)..(110): X is O
  MISC_FEATURE (130)..(130): X is O
METPAXLLFLLLLWLPDTTGEVQLLESGGGLVQPGGSLRLSCAASGFTFSSYAMSWVRQAPGKGLEWVSGITGSGGSTYYADSVKGRFTISRDNSKNTLYLQMNSLRAEXTAVYYCAKDPGTTVIMSWFXPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 250 - protein, 439 aa, Artificial Sequence; identical to SEQ ID NO: 242
  Nivolumab IgG2 Hole mutation, heavy chain

SEQ ID NO: 251 - protein, 214 aa, Homo sapiens; identical to SEQ ID NO: 90

SEQ ID NO: 252 - protein, 477 aa, Artificial Sequence
  Denosumab CrossMAb CH1-CL huIgG4 KNOB mutation, heavy chain
  MISC_FEATURE (18)..(18): X is O
  MISC_FEATURE (109)..(109): X is O
  MISC_FEATURE (129)..(129): X is O
MEFGLSWLFLVAILKGVXCEVQLLESGGGLVQPGGSLRLSCAASGFTFSSYAMSWVRQAPGKGLEWVSGITGSGGSTYYADSVKGRFTISRDNSKNTLYLQMNSLRAEXTAVYYCAKDPGTTVIMSWFXPWGQGTLVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 253 - protein, 226 aa, Artificial Sequence; identical to SEQ ID NO: 241
  Denosumab CrossMAb CH1-CL light chain
  MISC_FEATURE (6)..(6): X is O

SEQ ID NO: 254 - protein, 440 aa, Artificial Sequence
  Nivolumab IgG4 Hole mutation, heavy chain
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLASRLTVDKSRWQEGNVFSCSVMHEALHNHYTQKSLSLSLGK

SEQ ID NO: 255 - protein, 214 aa, Homo sapiens; identical to SEQ ID NO: 90

SEQ ID NO: 256 - protein, 454 aa, Artificial Sequence
  Denosumab CrossMAb VH-VL huIgG4 KNOB mutation, heavy chain
  MISC_FEATURE (18)..(18): X is O
MEFGLSWLFLVAILKGVXCEIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 257 - protein, 249 aa, Artificial Sequence; identical to SEQ ID NO: 245
  Denosumab CrossMAb VH-VL light chain
  MISC_FEATURE (6)..(6): X is O
  MISC_FEATURE (110)..(110): X is O
  MISC_FEATURE (130)..(130): X is O

SEQ ID NO: 258 - protein, 440 aa, Artificial Sequence; identical to SEQ ID NO: 254
  Nivolumab IgG4 Hole mutation, heavy chain

SEQ ID NO: 259 - protein, 214 aa, Homo sapiens; identical to SEQ ID NO: 90

SEQ ID NO: 260 - protein, 463 aa, Artificial Sequence
  Denosumab CrossMAb Fab huIgG4 KNOB mutation, heavy chain
  MISC_FEATURE (18)..(18): X is O
MEFGLSWLFLVAILKGVXCEIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLWCLVKGFYPSDIAVEWESNGQPENNYKTTPPVLDSDGSFFLYSRLTVDKSRWQEGNVFSCSVMHEALHNHYTQKSLSLSLGK

SEQ ID NO: 261 - protein, 240 aa, Artificial Sequence; identical to SEQ ID NO: 249
  Denosumab CrossMAb Fab light chain
  MISC_FEATURE (6)..(6): X is O
  MISC_FEATURE (110)..(110): X is O
  MISC_FEATURE (130)..(130): X is O

SEQ ID NO: 262 - protein, 440 aa, Artificial Sequence; identical to SEQ ID NO: 254
  Nivolumab IgG4 Hole mutation, heavy chain

SEQ ID NO: 263 - protein, 214 aa, Homo sapiens; identical to SEQ ID NO: 90

SEQ ID NO: 264 - protein, 480 aa, Artificial Sequence
  Denosumab CrossMAb CH1-CL huIgG1 KNOB mutation, heavy chain
  MISC_FEATURE (18)..(18): X is O
  MISC_FEATURE (109)..(109): X is O
  MISC_FEATURE (129)..(129): X is O
MEFGLSWLFLVAILKGVXCEVQLLESGGGLVQPGGSLRLSCAASGFTFSSYAMSWVRQAPGKGLEWVSGITGSGGSTYYADSVKGRFTISRDNSKNTLYLQMNSLRAEXTAVYYCAKDPGTTVIMSWFXPWGQGTLVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 265 - protein, 226 aa, Artificial Sequence; identical to SEQ ID NO: 241
  Denosumab CrossMAb CH1-CL light chain
  MISC_FEATURE (6)..(6): X is O

SEQ ID NO: 266 - protein, 443 aa, Artificial Sequence
  Nivolumab IgG1 Hole mutation, heavy chain
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLASKLTVDKSRWQQGNVFSCSVMHEALHNHYTQKSLSLSPGK

SEQ ID NO: 267 - protein, 214 aa, Homo sapiens; identical to SEQ ID NO: 90

SEQ ID NO: 268 - protein, 457 aa, Artificial Sequence
  Denosumab CrossMAb VH-VL huIgG1 KNOB mutation, heavy chain
  MISC_FEATURE (18)..(18): X is O
MEFGLSWLFLVAILKGVXCEIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 269 - protein, 249 aa, Artificial Sequence; identical to SEQ ID NO: 245
  Denosumab CrossMAb VH-VL light chain
  MISC_FEATURE (6)..(6): X is O
  MISC_FEATURE (110)..(110): X is O
  MISC_FEATURE (130)..(130): X is O

SEQ ID NO: 270 - protein, 443 aa, Artificial Sequence; identical to SEQ ID NO: 266
  Nivolumab IgG1 Hole mutation, heavy chain

SEQ ID NO: 271 - protein, 214 aa, Homo sapiens; identical to SEQ ID NO: 90

SEQ ID NO: 272 - protein, 466 aa, Artificial Sequence
  Denosumab CrossMAb Fab huIgG1 KNOB mutation, heavy chain
  MISC_FEATURE (18)..(18): X is O
MEFGLSWLFLVAILKGVXCEIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 273 - protein, 240 aa, Artificial Sequence; identical to SEQ ID NO: 249
  Denosumab CrossMAb Fab light chain
  MISC_FEATURE (6)..(6): X is O
  MISC_FEATURE (110)..(110): X is O
  MISC_FEATURE (130)..(130): X is O

SEQ ID NO: 274 - protein, 443 aa, Artificial Sequence; identical to SEQ ID NO: 266
  Nivolumab IgG1 Hole mutation, heavy chain

SEQ ID NO: 275 - protein, 214 aa, Homo sapiens; identical to SEQ ID NO: 90

SEQ ID NO: 276 - protein, 655 aa, Artificial Sequence
  RANKL/PD-1 FIT-Ig construct #1
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 277 - protein, 220 aa, Artificial Sequence
  RANKL/PD-1 FIT-Ig construct #2
  MISC_FEATURE (90)..(90): X is O
  MISC_FEATURE (110)..(110): X is O
EVQLLESGGGLVQPGGSLRLSCAASGFTFSSYAMSWVRQAPGKGLEWVSGITGSGGSTYYADSVKGRFTISRDNSKNTLYLQMNSLRAEXTAVYYCAKDPGTTVIMSWFXPWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 278 - protein, 214 aa, Artificial Sequence; identical to SEQ ID NO: 90
  RANKL/PD-1 FIT-Ig construct #3

SEQ ID NO: 279 - protein, 663 aa, Artificial Sequence
  RANKL/CTLA4 FIT-Ig construct #1
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECQVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 280 - protein, 215 aa, Artificial Sequence; identical to SEQ ID NO: 170
  RANKL/CTLA4 FIT-Ig construct #3

SEQ ID NO: 281 - protein, 663 aa, Artificial Sequence
  RANKL/PD-L1 FIT-Ig construct #1
EIVLTQSPGTLSLSPGERATLSCRASQSVRGRYLAWYQQKPGQAPRLLIYGASSRATGIPDRFSGSGSGTDFTLTISRLEPEDFAVFYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECEVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 282 - protein, 214 aa, Artificial Sequence; identical to SEQ ID NO: 150
  RANKL/PD-L1 FIT-Ig construct #3

SEQ ID NO: 283 - protein, 135 aa, Artificial Sequence
  Heavy chain IK22-5
MDVLVLWLCLLTFSSCVLSQVQLKESGPGLVLSSATLSLTCTVSGFSLTNYDVSWIRHLPGKGLEWMGGVWLSGNTEYNSDFKSRLSISRDISKSQVFLKMSNLKIEDTGTYYCARDIGTTSDYWGQGVTVTVSS

SEQ ID NO: 284 - protein, 126 aa, Artificial Sequence
  Light chain IK22-5
MMAPVQLLGLLLLWLPALRCDIQVTQSPSFLSASVGDRVTFNCKTSQNINKYLAWYQAKFGEGPKLLIFNADSLQSGIPPRFSGSGSGTDFTLTISGLQPEDFATYFCLQYNSWPTFGSGTKLEIK

SEQ ID NO: 285 - protein, 138 aa, Artificial Sequence
  Heavy chain RMP1-14
MRMLVLLYLLTALPGILSEVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSS

SEQ ID NO: 286 - protein, 131 aa, Artificial Sequence
  Light chain RMP1-14
MRCSLQFLGLLVLWIPGLNGDIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELK

SEQ ID NO: 287 - protein, 465 aa, Artificial Sequence
  IK22-5-huIgG1Fc WT Heavy chain
MGWSCIILFLVATATGVHSQVQLKESGPGLVLSSATLSLTCTVSGFSLTNYDVSWIRHLPGKGLEWMGGVWLSGNTEYNSDFKSRLSISRDISKSQVFLKMSNLKIEDTGTYYCARDIGTTSDYWGQGVTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVLHEALHNHYTQKSLSLSPGK

SEQ ID NO: 288 - protein, 232 aa, Artificial Sequence
  IK22-5-huIgG1Fc WT Light chain
MGWSCIILFLVATATGVHSDIQVTQSPSFLSASVGDRVTFNCKTSQNINKYLAWYQAKFGEGPKLLIFNADSLQSGIPPRFSGSGSGTDFTLTISGLQPEDFATYFCLQYNSWPTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 289 - protein, 473 aa, Artificial Sequence
  RMP1-14 CH-CL- huIgG1Fc Heavy chain
MGWSCIILFLVATATGVHSEVQLQESGPGLVKPSQSLSLTCSVTGYSITSSYRWNWIRKFPGNRLEWMGYINSAGISNYNPSLKRRISITRDTSKNQFFLQVNSVTTEDAATYYCARSDNMGTTPFTYWGQGTLVTVSSRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVLHEALHNHYTQKSLSLSPGK

SEQ ID NO: 290 - protein, 233 aa, Artificial Sequence
  RMP1-14 CH-CL- huIgG1Fc Light chain
MGWSCIILFLVATATGVHSDIVMTQGTLPNPVPSGESVSITCRSSKSLLYSDGKTYLNWYLQRPGQSPQLLIYWMSTRASGVSDRFSGSGSGTDFTLKISGVEAEDVGIYYCQQGLEFPTFGGGTKLELKASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 291 - protein, 107 aa, Artificial Sequence [sequence omitted]

SEQ ID NO: 292 - protein, 113 aa, Artificial Sequence [sequence omitted]

SEQ ID NO: 293 - protein, 108 aa, Artificial Sequence; identical to SEQ ID NO: 26
  Denosumab VL

SEQ ID NO: 294 - protein, 108 aa, Artificial Sequence; identical to SEQ ID NO: 26
  Denosumab VL

SEQ ID NO: 295 - protein, 122 aa, Artificial Sequence; identical to SEQ ID NO: 25
  Denosumab VH

SEQ ID NO: 296 - protein, 107 aa, Artificial Sequence [sequence omitted]

SEQ ID NO: 297 - protein, 107 aa, Artificial Sequence [sequence omitted]

SEQ ID NO: 298 - protein, 113 aa, Artificial Sequence [sequence omitted]

SEQ ID NO: 299 - protein, 103 aa, Artificial Sequence; identical to SEQ ID NO: 38
  VL of anti-RANKL antibody disclosed in EP 1257648

SEQ ID NO: 300 - protein, 103 aa, Artificial Sequence; identical to SEQ ID NO: 38
  VL of anti-RANKL antibody disclosed in EP 1257648

SEQ ID NO: 301 - protein, 126 aa, Artificial Sequence; identical to SEQ ID NO: 36
  VH of anti-RANKL antibody disclosed in EP 1257648

SEQ ID NO: 302 - protein, 107 aa, Artificial Sequence [sequence omitted]

SEQ ID NO: 303 - protein, 111 aa, Artificial Sequence [sequence omitted]

SEQ ID NO: 304 - protein, 120 aa, Artificial Sequence [sequence omitted]

SEQ ID NO: 305 - protein, 108 aa, Artificial Sequence; identical to SEQ ID NO: 26
  Denosumab VL

SEQ ID NO: 306 - protein, 108 aa, Artificial Sequence; identical to SEQ ID NO: 26
  Denosumab VL

SEQ ID NO: 307 - protein, 122 aa, Artificial Sequence; identical to SEQ ID NO: 25
  Denosumab VH

SEQ ID NO: 308 - protein, 111 aa, Artificial Sequence [sequence omitted]

SEQ ID NO: 309 - protein, 111 aa, Artificial Sequence [sequence omitted]

SEQ ID NO: 310 - protein, 120 aa, Artificial Sequence [sequence omitted]

SEQ ID NO: 311 - protein, 103 aa, Artificial Sequence; identical to SEQ ID NO: 38
  VL of anti-RANKL antibody disclosed in EP 1257648

SEQ ID NO: 312 - protein, 103 aa, Artificial Sequence; identical to SEQ ID NO: 38
  VL of anti-RANKL antibody disclosed in EP 1257648

SEQ ID NO: 313 - protein, 126 aa, Artificial Sequence; identical to SEQ ID NO: 36
  VH of anti-RANKL antibody disclosed in EP 1257648

SEQ ID NO: 314 - protein, 111 aa, Artificial Sequence [sequence omitted]

SEQ ID NO: 315 - protein, 108 aa, Artificial Sequence; identical to SEQ ID NO: 141
  Durvalumab VL

SEQ ID NO: 316 - protein, 120 aa, Artificial Sequence; identical to SEQ ID NO: 139
  Durvalumab VH

SEQ ID NO: 317 - protein, 108 aa, Artificial Sequence; identical to SEQ ID NO: 26
  Denosumab VL

SEQ ID NO: 318 - protein, 108 aa, Artificial Sequence; identical to SEQ ID NO: 26
  Denosumab VL

SEQ ID NO: 319 - protein, 122 aa, Artificial Sequence; identical to SEQ ID NO: 25
  Denosumab VH

SEQ ID NO: 320 - protein, 108 aa, Artificial Sequence; identical to SEQ ID NO: 141
  Durvalumab VL

SEQ ID NO: 321 - protein, 108 aa, Artificial Sequence; identical to SEQ ID NO: 141
  Durvalumab VL

SEQ ID NO: 322 - protein, 120 aa, Artificial Sequence; identical to SEQ ID NO: 139
  Durvalumab VH

SEQ ID NO: 323 - protein, 103 aa, Artificial Sequence; identical to SEQ ID NO: 38
  VL of anti-RANKL antibody disclosed in EP 1257648

SEQ ID NO: 324 - protein, 103 aa, Artificial Sequence; identical to SEQ ID NO: 38
  VL of anti-RANKL antibody disclosed in EP 1257648

SEQ ID NO: 325 - protein, 126 aa, Artificial Sequence; identical to SEQ ID NO: 36
  VH of anti-RANKL antibody disclosed in EP 1257648

SEQ ID NO: 326 - protein, 108 aa, Artificial Sequence; identical to SEQ ID NO: 141
  Durvalumab VL

SEQ ID NO: 327 - protein, 107 aa, Artificial Sequence [sequence omitted]

SEQ ID NO: 328 - protein, 118 aa, Artificial Sequence [sequence omitted]

SEQ ID NO: 329 - protein, 108 aa, Artificial Sequence; identical to SEQ ID NO: 26
  Denosumab VL

SEQ ID NO: 330 - protein, 108 aa, Artificial Sequence; identical to SEQ ID NO: 26
  Denosumab VL

SEQ ID NO: 331 - protein, 122 aa, Artificial Sequence; identical to SEQ ID NO: 25
  Denosumab VH

SEQ ID NO: 332 - protein, 107 aa, Artificial Sequence [sequence omitted]

SEQ ID NO: 333 - protein, 107 aa, Artificial Sequence [sequence omitted]

SEQ ID NO: 334 - protein, 118 aa, Artificial Sequence [sequence omitted]

SEQ ID NO: 335 - protein, 103 aa, Artificial Sequence; identical to SEQ ID NO: 38
  VL of anti-RANKL antibody disclosed in EP 1257648

SEQ ID NO: 336 - protein, 103 aa, Artificial Sequence; identical to SEQ ID NO: 38
  VL of anti-RANKL antibody disclosed in EP 1257648

SEQ ID NO: 337 - protein, 126 aa, Artificial Sequence; identical to SEQ ID NO: 36
  VH of anti-RANKL antibody disclosed in EP 1257648

SEQ ID NO: 338 - protein, 107 aa, Artificial Sequence [sequence omitted]

SEQ ID NO: 339 - protein, 108 aa, Artificial Sequence; identical to SEQ ID NO: 171
  Ipilimumab VL

SEQ ID NO: 340 - protein, 118 aa, Artificial Sequence; identical to SEQ ID NO: 169
  Ipilimumab VH

SEQ ID NO: 341 - protein, 108 aa, Artificial Sequence; identical to SEQ ID NO: 26
  Denosumab VL

SEQ ID NO: 342 - protein, 108 aa, Artificial Sequence; identical to SEQ ID NO: 26
  Denosumab VL

SEQ ID NO: 343 - protein, 122 aa, Artificial Sequence; identical to SEQ ID NO: 25
  Denosumab VH

SEQ ID NO: 344 - protein, 108 aa, Artificial Sequence; identical to SEQ ID NO: 171
  Ipilimumab VL

SEQ ID NO: 345 - protein, 108 aa, Artificial Sequence; identical to SEQ ID NO: 171
  Ipilimumab VL

SEQ ID NO: 346 - protein, 118 aa, Artificial Sequence; identical to SEQ ID NO: 169
  Ipilimumab VH

SEQ ID NO: 347 - protein, 103 aa, Artificial Sequence; identical to SEQ ID NO: 38
  VL of anti-RANKL antibody disclosed in EP 1257648

SEQ ID NO: 348 - protein, 103 aa, Artificial Sequence; identical to SEQ ID NO: 38
  VL of anti-RANKL antibody disclosed in EP 1257648

SEQ ID NO: 349 - protein, 126 aa, Artificial Sequence; identical to SEQ ID NO: 36
  VH of anti-RANKL antibody disclosed in EP 1257648

SEQ ID NO: 350 - protein, 108 aa, Artificial Sequence; identical to SEQ ID NO: 171
  Ipilimumab VL

SEQ ID NO: 351 - protein, 107 aa, Artificial Sequence; identical to SEQ ID NO: 181
  Tremelimumab VL

SEQ ID NO: 352 - protein, 118 aa, Artificial Sequence; identical to SEQ ID NO: 169
  Tremelimumab VH

SEQ ID NO: 353 - protein, 108 aa, Artificial Sequence; identical to SEQ ID NO: 26
  Denosumab VL

SEQ ID NO: 354 - protein, 108 aa, Artificial Sequence; identical to SEQ ID NO: 26
  Denosumab VL

SEQ ID NO: 355 - protein, 122 aa, Artificial Sequence; identical to SEQ ID NO: 25
  Denosumab VH

SEQ ID NO: 356 - protein, 107 aa, Artificial Sequence; identical to SEQ ID NO: 181
  Tremelimumab VL

SEQ ID NO: 357 - protein, 107 aa, Artificial Sequence; identical to SEQ ID NO: 181
  Tremelimumab VL

SEQ ID NO: 358 - protein, 118 aa, Artificial Sequence; identical to SEQ ID NO: 169
  Tremelimumab VH

SEQ ID NO: 359 - protein, 103 aa, Artificial Sequence; identical to SEQ ID NO: 38
  VL of anti-RANKL antibody disclosed in EP 1257648

SEQ ID NO: 360 - protein, 103 aa, Artificial Sequence; identical to SEQ ID NO: 38
  VL of anti-RANKL antibody disclosed in EP 1257648

SEQ ID NO: 361 - protein, 126 aa, Artificial Sequence; identical to SEQ ID NO: 36
  VH of anti-RANKL antibody disclosed in EP 1257648

SEQ ID NO: 362 - protein, 107 aa, Artificial Sequence; identical to SEQ ID NO: 181
  Tremelimumab VL